(19) United States
(12) Patent Application Publication
(10) Pub. No.: US 2011/0166132 A1
(43) Pub. Date: Jul. 7, 2011

(54) Gamma Secretase Modulators

(75) Inventors: Stephen A. Hitchcock, Jupiter, FL (US); Jian J. Chen, Camarillo, CA (US); Mqhele Vellah Ncube, Simi Valley, CA (US); Thomas Nixey, Newbury Park, CA (US); Albert Amegadzie, Moorpark, CA (US); Roxanne Kunz, Santa Monica, CA (US); Wenyan Qian, Camaillor, CA (US); Ning Chen, Thousand Oaks, CA (US); Christopher M. Tegley, Thousand Oaks, CA (US); Frenel F. De Morin, Thousand Oaks, CA (US); Chester Chenguang Yuan, Newbury Park, CA (US); Qingyian Liu, Camarillo, CA (US); Jiawang Zhu, Thousand Oaks, CA (US); Tanya A.N. Peterkin, St. Andrew (JM); Jeffrey A. Adams, Thousand Oaks, CA (US); Essa Hu, Camarillo, CA (US); Frank Chavez, JR., Camarillo, CA (US)

(73) Assignee: Amgen Inc., Thousand Oaks, CA (US)

(21) Appl. No.: 12/746,902

(22) PCT Filed: Dec. 12, 2008
(86) PCT No.: PCT/US08/13639

§ 371 Date: Feb. 23, 2011

Related U.S. Application Data
(60) Provisional application No. 61/007,486, filed on Dec. 13, 2007.

(51) Int. Cl. A61K 31/538; C07D 213/56; C07D 413/12; C07D 401/12; C07D 401/14; C07D 413/14; C07D 405/12; C07D 233/61; C07D 403/12; A61K 31/4418; A61K 31/4439; A61K 31/496; A61K 31/444; A61K 31/4725; A61K 31/4545; A61K 31/5377; A61K 31/4433; A61K 31/4709; A61K 31/4164; A61K 31/4178; A61P 25/28

(52) U.S. Cl. 514/230.5; 546/337; 544/105; 546/275.4; 544/364; 546/265; 546/146; 546/194; 544/131; 546/282.7; 546/277.4; 546/153; 548/343.5; 548/312.7; 514/357; 514/341; 514/253.01; 514/332; 514/307; 514/318; 514/235.5; 514/337; 514/339; 514/312; 514/399; 514/397

(57) ABSTRACT

The present invention provides compounds that are gamma secretase modulators and are therefore useful for the treatment of diseases treatable by modulation of gamma secretase such as Alzheimer's disease. Also provided are pharmaceutical compositions containing such compounds and processes for preparing such compounds.

### FIELD OF THE INVENTION

The present invention provides compounds that are gamma secretase modulators and are therefore useful for the treatment of diseases treatable by modulation of gamma secretase such as Alzheimer's disease. Also provided are pharmaceutical compositions containing such compounds and processes for preparing such compounds.

### BACKGROUND

Alzheimer's disease (AD) is the most common cause of dementia, resulting in loss of memory, cognition, reasoning, judgement, and orientation. AD is characterized by the presence of extracellular amyloid plaques, intracellular neurofibrillary tangles, in addition to loss of synapses and neurons in the brain. The main constituent of amyloid plaques is β-amyloid (Aβ) a 4 kDa peptide.

Accumulation of Aβ is thought to be an early and critical step in the pathogenesis of Alzheimer's Disease (AD). Aβ elicits a cascade of toxic and inflammatory events that ultimately lead to neuronal death and cognitive impairment. The Aβ peptide results from proteolysis of Amyloid Precursor Protein (APP). The APP protein is a transmembrane protein consisting of a large extracellular domain and a short cytoplasmic tail. Aβ sequence encompasses parts of the extracellular and transmembrane domains of APP.

APP can be processed via either of two routes, a non-amyloidogenic and an amyloidogenic pathway. Most of the APP is processed through the non-amyloidogenic pathway, whereby the protease α-secretase cleaves APP within the Aβ domain to release a large soluble N-terminal fragment (sAPPα) and a non-amyloidogenic C-terminal fragment (C83). This fragment is further processed by γ-secretase to produce a 22-24 residue peptide (p3).

In the amyloidogenic pathway, APP is cleaved by β-secretase (BACE1), generating a shorter N-terminal domain (sAPPβ) and an amyloidogenic C-terminal (C99). This C99 fragment is subsequently cleaved by γ-secretase. The γ-secretase is a protease formed by a complex of proteins: Presenilin-1 (PS-1), Nicastrin, PEN-2, and APH-1. Proteolysis of APP intermediates by γ-secretase yields Aβ peptides of varying length (Aβ37, Aβ38, Aβ39, Aβ40, Aβ42). Of these peptides, Aβ42 is the least soluble, most aggregating species and the principal component of toxic oligomers and amyloid plaques in AD brain. All known mutations causing early onset Familial AD either increase total Aβ formation or increase the ratio of Aβ42 to Aβ40. Therefore agents that can block the formation of Aβ42 should be useful for the treatment of AD.

One proposed treatment involves modulation of γ-secretase activity to selectively reduce the production of Aβ42 while increase the production of the shorter chain isoforms (such as Aβ37, 38, and 39). These isoforms are believed to be less prones to self-aggregate and are more easily cleared from the brain and or less toxic. Several classes of compounds are proposed as γ-secretase modulators (referred hereon as GSM), see, Imbimbo B. P, et al., J Pharmacol Exp Ther. 2007 December; 323 (3):822-30; WO2007054739; WO2006043064; and WO2007124351.

The present invention provides a new class of compounds that selectively reduce the production of Aβ42 peptide by modulation of γ-secretase and hence are useful in the treatment of Alzheimer's disease.

### SUMMARY

In one aspect, provided herein are compounds of Formula (I):

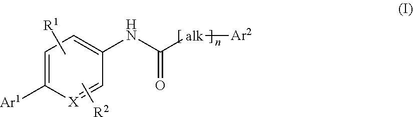

where:

n is 0 or 1;

alk is a straight or branched alkylene chain of 1 to 6 carbon atoms optionally substituted with cycloalkyl or one to three fluoro atoms;

X is —CH— or —N—;

R¹ and R² are independently hydrogen, alkyl, alkoxy, hydroxy, cyano, or halo;

Ar¹ is a ring selected from (i), (ii), (iii), or (iv);

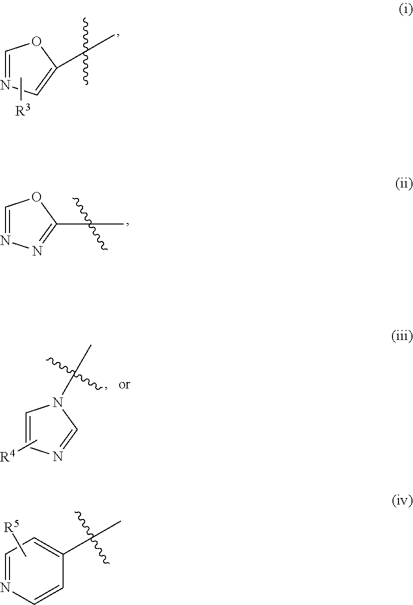

where:

R³ and R⁴ are hydrogen or alkyl;

R⁵ is alkyl; and

Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, hydroxyl, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, optionally substituted phenyl, optionally substituted heteroaryl, or optionally substituted heterocyclyl; or

a pharmaceutically acceptable salt thereof provided that (i) R^{b} or R^{c} is not substituted or unsubstituted hydantoin ring; (ii) R⁵ is not bonded to the nitrogen atom of the pyridinyl ring, (iii) when X is —CH—, R¹ and R² are hydrogen, n is 0, then Ar² is not 3,5-dichlorophenyl, 3-chlorophenyl, 1-phenylcycloprop-1-yl, 2,6-dichloropyridin-4-yl, 2,3-dihydro-1H-inden-2-yl or 1,2,3,4-tetrahydronaphth-2-yl; and (iv) when X is —CH—, R¹ and R² are hydrogen, n is 1, alk is —CHCH₃ or —CH(CH₂)CH₃, then Ar² is not 6-chloropyridin-2-yl or 3-methylphenyl.

In a second aspect, provided herein are compounds of Formula (I′):

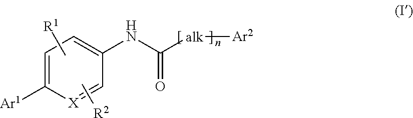

where:

n is 0 or 1;

alk is a straight or branched alkylene chain of 1 to 6 carbon atoms optionally substituted with one to three fluoro atoms;

X is —CH— or —N—;

R¹ and R² are independently hydrogen, alkyl, alkoxy, hydroxy, or halo;

Ar¹ is a ring selected from (i), (ii), (iii), or (iv);

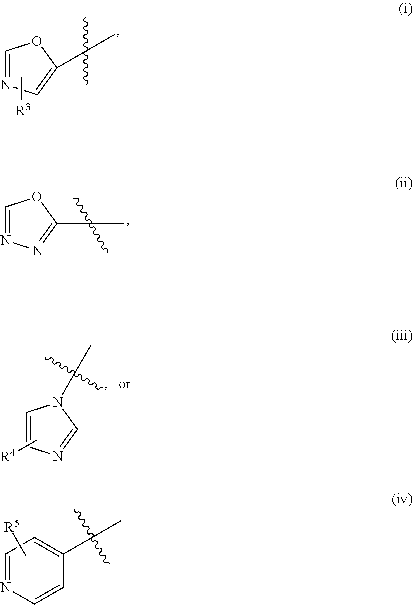

where:

R³ and R⁴ are hydrogen or alkyl;

R⁵ is alkyl; and

Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or

a pharmaceutically acceptable salt thereof provided that (i) R^{b} or R^{c} is not substituted or unsubstituted hydantoin ring; (ii) R⁵ is not bonded to the nitrogen atom of the pyridinyl ring, (iii) when X is —CH—, R¹ and R² are hydrogen, n is 0, then Ar² is not 3,5-dichlorophenyl, 3-chlorophenyl, 1-phenylcycloprop-1-yl, 2,6-dichloropyridin-4-yl, 2,3-dihydro-1H-inden-2-yl or 1,2,3,4-tetrahydronaphth-2-yl; and (iv) when X is —CH—, R¹ and R² are hydrogen, n is 1, alk is —CHCH₃ or —CH(CH₂)CH₃, then Ar² is not 6-chloropyridin-2-yl or 3-methylphenyl.

Compounds of Formula (I′) are a subset of the compounds of Formula (I).

In a third aspect, provided is a pharmaceutical composition comprising a compound of Formula (I), a pharmaceutically acceptable salt thereof or a mixture a compound of Formula (I) and a pharmaceutically acceptable salt thereof; and a pharmaceutically acceptable excipient.

In a fourth aspect, this invention is directed to a method of treating Alzheimer's disease by modulation of γ-secretase in a patient which method comprises administering to the patient a pharmaceutical composition comprising a compound of Formula (I) a pharmaceutically acceptable salt thereof, or a mixture a compound of Formula (I) and a pharmaceutically acceptable salt thereof and a pharmaceutically acceptable excipient.

In a fifth aspect, this invention provides compounds of Formula (I) for use as a medicament.

In a sixth aspect, this invention provides compounds of Formula (I) for use in modulating γ-secretase.

In a sixth aspect, this invention provides compounds of Formula (I) for preparing a medicament for treating Alzheimer's disease by modulation of γ-secretase.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring e.g., cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means a —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” or “alkoxyalkoxy” means a —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, each as defined above, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means a —OR radical where R is aminoalkyl as defined above, e.g., 2-aminoethoxy, 2-dimethylaminopropoxy, and the like.

“Aminocarbonyl” means a —CONRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —CONH₂, methylaminocarbonyl, 2-dimethylaminocarbonyl, and the like.

“Aminosulfonyl” means a —SO₂NRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —SO₂NH₂, methylaminosulfonyl, 2-dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetyl, propionyl, benzoyl, pyridinylcarbonyl, and the like. When R is alkyl, the radical is also referred to herein as alkylcarbonyl.

“Acylamino” means a —NHCOR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetylamino, propionylamino, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above.

“Aryloxy” means a —OR radical where R is aryl as defined above, e.g., phenoxy, naphthyloxy.

“Aralkyloxy” means a —OR radical where R is aralkyl as defined above, e.g., benzyloxy, and the like.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkylalkyl” means a -(alkylene)-R radical where R is cycloalkyl as defined above; e.g., cyclopropylmethyl, cyclobutylmethyl, cyclopentylethyl, or cyclohexylmethyl, and the like.

“Cycloalkoxy” means a —OR radical where R is cycloalkyl as defined above, e.g., cyclopropoxy, cyclobutoxy, and the like.

“Carboxy” means —COOH.

“Disubstituted amino” means a —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., dimethylamino, phenylmethylamino, and the like. When R and R′ are alky, it is also referred to herein as dialkylamino.

“Halo” means fluoro, chloro, bromo, or iodo, preferably fluoro or chloro.

“Fused cycloalkyl” means cycloalkyl ring as defined above that is fused to one or two aryl or heteroaryl ring as defined herein e.g., tetrahydronaphthyl, 1,2,3,4-tetrahydroquinolinyl, and the like.

“Fused heterocyclyl” means heterocyclyl ring as defined above that is fused to one or two monocyclic aryl or heteroaryl ring or one bicyclic aryl or heteroaryl ring as defined herein provided the fused heterocyclyl ring is attached to the —NHCO— group via a carbon atom e.g., carbazolyl, and the like.

“Haloalkyl” means alkyl radical as defined above, which is substituted with one or more halogen atoms, preferably one to five halogen atoms, preferably fluorine or chlorine, including those substituted with different halogens, e.g., —CH₂Cl, —CF₃, —CHF₂, —CH₂CF₃, —CF₂CF₃, —CF(CH₃)₃, and the like. When the alkyl is substituted with only fluoro, it is referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means a —OR radical where R is hydroxyalkyl as defined above.

“Heterocyclyl” means a saturated or unsaturated monovalent monocyclic group of 5 to 8 ring atoms in which one or two ring atoms are heteroatom selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring and is a subset of fused heterocyclyl. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Heterocyclylalkyl” means a -(alkylene)-R radical where R is heterocyclyl ring as defined above e.g., tetraydrofuranylmethyl, piperazinylmethyl, morpholinylethyl, and the like.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Heteroaralkyl” means a -(alkylene)-R radical where R is heteroaryl as defined above.

“Heteraryloxy” means a —OR radical where R is heteroaryl as defined above, e.g., pyridinyloxy, thiophenyloxy, and the like.

“Monosubstituted amino” means a —NHR radical where R is alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., methylamino, 2-phenylamino, hydroxyethylamino, and the like.

“Modulation of γ-secretase activity” as used herein means the compounds reduce the production of Aβ42 produced by γ-secretase in the presence of the compounds of the Invention.

The present invention also includes the prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

“Optional substituted phenyl” means phenyl ring that is optionally substituted with one, two, or three substitutents independently selected from alkyl, halo, haloalkyl, haloalkoxy, alkoxy, amino, alkylamino, cyano, or dialkylamino.

“Optional substituted heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon, that is optionally substituted with one, two, or three substitutents independently selected from alkyl, halo, haloalkyl, haloalkoxy, alkoxy, amino, alkylamino, cyano, or dialkylamino.

“Optional substituted heterocyclyl” means heterocyclyl as defined above, that is optionally substituted with one, two, or three substitutents independently selected from alkyl, halo, haloalkyl, haloalkoxy, alkoxy, alkoxycarbonyl, amino, alkylamino, cyano, or dialkylamino.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfonyl” means a —SO₂R radical where R is alkyl, haloalkyl, aryl, aralkyl, heteroaryl, heteroaralkyl, heterocyclyl, heterocyclylalkyl, each as defined herein, e.g., methylsulfonyl, phenylsulfonyl, benzylsulfonyl, pyridinylsulfonyl, and the like.

“Spirocycloalkyl” means a bicyclic compound ring of 6 to 12 carbon ring atoms where the rings are connected through one carbon atom. Representative examples include, but are not limited to,

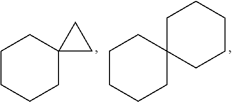

and the like.

The phrase in the definition of groups Ar¹ and Ar² in the claims and in the specification of this Application “ . . . wherein the aforementioned rings are optionally substituted with R^{a}, R^{b}, or R^{c} independently selected from . . . ” and similar phrases used for others groups in the claims and in the specification with respect to the compound of Formula (I) means that the rings can be mono-, di-, or trisubstituted unless indicated otherwise.

“Treating” or “treatment” of a disease includes:

preventing the disease, i.e. causing the clinical symptoms of the disease not to develop in a mammal that may be exposed to or predisposed to the disease but does not yet experience or display symptoms of the disease;

inhibiting the disease, i.e., arresting or reducing the development of the disease or its clinical symptoms; or

relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

TABLE 1
shows representative compounds of Formula (I) are shown below.

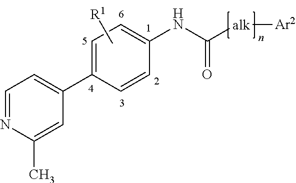

| | | | | | | Mass |
|---|---|---|---|---|---|---|
| Cpd | | | | | | Spec. |
| # | n | R¹ | Alk | Ar² | Name | data |
| 1 | 1 | H | (R)-CHCH₃ | 3,5-diCF₃phenyl | (2R)-2-(3,5-bis(trifluoromethyl)phenyl)-N- | 453.1 |
| | | | | | (4-(2-methyl-4-pyridinyl)phenyl)- | |
| | | | | | propanamide | |
| 2 | 0 | H | | 1-(4-F-phenyl)cyclopent-1-yl | 1-(4-fluorophenyl)-N-(4-(2-methyl-4- | 375 |
| | | | | | pyridinyl)phenyl)cyclopentanecarboxamide | |
| 3 | 1 | H | CH₂ | 4-trifluoromethylphenyl | N-(4-(2-methyl-4-pyridinyl)phenyl)-2-(4- | 371 |
| | | | | | (trifluoromethyl)phenyl)acetamide | |
| 4 | 1 | H | CH₂ | 3,4-dichlorophenyl | 2-(3,4-dichlorophenyl)-N-(4-(2-methyl-4- | 371 |
| | | | | | pyridinyl)phenyl)acetamide | |
| 5 | 1 | H | CHCH₃ | 3,5-diCF₃phenyl | 2-(3,5-bis(trifluoromethyl)phenyl)-N-(4-(2- | 453.3 |
| | | | | | methyl-4-pyridinyl)phenyl)propanamide | |
| 6 | 1 | H | CH₂ | 2,4-dichlorophenyl | 2-(2,4-dichlorophenyl)-N-(4-(2-methyl-4- | 371 |
| | | | | | pyridinyl)phenyl)acetamide | |
| 7 | 1 | H | CH₂ | 2-trifluoromethylphenyl | N-(4-(2-methyl-4-pyridinyl)phenyl)-2-(2- | 371 |
| | | | | | (trifluoromethyl)phenyl)acetarnid | |
| 8 | 1 | H | CH₂ | 3-chlorophenyl | 2-(3-chlorophenyl)-N-(4-(2-methyl-4- | 337 |
| | | | | | pyridinyl)phenyl)acetamide | |
| 10 | 0 | H | | 5-chloro-2-methoxyphenyl | 5-chloro-2-methoxy-N-(4-(2-methyl-4- | 353 |
| | | | | | pyridinyl)phenyl)benzamide | |
| 11 | 1 | H | CH₂ | 3-trifluoromethylphenyl | N-(4-(2-methyl-4-pyridinyl)phenyl)-2-(3- | 371 |
| | | | | | (trifluoromethyl)phenyl)acetamide | |
| 12 | 1 | H | CHCH₃ | 3-chlorophenyl | 2-(3-chlorophenyl)-N-(4-(2-methyl-4- | 351.1 |
| | | | | | pyridinyl)phenyl)propanamide | |
| 13 | 1 | H | CH₂ | 3,5-diCF₃phenyl | 2-(3,5-ditrifluoromethylphenyl)-N-(4-(2- | |
| | | | | | methyl-4-pyridinyl)phenypacetamide | |
| 14 | 1 | H | CH₂ | 2,6-dichlorophenyl | 2-(2,6-dichlorophenyl)-N-(4-(2-methyl-4- | 371 |
| | | | | | pyridinyl)phenyl)acetamide | |
| 15 | 1 | H | CH₂ | naphth-1-yl | N-(4-(2-methyl-4-pyridinyl)phenyl)-2-(1- | 353.2 |
| | | | | | naphthalenyl)acetamide | |
| 16 | 1 | H | CH₂ | 3,4-methylenedioxyphenyl | 2-(1,3-benzodioxol-5-yl)-N-(4-(2-methyl-4- | 347 |
| | | | | | pyridinyl)phenyl)acetamide | |
| 17 | 1 | H | CHCH₃ | 4-chlorophenyl | 2-(4-chlorophenyl)-N-(4-(2-methyl-4- | 351 |
| | | | | | pyridinyl)phenyl)propanamide | |
| 19 | 1 | H | CH₂ | biphen-4-yl | 2-(4-biphenylyl)-N-(4-(2-methyl-4- | 379 |
| | | | | | pyridinyl)phenyl)acetamide | |
| 20 | 0 | H | | 5-methylH-imidazo[1,2- | 5-methyl-N-(4-(2-methyl-4-pyridinyl)- | 343 |
| | | | | a]pyridin2-yl | phenyl)imidazo[1,2-a]pyridine-2- | |
| | | | | | carboxamide | |
| 21 | 1 | H | (S)-CHCH₃ | phenyl | (2S)-N-(4-(2-methyl-4-pyridinyl)phenyl)-2- | 317.1 |
| | | | | | phenylpropanamide | |
| 22 | 0 | H | | 5-cyclopropylisoxazol-3-yl | 5-cyclopropyl-N-(4-(2-methyl-4-pyridinyl)- | 320 |
| | | | | | phenyl)-3-isoxazolecarboxamide | |
| 24 | 1 | H | CHCH₃ | 6-chloropyridin-2-yl | 2-(6-chloro-2-pyridinyl)-N-(4-(2-methyl-4- | 352.1 |
| | | | | | pyridinyl)phenyl)propanamide | |
| 25 | 1 | H | CH₂ | 5-chlorothiophen-2-yl | 2-(5-chloro-2-thiophenyl)-N-(4-(2-methyl- | 343 |
| | | | | | 4-pyridinyl)phenyl)acetamide | |
| 26 | 1 | H | CHCH₃ | 3,5-difluorophenyl | 2-(3,5-difluorophenyl)-N-(4-(2-methyl-4- | 353.1 |
| | | | | | pyridinyl)phenyl)propanamide | |
| 27 | 1 | H | CH₂ | 5-bromopyridin-3-yl | 2-(5-bromo-3-pyridinyl)-N-(4-(2-methyl-4- | 382 |
| | | | | | pyridinyl)phenyl)acetamide | |
| 28 | 1 | H | CHCH₃ | phenyl | N-(4-(2-methyl-4-pyridinyl)phenyl)-2- | 317 |
| | | | | | phenylpropanamide | |
| 29 | 1 | H | CH₂ | 4-methoxyphenyl | 2-(4-methoxyphenyl)-N-(4-(2-methyl-4- | 333 |
| | | | | | pyridinyl)phenyl)acetamide | |
| 30 | 1 | H | CH₂ | phenyl | N-(4-(2-methyl-4-pyridinyl)phenyl)-2- | 303 |
| | | | | | phenylacetamide | |
| 31 | 0 | H | | 3-phenoxyphenyl | N-(4-(2-methyl-4-pyridinyl)phenyl)-3- | 381 |
| | | | | | phenoxybenzamid | |
| 32 | 1 | H | CH₂ | naphth-2-yl | N-(4-(2-methyl-4-pyridinyl)phenyl)-2-(2- | 353 |
| | | | | | naphthalenypacetamide | |
| 33 | 1 | H | CH₂ | 3-methoxyphenyl | 2-(3-methoxyphenyl)-N-(4-(2-methyl-4- | 333 |
| | | | | | pyridinyl)phenyl)acetamid | |
| 34 | 0 | H | | 3-methoxyphenyl | 3-methoxy-N-(4-(2-methyl-4-pyridinyl)- | 319 |
| | | | | | phenyl)benzamide | |
| 35 | 0 | H | | 3-methoxycyclohexyl | 3-methoxy-N-(4-(2-methyl-4-pyridinyl)- | 325 |
| | | | | | phenyl)cyclohexanecarboxamide | |
| 36 | 0 | H | | 2-chloro-5-bromophenyl | 5-bromo-2-chloro-N-(4-(2-methyl-4- | 402.9 |
| | | | | | pyridinyl)phenyl)benzamide | |
| 37 | 0 | H | | 3-chloro-4-methoxyphenyl | 3-chloro-4-methoxy-N-(4-(2-methyl-4- | 353 |
| | | | | | pyridinyl)phenyl)benzamide | |
| 38 | 1 | H | CH₂ | 5-methylthiophen-2-yl | N-(4-(2-methyl-4-pyridinyl)phenyl)-2-(5- | 333 |
| | | | | | methyl-2-thiophenyl)acetamid | |
| 39 | 1 | H | CHCH(CH₃)₂ | phenyl | 3-methyl-N-(4-(2-methyl-4-pyridinyl)- | 345 |
| | | | | | phenyl)-2-phenylbutanamide | |
| 40 | 1 | H | (R)-CHCH₃ | phenyl | (2R)-N-(4-(2-methyl-4-pyridinyl)phenyl)-2- | 317.1 |
| | | | | | phenylpropanamide | |
| 41 | 1 | H | CH₂ | 2-chlorophenyl | 2-(2-chlorophenyl)-N-(4-(2-methyl-4- | 337 |
| | | | | | pyridinyl)phenyl)acetamide | |
| 42 | 0 | H | | 3,4-ethylenedioxyphenyl | N-(4-(2-methyl-4-pyridinyl)phenyl)-2,3- | 347 |
| | | | | | dihydro-1,4-benzodioxine-6-carboxamide | |
| 43 | 0 | H | | 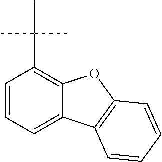 | N-(4-(2-methyl-4-pyridinyl)phenyl)- dibenzo[b,d]furan-4-carboxamide | 379 |
| 44 | 0 | H | | 3,5-dimethylphenyl | 3,5-dimethyl-N-(4-(2-methyl-4-pyridinyl)- | 317 |
| | | | | | phenyl)benzamide | |
| 45 | 0 | H | | 2,3-dichlorophenyl | 2,3-dichloro-N-(4-(2-methyl-4-pyridinyl)- | 357 |
| | | | | | phenyl)benzamide | |
| 46 | 0 | H | | 3-dimethylaminophenyl | 3-(dimethylamino)-N-(4-(2-methyl-4- | 332 |
| | | | | | pyridinyl)phenyl)benzamide | |
| 47 | 0 | H | | 3-cyanophenyl | 3-cyano-N-(4-(2-methyl-4-pyridinyl)- | 314 |
| | | | | | phenyl)benzamide | |
| 48 | 1 | H | C(CH₃)₂ | 4-chlorophenyl | 2-(4-chlorophenyl)-2-methyl-N-(4-(2- | 365.1 |
| | | | | | methyl-4-pyridinyl)phenyl)propanamide | |
| 49 | 1 | H | (R)-CH(CH₃) | 3,5-diCF₃phenyl | (2S)-2-(3,5-bis(trifluoromethyl)phenyl)-N- | 453.1 |
| | | | | | (4-(2-methyl-4-pyridinyl)phenyl)- | |
| | | | | | propanamide | |
| 50 | 0 | H | | 3-chloro-4,5-dimethoxy- | 3-chloro-4,5-dimethoxy-N-(4-(2-methyl-4- | 383 |
| | | | | phenyl | pyridinyl)phenyl)benzamide | |
| 51 | 0 | H | | 2,6-dichloropyridin-4-yl | 2,6-dichloro-N-(4-(2-methyl-4-pyridinyl)- | 358 |
| | | | | | phenyl)-4-pyridinecarboxamide | |
| 52 | 0 | H | | tetrahydrofuran-2-yl | N-(4-(2-methyl-4-pyridinyl)phenyl)- | 283 |
| | | | | | tetrahydro-2-furancarboxamide | |
| 53 | 0 | H | | 1-phenyl-5-oxopyrrolidin-3-yl | N-(4-(2-methyl-4-pyridinyl)phenyl)-5-oxo- | 372 |
| | | | | | 1-phenyl-3-pyrrolidinecarboxamide | |
| 54 | 0 | H | | cyclopropyl | N-(4-(2-methyl-4-pyridinyl)phenyl)- | 253 |
| | | | | | cyclopropanecarboxamide | |
| 55 | 0 | H | | 1-acetylpiperidin-4-yl | 1-acetyl-N-(4-(2-methyl-4-pyridinyl)- | 338 |
| | | | | | phenyl)-4-piperidinecarboxamide | |
| 56 | 1 | H | CH₂ | cyclopropyl | 2-cyclopropyl-N-(4-(2-methyl-4-pyridinyl)- | 267 |
| | | | | | phenypacetamide | |
| 57 | 1 | H | (CH₂)₂ | pyrazol-1-yl | N-(4-(2-methyl-4-pyridinyl)phenyl)-3-(1H- | 307 |
| | | | | | pyrazol-1-yl)propanamide | |
| 58 | 1 | H | (CH₂)₂ | 3,5-dimethylpyrazol-1-yl | 3-(3,5-dimethyl-1H-pyrazol-1-yl)-N-(4-(2- | 335 |
| | | | | | methyl-4-pyridinyl)phenyl)propanamide | |
| 59 | 1 | H | (CH₂)₂ | 3,5-dimethylisoxazol-4-yl | 3-(3,5-dimethyl-4-isoxazolyl)-N-(4-(2- | 336 |
| | | | | | methyl-4-pyridinyl)phenyl)propanamide | |
| 60 | 0 | H | | 1-cyclohexyl-5-oxopyrrolidin- | 1-cyclohexyl-N-(4-(2-methyl-4-pyridinyl)- | 378 |
| | | | | 3-yl | phenyl)-5-oxo-3-pyrrolidinecarboxamide | |
| 61 | 0 | H | | 1-tert-butyl-5-oxopyrrolidin-3- | 1-tert-butyl-N-(4-(2-methyl-4-pyridinyl)- | 352 |
| | | | | yl | phenyl)-5-oxo-3-pyrrolidinecarboxamide | |
| 62 | 0 | H | | 1-benzyl-5-oxopyrrolidin-3-yl | 1-benzyl-N-(4-(2-methyl-4-pyridinyl)- | 386 |
| | | | | | phenyl)-5-oxo-3-pyrrolidinecarboxamid | |
| 64 | 1 | H | CH[CH₂CH(CH₃)₂] | 3-methylphenyl | 4-methyl-2-(3-methylphenyl)-N-(4-(2- | 373.2 |
| | | | | | methyl-4-pyridinyl)phenyl)pentanamide | |
| 65 | 1 | H | CH(CH₃) | 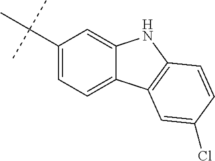 | 2-(6-chloro-9H-carbazol-2-yl)-N-(4-(2- methyl-4-pyridinyl)phenyl)propanamide | 440 |
| 66 | 1 | H | (R)-CH(CH₃) | 6-methoxynaphth-2-yl | (2R)-2-(6-methoxy-2-naphthalenyl)-N-(4- | 397.1 |
| | | | | | (2-methyl-4-pyridinyl)phenyl)propanamide | |
| 69 | 0 | H | | naphth-1-yl | N-(4-(2-methyl-4-pyridinyl)phenyl)-1- | 339 |
| | | | | | naphthalenecarboxamide | |
| 70 | 0 | H | | 4-methoxyphenyl | 4-methoxy-N-(4-(2-methyl-4-pyridinyl)- | 319 |
| | | | | | phenyl)benzamide | |
| 72 | 1 | H | CH(CH₃) | 3-methylphenyl | 2-(3-methylphenyl)-N-(4-(2-methyl-4- | 331.1 |
| | | | | | pyridinyl)phenyl)propanamide | |
| 73 | 1 | H | CH(CH₃) | 3,4-dichlorophenyl | 2-(3,4-dichlorophenyl)-N-(4-(2-methyl-4- | 385 |
| | | | | | pyridinyl)phenyl)propanamide | |
| 74 | 1 | H | CH(CH₃) | 3-trifluoromethylphenyl | N-(4-(2-methyl-4-pyridinyl)phenyl)-2-(3- | 385.1 |
| | | | | | (trifluoromethyl)phenyl)propanamide | |
| 75 | 1 | H | (S)-CH(CH₃) | 3-benzoylphenyl | (2S)-N-(4-(2-methyl-4-pyridinyl)phenyl)-2- | 421 |
| | | | | | (3-(phenylcarbonyl)phenyl)propanamide | |
| 76 | 1 | H | (S)-CH(CH₃) | 4-isobutylphenyl | (2S)-2-(4-(2-methylpropyl)phenyl)-N-(4-(2- | 373 |
| | | | | | methyl-4-pyridinyl)phenyl)propanamide | |
| 77 | 1 | H | (S)-CH(CH₃) | 3-fluoro-4-phenylphenyl | (2S)-2-(2-fluoro-4-biphenylyl)-N-(4-(2- | 411 |
| | | | | | methyl-4-pyridinyl)phenyl)propanamide | |
| 78 | 1 | H | CH₂ | benzothiophen-3-yl | 2-(1-benzothiophen-3-yl)-N-(4-(2-methyl-4- | 359 |
| | | | | | pyridinyl)phenypacetamide | |
| 79 | 1 | H | CH₂ | 5-bromo-1H-indazol-3-yl | 2-(5-bromo-1H-indazol-3-yl)-N-(4-(2- | 421 |
| | | | | | methyl-4-pyridinyl)phenyl)acetamide | |
| 80 | 1 | H | CH₂ | 1H-indol-3-yl | 2-(1H-indol-3-yl)-N-(4-(2-methyl-4- | 342 |
| | | | | | pyridinyl)phenyl)acetamide | |
| 82 | 1 | H | (S)-CHCH₃ | 6-methoxynaphth-2-yl | (2S)-2-(6-methoxy-2-naphthalenyl)-N-(4- | 397 |
| | | | | | (2-methyl-4-pyridinyl)phenyl)propanamide | |
| 83 | 1 | H | (R)-CHCH₃ | (3-fluoro-4-phenyl)phenyl | (2R)-2-(2-fluoro-4-biphenylyl)-N-(4-(2- | 411 |
| | | | | | methyl-4-pyridinyl)phenyl)propanamide | |
| 84 | 1 | H | CH(n-propyl) | 3-methylphenyl | 2-(3-methylphenyl)-N-(4-(2-methyl-4- | 359.1 |
| | | | | | pyridinyl)phenyl)pentanamide | |
| 85 | 1 | H | CH(n-butyl) | 3-methylphenyl | 2-(3-methylphenyl)-N-(4-(2-methyl-4- | 373.2 |
| | | | | | pyridinyl)phenyl)hexanamide | |
| 86 | 0 | 2-F | — | 3,5-diClphenyl | 3,5-dichloro-N-(2-fluoro-4-(2-methyl-4- | 375.1 |
| | | | | | pyridinyl)phenyl)benzamide | |
| 87 | 1 | H | CH₂ | 3,5-di-trifluoromethylphenyl | 2-(3,5-bis(trifluoromethyl)phenyl)-N-(4-(2- | 439.1 |
| | | | | | methyl-4-pyridinyl)phenyl)acetamide | |
| 88 | 1 | H | CH₂ | pyridin-2-yl | N-(4-(2-methyl-4-pyridinyl)phenyl)-2-(2- | 304 |
| | | | | | pyridinyl)acetamide | |
| 89 | 1 | H | CH₂ | 3,5-dimethylisoxazol-4-yl | 2-(3,5-dimethyl-4-isoxazolyl)-N-(4-(2- | 322 |
| | | | | | methyl-4-pyridinyl)phenyl)acetamide | |
| 90 | 1 | 2-F | CH₂ | 3,5-di-trifluoromethylphenyl | 2-(3,5-bis(trifluoromethyl)phenyl)-N-(2- | 457.1 |
| | | | | | fluoro-4-(2-methyl-4-pyridinyl)phenyl)- | |
| | | | | | acetamide | |
| 91 | 0 | H | — | 2-biphenyl | N-(4-(2-methyl-4-pyridinyl)phenyl)-2- | 365 |
| | | | | | biphenylcarboxamide | |
| 92 | 0 | H | — | 4-dimethylaminophenyl | 4-(dimethylamino)-N-(4-(2-methyl-4- | 332 |
| | | | | | pyridinyl)phenyl)benzamide | |
| 93 | 0 | H | — | 3,4-methylenedioxyphenyl | N-(4-(2-methyl-4-pyridinyl)phenyl)-1,3- | 333 |
| | | | | | benzodioxole-5-carboxamide | |
| 94 | 0 | H | — | 4,5,6,7- | N-(4-(2-methyl-4-pyridinyl)phenyl)-4,5,6,7- | 349 |
| | | | | tetrahydrobenzthiophen-3-yl | tetrahydro-1-benzothiophene-3- | |
| | | | | | carboxamide | |
| 95 | 0 | H | — | cyclohexyl | N-(4-(2-methyl-4-pyridinyl)phenyl)cyclo- | 295 |
| | | | | | hexanecarboxamide | |
| 96 | 1 | H | CH₂ | cyclohexyl | 2-cyclohexyl-N-(4-(2-methyl-4-pyridinyl)- | 309 |
| | | | | | phenyl)acetamide | |
| 97 | 0 | H | — | 3-oxo-2-phenylisoindolin-4-yl | N-(4-(2-methyl-4-pyridinyl)phenyl)-3-oxo- | 420 |
| | | | | | 2-phenyl-2,3-dihydro-1H-isoindole-4- | |
| | | | | | carboxamide | |
| 98 | 0 | H | — | 2-cyclopentyl-3-oxoisoindolin- | 2-cyclopentyl-N-(4-(2-methyl-4-pyridinyl)- | 412 |
| | | | | 4-yl | phenyl)-3-oxo-2,3-dihydro-1H-isoindole-4- | |
| | | | | | carboxamide | |
| 99 | 1 | H | CH₂CH(CH₃) | phenyl | N-(4-(2-methyl-4-pyridinyl)phenyl)-3- | 331.1 |
| | | | | | phenylbutanamide | |
| 100 | 1 | H | CH(CH₃)CH₂ | phenyl | 2-methyl-N-(4-(2-methyl-4-pyridinyl)- | 331.1 |
| | | | | | phenyl)-3-phenylpropanamide | |
| 101 | 1 | H | S-CH(CH₃) | 3-fluorophenyl | (2S)-2-(3-fluorophenyl)-N-(4-(2-methyl-4- | 335.1 |
| | | | | | pyridinyl)phenyl)propanamide | |
| 102 | 1 | H | R-CH(CH₃) | 3-fluorophenyl | (2R)-2-(3-fluorophenyl)-N-(4-(2-methyl-4- | 335.1 |
| | | | | | pyridinyl)phenyl)propanamide | |
| 103 | 1 | H | S-CH(CH₃) | 6-chloropyridin-2-yl | (2S)-2-(6-chloro-2-pyridinyl)-N-(4-(2- | 352.1 |
| | | | | | methyl-4-pyridinyl)phenyl)propanamide | |
| 104 | 1 | H | R-CH(CH₃) | 6-chloropyridin-2-yl | (2R)-2-(6-chloro-2-pyridinyl)-N-(4-(2- | 352.1 |
| | | | | | methyl-4-pyridinyl)phenyl)propanamide | |
| 105 | 1 | H | S- | 3-methylphenyl | (2S)-4-methyl-2-(3-methylphenyl)-N-(4-(2- | 373.1 |
| | | | CH[CH₂CH(CH₃)₂] | | methyl-4-pyridinyl)phenyl)pentanamide | |
| 106 | 1 | H | CH₂ | 2-chloro-6-fluorophenyl | 2-(2-chloro-6-fluorophenyl)-N-(4-(2- | 355 |
| | | | | | methyl-4-pyridinyl)phenyl)acetamide | |
| 107 | 1 | H | CH₂ | 3,5-dimethoxyphenyl | 2-(3,5-dimethoxyphenyl)-N-(4-(2-methyl-4- | 363 |
| | | | | | pyridinyl)phenyl)acetamide | |
| 108 | 1 | H | CH₂ | 2,5-ditrifluoromethylphenyl | 2-(2,5-bis(trifluoromethyl)phenyl)-N-(4-(2- | 439 |
| | | | | | methyl-4-pyridinyl)phenyl)acetamide | |
| 109 | 1 | H | CH₂ | 2-fluoro-3-trifluoromethyl- | 2-(2-fluoro-3-(trifluoromethyl)phenyl)-N- | 389 |
| | | | | phenyl | (4-(2-methyl-4-pyridinyl)phenyl)acetamide | |
| 110 | 1 | H | CH₂ | 4-trifluoromethoxyphenyl | N-(4-(2-methyl-4-pyridinyl)phenyl)-2-(4- | 387 |
| | | | | | (trifluoromethoxy)phenyl)acetamide | |
| 111 | 1 | H | CH₂ | 4-benzyloxyphenyl | 2-(4-(benzyloxy)phenyl)-N-(4-(2-methyl-4- | 409 |
| | | | | | pyridinyl)phenyl)acetamide | |
| 112 | 1 | H | CH₂ | 4-phenoxyphenyl | N-(4-(2-methyl-4-pyridinyl)phenyl)-2-(4- | 395 |
| | | | | | phenoxyphenypacetamide | |
| 113 | 1 | H | CH₂ | 2-benzyloxyphenyl | 2-(2-(benzyloxy)phenyl)-N-(4-(2-methyl-4- | 409 |
| | | | | | pyridinyl)phenyl)acetamide | |
| 114 | 1 | H | CH₂ | 2-ethoxyphenyl | 2-(2-ethoxyphenyl)-N-(4-(2-methyl-4- | 347 |
| | | | | | pyridinyl)phenypacetamide | |
| 115 | 1 | H | CH₂ | 9-methyl-9H-carbazol-2-yl | 2-(9-methyl-9H-carbazol-2-yl)-N-(4-(2- | 406 |
| | | | | | methyl-4-pyridinyl)phenypacetamide | |
| 116 | 1 | H | CH₂ | 2,3-dihydrobenzofuran-5-yl | 2-(2,3-dihydro-1-benzofuran-5-yl)-N-(4-(2- | 345 |
| | | | | | methyl-4-pyridinyl)phenyl)acetamide | |
| 117 | 0 | H | | 2,3-dihydroinden-1-yl | N-(4-(2-methyl-4-pyridinyl)phenyl)-2,3- | 329 |
| | | | | | dihydro-1H-indene-1-carboxamide | |
| 118 | 1 | H | CH₂ | 4-methoxy-3-methylphenyl | 2-(4-methoxy-3-methylphenyl)-N-(4-(2- | 347 |
| | | | | | methyl-4-pyridinyl)phenypacetamide | |
| 119 | 1 | H | CH₂ | 3-chloro-2,6-difluorophenyl | 2-(3-chloro-2,6-difluorophenyl)-N-(4-(2- | 373 |
| | | | | | methyl-4-pyridinyl)phenyl)acetamide | |
| 120 | 0 | H | | 3-oxo-2,3-dihydroinden-1-yl | N-(4-(2-methyl-4-pyridinyl)phenyl)-3-oxo- | 343.1 |
| | | | | | 2,3-dihydro-1H-indene-1-carboxamide | |
| 121 | 1 | H | CH(isobutyl) | 3,5-trifluoromethylphenyl | 2-(3,5-bis(trifluoromethyl)phenyl)-4- | 495.1 |
| | | | | | methyl-N-(4-(2-methyl-4-pyridinyl)phenyl)- | |
| | | | | | pentanamide | |
| 122 | 1 | H | R-CHCH₃ | 3-chloro-9H-carbazol-7-yl | (2R)-2-(6-chloro-9H-carbazol-2-yl)-N-(4- | 440.1 |
| | | | | | (2-methyl-4-pyridinyl)phenyl)propanamide | |
| 123 | 1 | H | CHCH₃ | 1-oxoisoindolin-2-yl | N-(4-(2-methyl-4-pyridinyl)phenyl)-2-(4- | 448.2 |
| | | | | | (1-oxo-1,3-dihydro-2H-isoindol-2-yl)- | |
| | | | | | phenyl)propanamide | |
| 124 | 1 | H | CHCH₂cyclobutyl | 3-bromophenyl | 2-(3-bromophenyl)-3-cyclobutyl-N-(4-(2- | 449.1 |
| | | | | | methyl-4-pyridinyl)phenyl)propanamide | |
| 125 | 1 | H | CH₂ | 2-phenylbenzoxazol-5-yl | N-(4-(2-methyl-4-pyridinyl)phenyl)-2-(2- | 420.2 |
| | | | | | phenyl-1,3-benzoxazol-5-yl)acetamide | |
| 126 | 1 | H | CH₂ | quinolin-6-yl | N-(4-(2-methyl-4-pyridinyl)phenyl)-2-(6- | 354 |
| | | | | | quinolinyl)acetamide | |
| 127 | 1 | H | S- | 3-(4-morpholin-4-ylphenyl)- | (2S)-4-methyl-N-(4-(2-methyl-4- | 520.2 |
| | | | CH[CH₂CH(CH₃)₂] | phenyl | pyridinyl)phenyl)-2-(4′-(4-morpholinyl)-3- | |
| | | | | | biphenylyl)pentanamide | |
| 128 | 1 | H | R- | 3-(4-morpholin-4-ylphenyl)- | (2R)-4-methyl-N-(4-(2-methyl-4- | 520.2 |
| | | | CH[CH₂CH(CH₃)₂] | phenyl | pyridinyl)phenyl)-2-(4′-(4-morpholinyl)-3- | |
| | | | | | biphenylyl)pentanamide | |
| 129 | 1 | H | CHCH₂CH₂F | 3-bromophenyl | 2-(3-bromophenyl)-4-fluoro-N-(4-(2- | 427.1 |
| | | | | | methyl-4-pyridinyl)phenyl)butanamide | |
| 130 | 1 | H | CHCH₃ | 5-bromo-3-methylindazol-1-yl | 2-(5-bromo-3-methyl-1H-indazol-1-yl)-N- | 449 |
| | | | | | (4-(2-methyl-4-pyridinyl)phenyl)- | |
| | | | | | propanamide | |
| 131 | 1 | H | R- | 4-(4-morpholin-4-ylphenyl)- | (2R)-4-methyl-N-(4-(2-methyl-4-pyridinyl)- | 520.2 |
| | | | CH[CH₂CH(CH₃)₂] | phenyl | phenyl)-2-(4′-(4-morpholinyl)-4- | |
| | | | | | biphenylyl)pentanamide | |
| 132 | 1 | H | S- | 4-(4-morpholin-4-ylphenyl)- | (2S)-4-methyl-N-(4-(2-methyl-4- | 520.2 |
| | | | CH[CH₂CH(CH₃)₂] | phenyl | pyridinyl)phenyl)-2-(4′-(4-morpholinyl)-4- | |
| | | | | | biphenylyl)pentanamide | |
| 133 | 1 | H | CHCH₃ | 3-(4-morpholin-4-ylphenyl)- | N-(4-(2-methyl-4-pyridinyl)phenyl)-2-(4′- | 478.2 |
| | | | | phenyl | (4-morpholinyl)-3-biphenylyl)propanamide | |
| 134 | 1 | H | CH[CH₂CH(CH₃)₂] | 4-piperidin-1-ylphenyl | 4-methyl-N-(4-(2-methyl-4-pyridinyl)- | 442.2 |
| | | | | | phenyl)-2-(4-(1-piperidinyl)phenyl)- | |
| | | | | | pentanamide | |
| 135 | 1 | H | CHCH₃ | 3(2-morpholin-4-ylpyridin-5- | N-(4-(2-methyl-4-pyridinyl)phenyl)-2-(3- | 479 |
| | | | | yl)-phenyl | (6-(4-morpholinyl)-3-pyridinyl)phenyl)- | |
| | | | | | propanamide | |
| 136 | 1 | H | CHCH₃ | 3-(1-tert-butoxycarbonyl- | tert-butyl 4-(3-1-methyl-2-((4-(2-methyl-4- | 498 |
| | | | | 1,2,3,6-tetrahydropyridin-4- | pyridinyl)phenyl)amino)-2-oxoethyl)- | |
| | | | | yl)-phenyl | phenyl)-3,6-dihydro-1(2H)- | |
| | | | | | pyridinecarboxylate | |
| 137 | 1 | H | CH[CH₂CH(CH₃)₂] | 3-morpholin-4-ylphenyl | 4-methyl-N-(4(2-methyl-4-pyridinyl)- | 444.2 |
| | | | | | phenyl)-2-(3-(4-morpholinyl)phenyl)- | |
| | | | | | pentanamide | |
| 138 | 1 | H | CHCH₃ | 3-(4-methylpiperazin-l-yl)- | 243-(4-methyl-1-piperazinyl)phenyl)-N-(4- | 415.2 |
| | | | | phenyl | (2-methyl-4-pyridinyl)phenyl)propanamide | |
| 139 | 1 | H | CHCH₃ | 3-(4-pyridin-2-ylpiperazin-1- | N-(4-(2-methyl-4-pyridinyl)phenyl)-2-(3- | 478.2 |
| | | | | yl)-phenyl | (4-(2-pyridinyl)-1-piperazinyl)phenyl)- | |
| | | | | | propanamide | |
| 140 | 1 | H | CHCH₃ | 3-[1-(2-hydroxyethyl)- | 2-(3-(1-(2-hydroxyethyl)-4-piperidinyl)- | 444 |
| | | | | piperidin-4-yl]phenyl | phenyl)-N-(4-(2-methyl-4- | |
| | | | | | pyridinyl)phenyl)propanamide | |
| 141 | 1 | H | CHCH₃ | 6-morpholin-4-ylpyridin-2-yl | N-(4-(2-methyl-4-pyridinyl)phenyl)-2(6- | 403.2 |
| | | | | | (4-mozpholinyl)-2-pyridinyl)propanamide | |
| 142 | 1 | H | CHCH₃ | 3-methylthiophenyl | N-(4-(2-methyl-4-pyridinyl)phenyl)-2-(3- | 363.2 |
| | | | | | (methylsulfanyl)phenyl)propanamide | |
| 143 | 1 | 3- | CHCH₃ | 3-bromophenyl | 2(3-bromophenyl)-N-(3-methoxy-4-(2- | 428 |
| | | OCH₃ | | | methyl-4-pyridinyl)phenyl)propanamide | |
| 144 | 1 | 3-CH₃ | CHCH₃ | 3-bromophenyl | 2-(3-bromophenyl)-N-(3-methyl-4-(2- | 411 |
| | | | | | methyl-4-pyridinyl)phenyl)propanamide | |
| 145 | 1 | H | CH[CH₂CH(CH₃)₂] | 3-methylsulfonylphenyl | 4-methyl-N-(4(2-methyl-4-pyridinyl)- | 437.2 |
| | | | | | phenyl)-2-(3-(methylsulfonyl)phenyl)- | |
| | | | | | pentanamide | |
| 147 | 1 | H | CHCH₃ | 3-(4-dimethylaminophenyl)- | 2-(4'-(dimethylamino)-3-biphenylyl)-N-(4- | 436.2 |
| | | | | phenyl | (2-methyl-4-pyridinyl)phenyl)propanamide | |
| 148 | 1 | H | CHCH₃ | 3-(4-morpholin-4-ylphenyl)- | 2-hydroxy-N-(4-(2-methyl-4-pyridinyl)- | 480.2 |
| | | | | phenyl | phenyl)-2-(4′-(4-morpholinyl)-3- | |
| | | | | | biphenylyl)ethanamide | |
| 149 | 1 | H | CHCH₃ | 6-(4-morpholin-4-ylphenyl)- | N-(4-(2-methyl-4-pyridinyl)phenyl)-2-(6- | 479.2 |
| | | | | pyridin-2-yl | (4-(4-morpholinyl)phenyl)-2-pyridinyl)- | |
| | | | | | propanamide | |
| 150 | 1 | H | CH₂ | 3-(4-morpholin-4-ylphenyl)- | N-(4-(2-methyl-4-pyridinyl)phenyl)-2-(4'- | 464.2 |
| | | | | phenyl | (4-morpholinyl)-3-biphenylyl)acetamide | |
| 151 | 1 | H | CHCH₃ | 4-phenylimidazol-1-yl | N-(4-(2-methyl-4-pyridinyl)phenyl)-2-(4- | 383.2 |
| | | | | | phenyl-1H-imidazol-1-yl)propanamide | |
| 152 | 1 | H | R-CHCH₃ | 3-(4-morpholin-4-ylphenyl)- | (2R)-N-(4-(2-methyl-4-pyridinyl)phenyl)-2- | 478.2 |
| | | | | phenyl | (4′-(4-morpholinyl)-3-biphenylyl)- | |
| | | | | | propanamide | |
| 153 | 1 | H | S-CHCH₃ | 3-(4-morpholin-4-ylphenyl)- | (2S)-N-(4-(2-methyl-4-pyridinyl)phenyl)-2- | 478.2 |
| | | | | phenyl | (4′-(4-morpholinyI)-3-biphenylyl)- | |
| | | | | | propanamide | |
| 154 | 1 | H | CHCH₃ | 3-(3-cyanophenyl)phenyl | 2-(3′-cyano-3-biphenylyl)-N-(4-(2-methyl- | 418.1 |
| | | | | | 4-pyridinyl)phenyl)propanamide | |
| 155 | 1 | H | CHCH₃ | 3-(3-methoxyphenyl)phenyl | 2-(3′-methoxy-3-biphenylyl)-N-(4-(2- | 423.1 |
| | | | | | methyl-4-pyridinyl)phenyl)propanamide | |
| 156 | 1 | H | CHCH₃ | 3-(2-methoxypyrimidin-5-yl)- | 2-(3-(2-methoxy-5-pyrimidinyl)phenyl)-N- | 425.1 |
| | | | | phenyl | (4-(2-methyl-4-pyridinyl)phenyl)- | |
| | | | | | propanamide | |
| 157 | 1 | H | CHCH₃ | 3-(4-dimethylaminocarbonyl- | N,N-dimethyl-3′-(1-methyl-2-((4-(2-methyl- | 464.3 |
| | | | | phenyl)phenyl | 4-pyridinyl)phenypamino)-2-oxoethyl)-4- | |
| | | | | | biphenylcarboxamide | |
| 158 | 1 | H | CHCH₃ | 3-(4-methylcarbonyl- | 2-(4′-acetyl-3-biphenylyl)-N-(4-(2-methyl- | 435.1 |
| | | | | phenyl)phenyl | 4-pyridinyl)phenyl)propanamide | |
| 159 | 1 | 3- | CHCH₃ | 3-fluoro-5-trifluoromethyl- | 2-(3-fluoro-5-(trifluoromethyl)phenyl)-N- | 433 |
| | | OCH₃ | | phenyl | (3-methoxy-4-(2-methyl-4-pyridinyl)- | |
| | | | | | phenyl)propanamide | |
| 160 | 1 | 3- | CHCH₃ | 5-fluoro-3-(4-morpholin-4- | 2-(5-fluoro-4′-(4-morpholinyl)-3- | 526 |
| | | OCH₃ | | ylphenyl)phenyl | biphenylyl)-N-(3-methoxy-4-(2-methyl-4- | |
| | | | | | pyridinyl)phenyl)propanamide | |
| 161 | 1 | 2-CN | CHCH₃ | 3-bromophenyl | 2-(3-bromophenyl)-N-(2-cyano-4-(2- | 422 |
| | | | | | methyl-4-pyridinyl)phenyl)propanamide | |
| 162 | 1 | 3-Cl | CHCH₃ | 3-bromophenyl | 2-(3-bromophenyl)-N-(3-chloro-4-(2- | 431 |
| | | | | | methyl-4-pyridinyl)phenyl)propanamide | |
| 163 | 1 | H | CHCH₂cyclopropyl | 3-bromophenyl | 2-(3-bromophenyl)-3-cyclopropyl-N-(4-(2- | 435 |
| | | | | | methyl-4-pyridinyl)phenyl)propanamide | |
| 164 | 1 | H | CH₂ | 2-(4-trifluoromethylphenyl)- | N-(4-(2-methyl-4-pyridinyl)phenyl)-2-(2- | 448.1 |
| | | | | pyridin-4-yl | (4-(trifluoromethyl)phenyl)-4-pyridinyl)- | |
| | | | | | acetamide | |
| 165 | 1 | H | CHCH₃ | 3-phenylaminophenyl | N-(4-(2-methyl-4-pyridinyl)phenyl)-2-(3- | 408.2 |
| | | | | | (phenylamino)phenyl)propanamide | |
| 166 | 1 | H | CHCH₃ | 3-(2-methoxyethylamino)- | 2-(3-((2-methoxyethyl)amino)phenyl)-N-(4- | 390.2 |
| | | | | phenyl | (2-methyl-4-pyridinyl)phenyl)propanamide | |
| 167 | 1 | H | CHCH₃ | 4-fluoro-3-morpholin-4-yl- | 2-(4-fluoro-3-(4-morpholinyl)phenyl)-N-(4- | 420.1 |
| | | | | phenyl | (2-methyl-4-pyridinyl)phenyl)propanamide | |
| 168 | 0 | H | — | R-1-tert-butoxycarbonyl- | tert-butyl (2R)-2-((4-(2-methyl-4- | 382.2 |
| | | | | pyrrolidin-2-yl | pyridinyl)-phenyl)carbamoyl)-1- | |
| | | | | | pyrrolidinecarboxylate | |
| 169 | 1 | 3- | S-CHCH₃ | 5-fluoro-3-(4-morpholin-4- | (2S)-2-(3-bromo-5-fluorophenyl)-N-(3- | 443 |
| | | OCH₃ | | ylphenyl)phenyl | methoxy-4-(2-methyl-4-pyridinyl)- | |
| | | | | | phenyl)propanamide | |
| 170 | 1 | 3- | S-CHCH₃ | 5-fluoro-3-(4-morpholin-4- | (2S)-2-(5-fluoro-4′-(4-morpholinyl)-3- | 526 |
| | | OCH₃ | | ylphenyl)phenyl | biphenylyl)-N-(3-methoxy-4-(2-methyl-4- | |
| | | | | | pyridinyl)phenyl)propanamide | |
| 171 | 1 | H | CHCH₃ | 4-(cyclopropylmethylamino)- | 2-(4-((cyclopropylmethyl)amino)phenyl)-N- | 386 |
| | | | | phenyl | (4-(2-methyl-4-pyridinyl)phenyl)- | |
| | | | | | propanamide | |
| 172 | 1 | H | CHCH₃ | 4-(N,N-dicyclopropylmethyl- | 2-(4-(bis(cyclopropylmethyl)amino)- | 440 |
| | | | | amino)-phenyl | phenyl)-N-(4-(2-methyl-4-pyridinyl)- | |
| | | | | | phenyl)propanamide | |
| 173 | 1 | H | CH[CH₂CH(CH3)₂] | tetrahydropyran-4-yl | 4-methyl-N-(4-(2-methyl-4-pyridinyl)- | 367.1 |
| | | | | | phenyl)-2-(tetrahydro-2H-pyran-4- | |
| | | | | | yl)pentanamide | |
| 174 | 1 | H | CHCH₃ | 3-thiophen-3-ylphenyl | N-(4-(2-methyl-4-pyridinyl)phenyl)-2-(3- | 399.1 |
| | | | | | (3-thiophenyl)phenyl)propanamide | |
| 175 | 1 | H | CHCH₃ | 3-indol-5-ylphenyl | 2-(3-(1H-indol-5-yl)phenyl)-N-(4-(2- | 432.2 |
| | | | | | methyl-4-pyridinyl)phenyl)propanamide | |
| 176 | 1 | 3- | CHCH₃ | 4-N,N-dicyclobutyl- | 2-(4-(dicyclobutylamino)phenyl)-N-(3- | 470.2 |
| | | OCH₃ | | aminophenyl | methoxy-4-(2-methyl-4-pyridinyl)- | |
| | | | | | phenyl)propanamide | |
| 177 | 1 | H | CHCH₃ | 3-(2-morpholin-4-ylpyrimidin- | N-(4-(2-methyl-4-pyridinyl)phenyl)-2-(3- | 480.2 |
| | | | | 5-yl)phenyl | (2-(4-morpholinyl)-5-pyrimidinyl)- | |
| | | | | | phenyl)propanamide | |
| 178 | 1 | H | CHCH₃ | 2-cyclopropylpyridin-4-yl | 2-(2-cyclopropyl-4-pyridinyl)-N-(4-(2- | 358.1 |
| | | | | | methyl-4-pyridinyl)phenyl)propanamide | |
| 179 | 1 | H | CHCH₃ | 4-(4-methoxybenzylamino)- | 2-(4-((4-methoxybenzyl)amino)phenyl)-N- | 452 |
| | | | | phenyl | (4-(2-methyl-4-pyridinyl)phenyl)- | |
| | | | | | propanamide | |
| 180 | 1 | H | R- | 3-bromophenyl | (2R)-2-(3-bromophenyl)-3-cyclopropyl-N- | 435 |
| | | | CHCH₂cyclopropyl | | (4-(2-methyl-4-pyridinyl)phenyl)- | |
| | | | | | propanamide | |
| 181 | 1 | H | S- | 3-bromophenyl | (2S)-2-(3-bromophenyl)-3-cyclopropyl-N- | 435 |
| | | | CHCH₂cyclopropyl | | (4-(2-methyl-4-pyridinyl)phenyl)- | |
| | | | | | propanamide | |
| 182 | 1 | H | CH[CH₂CH(CH₃)₂] | 2-(6-methoxyethylamino)- | 2-(6-((2-methoxyethyl)amino)-2-pyridinyl)- | 433.2 |
| | | | | pyridin-2-yl | 4-methyl-N-(4-(2-methyl-4-pyridinyl)- | |
| | | | | | phenyl)pentanamide | |
| 183 | 1 | H | CHCH₃ | 3-(2-morpholin-4-ylthiazol-4- | N-(4-(2-methyl-4-pyridinyl)phenyl)-2-(3- | 485.1 |
| | | | | yl)phenyl | (2-(4-morpholinyl)-1,3-thiazol-4-yl)- | |
| | | | | | phenyl)propanamide | |
| 184 | 0 | H | — | 2,2-dimethylcycloprop-1-yl | 2,2-dimethyl-N-(4-(2-methyl-4-pyridinyl)- | 281 |
| | | | | | phenyl)cyclopropanecarboxamide | |
| 185 | 0 | H | — | 2,2-difluorocycloprop-1-yl | 2,2-difluoro-N-(4-(2-methyl-4-pyridinyl)- | 289 |
| | | | | | phenyl)cyclopropanecarboxamide | |
| 186 | 0 | H | — | 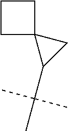 | N-(4-(2-methyl-4-pyridinyl)phenyl)- spiro[23]hexane-1-carboxamide | 293 |
| 187 | 1 | H | CH₂ | 2-pyridin-2-ylbenzoxazol-5-yl | N-(4-(2-methyl-4-pyridinyl)phenyl)-2-(2- | 421.1 |
| | | | | | (2-pyridinyl)-1,3-benzoxazol-5- | |
| | | | | | yl)acetamide | |
| 188 | 1 | H | CHCH₃ | 4-tetrahydropyran-4-ylamino- | N-(4-(2-methyl-4-pyridinyl)phenyl)-2-(4- | 416 |
| | | | | phenyl | (tetrahydro-2H-pyran-4-ylamino)phenyl)- | |
| | | | | | propanamide | |
| 189 | 1 | H | R- | 3-bromophenyl | (2R)-2-(3-bromophenyl)-4-methyl-N-(4-(2- | 438 |
| | | | CH[CH₂CH(CH₃)₂] | | methyl-4-pyridinyl)phenyl)pentanamide | |
| 190 | 1 | H | R- | 3,5-difluorophenyl | (2R)-2-(3,5-difluorophenyl)-4-methyl-N-(4- | 395.1 |
| | | | CH[CH₂CH(CH₃)₂] | | (2-methyl-4-pyridinyl)phenyppentanamide | |
| 191 | 1 | 3- | CHCH₃ | 3-(4-morpholin-4-ylphenyl)- | N-(3-methoxy-4-(2-methyl-4-pyridinyl)- | 508 |
| | | OCH₃ | | phenyl | phenyl)-2-(4′-(4-morpholinyl)-3- | |
| | | | | | biphenylyl)propanamide | |
| 192 | 0 | H | — | 6-chloroimidazo[1,2-a]pyridin- | 6-chloro-N-(4-(2-methyl-4-pyridinyl)- | 363 |
| | | | | 2-yl | phenyl)imidazo[1,2-a]pyridine-2- | |
| | | | | | carboxamide | |
| 193 | 0 | H | — | imidazo[1,2-a]pyridin-2-yl | N-(4-(2-methyl-4-pyridinyl)phenyl)- | 320 |
| | | | | | imidazo[1,2-a]pyridine-2-carboxamide | |
| 194 | 0 | H | — | 1-methylindol-2-yl | 1-methyl-N-(4-(2-methyl-4-pyridinyl)- | 342 |
| | | | | | phenyl)-1H-indole-2-carboxamide | |
| 195 | 0 | H | — | 4,5,6,7-tetrahydro-1,2- | N-(4-(2-methyl-4-pyridinyl)phenyl)-4,5,6,7- | 334 |
| | | | | benzisoxazol-3-yl | tetrahydro-1,2-benzisoxazole-3- | |
| | | | | | carboxamide | |
| 196 | 0 | H | — | 6-bromoimidazo[1,2-a]pyridin- | 6-bromo-N-(4-(2-methyl-4-pyridinyl)- | 407 |
| | | | | 2-yl | phenyl)imidazo[1,2-a]pyridine-2- | |
| | | | | | carboxamide | |
| 197 | 1 | H | S-CHCH₃ | 3-chlorophenyl | (2S)-2-(3-chlorophenyl)-N-(4-(2-methyl-4- | 351.1 |
| | | | | | pyridinyl)phenyl)propanamide | |
| 198 | 1 | H | R-CHCH₃ | 3-chlorophenyl | (2R)-2-(3-chlorophenyl)-N-(4-(2-methyl-4- | 351.1 |
| | | | | | pyridinyl)phenyl)propanamide | |
| 199 | 0 | H | — | 1,2,3,4-tetrahydronaphth-1-yl | N-(4-(2-methyl-4-pyridinyl)phenyl)-1,2,3,4- | 343.2 |
| | | | | | tetrahydro-1-naphthalenecarboxamide | |
| 200 | 1 | H | CHCH₂CH₃ | 3-methylphenyl | 2-(3-methylphenyl)-N-(4-(2-methyl-4- | 345.1 |
| | | | | | pyridinyl)phenyl)butanamide | |
| 201 | 1 | H | R-CHCH₃ | 3,5-difluorophenyl | (2R)-2-(3,5-difluorophenyl)-N-(4-(2- | 353.1 |
| | | | | | methyl-4-pyridinyl)phenyl)propanamide | |
| 202 | 1 | H | S-CHCH₃ | 3,5-difluorophenyl | (2S)-2-(3,5-difluorophenyl)-N-(4-(2- | 353.1 |
| | | | | | methyl-4-pyridinyl)phenyl)propanamide | |
| 203 | 1 | H | R-CH(CH₂)₃CH₃ | 3-methylphenyl | (2R)-2-(3-methylphenyl)-N-(4-(2-methyl-4- | 373.2 |
| | | | | | pyridinyl)phenyl)hexanamide | |
| 204 | 1 | H | S-CH(CH₂)₃CH₃ | 3-methylphenyl | (2S)-2-(3-methylphenyl)-N-(4-(2-methyl-4- | 373.2 |
| | | | | | pyridinyl)phenyl)hexanamide | |
| 205 | 1 | H | R- | 3-methylphenyl | (2R)-4-methyl-2-(3-methylphenyl)-N-(4-(2- | 373.2 |
| | | | CH[CH2CH(CH3)2] | | methyl-4-pyridinyl)phenyl)pentanamide | |
| 206 | 1 | H | S- | 3-methylphenyl | (2S)-4-methyl-2-(3-methylphenyl)-N-(4-(2- | 373.2 |
| | | | CH[CH₂CH(CH₃)₂] | | methyl-4-pyridinyl)phenyl)pentanamide | |
| 207 | 0 | H | — | biphen-3-yl | N-(4-(2-methyl-4-pyridinyl)phenyl)-3- | 365 |
| | | | | | biphenylcarboxamide | |
| 208 | 1 | H | S- | 3,5-difluorophenyl | (2S)-2-(3,5-difluorophenyl)-4-methyl-N-(4- | 395.1 |
| | | | CH[CH₂CH(CH₃)₂] | | (2-methyl-4-pyridinyl)phenyl)pentanamide | |
| 209 | 1 | H | R- | 3,5-difluorophenyl | (2R)-2-(3,5-difluorophenyl)-4-methyl-N-(4- | 395.1 |
| | | | CH[CH₂CH(CH₃)₂] | | (2-methyl-4-pyridinyl)phenyl)pentanamide | |
| 210 | 1 | H | CHCH₃ | 3,5-dichlorophenyl | 2-(3,5-dichlorophenyl)-N-(4-(2-methyl-4- | 385.1 |
| | | | | | pyridinyl)phenyl)propanamide | |
| 211 | 1 | H | CH[CH₂CH(CH₃)₂] | 3,5-dichlorophenyl | 2-(3,5-dichlorophenyl)-4-methyl-N-(4-(2- | 427.1 |
| | | | | | methyl-4-pyridinyl)phenyl)pentanamide | |
| 212 | 1 | H | R- | 3-bromophenyl | (2R)-2-(3-bromophenyl)-4-methyl-N-(4-(2- | 438.1 |
| | | | CH[CH₂CH(CH₃)₂] | | methyl-4-pyridinyl)phenyl)pentanatnide | |
| 213 | 1 | H | S- | 3-bromophenyl | (2S)-2-(3-bromophenyl)-4-methyl-N-(4-(2- | 438.1 |
| | | | CH[CH₂CH(CH₃)₂] | | methyl-4-pyridinyl)phenyl)pentanatnide | |
| 214 | 1 | H | R- | 4-bromophenyl | (2R)-2-(4-bromophenyl)-4-methyl-N-(4-(2- | 437.1 |
| | | | CH[CH₂CH(CH₃)₂] | | methyl-4-pyridinyl)phenyl)pentanamide | |
| 215 | 1 | H | S- | 4-bromophenyl | (2S)-2-(4-bromophenyl)-4-methyl-N-(4-(2- | 437.1 |
| | | | CH[CH₂CH(CH₃)₂] | | methyl-4-pyridinyl)phenyl)pentanamide | |
| 216 | 1 | H | R- | 3-cyanophenyl | (2R)-2-(3-cyanophenyl)-4-methyl-N-(4-(2- | 384.2 |
| | | | CH[CH₂CH(CH₃)₂] | | methyl-4-pyridinyl)phenyppentanamide | |
| 217 | 1 | H | S- | 3-cyanophenyl | (2S)-2-(3-cyanophenyl)-4-methyl-N-(4-(2- | 384.2 |
| | | | CH[CH₂CH(CH₃)₂] | | methyl-4-pyridinyl)phenyl)pentanamide | |
| 218 | 1 | H | CHCH₃ | 6-chloro-9-methyl-9H- | 2-(6-chloro-9-methyl-9H-carbazol-2-yl)-N- | 454.1 |
| | | | | carbazol-2-yl | (4-(2-methyl-4-pyridinyl)phenyl)- | |
| | | | | | propanamide | |
| 219 | 1 | H | CHCH₃ | 3-(4-phenylpiperazin-1-yl)- | N-(4-(2-methyl-4-pyridinyl)phenyl)-2-(3- | 477.2 |
| | | | | phenyl | (4-phenyl-1-piperazinyl)phenyl)- | |
| | | | | | propanamide | |
| 220 | 1 | H | CHCH₃ | 3-iodophenyl | 2-(3-iodophenyl)-N-(4-(2-methyl-4- | 477.2 |
| | | | | | pyridinyl)phenyl)propanamide | |
| 221 | 1 | H | CHCH₃ | 4-isopropylaminophenyl | 2-(4-((1-methylethyl)amino)phenyl)-N-(4- | 374 |
| | | | | | (2-methyl-4-pyridinyl)phenyl)propanamide | |
| 222 | 1 | H | CHCH₃ | 3-benzyloxyphenyl | 2-(3-(benzyloxy)phenyl)-N-(4-(2-methyl-4- | 423.2 |
| | | | | | pyridinyl)phenyl)propanamide | |
| 223 | 1 | H | CHCH₃ | 3-(4-cyanophenyl)phenyl | 2-(4′-cyano-3-biphenylyl)-N-(4-(2-methyl- | 418.1 |
| | | | | | 4-pyridinyl)phenyppropanamide | |
| 224 | 1 | H | CHCH₃ | 3-(2-cyanophenyl)phenyl | 2-(2′-cyano-3-biphenylyl)-N-(4-(2-methyl- | 418.1 |
| | | | | | 4-pyridinyl)phenyl)propanamide | |
| 225 | 1 | H | CHCH₃ | 3-(4-methoxyphenyl)phenyl | 2-(4′-methoxy-3-biphenylyl)-N-(4-(2- | 423.1 |
| | | | | | methyl-4-pyridinyl)phenyl)propanamide | |
| 226 | 1 | H | CHCH₃ | 3-(2-methoxyphenyl)phenyl | 2-(2′-methoxy-3-biphenylyl)-N-(4-(2- | 423.1 |
| | | | | | methyl-4-pyridinyl)phenyl)propanamide | |
| 227 | 1 | H | CHCH₃ | 3-(4-trifluoromethoxy- | N-(4-(2-methyl-4-pyridinyl)phenyl)-2-(4′- | 477.1 |
| | | | | phenyl)phenyl | (trifluoromethoxy)-3-biphenylyl)- | |
| | | | | | propanamide | |
| 228 | 1 | H | CH[CH₂CH(CH₃)₂] | 3-[4-(1-tert-butoxypiperazin-1- | tert-butyl 4-(3′-(3-methyl-1-((4-(2-methyl- | 619.1 |
| | | | | yl)phenyl]-phenyl | 4-pyridinyl)phenyl)carbamoyl)butyl)-4- | |
| | | | | | biphenylyl)-1-piperazinecarboxylate | |
| 229 | 1 | H | CH[CH₂CH(CH₃)₂] | 3-(4-piperazin-l-ylphenyl)- | 4-methyl-N-(4-(2-methyl-4-pyridinyl)- | 519.2 |
| | | | | phenyl | phenyl)-2-(4′-(1-piperazinyl)-3-biphenylyl)- | |
| | | | | | pentanamide | |
| 230 | 1 | H | CHCH₃ | 5-fluoro-3-(4-morpholin-4-yl- | 2-(5-fluoro-4′-(4-morpholinyl)-3- | 496 |
| | | | | phenyl)phenyl | biphenylyl)-N-(4-(2-methyl-4- | |
| | | | | | pyridinyl)phenyl)propanamide | |
| 231 | 1 | H | R-CHCH₃ | 3-fluoro-5- | (2R)-2-(3-fluoro-5-(trifluoromethyl)- | 403 |
| | | | | trifluoromethylphenyl | phenyl)-N-(4-(2-methyl-4- | |
| | | | | | pyridinyl)phenyl)propanamide | |
| 232 | 1 | H | S-CHCH₃ | 3-fluoro-5- | (2S)-2-(3-fluoro-5-(trifluoromethyl)phenyl)- | 403 |
| | | | | trifluoromethylphenyl | N-(4-(2-methyl-4-pyridinyl)phenyl)- | |
| | | | | | propanamide | |
| 233 | 1 | H | CHCH₃ | 1-methylindol-3-yl | 2-(1-methyl-1H-indol-3-yl)-N-(4-(2-methyl- | 370.1 |
| | | | | | 4-pyridinyl)phenyl)propanamide | |
| 234 | 1 | H | CH[CH₂CH(CH₃)₂] | 4-morpholin-4-ylphenyl | 4-methyl-N-(4-(2-methyl-4-pyridinyl)- | 444.2 |
| | | | | | phenyl)-2-(4-(4-morpholinyl)phenyl)- | |
| | | | | | pentanamide | |
| 235 | 1 | H | CH₂ | 9-methyl-9H-carbazol-3yl | 2-(9-methyl-9H-carbazol-3-yl)-N-(4-(2- | 406 |
| | | | | | methyl-4-pyridinyl)phenyl)acetamide | |
| 236 | 1 | H | R-CHCH₃ | 3,5-dichlorophenyl | (2R)-2-(3,5-dichlorophenyl)-N-(4-(2- | 385 |
| | | | | | methyl-4-pyridinyl)phenyl)propanamide | |
| 237 | 1 | H | S- | 3,5-dichlorophenyl | (2S)-2-(3,5-dichlorophenyl)-4-methyl-N-(4- | 427.1 |
| | | | CH[CH₂CH(CH₃)₂] | | (2-methyl-4-pyridinyl)phenyl)pentanamide | |
| 238 | 1 | H | R- | 3,5-dichlorophenyl | (2R)-2-(3,5-dichlorophenyl)-4-methyl-N-(4- | 427.1 |
| | | | CH[CH₂CH(CH₃)₂] | | (2-methyl-4-pyridinyl)phenyl)pentanamide | |
| 239 | 1 | 3- | CHCH₃ | 1,3,4,9-tetrahydro-2H-beta- | N-(3-methoxy-4-(2-methyl-4-pyridinyl)- | 441 |
| | | OCH₃ | | carbolin-2-yl) | phenyl)-2-(1,3,4,9-tetrahydro-2H-beta- | |
| | | | | | carbolin-2-yl)propanamide | |
| 240 | 1 | 3- | CHCH₃ | 8-fluoro-1,3,4,5-tetrahydro- | 2-(8-fluoro-1,3,4,5-tetrahydro-2H- | 459 |
| | | OCH₃ | | 2H-pyrido[4,3-b]indol-2-yl | pyrido[4,3-b]indol-2-yl)-N-(3-methoxy-4- | |
| | | | | | (2-methyl-4-pyridinyl)phenyl)propanamide | |
| 241 | 1 | 3- | R-CHCH₃ | 5-fluoro-3-(4-morpholin-4-yl- | (2R)-2-(5-fluoro-4′-(4-morpholinyl)-3- | 526 |
| | | OCH₃ | | phenyl)phenyl | biphenylyl)-N-(3-methoxy-4-(2-methyl-4- | |
| | | | | | pyridinyl)phenyl)propanamide | |
| 242 | 1 | H | S-CHCH₃ | 4-isopropylaminophenyl | (2S)-2-(4-((1-methylethyl)amino)phenyl)- | 374 |
| | | | | | N-(4-(2-methyl-4-pyridinyl)phenyl)- | |
| | | | | | propanamide | |
| 243 | 1 | H | R-CHCH₃ | 4-isopropylaminophenyl | (2R)-2-(4-((1-methylethypamino)phenyl)- | 374 |
| | | | | | N-(4-(2-methyl-4-pyridinyl)phenyl)- | |
| | | | | | propanamide | |
| 244 | 1 | 3- | S-CHCH₃ | 3,5-di(trifluoromethyl)phenyl | (2S)-2-(3,5-bis(trifluoromethyl)phenyl)-N- | 483 |
| | | OCH₃ | | | (3-methoxy-4-(2-methyl-4-pyridinyl)- | |
| | | | | | phenyl)propanamide | |
| 245 | 1 | 3- | R-CHCH₃ | 3,5-di(trifluoromethyl)phenyl | (2R)-2-(3,5-bis(trifluoromethyl)phenyl)-N- | 483 |
| | | OCH₃ | | | (3-methoxy-4-(2-methyl-4-pyridinyl)- | |
| | | | | | phenyl)propanamide | |
| 246 | 1 | H | R-CH(CH₂)₂CH₃ | 3-bromophenyl | (2R)-2-(3-bromophenyl)-N-(4-(2-methyl-4- | 423.1 |
| | | | | | pyridinyl)phenyl)pentanamide | |
| 247 | 1 | H | S-CH(CH₂)₂CH₃ | 3-bromophenyl | (2S)-2-(3-bromophenyl)-N-(4-(2-methyl-4- | 423.1 |
| | | | | | pyridinyl)phenyl)pentanamide | |
| 248 | 1 | H | CH[CH₂CH(CH₃)₂] | 6-chloropyridin-2-yl | 2-(6-chloro-2-pyridinyl)-4-methyl-N-(4-(2- | 394.1 |
| | | | | | methyl-4-pyridinyl)phenyl)pentanamide | |
| 249 | 1 | H | CH[CH₂CH(CH₃)₂] | 6-morpholin-4-ylpyridin-2-yl | 4-methyl-N-(4-(2-methyl-4-pyridinyl)- | 445.2 |
| | | | | | phenyl)-2-(6-(4-morpholinyl)-2-pyridinyl)- | |
| | | | | | pentanamide | |
| 250 | 1 | H | CHCH₃ | 3-pyridin-3-ylphenyl | N-(4-(2-methyl-4-pyridinyl)phenyl)-2-(3- | 394.1 |
| | | | | | (3-pyridinyl)phenyl)propanamide | |
| 251 | 1 | H | CHCH₃ | 3-pyridin-4-ylphenyl | N-(4-(2-methyl-4-pyridinyl)phenyl)-2-(3- | 394.1 |
| | | | | | (4-pyridinyl)phenyl)propanamide | |
| 252 | 1 | 3- | R-CHCH₃ | 8-fluoro-1,3,4,5-tetrahydro- | (2R)-2-(8-fluoro-1,3,4,5-tetrahydro-2H- | 459 |
| | | OCH₃ | | 2H-pyrido[4,3-b]indol-2-yl | pyrido[4,3-1)]indol-2-yl)-N-(3-methoxy-4- | |
| | | | | | (2-methyl-4-pyridinyl)phenyl)propanamide | |
| 253 | 1 | 3- | S-CHCH₃ | 8-fluoro-1,3,4,5-tetrahydro- | (2S)-2-(8-fluoro-1,3,4,5-tetrahydro-2H- | 459 |
| | | OCH₃ | | 2H-pyrido[4,3-b]indol-2-yl | pyrido[4,3-b]indol-2-yl)-N-(3-methoxy-4- | |
| | | | | | (2-methyl-4-pyridinyl)phenyl)propanamide | |
| 254 | 1 | H | CHCH₃ | 4-di-n-propylaminophenyl | 2-(4-(dipropylamino)phenyl)-N-(4-(2- | 416 |
| | | | | | methyl-4-pyridinyl)phenyl)propanamide | |
| 255 | 1 | H | CHCH₃ | 4-diethylaminophenyl | 2-(4-(diethylamino)phenyl)-N-(4-(2-methyl- | 388 |
| | | | | | 4-pyridinyl)phenyl)propanamide | |
| 256 | 1 | H | CHCH₃ | 4-(4-morpholin-4-ylphenyl)- | N-(4-(2-methyl-4-pyridinyl)phenyl)-2-(4- | 479.1 |
| | | | | pyridin-2-yl | (4-(4-morpholinyl)phenyl)-2-pyridinyl)- | |
| | | | | | propanamide | |
| 257 | 1 | H | CHCH₃ | 2-(4-morpholin-4-ylphenyl)- | N-(4-(2-methyl-4-pyridinyl)phenyl)-2-(2- | 479.1 |
| | | | | pyridin-4-yl | (4-(4-morpholinyl)phenyl)-4-pyridinyl)- | |
| | | | | | propanamide | |
| 258 | 1 | H | S-CH₂cyclopropyl | 3,5-difluorophenyl | (2S)-3-cyclopropyl-2-(3,5-difluorophenyl)- | 393.1 |
| | | | | | N-(4-(2-methyl-4-pyridinyl)phenyl)- | |
| | | | | | propanamide | |
| 259 | 1 | H | R-CH₂cyclopropyl | 3,5-difluorophenyl | (2R)-3-cyclopropyl-2-(3,5-difluorophenyl)- | 393.1 |
| | | | | | N-(4-(2-methyl-4-pyridinyl)phenyl)- | |
| | | | | | propanamide | |
| 260 | 1 | H | CHCH₃ | 3-methyl-5-(2-methyl- | 2-(3-methyl-5-(2-methyl-5-pyrimidinyl)- | 423 |
| | | | | pyrimidin-5-yl)-phenyl | phenyl)-N-(4-(2-methyl-4-pyridinyl)- | |
| | | | | | phenyl)propanamide | |
| 261 | 0 | H | — | 5-cyclopropyl-1H-pyrazol-3-yl | 5-cyclopropyl-N-(4-(2-methyl-4-pyridinyl)- | |
| | | | | | phenyl)-1H-pyrazole-3-carboxamide | |
| 262 | 0 | H | — | 7-methylimidazo[1,2-a]- | 7-methyl-N-(4-(2-methyl-4-pyridinyl)- | |
| | | | | pyridin-2-yl | phenyl)imidazo[1,2-a]pyridine-2- | |
| | | | | | carboxamide | |
| 263 | 0 | H | — | 8-methylimidazo[1,2-a]- | 8-methyl-N-(4-(2-methyl-4-pyridinyl)- | |
| | | | | pyridin-2-yl | phenyl)imidazo[1,2-a]pyridine-2- | |
| | | | | | carboxamide | |
| 264 | 0 | H | — | 3,5-ditrifluoromethylphenyl | N-(4-(2-methyl-4-pyridinyl)phenyl)-3,5- | 289 |
| | | | | | bis(trifluoromethyl)benzamide | |
| 265 | 0 | H | — | phenyl | N-(4-(2-methyl-4-pyridinyl)phenyl)- | 290 |
| | | | | | benzamide | |
| 266 | 0 | H | — | pyridin-3-yl | N-(4-(2-methyl-4-pyridinyl)phenyl)-3- | 329 |
| | | | | | pyridinecarboxamide | |
| 268 | 1 | H | CH₂ | pyridin-3-yl | N-(4-(2-methyl-4-pyridinyl)phenyl)-2-(3- | 304 |
| | | | | | pyridinyl)acetamide | |
| 269 | 0 | H | — | 1-(4-Clphenyl)cycloprop-1-yl | 1-(4-chlorophenyl)-N-(4-(2-methyl-4- | 363.1 |
| | | | | | pyridinyl)phenyl)cyclopropanecarboxamide | |
| 270 | 0 | H | — | 2,4-dichlorophenyl | 2,4-dichloro-N-(4-(2-methyl-4-pyridinyl)- | 357 |
| | | | | | phenyl)benzamide | |
| 271 | 0 | H | — | 2,4-dimethoxyphenyl | 2,4-dimethoxy-N-(4-(2-methyl-4- | 349 |
| | | | | | pyridinyl)-phenyl)benzamide | |
| 272 | 0 | H | — | 2,5-dimethoxyphenyl | 2,5-dimethoxy-N-(4-(2-methyl-4- | 349 |
| | | | | | pyridinyl)phenyl)benzamide | |
| 273 | 0 | H | — | 2-trifluoromethyiphenyl | N-(4-(2-methyl-4-pyridinyl)phenyl)-2- | 357 |
| | | | | | (trifluoromethyl)benzamide | |
| 274 | 0 | H | — | 2,5-dimethylphenyl | 2,5-dimethyl-N-(4-(2-methyl-4-pyridinyl)- | 317 |
| | | | | | phenyl)benzamide | |
| 275 | 0 | H | — | 3-cyanophenyl | 3-cyano-N-(4-(2-methyl-4-pyridinyl)- | 314 |
| | | | | | phenyl)benzamide | |
| 276 | 0 | H | — | 3,4-dichlorophenyl | 3,4-dichloro-N-(4-(2-methyl-4-pyridinyl)- | 357 |
| | | | | | phenyl)benzamide | |
| 277 | 0 | H | — | 3,4-dimethoxyphenyl | 3,4-dimethoxy-N-(4-(2-methyl-4- | 349 |
| | | | | | pyridinyl)-phenyl)benzamide | |
| 278 | 0 | H | — | 3,5-dimethoxyphenyl | 3,5-dimethoxy-N-(4-(2-methyl-4- | 349 |
| | | | | | pyridinyl)-phenyl)benzamide | |
| 279 | 0 | H | — | 3-trifluoromethyiphenyl | N-(4-(2-methyl-4-pyridinyl)phenyl)-3- | 357 |
| | | | | | (trifluoromethyl)benzamide | |
| 280 | 0 | H | — | 4-biphenyl | N-(4-(2-methyl-4-pyridinyl)phenyl)-4- | 365 |
| | | | | | biphenylcarboxamide | |
| 281 | 0 | H | — | 4-trifluoromethylphenyl | N-(4-(2-methyl-4-pyridinyl)phenyl)-4- | 357 |
| | | | | | (trifluoromethyl)benzamide | |
| 282 | 0 | H | — | 3-methoxy-4-methylphenyl | 3-methoxy-4-methyl-N-(4-(2-methyl-4- | 333 |
| | | | | | pyridinyl)phenyl)benzamide | |
| 283 | 0 | H | — | naphth-lyl | N-(4-(2-methyl-4-pyridinyl)phenyl)-1- | 339 |
| | | | | | naphthalenecarboxamide | |
| 284 | 0 | H | — | naphth-2-yl | N-(4-(2-methyl-4-pyridinyl)phenyl)-2- | 339 |
| | | | | | naphthalenecarboxamide | |
| 285 | 1 | H | CH₂ | 3,4-dichlorophenyl | 2-(3,4-dichlorophenyl)-N-(4-(2-methyl-4- | 371 |
| | | | | | pridinyl)phenyl)acetamide | |
| 286 | 0 | H | — | dibenzo[b,d]furan-4-yl | N-(4-(2-methyl-4-pyridinyl)phenyl)- | 379 |
| | | | | | dibenzo[b,dlfuran-4-carboxamide | |
| 287 | 1 | H | C(CH₃)₂ | phenyl | 2-methyl-N-(4-(2-methyl-4-pyridinyl)- | 331.1 |
| | | | | | phenyl)-2-phenylpropanamide | |
| 288 | 0 | H | — | 1-(2-Fphenyl)cycloprop-1-yl | 1-(2-fluorophenyl)-N-(4-(2-methyl-4- | 347 |
| | | | | | pyridinyl)phenypcyclopropanecarboxamide | |
| 289 | 0 | H | — | 1-(4-Fphenyl)cycloprop-1-yl | 1-(4-fluorophenyl)-N-(4-(2-methyl-4- | 347 |
| | | | | | pyridinyl)phenyl)cyclopropanecarboxatnide | |
| 290 | 1 | H | S-CH(CH₃) | 3,5-ditrifluoromethylphenyl | (2S)-2-(3,5-bis(trifluoromethyl)phenyl)-N- | 453 |
| | | | | | (4-(2-methyl-4-pyridinyl)phenyl)- | |
| | | | | | propanamide | |
| 291 | 0 | H | — | 1-(3-Fphenyl)cycloprop-1-yl | 1-(3-fluorophenyl)-N-(4-(2-methyl-4- | 347.1 |
| | | | | | pyridinyl)phenyl)cyclopropanecarboxamide | |
| 292 | 0 | H | — | 1-(phenyl)cyclobut-l-yl | N-(4-(2-methyl-4-pyridinyl)phenyl)-1- | 343.2 |
| | | | | | phenylcyclobutanecarboxamide | |
| 293 | 0 | H | — | 1-(phenyl)cyclohex-1-yl | N-(4-(2-methyl-4-pyridinyl)phenyl)-1- | 371.1 |
| | | | | | phenylcyclohexanecarboxamide | |
| 294 | 1 | H | CH₂ | 3,5-difluorophenyl | 2-(3,5-difluorophenyl)-N-(4-(2-methyl-4- | 339.1 |
| | | | | | pyridinyl)phenyl)acetamide | |
| 295 | 1 | H | CH(CH₃) | 4-chloropyridin-2-yl | 2-(4-chloro-2-pyridinyl)-N-(4-(2-methyl-4- | 352.1 |
| | | | | | pyridinyl)phenyl)propanamide | |
| 296 | 1 | H | R-CH(CH₃) | phenyl | (2R)-N-(4-(2-methyl-4-pyridinyl)phenyl)-2- | 317.1 |
| | | | | | phenylpropanamide | |
| 297 | 1 | H | S-CH(CH₃) | phenyl | (2S)-N-(4-(2-methyl-4-pyridinyl)phenyl)-2- | 317.1 |
| | | | | | phenylpropanamide | |
| 298 | 0 | H | — | 1-(3-F-phenyl)cyclopent-l-yl | 1-(3-fluorophenyl)-N-(4-(2-methyl-4- | 375 |
| | | | | | pyridinyl)phenyl)cyclopentanecarboxamide | |
| 299 | 0 | H | — | 1-(2-phenylethyl)-2- | N-(4-(2-methyl-4-pyridinyl)phenyl)-5-oxo- | 400 |
| | | | | oxopyrrolidin-4-yl | 1-(2-phenylethyl)-3-pyrrolidinecarboxamide | |
| 300 | 0 | H | — | 6-chloro-1H-imidazo[1,2- | 6-chloro-N-(4-(2-methyl-4-pyridinyl)- | 363 |
| | | | | a]pyridin-2-yl | phenyl)imidazo[1,2-a]pyridine-2- | |
| | | | | | carboxamide | |
| 301 | 0 | H | — | 3-methylpyridin-2-yl | 3-methyl-N-(4-(2-methyl-4-pyridinyl)- | 304 |
| | | | | | phenyl)-2-pyridinecarboxamide | |
| 302 | 0 | H | — | 1H-imidazo[1,2-a]pyridin-2-yl | N-(4-(2-methyl-4-pyridinyl)phenyl)- | 329 |
| | | | | | imidazo[1,2-a]pyridine-2-carboxamide | |
| 303 | 1 | H | CH(CH₃) | 3-fluorophenyl | 2-(3-fluorophenyl)-N-(4-(2-methyl-4- | 335.1 |
| | | | | | pyridinyl)phenyl)propanamide | |
| 304 | 1 | H | CH(CH₃) | 2-fluorophenyl | 2-(2-fluorophenyl)-N-(4-(2-methyl-4- | 335.1 |
| | | | | | pyridinyl)phenyl)propanamide | |
| 305 | 0 | H | — | 1-methylindol-2-yl | 1-methyl-N-(4-(2-methyl-4-pyridinyl)- | 342 |
| | | | | | phenyl)-1H-indole-2-carboxamide | |
| 306 | 1 | H | CH₂ | 2-methoxyphenyl | 2-(2-methoxyphenyl)-N-(4-(2-methyl-4- | 333 |
| | | | | | pyridinyl)phenyl)acetamide | |
| 307 | 0 | H | — | 2-chlorophenyl | 2-chloro-N-(4-(2-methyl-4-pyridinyl)- | 323 |
| | | | | | phenyl)benzamide | |
| 308 | 0 | H | — | 1-(4-chlorophenyl)-2- | 1-(4-chlorophenyl)-N-(4-(2-methyl-4- | 406 |
| | | | | oxopyrrolidin-4-yl | pyridinyl)phenyl)-5-oxo-3-pyrrolidine- | |
| | | | | | carboxamide | |
| 309 | 0 | H | — | 1-benzylpyrrolidin-2-yl | 1-benzyl-N-(4-(2-methyl-4-pyridinyl)- | 372 |
| | | | | | phenyl)-L-prolinamide | |
| 310 | 0 | H | — | 1-(4-Cl-phenyl)cyclobut-1-yl | 1-(4-chlorophenyl)-N-(4-(2-methyl-4- | 377 |
| | | | | | pyridinyl)phenyl)cyclobutanecarboxamide | |
| 311 | 0 | H | — | 1-cyclohexylazetidin-2-yl | 1-cyclohexyl-N-(4-(2-methyl-4-pyridinyl)- | 350 |
| | | | | | phenyl)-2-azetidinecarboxamide | |
| 312 | 0 | H | — | 1-benzylpyridin-4-yl | 1-benzyl-N-(4-(2-methyl-4-pyridinyl)- | 386 |
| | | | | | phenyl)-4-piperidinecarboxamide | |
| 313 | 0 | H | — | 1-(3-chlorophenyl)-2- | 1-(3-chlorophenyl)-N-(4-(2-methyl-4- | 406 |
| | | | | oxopyrrolidin-4-yl | pyridinyl)phenyl)-5-oxo-3-pyrrolidine- | |
| | | | | | carboxamide | |
| 314 | 0 | H | — | 6-fluorobenzthiophen-2-yl | 6-fluoro-N-(4-(2-methyl-4-pyridinyl)- | 363 |
| | | | | | phenyl)-1-benzothiophene-2-carboxamide | |
| 315 | 0 | H | — | 6-bromo-1H-imidazo[1,2- | 6-bromo-N-(4-(2-methyl-4-pyridinyl)- | 407 |
| | | | | a]pyridin-2-yl | phenyl)imidazo[1,2-a]pyridine-2- | |
| | | | | | carboxamide | |
| 316 | 0 | H | — | (1R,2R)-2-phenylcyclopropyl | (1R,2R)-N-(4-(2-methyl-4-pyridinyl)- | 329.1 |
| | | | | | phenyl)-2-phenylcyclopropanecarboxamide | |
| 317 | 1 | H | S-CH(CH₃) | 3-chlorophenyl | (2S)-2-(3-chlorophenyl)-N-(4-(2-methyl-4- | 351.1 |
| | | | | | pyridinyl)phenyl)propanamide | |
| 318 | 1 | H | R-CH(CH₃) | 3-chlorophenyl | (2R)-2-(3-chlorophenyl)-N-(4-(2-methyl-4- | 351.1 |
| | | | | | pyridinyl)phenyl)propanamide | |
| 319 | 1 | H | S-CH(CH₃) | 2-fluorophenyl | (2S)-2-(2-fluorophenyl)-N-(4-(2-methyl-4- | 335.1 |
| | | | | | pyridinyl)phenyl)propanamide | |
| 320 | 1 | H | R-CH(CH₃) | 2-fluorophenyl | (2R)-2-(2-fluorophenyl)-N-(4-(2-methyl-4- | 335.1 |
| | | | | | pyridinyl)phenyl)propanamide | |
| 321 | 1 | H | CH₂ | 3-cyanophenyl | 2-(3-cyanophenyl)-N-(4-(2-methyl-4- | 328.1 |
| | | | | | pyridinyl)phenyl)acetamide | |
| 322 | 0 | H | — | 1,2,3,4-tetrahydronaphth-1-yl | N-(4-(2-methyl-4-pyridinyl)phenyl)-1,2,3,4- | 459.1 |
| | | | | | tetrahydro-1-naphthalenecarboxamide | (M + 19) |
| 324 | 0 | H | — | 4H-chromen-4-one-3-yl | N-(4-(2-methyl-4-pyridinyl)phenyl)-4-oxo- | 357 |
| | | | | | 4H-chromene-3-carboxamide | |
| 325 | 1 | H | CH(CH₃) | 3-methylphenyl | 2-(3-methylphenyl)-N-(4-(2-methyl-4- | |
| | | | | | pyridinyl)phenyl)propanamide | |
| 326 | 1 | H | CH(CH₃) | 4-methylphenyl | 2-(4-methylphenyl)-N-(4-(2-methyl-4- | 345.2 |
| | | | | | pyridinyl)phenyl)propanamide | |
| 327 | 1 | H | CH₂ | thien-2-yl | N-(4-(2-methyl-4-pyridinyl)phenyl)-2-(2- | 309 |
| | | | | | thiophenyl)acetamide | |
| 328 | 1 | H | CH₂ | thien-3-yl | N-(4-(2-methyl-4-pyridinyl)phenyl)-2-(2- | 309 |
| | | | | | thiophenyl)acetamide | |
| 329 | 1 | H | CH₂ | 2,5-dimethoxyphenyl | 2-(2,5-dimethoxyphenyl)-N-(4-(2-methyl-4- | 363 |
| | | | | | pyridinyl)phenyl)acetamide | |
| 330 | 1 | H | CH₂ | 2,3-dimethoxyphenyl | 2-(2,3-dimethoxyphenyl)-N-(4-(2-methyl-4- | 363 |
| | | | | | pyridinyl)phenyl)acetamide | |
| 331 | 0 | H | — | 1-(4- | 1-(4-methoxyphenyl)-N-(4-(2-methyl-4- | 359 |
| | | | | methoxyphenyl)cycloprop-1-yl | pyridinyl)phenyl)cyclopropanecarboxamide | |
| 332 | 0 | H | — | 1-(2,4-dichlorophenyl)- | 1-(2,4-dichlorophenyl)-N-(4-(2-methyl-4- | 397 |
| | | | | cycloprop-1-yl | pyridinyl)phenyl)cyclopropanecarboxamide | |
| 333 | 1 | H | CH(CH₃) | 2,3-dichlorophenyl | 2-(2,3-dichlorophenyl)-N-(4-(2-methyl-4- | 385 |
| | | | | | pyridinyl)phenyl)propanamide | |
| 334 | 1 | H | CH(CH₃) | 2,4-dichlorophenyl | 2-(2,4-dichlorophenyl)-N-(4-(2-methyl-4- | 385 |
| | | | | | pyridinyl)phenyl)propanamide | |
| 335 | 1 | H | CH(CH₃) | 2,6-dichlorophenyl | 2-(2,6-dichlorophenyl)-N-(4-(2-methyl-4- | 385 |
| | | | | | pyridinyl)phenyl)propanamide | |
| 336 | 1 | H | C(CH₃)₂ | 3,5-ditrifluoromethylphenyl | 2-(3,5-bis(trifluoromethyl)phenyl)-2- | 467.1 |
| | | | | | methyl-N-(4-(2-methyl-4- | |
| | | | | | pyridinyl)phenyl)propanamide | |
| 337 | 1 | H | CH(CH₂CH₃) | 3-methylphenyl | 2-(3-methylphenyl)-N-(4-(2-methyl-4- | 345.1 |
| | | | | | pyridinyl)phenyl)butanamide | |
| 338 | 1 | H | CH[CH(CH₃)₂] | 3-methylphenyl | 3-methyl-2-(3-methylphenyl)-N-(4-(2- | 359.2 |
| | | | | | methyl-4-pyridinyl)phenyl)butanamide | |
| 339 | 1 | H | R-CH(CH₃) | 3,5-difluorophenyl | (2R)-2-(3,5-difluorophenyl)-N-(4-(2- | 353.1 |
| | | | | | methyl-4-pyridinyl)phenyl)propanamide | |
| 340 | 1 | H | S-CH(CH₃) | 3,5-difluorophenyl | (2S)-2-(3,5-difluorophenyl)-N-(4-(2- | 353.1 |
| | | | | | methyl-4-pyridinyl)phenyl)propanamide | |
| 341 | 1 | H | CH₂ | 4-bromophenyl | 2-(4-bromophenyl)-N-(4-(2-methyl-4- | 381 |
| | | | | | pyridinyl)phenyl)acetamide | |
| 342 | 1 | H | CH₂CH(CH₃)CH₂C | phenyl | 3-methyl-N-(4-(2-methyl-4-pyridinyl)- | 359 |
| | | | H₃ | | phenyl)-2-phenylpentanamide | |
| 343 | 1 | H | CH₂ | 2-bromophenyl | 2-(2-bromophenyl)-N-(4-(2-methyl-4- | 381 |
| | | | | | pyridinyl)phenyl)acetamide | |
| 344 | 1 | H | R-CH(CH₂CH₃) | phenyl | (2R)-N-(4-(2-methyl-4-pyridinyl)phenyl)-2- | 331 |
| | | | | | phenylbutanamide | |
| 345 | 1 | H | S-CH(CH₂CH₃) | phenyl | (2S)-N-(4-(2-methyl-4-pyridinyl)phenyl)-2- | 331 |
| | | | | | phenylbutanamide | |
| 346 | 1 | H | CH₂ | 3-bromophenyl | 2-(3-bromophenyl)-N-(4-(2-methyl-4- | 381 |
| | | | | | pyridinyl)phenyl)acetamide | |
| 347 | 1 | H | CH₂ | 2-iodophenyl | 2-(2-iodophenyl)-N-(4-(2-methyl-4- | 429 |
| | | | | | pyridinyl)phenyl)acetamide | |
| 348 | 1 | H | CH₂ | 2-fluoro-6- | 2-(2-fluoro-6-(trifluoromethyl)phenyl)-N- | 389 |
| | | | | trifluoromethylphenyl | (4-(2-methyl-4-pyridinyl)phenyl)acetamide | |
| 349 | 1 | H | CH₂ | 2,4,6-trimethylphenyl | N-(4-(2-methyl-4-pyridinyl)phenyl)-2- | 345 |
| | | | | | (2,4,6-trimethylphenyl)acetamide | |
| 350 | 1 | 1-1 | CH₂ | 3-iodophenyl | 2-(3-iodomethyl-N-(4-(2-methyl-4- | 429 |
| | | | | | pyridinyl)phenyl)acetamide | |
| 351 | 1 | H | CH₂ | 4-fluoro-3-trifluoromethyl- | 2-(4-fluoro-3-(trifluoromethyl)phenyl)-N- | 389 |
| | | | | phenyl | (4-(2-methyl-4-pyridinyl)phenyl)acetamide | |
| 352 | 1 | H | CH(CH₂CH₃) | phenyl | N-(4-(2-methyl-4-pyridinyl)phenyl)-2- | 331 |
| | | | | | phenylbutanamide | |
| 353 | 1 | H | CH₂ | 2-methylphenyl | 2-(2-methylphenyl)-N-(4-(2-methyl-4- | 317 |
| | | | | | pyridinyl)phenyl)acetamide | |
| 354 | 1 | H | CH₂ | 3-methylphenyl | 2-(3-methylphenyl)-N-(4-(2-methyl-4- | 317 |
| | | | | | pyridinyl)phenyl)acetamide | |
| 355 | 1 | H | CH₂ | 4-methylphenyl | 2-(4-methylphenyl)-N-(4-(2-methyl-4- | 317 |
| | | | | | pyridinyl)phenyl)acetamide | |
| 356 | 1 | H | CH₂ | 2,4-ditrifluoromethylphenyl | 2-(2,4-bis(trifluoromethyl)phenyl)-N-(4-(2- | 439 |
| | | | | | methyl-4-pyridinyl)phenyl)acetamide | |
| 357 | 1 | H | CH₂ | 3-fluoro-4-methoxyphenyl | 2-(3-fluoro-4-methoxyphenyl)-N-(4-(2- | 351 |
| | | | | | methyl-4-pyridinyl)phenyl)acetamide | |
| 358 | 1 | H | CH₂ | 2-chloro-5-fluorophenyl | 2-(2-chloro-5-fluorophenyl)-N-(4-(2- | 355 |
| | | | | | methyl-4-pyridinyl)phenyl)acetamide | |
| 359 | 1 | H | CH₂ | 3-chloro-4-fluorophenyl | 2-(3-chloro-4-fluorophenyl)-N-(4-(2- | 355 |
| | | | | | methyl-4-pyridinyl)phenyl)acetamide | |
| 360 | 1 | H | CH₂ | 2-bromo-5-chlorophenyl | 2-(2-bromo-5-chlorophenyl)-N-(4-(2- | 415 |
| | | | | | methyl-4-pyridinyl)phenyl)acetamide | |
| 361 | 1 | H | CH₂ | 4-iodophenyl | 2-(4-iodophenyl)-N-(4-(2-methyl-4- | 429 |
| | | | | | pyridiny)phenyl)acetamide | |
| 362 | 1 | H | CH₂ | 2-fluoro-5-trifluoromethyl- | 2-(2-fluoro-5-(trifluoromethyl)phenyl)-N- | 389 |
| | | | | phenyl | (4-(2-methyl-4-pyridinyl)phenyl)acetamide | |
| 363 | 1 | H | CH₂ | 3-chloro-5-fluorophenyl | 2-(3-chloro-5-fluorophenyl)-N-(4-(2- | 355 |
| | | | | | methyl-4-pyridinyl)phenyl)acetamide | |
| 364 | 1 | H | CH₂ | 3-benzyloxyphenyl | 2-(3-(benzyloxy)phenyl)-N-(4-(2-methyl-4- | 409 |
| | | | | | pyridinyl)phenyl)acetamide | |
| 365 | 1 | H | CH₂ | 4-n-butoxyphenyl | 2-(4-butoxyphenyl)-N-(4-(2-methyl-4- | 375 |
| | | | | | pyridinyl)phenyl)acetamide | |
| 366 | 1 | H | CH₂ | 2-chloro-4-fluorophenyl | 2-(2-chloro-4-fluorophenyl)-N-(4-(2- | 355 |
| | | | | | methyl-4-pyridinyl)phenyl)acetamide | |
| 367 | 1 | H | CH₂ | 4-tert-butylphenyl | 2-(4-tert-butylphenyl)-N-(4-(2-methyl-4- | 359 |
| | | | | | pyridinyl)phenyl)acetamide | |
| 368 | 1 | H | CH(CH₂)₂CH₃ | phenyl | N-(4-(2-methyl-4-pyridinyl)phenyl)-2- | 345 |
| | | | | | phenylpentanamide | |
| 369 | 1 | H | CH₂ | 2,5-dimethylphenyl | 2-(2,5-dimethylphenyl)-N-(4-(2-methyl-4- | 331 |
| | | | | | pyridinyl)phenyl)acetamide | |
| 370 | 1 | H | CH₂ | 4-isopropylphenyl | 2-(4-(1-methylethyl)phenyl)-N-(4-(2- | 345 |
| | | | | | methyl-4-pyridinyl)phenypacetamide | |
| 371 | 1 | H | CH₂ | 3,5-dimethylphenyl | 2-(3,5-dimethylphenyl)-N-(4-(2-methyl-4- | 331 |
| | | | | | pyridinyl)phenyl)acetamide | |
| 372 | 1 | H | CH₂ | 5-bromo-2-methoxyphenyl | 2-(5-bromo-2-methoxyphenyl)-N-(4-(2- | 411 |
| | | | | | methyl-4-pyridinyl)phenyl)acetamide | |
| 373 | 1 | H | CH₂ | 3-chloro-2-fluorophenyl | 2-(3-chloro-2-fluorophenyl)-N-(4-(2- | 355 |
| | | | | | methyl-4-pyridinyl)phenyl)acetamide | |
| 374 | 1 | H | CH₂ | 2-fluoro-4- | 2-(2-fluoro-4-(trifluoromethyl)phenyl)-N- | 389 |
| | | | | trifluoromethylphenyl | (4-(2-methyl-4-pyridinyl)phenypacetamide | |
| 375 | 1 | H | CH₂ | 3-fluoro-4- | 2-(3-fluoro-4-(trifluoromethyl)phenyl)-N- | 389 |
| | | | | trifluoromethylphenyl | (4-(2-methyl-4-pyridinyl)phenypacetamide | |
| 376 | 1 | H | CH₂ | 2,6-difluoro-3-methylphenyl | 2-(2,6-difluoro-3-methylphenyl)-N-(4-(2- | 353 |
| | | | | | methyl-4-pyridinyl)phenypacetamide | |
| 377 | 1 | H | CH₂ | 3-trifluoromethoxyphenyl | N-(4-(2-methyl-4-pyridinyl)phenyl)-2-(3- | 387 |
| | | | | | (trifluoromethoxy)phenyl)acetamide | |
| 378 | 1 | H | CH₂ | 2-chloro-5- | 2-(2-chloro-5-(trifluoromethyl)phenyl)-N- | 405 |
| | | | | trifluoromethylphenyl | (4-(2-methyl-4-pyridinyl)phenyl)acetamide | |
| 379 | 1 | H | CH[CH₂CH(CH₃)₂] | 3,5-difluorophenyl | 2-(3,5-difluorophenyl)-4-methyl-N-(4-(2- | 395.2 |
| | | | | | methyl-4-pyridinyl)phenyl)pentanamide | |
| 380 | 1 | H | CH[CH₂CH(CH₃)₂] | 3-trifluoromethylphenyl | 4-methyl-N-(4-(2-methyl-4-pyridinyl)- | 427.2 |
| | | | | | phenyl)-2-(3-(trifluoromethyl)phenyl)- | |
| | | | | | pentanamide | |
| 381 | 1 | H | CH[CH₂CH(CH₃)₂] | 3-chlorophenyl | 2-(3-chlorophenyl)-4-methyl-N-(4-(2- | 393.3 |
| | | | | | methyl-4-pyridinyl)phenyl)pentanamide | |
| 382 | 1 | H | R-CH(CH₂)₃CH₃ | 3-methylphenyl | (2R)-2-(3-methylphenyl)-N-(4-(2-methyl-4- | 373.2 |
| | | | | | pyridinyl)phenyl)hexanamide | |
| 383 | 1 | H | S-CH(CH₂)₃CH₃ | 3-methylphenyl | (2S)-2-(3-methylphenyl)-N-(4-(2-methyl-4- | 373.2 |
| | | | | | pyridinyl)phenyl)hexanamide | |
| 384 | 1 | H | R- | 3-methylphenyl | (2R)-4-methyl-2-(3-methylphenyl)-N-(4-(2- | 373.2 |
| | | | CH[CH₂CH(CH₃)₂] | | methyl-4-pyridinyl)phenyl)pentanamide | |
| 385 | 1 | H | S- | 3-methylphenyl | (2S)-4-methyl-2-(3-methylphenyl)-N-(4-(2- | 373.2 |
| | | | CH[CH₂CH(CH₃)₂] | | methyl-4-pyridinyl)phenyl)pentanamide | |
| 386 | 0 | H | — | 3-biphenyl | N-(4-(2-methyl-4-pyridinyl)phenyl)-3- | 365 |
| | | | | | biphenylcarboxamide | |
| 387 | 1 | H | CH₂ | 2-trifluoromethoxyphenyl | N-(4-(2-methyl-4-pyridinyl)phenyl)-2-(2- | 387 |
| | | | | | (trifluoromethoxy)phenyl)acetamide | |
| 388 | 1 | H | CH₂ | 5-fluoro-2- | 2-(5-fluoro-2-(trifluoromethyl)phenyl)-N- | 389 |
| | | | | trifluoromethylphenyl | (4-(2-methyl-4-pyridinyl)phenyl)acetamide | |
| 389 | 1 | H | CH[CH₂CH(CH₃)₂] | 4-bromophenyl | 2-(4-bromophenyl)-4-methyl-N-(4-(2- | 437.1 |
| | | | | | methyl-4-pyridinyl)phenyl)pentanamide | |
| 390 | 1 | H | CH[CH₂CH(CH₃)₂] | 3-bromophenyl | 2-(3-bromophenyl)-4-methyl-N-(4-(2- | 437.1 |
| | | | | | methyl-4-pyridinyl)phenyl)pentanamide | |
| 391 | 1 | H | CH[CH₂CH(CH₃)₂] | 2-bromophenyl | 2-(2-bromophenyl)-4-methyl-N-(4-(2- | 437.1 |
| | | | | | methyl-4-pyridinyl)phenyl)pentanamide | |
| 392 | 1 | H | CH[CH₂CH(CH₃)₂] | 4-biphenyl | 2-(4-biphenylyl)-4-methyl-N-(4-(2-methyl- | 435.2 |
| | | | | | 4-pyridinyl)phenyl)pentanamide | |
| 393 | 1 | H | CH[CH₂CH(CH₃)₂] | 2-biphenyl | 2-(2-biphenylyl)-4-methyl-N-(4-(2-methyl- | 435.2 |
| | | | | | 4-pyridinyl)phenyl)pentanamide | |
| 394 | 1 | H | R-CHCH₃ | 3-trifluoromethylphenyl | (2R)-N-(4-(2-methyl-4-pyridinyl)phenyl)-2- | 385 |
| | | | | | (3-(trifluoromethyl)phenyl)propanamide | |
| 395 | 1 | H | S-CHCH₃ | 3-trifluoromethylphenyl | (2S)-N-(4-(2-methyl-4-pyridinyl)phenyl)-2- | 385 |
| | | | | | (3-(trifluoromethyl)phenyl)propanamide | |
| 396 | 1 | H | CH[CH₂CH(CH₃)₂] | 3-biphenyl | 2-(3-biphenylyl)-4-methyl-N-(4-(2-methyl- | 435.2 |
| | | | | | 4-pyridinyl)phenyl)pentanamide | |
| 397 | 1 | H | CH[CH₂CH(CH₃)₂] | 3-cyanophenyl | 2-(3-cyanophenyl)-4-methyl-N-(4-(2- | 384.2 |
| | | | | | methyl-4-pyridinyl)phenyl)pentanamide | |
| 398 | 1 | H | CHCH₃ | naphth-2-yl | N-(4-(2-methyl-4-pyridinyl)phenyl)-2-(2- | 367.1 |
| | | | | | naphthalenyl)propanamide | |
| 399 | 1 | H | S- | 3,5-difluorophenyl | (2S)-2-(3,5-difluorophenyl)-4-methyl-N-(4- | 395.1 |
| | | | CH[CH₂CH(CH₃)₂] | | (2-methyl-4-pyridinyl)phenyl)pentanamide | |
| 400 | 1 | H | R- | 3,5-difluorophenyl | (2R)-2-(3,5-difluorophenyl)-4-methyl-N-(4- | 395.1 |
| | | | CH[CH₂CH(CH₃)₂] | | (2-methyl-4-pyridinyl)phenyl)pentanamide | |
| 402 | 1 | H | CHCH₃ | 3,5-dichlorophenyl | 2-(3,5-dichlorophenyl)-N-(4-(2-methyl-4- | 385.1 |
| | | | | | pyridinyl)phenyl)propanamide | |
| 403 | 1 | H | CH[CH₂CH(CH₃)₂] | 3,5-dichlorophenyl | 2-(3,5-dichlorophenyl)-4-methyl-N-(4-(2- | 427.1 |
| | | | | | methyl-4-pyridinyl)phenyl)pentanamide | |
| 404 | 1 | H | CHCH₃ | dibenzo[b,d]thiophen-4-yl | 2-dibenzo[b,d]thiophen-4-yl-N-(4-(2- | 423 |
| | | | | | methyl-4-pyridinyl)phenyl)propanamide | |
| 405 | 1 | H | CHCH₃ | 9H-carbazol-1-yl | 2-(9H-carbazol-2-yl)-N-(4-(2-methyl-4- | 406 |
| | | | | | pyridinyl)phenyl)propanamide | |
| 406 | 1 | H | R- | 3-bromophenyl | (2R)-2-(3-bromophenyl)-4-methyl-N-(4-(2- | 438 |
| | | | CH[CH₂CH(CH₃)₂] | | methyl-4-pyridinyl)phenyl)pentanamide | |
| 407 | 1 | H | S- | 3-bromophenyl | (2S)-2-(3-bromophenyl)-4-methyl-N-(4-(2- | 438 |
| | | | CH[CH₂CH(CH₃)₂] | | methyl-4-pyridinyl)phenyl)pentanamide | |
| 408 | 1 | H | R- | 4-bromophenyl | (2R)-2-(4-bromophenyl)-4-methyl-N-(4-(2- | 437.1 |
| | | | CH[CH₂CH(CH₃)₂] | | methyl-4-pyridinyl)phenyl)pentanamide | |
| 409 | 1 | H | S- | 4-bromophenyl | (2S)-2-(4-bromophenyl)-4-methyl-N-(4-(2- | 437.1 |
| | | | CH[CH₂CH(CH₃)₂] | | methyl-4-pyridinyl)phenyl)pentanamide | |
| 410 | 1 | H | CHCH₃ | 3-bromophenyl | 2-(3-bromophenyl)-N-(4-(2-methyl-4- | 395.1 |
| | | | | | pyridinyl)phenyl)propanamide | |
| 411 | 1 | H | CHCH₂CH₃ | 3-bromophenyl | 2-(3-bromophenyl)-N-(4-(2-methyl-4- | 409.1 |
| | | | | | pyridinyl)phenyl)butanamide | |
| 412 | 1 | H | CH(CH₂)₂CH₃ | 3-bromophenyl | 2-(3-bromophenyl)-N-(4-(2-methyl-4- | 423.1 |
| | | | | | pyridinyl)phenyl)pentanamide | |
| 413 | 1 | H | CHCH₃ | naphth-1-yl | N-(4-(2-methyl-4-pyridinyl)phenyl)-2-(1- | 367.1 |
| | | | | | naphthalenyl)propanamide | |
| 414 | 1 | H | CHCH₂CH₃ | indol-1-yl | 2-(1H-indol-1-yl)-N-(4-(2-methyl-4- | 356.1 |
| | | | | | pyridinyl)phenyl)propanamide | |
| 415 | 1 | H | R- | 3-cyanophenyl | (2R)-2-(3-cyanophenyl)-4-methyl-N-(4-(2- | 384.2 |
| | | | CH[CH₂CH(CH₃)₂] | | methyl-4-pyridinyl)phenyl)pentanamide | |
| 416 | 1 | H | S- | 3-cyanophenyl | (2S)-2-(3-cyanophenyl)-4-methyl-N-(4-(2- | 384.2 |
| | | | CH[CH₂CH(CH₃)₂] | | methyl-4-pyridinyl)phenyl)pentanamide | |
| 417 | 1 | H | CHCH₃ | 3-(4-morpholin-4- | N-(4-(2-methyl-4-pyridinyl)phenyl)-2-(4′- | 478.2 |
| | | | | ylphenyl)phenyl | (4-morpholinyl)-3-biphenylyl)propanamide | |
| 418 | 1 | H | CHCH₃ | 6-chloro-9-methyl-9H- | 2-(6-chloro-9-methyl-9H-carbazol-2-yl)-N- | 454.1 |
| | | | | carbazol-2-yl | (4-(2-methyl-4-pyridinyl)phenyl)- | |
| | | | | | propanamide | |
| 419 | 1 | H | CHCH₃ | 4-aminophenyl | 2-(4-aminophenyl)-N-(4-(2-methyl-4- | 332 |
| | | | | | pyridinyl)phenyl)propanamide | |
| 420 | 1 | H | CH[CH₂CH(CH₃)₂] | 4-phenoxyphenyl | 4-methyl-N-(4-(2-methyl-4-pyridinyl)- | 451 |
| | | | | | phenyl)-2-(4-phenoxyphenyl)pentanamide | |
| 421 | 1 | H | CH[CH₂CH(CH₃)₂] | 3-phenoxyphenyl | 4-methyl-N-(4-(2-methyl-4-pyridinyl)- | 451 |
| | | | | | phenyl)-2-(3-phenoxyphenyl)pentanamide | |
| 422 | 1 | H | CHCH₃ | 3,4-ethylenedioxyphenyl | 2-(2,3-dihydro-1,4-benzodioxin-6-yl)-N-(4- | 375.1 |
| | | | | | (2-methyl-4-pyridinyl)phenyl)propanamide | |
| 423 | 1 | H | CH[CH₂CH(CH₃)₂] | 3,4-ethylenedioxyphenyl | 2-(2,3-dihydro-1,4-benzodioxin-6-yl)-4- | 356.1 |
| | | | | | methyl-N-(4-(2-methyl-4-pyridinyl)phenyl)- | |
| | | | | | pentanamide | |
| 424 | 1 | H | CH₂ | 9-methyl-9H-carbazol-3-yl | 2-(9-methyl-9H-carbazol-3-yl)-N-(4-(2- | 406.2 |
| | | | | | methyl-4-pyridinyl)phenyl)acetamide | |
| 425 | 1 | H | CHCH₃ | 3-(1,2,3,6-tetrahydropyridin-4- | N-(4-(2-methyl-4-pyridinyl)phenyl)-2-(3- | 398 |
| | | | | yl)phenyl | (1,2,3,6-tetrahydro-4-pyridinyl)phenyl)- | |
| | | | | | propanamide | |
| 426 | 1 | H | CH[CH₂CH(CH₃)₂] | 4-iodophenyl | 2-(4-iodophenyl)-4-methyl-N-(4-(2-methyl- | 485 |
| | | | | | 4-pyridinyl)phenyl)pentanamide | |
| 427 | 1 | H | CHCH₃ | 3-(4-phenylpiperazin-1- | N-(4-(2-methyl-4-pyridinyl)phenyl)-2-(3- | 477.2 |
| | | | | yl)phenyl | (4-phenyl-1-piperazinyl)phenyl)- | |
| | | | | | propanamide | |
| 428 | 1 | H | CHCH₃ | 3-(piperidin-4-yl)phenyl | N-(4-(2-methyl-4-pyridinyl)phenyl)-2-(3- | 400 |
| | | | | | (4-piperidinyl)phenyl)propanamide | |
| 429 | 1 | H | CHCH₃ | 3-(morpholin-4-yl)phenyl | N-(4-(2-methyl-4-pyridinyl)phenyl)-2-(3- | 402 |
| | | | | | (4-morpholinyl)phenyl)propanamide | |
| 430 | 1 | H | CH₂ | 3-fluoro-2-trifluoromethyl- | 2-(3-fluoro-5-(trifluoromethyl)phenyl)-N- | 389 |
| | | | | phenyl | (4-(2-methyl-4-pyridinyl)phenyl)acetamide | |
| 431 | 1 | H | CHCH₃ | 3-fluoro-5-trifluoromethyl- | 2-(3-fluoro-5-(trifluoromethyl)phenyl)-N- | 403 |
| | | | | phenyl | (4-(2-methyl-4-pyridinyl)phenyl)- | |
| | | | | | propanamide | |
| 432 | 1 | 2-F | CHCH₃ | 3-bromophenyl | 2-(3-bromophenyl)-N-(3-fluoro-4-(2- | 415 |
| | | | | | methyl-4-pyridinyl)phenyl)propanamide | |
| 433 | 1 | 3-F | CHCH₃ | 3-bromophenyl | 2-(3-bromophenyl)-N-(2-fluoro-4-(2- | 415 |
| | | | | | methyl-4-pyridinyl)phenyl)propanamide | |
| 434 | 1 | H | CHCH₃ | 6-fluoropyridin-2-yl | 2-(6-fluoro-2-pyridinyl)-N-(4-(2-methyl-4- | 336 |
| | | | | | pyridinyl)phenyl)propanamide | |
| 435 | 1 | H | CHCH₃ | 3-(2-dimethylaminophenyl)- | 2-(4-(dimethylamino)-3-biphenylyl)-N-(4- | 436.2 |
| | | | | phenyl | (2-methyl-4-pyridinyl)phenyl)propanamide | |
| 436 | 1 | H | CHCH₃ | 3-iodophenyl | 2-(3-iodophenyl)-N-(4-(2-methyl-4- | 443 |
| | | | | | pyridinyl)phenyl)propanamide | |
| 437 | 1 | H | CHCH₃ | 4-fluoro-3-trifluoromethyl- | 2-(4-fluoro-3-(trifluoromethyl)phenyl)-N- | 403 |
| | | | | phenyl | (4-(2-methyl-4-pyridinyl)phenyl)- | |
| | | | | | propanamide | |
| 438 | 1 | H | CHCH₃ | 4-dimethylaminophenyl | 2-(4-(dimethylamino)phenyl)-N-(4-(2- | 360 |
| | | | | | methyl-4-pyridinyl)phenyl)propanamide | |
| 439 | 1 | H | CHCH₃ | 3-chloro-2-fluorophenyl | 2-(3-chloro-2-fluorophenyl)-N-(4-(2- | 369.1 |
| | | | | | methyl-4-pyridinyl)phenyl)propanamide | |
| 440 | 1 | H | CHCH₃ | 5-chloro-2-fluorophenyl | 2-(5-chloro-2-fluorophenyl)-N-(4-(2- | 369.1 |
| | | | | | methyl-4-pyridinyl)phenyl)propanamide | |
| 441 | 1 | H | CHCH₃ | 3-molpholin-4-yl-5- | N-(4-(2-methyl-4-pyridinyl)phenyl)-2-(3- | 470 |
| | | | | trifluoromethylphenyl | (4-morpholinyl)-5-(trifluoromethyl)phenyl)- | |
| | | | | | propanamide | |
| 442 | 1 | H | CHCH₃ | 3-(4-methylsulfonylphenyl)- | N-(4-(2-methyl-4-pyridinyl)phenyl)-2-(4′- | 471.1 |
| | | | | phenyl | (methylsulfonyl)-3-biphenylyl)propanamide | |
| 443 | 1 | H | CHCH₃ | 4-isopropylaminophenyl | 2-(4-((1-methylethypamino)phenyl)-N-(4- | 374 |
| | | | | | (2-methyl-4-pyridinyl)phenyl)propanamide | |
| 444 | 1 | 3- | CHCH₃ | 2,5-ditrifluoromethylphenyl | 2-(3,5-bis(trifluoromethyl)phenyl)-N-(3- | 483 |
| | | OCH₃ | | | methoxy-4-(2-methyl-4-pyridinyl)phenyl)- | |
| | | | | | propanamide | |
| 445 | 1 | H | CHCH₃ | 4-phenylimidazol-1-yl | N-(4-(2-methyl-4-pyridinyl)phenyl)-2-(4- | 483 |
| | | | | | phenyl-1H-imidazol-1-yl)propanamide | |
| 446 | 1 | H | CHCH₃ | 3-benzyloxyphenyl | 2-(3-(benzyloxy)phenyl)-N-(4-(2-methyl-4- | 423.1 |
| | | | | | pyridinyl)phenyl)propanamide | |
| 447 | 1 | H | CHCH₃ | 3-methoxyphenyl | 2-(3-methoxyphenyl)-N-(4-(2-methyl-4- | 347.1 |
| | | | | | pyridinyl)phenyl)propanamide | |
| 448 | 1 | H | CHCH₃ | 3-fluoro-4-methoxyphenyl | 2-(3-fluoro-4-methoxyphenyl)-N-(4-(2- | 365.1 |
| | | | | | methyl-4-pyridinyl)phenyl)propanamide | |
| 449 | 1 | H | CHCH₃ | 3-(3-cyanophenyl)phenyl | 2-(3′-cyano-3-biphenylyl)-N-(4-(2-methyl- | 418.1 |
| | | | | | 4-pyridinyl)phenyl)propanamide | |
| 450 | 1 | H | CHCH₃ | 3-(2-cyanophenyl)phenyl | 2-(2′-cyano-3-biphenylyl)-N-(4-(2-methyl- | 418.1 |
| | | | | | 4-pyridinyl)phenyl)propanamide | |
| 451 | 1 | H | CHCH₃ | 3-(4-methoxyphenyl)phenyl | 2-(4′-methoxy-3-biphenylyl)-N-(4-(2- | 423.1 |
| | | | | | methyl-4-pyridinyl)phenyl)propanamide | |
| 452 | 1 | H | CHCH₃ | 3-(2-methoxyphenyl)phenyl | 2-(2′-methoxy-3-biphenylyl)-N-(4-(2- | 423.1 |
| | | | | | methyl-4-pyridinyl)phenyl)propanamide | |
| 453 | 1 | H | CHCH₃ | 3-(4-trifluoromethoxyphenyl)- | N-(4-(2-methyl-4-pyridinyl)phenyl)-2-(4′- | 477.1 |
| | | | | phenyl | (trifluoromethoxy)-3-biphenylyl)- | |
| | | | | | propanamide | |
| 454 | 1 | 3- | CHCH₃ | 3-fluoro-5- | 2-(3-fluoro-5-(trifluoromethyl)phenyl)-N- | 433 |
| | | OCH₃ | | trifluoromethylphenyl | (3-methoxy-4-(2-methyl-4-pyridinyl)- | |
| | | | | | phenyl)propanamide | |
| 455 | 1 | H | CHCH₃ | 3-methyl-4-phenylpyrazol-1-yl | 2-(3-methyl-4-phenyl-1H-pyrazol-1-yl)-N- | 397 |
| | | | | | (4-(2-methyl-4-pyridinyl)phenyl)- | |
| | | | | | propanamide | |
| 456 | 1 | H | CHCH₃ | 4-(4-bromophenyl)pyrazol-1- | 2-(4-(4-bromophenyl)-1H-pyrazol-1-yl)-N- | 461 |
| | | | | yl | (4-(2-methyl-4-pyridinyl)phenyl)- | |
| | | | | | propanamide | |
| 457 | 1 | H | CHCH₃ | 3-chloro-5-fluorophenyl | 2-(3-chloro-5-fluorophenyl)-N-(4-(2- | 369.1 |
| | | | | | methyl-4-pyridinyl)phenyl)propanamide | |
| 458 | 1 | H | CHCH₃ | 3-bromo-4-fluorophenyl | 2-(3-bromo-4-fluorophenyl)-N-(4-(2- | 413 |
| | | | | | methyl-4-pyridinyl)phenyl)propanamide | |
| 459 | 1 | 3- | CHCH₃ | 3-piperidin-1-ylphenyl | N-(3-methoxy-4-(2-methyl-4-pyridinyl)- | 430 |
| | | OCH₃ | | | phenyl)-2-(3-(1-piperidinyl)phenyl)- | |
| | | | | | propanamide | |
| 460 | 1 | 3-CN | CHCH₃ | 3-bromophenyl | 2-(3-bromophenyl)-N-(3-cyano-4-(2- | 422 |
| | | | | | methyl-4-pyridinyl)phenyl)propanamide | |
| 461 | 1 | 3- | R-CHCH₃ | 3-bromophenyl | (2R)-2-(3-bromophenyl)-N-(3-methoxy-4- | 427 |
| | | OCH₃ | | | (2-methyl-4-pyridinyl)phenyl)propanamide | |
| 462 | 1 | 3- | S-CHCH₃ | 3-bromophenyl | (2S)-2-(3-bromophenyl)-N-(3-methoxy-4- | 427 |
| | | OCH₃ | | | (2-methyl-4-pyridinyl)phenyl)propanamide | |
| 463 | 1 | H | CHCH₃ | 3-bromo-5-fluorophenyl | 2-(3-bromo-5-fluorophenyl)-N-(4-(2- | 414 |
| | | | | | methyl-4-pyridinyl)phenyl)propanamide | |
| 464 | 1 | H | R-CHCH₃ | 3-fluoro-5- | (2R)-2-(3-fluoro-5-trifluoromethyl)phenyl)- | 403 |
| | | | | trifluoromethylphenyl | N-(4-(2-methyl-4-pyridinyl)phenyl)- | |
| | | | | | propanamide | |
| 465 | 1 | H | S-CHCH₃ | 3-fluoro-5- | (2S)-2-(3-fluoro-5-(trifluoromethyl)phenyl)- | 403 |
| | | | | trifluoromethylphenyl | N-(4-(2-methyl-4-pyridinyl)phenyl)- | |
| | | | | | propanamide | |
| 467 | 1 | H | CHCH₃ | 1-methylindol-3-yl | 2-(1-methyl-1H-indol-3-yl)-N-(4-(2-methyl- | 370.1 |
| | | | | | 4-pyridinyl)phenyl)propanamide | |
| 468 | 1 | H | CH₂ | 2-chloropyridin-4-yl | 2-(2-chloro-4-pyridinyl)-N-(4-(2-methyl-4- | 338.1 |
| | | | | | pyridinyl)phenyl)acetamide | |
| 469 | 1 | 11 | CHCH₃ | 2-chloropyridin-4-yl | 2-(2-chloro-4-pyridinyl)-N-(4-(2-methyl-4- | 352.1 |
| | | | | | pyridinyl)phenyl)propanamide | |
| 470 | 1 | H | CHCH₃ | 4-fluoro-3-(2-methoxy- | 2-(4-fluoro-3-((2-methoxyethyl)amino)- | 408.2 |
| | | | | ethylamino)phenyl | phenyl)-N-(4-(2-methyl-4-pyridinyl)- | |
| | | | | | phenyl)propanamide | |
| 471 | 1 | H | CH₂ | 3-cyano-5- | 2-(3-cyano-5-(trifluoromethyl)phenyl)-N- | 396.1 |
| | | | | trifluoromethylphenyl | (4-(2-methyl-4-pyridinyl)phenyl)acetamide | |
| 472 | 1 | H | CHCH₃ | 3-bromo-5- | 2-(3-bromo-5-(trifluoromethyl)phenyl)-N- | 465 |
| | | | | trifluoromethylphenyl | (4-(2-methyl-4-pyridinyl)phenyl)- | |
| | | | | | propanamide | |
| 473 | 1 | il | CH₂ | 3-bromo-5-trifluoromethyl- | 2-(3-bromo-5-(trifluoromethyl)phenyl)-N- | 451 |
| | | | | phenyl | (4-(2-methyl-4-pyridinyl)phenyl)acetamide | |
| 474 | 1 | H | S-CHCH₃ | 3,5-dichlorophenyl | (2S)-2-(3,5-dichlorophenyl)-N-(4-(2- | 385 |
| | | | | | methyl-4-pyridinyl)phenyl)propanamide | |
| 475 | 1 | H | R-CHCH₃ | 3,5-dichlorophenyl | (2R)-2-(3,5-dichlorophenyl)-N-(4-(2- | 385 |
| | | | | | methyl-4-pyridinyl)phenyl)propanamide | |
| 476 | 1 | H | S- | 3,5-dichlorophenyl | (2S)-2-(3,5-dichlorophenyl)-4-methyl-N-(4- | 427.1 |
| | | | CH[CH₂CH(CH₃)₂] | | (2-methyl-4-pyridinyl)phenyl)pentanamide | |
| 477 | 1 | H | R- | 3,5-dichlorophenyl | (2R)-2-(3,5-dichlorophenyl)-4-methyl-N-(4- | 427.1 |
| | | | CH[CH₂CH(CH₃)₂] | | (2-methyl-4-pyridinyl)phenyl)pentanamide | |
| 478 | 1 | H | R-CHCH₃ | 3-bromophenyl | (2R)-2-(3-bromophenyl)-N-(4-(2-methyl-4- | 395 |
| | | | | | pyridinyl)phenyl)propanamide | |
| 479 | 1 | H | S-CHCH₃ | 3-bromophenyl | (2S)-2-(3-bromophenyl)-N-(4-(2-methyl-4- | 395 |
| | | | | | pyridinyl)phenyl)propanamide | |
| 480 | 1 | 3-CH₃ | CHCH₃ | 3-(4-morpholin-4-ylphenyl)- | N-(3-methyl-4-(2-methyl-4-pyridinyl)- | 492 |
| | | | | phenyl | phenyl)-2-(4′-(4-morpholinyl)-3- | |
| | | | | | biphenylyl)propanamide | |
| 481 | 1 | H | CHCH₃ | 4- | 2-(4-(methyl(1-methylethyl)amino)phenyl)- | 388 |
| | | | | isopropylmethylaminophenyl | N-(4-(2-methyl-4-pyridinyl)phenyl)- | |
| | | | | | propanamide | |
| 482 | 1 | H | CHCH₃ | (9-methyl-9H-carbazol-3-yl | 2-(9-methyl-9H-carbazol-3-yl)-N-(4-(2- | 420 |
| | | | | | methyl-4-pyridinyl)phenyl)propanamide | |
| 483 | 1 | 3- | CHCH₃ | (9-methyl-9H-carbazol-3-yl | N-(3-methoxy-4-(2-methyl-4-pyridinyl)- | 450 |
| | | OCH₃ | | | phenyl)-2-(9-methyl-9H-carbazol-3-yl)- | |
| | | | | | propanamide | |
| 484 | 1 | H | CHCH₃ | 2-trifluoromethoxyphenyl | N-(4-(2-methyl-4-pyridinyl)phenyl)-2-(2- | 401.1 |
| | | | | | (trifluoromethoxy)phenyl)propanamide | |
| 485 | 1 | H | CHCH₃ | 3-trifluoromethoxyphenyl | N-(4-(2-methyl-4-pyridinyl)phenyl)-2-(3- | 401.1 |
| | | | | | (trifluoromethoxy)phenyl)propanamide | |
| 486 | 1 | H | CHCH₃ | 4-trifluoromethoxyphenyl | N-(4-(2-methyl-4-pyridinyl)phenyl)-2-(4- | 401.1 |
| | | | | | (trifluoromethoxy)phenyl)propanamide | |
| 487 | 1 | H | CHCH₃ | indol-5-yl | 2-(1H-indol-5-yl)-N-(4-(2-methyl-4- | 356.1 |
| | | | | | pyridinyl)phenyl)propanamide | |
| 488 | 1 | H | R-CHCH₃ | 3-methyl-4-phenylpyrazol-1-yl | (2R)-2-(3-methyl-4-phenyl-1H-pyrazol-1- | 397 |
| | | | | | yl)-N-(4-(2-methyl-4-pyridinyl)phenyl)- | |
| | | | | | propanamide | |
| 489 | 1 | H | S-CHCH₃ | 3-methyl-4-phenylpyrazol-1-yl | (2S)-2-(3-methyl-4-phenyl-1H-pyrazol-1- | 397 |
| | | | | | yl)-N-(4-(2-methyl-4-pyridinyl)phenyl)- | |
| | | | | | propanamide | |
| 490 | 1 | 3- | R-CHCH₃ | 3-bromo-5-fluorophenyl | (2R)-2-(3-bromo-5-fluorophenyl)-N-(3- | 443 |
| | | OCH₃ | | | methoxy-4-(2-methyl-4-pyridinyl)phenyl)- | |
| | | | | | propanamide | |
| 491 | 1 | H | S-CHCH₃ | 4-isopropylaminophenyl | (2S)-2-(4-((1-methylethyl)amino)phenyl)- | 374 |
| | | | | | N-(4-(2-methyl-4-pyridinyl)phenyl)- | |
| | | | | | propanamide | |
| 492 | 1 | H | R-CHCH₃ | 4-isopropylaminophenyl | (2R)-2-(4-((1-methylethyl)amino)phenyl)- | 374 |
| | | | | | N-(4-(2-methyl-4-pyridinyl)phenyl)- | |
| | | | | | propanamide | |
| 493 | 1 | 3- | CHCH₃ | 4-aminophenyl | 2-(4-aminophenyl)-N-(3-methoxy-4-(2- | 362 |
| | | OCH₃ | | | methyl-4-pyridinyl)phenyl)propanamide | |
| 494 | 1 | 3- | S-CHCH₃ | 3,5-ditrifluoromethylphenyl | (2S)-2-(3,5-bis(trifluoromethyl)phenyl)-N- | 483 |
| | | OCH₃ | | | (3-methoxy-4-(2-methyl-4-pyridinyl)- | |
| | | | | | phenyl)propanamide | |
| 495 | 1 | 3- | R-CHCH₃ | 3,5-ditrifluoromethylphenyl | (2R)-2-(3,5-bis(trifluoromethyl)phenyl)-N- | 483 |
| | | OCH₃ | | | (3-methoxy-4-(2-methyl-4-pyridinyl)- | |
| | | | | | phenyl)propanamide | |
| 496 | 1 | H | CH(CH₂)₃CH₃ | 4-bromoimidazol-1-yl | 2-(4-bromo-1H-imidazol-1-yl)-N-(4-(2- | 429 |
| | | | | | methyl-4-pyridinyl)phenyl)hexanamide | |
| 497 | 1 | H | CHCH₃ | 1,2,3,4-tetrahydroindol-5-yl | 2-(2,3-dihydro-1H-indol-5-yl)-N-(4-(2- | 358.2 |
| | | | | | methyl-4-pyridinyl)phenyl)propanamide | |
| 498 | 1 | H | S-CHCH₃ | 4-chlorophenyl | (2S)-2-(4-chlorophenyl)-N-(4-(2-methyl-4- | 351.1 |
| | | | | | pyridinyl)phenyl)propanamide | |
| 499 | 1 | H | R-CHCH₃ | 4-chlorophenyl | (2R)-2-(4-chlorophenyl)-N-(4-(2-methyl-4- | 351.1 |
| | | | | | pyridinyl)phenyl)propanamide | |
| 500 | 1 | H | C(CH₃)₂ | 4-trifluoromethylphenyl | 2-methyl-N-(4-(2-methyl-4-pyridinyl)- | 398.8 |
| | | | | | phenyl)-2-(4-(trifluoromethyl)phenyl)- | |
| | | | | | propanamide | |
| 501 | 1 | H | R-CHCH₃ | 3-pyrimidin-5-ylphenyl | (2R)-N-(4-(2-methyl-4-pyridinyl)phenyl)-2- | 395.1 |
| | | | | | (3-(5-pyrimidinyl)phenyl)propanamide | |
| 502 | 1 | H | R-CHCH₃ | 3-(2-methoxypyrimidin-5- | (2R)-2-(3-(2-methoxy-5-pyrimidinyl)- | 425.1 |
| | | | | yl)phenyl | phenyl)-N-(4-(2-methyl-4-pyridinyl)- | |
| | | | | | phenyl)propanamide | |
| 503 | 1 | H | R-CHCH₃ | 3-(methylthiopyrimidin-5- | (2R)-N-(4-(2-methyl-4-pyridinyl)phenyl)-2- | 441.1 |
| | | | | yl)phenyl | (3-(2-(methylsulfanyl)-5-pyrimidinyl)- | |
| | | | | | phenyl)propanamide | |
| 504 | 1 | H | R-CHCH₃ | 3-(2-aminopyrimidin-5- | (2R)-2-(3-(2-amino-5-pyrimidinyl)phenyl)- | 410.1 |
| | | | | yl)phenyl | N-(4-(2-methyl-4-pyridinyl)phenyl)- | |
| | | | | | propanamide | |
| 505 | 1 | H | R-CHCH₃ | 3-(1-methylindol-5-yl)phenyl | (2R)-2-(3-(1-methyl-1H-indol-5-yl)phenyl)- | 446.2 |
| | | | | | N-(4-(2-methyl-4-pyridinyl)phenyl)- | |
| | | | | | propanamide | |
| 506 | 1 | H | CHCH₃ | 4-trifluoromethylphenyl | N-(4-(2-methyl-4-pyridinyl)phenyl)-2-(4- | 384.8 |
| | | | | | (trifluoromethyl)phenyl)propanamide | |
| 507 | 1 | 3- | CHCH₃ | 4-(cyclopentylamino)phenyl | 2-(4-(cyclopentylamino)phenyl)-N-(3- | 430 |
| | | OCH₃ | | | methoxy-4-(2-methyl-4-pyridinyl)phenyl)- | |
| | | | | | propanamide | |
| 508 | 1 | 3- | CHCH₃ | 4-isopropylaminophenyl | N-(3-methoxy-4-(2-methyl-4-pyridinyl)- | 404 |
| | | OCH₃ | | | phenyl)-2-(4-((1- | |
| | | | | | methylethypamino)phenyl)propanamide | |
| 509 | 1 | 3- | CHCH₃ | 4-cyclobutylaminophenyl | 2-(4-(cyclobutylamino)phenyl)-N-(3- | 416 |
| | | OCH₃ | | | methoxy-4-(2-methyl-4-pyridinyl)- | |
| | | | | | phenyl)propanamide | |
| 510 | 1 | H | CH₂ | 3R,5S-(5-phenyl)piperidin-3-yl | N-(4-(2-methyl-4-pyridinyl)phenyl)-2- | 386 |
| | | | | | ((3R,5S)-5-phenyl-3-piperidinyl)acetamide | |
| 511 | 1 | H | S-CHCH₃ | 3-chloro-5-fluorophenyl | (2S)-2-(3-chloro-5-fluorophenyl)-N-(4-(2- | 369.1 |
| | | | | | methyl-4-pyridinyl)phenyl)propanamide | |
| 512 | 1 | H | R-CHCH₃ | 3-chloro-5-fluorophenyl | (2R)-2-(3-chloro-5-fluorophenyl)-N-(4-(2- | 369.1 |
| | | | | | methyl-4-pyridinyl)phenyl)propanamide | |
| 513 | 1 | H | S-CHCH₃ | 4-phenylimidazol-1-yl | (2S)-N-(4-(2-methyl-4-pyridinyl)phenyl)-2- | 383 |
| | | | | | (4-phenyl-1H-imidazol-1-yl)propanamide | |
| 514 | 1 | H | R-CHCH₃ | 4-phenylimidazol-1-yl | (2R)-N-(4-(2-methyl-4-pyridinyl)phenyl)-2- | 383 |
| | | | | | (4-phenyl-1H-imidazol-1-yl)propanamide | |
| 515 | 1 | 3- | CHCH₃ | 4-N,N-diethylaminophenyl | 2-(4-(diethylamino)phenyl)-N-(3-methoxy- | 418 |
| | | OCH₃ | | | 4-(2-methyl-4-pyridinyl)phenyl)- | |
| | | | | | propanamide | |
| 516 | 1 | H | S-CHCH₃ | 3-(2-methoxypyrimidin-5- | (2S)-2-(3-(2-methoxy-5-pyrimidinyl)- | 425.1 |
| | | | | yl)phenyl | phenyl)-N-(4-(2-methyl-4-pyridinyl)- | |
| | | | | | phenyl)propanamide | |
| 517 | 1 | H | CHCH₃ | 3-(2-methylpyridin-4- | 2-(3-(2-methyl-4-pyridinyl)phenyl)-N-(4- | 408.2 |
| | | | | yl)phenyl | (2-methyl-4-pyridinyl)phenyl)propanamide | |
| 518 | 1 | H | CHCH₃ | 4-cyclobutylaminophenyl | 2-(4-(cyclobutylamino)phenyl)-N-(4-(2- | 386 |
| | | | | | methyl-4-pyridinyl)phenyl)propanamide | |
| 519 | 1 | H | CHCH₃ | 4-(3,4,5-dimethoxybenzyl- | N-(4-(2-methyl-4-pyridinyl)phenyl)-2-(4- | 512 |
| | | | | amino)phenyl | ((3,4,5-trimethoxybenzypamino)- | |
| | | | | | phenyl)propanamide | |
| 521 | 1 | H | R-CHCH₃ | 3-(4-piperazin-1-ylphenyl)- | (2R)-N-(4-(2-methyl-4-pyridinyl)phenyl)-2- | 477.2 |
| | | | | phenyl | (4′-(1-piperazinyl)-3-biphenylyl)- | |
| | | | | | propanamide | |
| 522 | 1 | H | R-CHCH₃ | 3-(4-(4-acetylpiperazin-1-yl)- | (2R)-2-(4′-(4-acetyl-1-piperazinyl)-3- | 519.2 |
| | | | | phenyl)-phenyl | biphenylyl)-N-(4-(2-methyl-4-pyridinyl)- | |
| | | | | | phenyl)propanamide | |
| 523 | 1 | H | CH₂ | 4-cyanophenyl | 2-(4-cyanophenyl)-N-(4-(2-methyl-4- | 328 |
| | | | | | pyridinyl)phenyl)acetamide | |
| 524 | 1 | H | CHCH₃ | 3-cyclopropylphenyl | 2-(3-cyclopropylphenyl)-N-(4-(2-methyl-4- | 357.2 |
| | | | | | pyridinyl)phenyl)propanamide | |
| 525 | 1 | 3- | CHCH₃ | 4-(ethylamino)phenyl | 2-(4-(ethylamino)phenyl)-N-(3-methoxy-4- | 390 |
| | | OCH₃ | | | (2-methyl-4-pyridinyl)phenyl)propanamide | |
| 526 | 1 | H | CHCH₃ | 4-(N-3,4,5-dimethoxybenzyl- | 2-(4-(ethyl(3,4,5-trimethoxybenzyl)amino)- | 540 |
| | | | | N-ethylamino)phenyl | phenyl)-N-(4-(2-methyl-4-pyridinyl)- | |
| | | | | | phenyl)propanamide | |
| 527 | 1 | H | CHCH₃ | 4-(cyclopropylamino)phenyl | 2-(4-(cyclopropylamino)phenyl)-N-(4-(2- | 372 |
| | | | | | methyl-4-pyridinyl)phenyl)propanamide | |
| 528 | 1 | H | CHCH₃ | 4-(N,N- | 2-(4-(dicyclopropylamino)phenyl)-N-(4-(2- | 412 |
| | | | | dicyclopropylamino)phenyl | methyl-4-pyridinyl)phenyl)propanamide | |
| 529 | 1 | H | C(CH₃)₂ | 3-bromophenyl | 2-(3-bromophenyl)-2-methyl-N-(4-(2- | 409 |
| | | | | | methyl-4-pyridinyl)phenyl)propanamide | |
| 530 | 1 | H | R-CH(CH₂)₂CH₃ | 3-bromophenyl | (2R)-2-(3-bromophenyl)-N-(4-(2-methyl-4- | 423.1 |
| | | | | | pyridinyl)phenyl)pentanamide | |
| 531 | 1 | H | S-CH(CH₂)₂CH₃ | 3-bromophenyl | (2S)-2-(3-bromophenyl)-N-(4-(2-methyl-4- | 423.1 |
| | | | | | pyridinyl)phenyl)pentanamide | |
| 532 | 1 | H | CH[CH₂CH(CH₃)₂] | 6-chloropyridin-2-yl | 2-(6-chloro-2-pyridinyl)-4-methyl-N-(4-(2- | 394.1 |
| | | | | | methyl-4-pyridinyl)phenyl)pentanamide | |
| 533 | 1 | H | CH[CH₂CH(CH₃)₂] | 6-morpholin-4-ylpyridin-2-yl | 4-methyl-N-(4-(2-methyl-4-pyridinyl)- | 445.1 |
| | | | | | phenyl)-2-(6-(4-morpholinyl)-2-pyridinyl)- | |
| | | | | | pentanamide | |
| 534 | 1 | H | CH[CH₂CH(CH₃)₂] | 6-(4-morpholin-4-ylphenyl)- | 4-methyl-N-(4-(2-methyl-4-pyridinyl)- | 521.2 |
| | | | | pyridin-2-yl | phenyl)-2-(6-(4-(4-morpholinyl)phenyl)-2- | |
| | | | | | pyridinyl)pentanamide | |
| 535 | 1 | H | CHCH₃ | 3-(6-fluoropyridin-3-yl)phenyl | 2-(3-(6-fluoro-3-pyridinyl)phenyl)-N-(4-(2- | 412.1 |
| | | | | | methyl-4-pyridinyl)phenyl)propanamide | |
| 536 | 1 | H | CHCH₃ | 3-biphenyl | 2-(3-biphenylyl)-N-(4-(2-methyl-4- | 393.2 |
| | | | | | pyridinyl)phenyl)propanamide | |
| 537 | 1 | H | CHCH₃ | 6-bromopyridin-2-yl | 2-(6-bromo-2-pyridinyl)-N-(4-(2-methyl-4- | 396 |
| | | | | | pyridinyl)phenyl)propanamide | |
| 539 | 1 | H | CHCH₃ | 3-(pyridin-3-yl)phenyl | N-(4-(2-methyl-4-pyridinyl)phenyl)-2-(3- | 394.1 |
| | | | | | (3-pyridinyl)phenyl)propanamide | |
| 540 | 1 | H | CHCH₃ | 3-(pyridin-4-yl)phenyl | N-(4-(2-methyl-4-pyridinyl)phenyl)-2-(3- | 394.1 |
| | | | | | (4-pyridinyl)phenyl)propanamide | |
| 541 | 1 | H | CHCH₃ | 3-(2-chloropyridin-4-yl)phenyl | 2-(3-(3-chloro-4-pyridinyl)phenyl)-N-(4-(2- | 428 |
| | | | | | methyl-4-pyridinyl)phenyl)propanamide | |
| 542 | 1 | H | CHCH₃ | 3,4-methylenedioxyphenyl | 2-(1,3-benzodioxol-5-yl)-N-(4-(2-methyl-4- | 360.8 |
| | | | | | pyridinyl)phenyl)propanamide | |
| 543 | 1 | H | CHCH₃ | 7-pyridin-3-yl-1,2,3,4- | N-(4-(2-methyl-4-pyridinyl)phenyl)-2-(7- | 449 |
| | | | | tetrahydroisoquinolin-2-yl | (3-pyridinyl)-3,4-dihydro-2(1H)- | |
| | | | | | isoquinolinyl)propanamide | |
| 544 | 1 | H | CHCH₃ | 4-ethylaminophenyl | 2-(4-(ethylamino)phenyl)-N-(4-(2-methyl- | 360 |
| | | | | | 4-pyridinyl)phenyl)propanamide | |
| 545 | 1 | H | CHCH₃ | 4-sec-butylaminophenyl | 2-(4-((1-methylpropypamino)phenyl)-N-(4- | 388 |
| | | | | | (2-methyl-4-pyridinyl)phenyl)propanamide | |
| 546 | 1 | H | CHCH₃ | 4-methylaminophenyl | 2-(4-(methylamino)phenyl)-N-(4-(2-methyl- | 346 |
| | | | | | 4-pyridinyl)phenyl)propanamide | |
| 547 | 1 | H | CHCH₃ | 4-n-propylaminophenyl | N-(4-(2-methyl-4-pyridinyl)phenyl)-2-(4- | 374 |
| | | | | | (propylamino)phenyl)propanamide | |
| 548 | 1 | H | CHCH₃ | 4-N,N-di-n- | (2-(4-(dipropylamino)phenyl)-N-(4-(2- | 416 |
| | | | | propylaminophenyl | methyl-4-pyridinyl)phenyl)propanamide | |
| 549 | 1 | H | CHCH₃ | 4-N,N-diethylaminophenyl | 2-(4-(diethylamino)phenyl)-N-(4-(2-methyl- | 388 |
| | | | | | 4-pyridinyl)phenyl)propanamide | |
| 550 | 0 | H | — | 2-methylbenzimidazol-6-yl | 2-methyl-N-(4-(2-methyl-4-pyridinyl)- | 343.1 |
| | | | | | phenyl)-1H-benzimidazole-6-carboxamide | |
| 551 | 1 | H | CH₂ | 2-ethylbenzoxazol-5-yl | 2-(2-ethyl-1,3-benzoxazol-5-yl)-N-(4-(2- | 372.1 |
| | | | | | methyl-4-pyridinyl)phenyl)acetamide | |
| 552 | 1 | H | CH₂ | 2-methylbenzoxazol-5-yl | 2-(2-methyl-1,3-benzoxazol-5-yl)-N-(4-(2- | 358.1 |
| | | | | | methyl-4-pyridinyl)phenyl)acetamide | |
| 553 | 1 | H | CHF | quinolin-6-yl | (2R)-2-fluoro-N-(4-(2-methyl-4-pyridinyl)- | 372 |
| | | | | | phenyl)-2-(6-quinolinyl)ethanamide | |
| 554 | 1 | H | CHCH₃ | 3-methoxyquinolin-6-yl | 2-(3-methoxy-6-quinolinyl)-N-(4-(2- | 398 |
| | | | | | methyl-4-pyridinyl)phenyl)propanamide | |
| 555 | 0 | H | — | cyclobutyl | N-(4-(2-methyl-4-pyridinyl)phenyl)- | 267 |
| | | | | | cyclobutanecarboxamide | |
| 556 | 0 | H | — | 2-methylcycloprop-1-yl | 2-methyl-N-(4-(2-methyl-4-pyridinyl)- | 267 |
| | | | | | phenyl)cyclopropanecarboxamide | |
| 557 | 0 | H | — | cyclopentyl | N-(4-(2-methyl-4-pyridinyl)phenyl)- | 281 |
| | | | | | cyclopentanecarboxamide | |
| 558 | 1 | H | CH₂ | cyclopentyl | 2-cyclopentyl-N-(4-(2-methyl-4-pyridinyl)- | 281 |
| | | | | | phenyl)acetamide | |
| 559 | 1 | H | CH₂CH₂ | cyclopropyl | 3-cyclopropyl-N-(4-(2-methyl-4-pyridinyl)- | 295 |
| | | | | | phenyl)propanamide | |
| 560 | 0 | H | — | tetrahydropyran-4-yl | N-(4-(2-methyl-4-pyridinyl)phenyl)- | 297 |
| | | | | | tetrahydro-2H-pyran-4-carboxamide | |
| 561 | 1 | H | CHCH₃ | 3-pyrimidin-2-ylphenyl | N-(4-(2-methyl-4-pyridinyl)phenyl)-2-(3- | 395.1 |
| | | | | | (2-pyrimidinyl)phenyl)propanamide | |
| 562 | 1 | H | CHCH₃ | 3-pyridin-2-ylphenyl | N-(4-(2-methyl-4-pyridinyl)phenyl)-2-(3- | 394.1 |
| | | | | | (2-pyridinyl)phenyl)propanamide | |
| 563 | 0 | H | — | tetrahydrofuran-3-yl | N-(4-(2-methyl-4-pyridinyl)phenyl)- | 283 |
| | | | | | tetrahydro-3-furancarboxamide | |
| 564 | 1 | H | (CH₂)₂ | cyclobutyl | 3-cyclobutyl-N-(4-(2-methyl-4-pyridinyl)- | 295 |
| | | | | | phenyl)propanamide | |
| 565 | 1 | H | CH₂ | cyclobutyl | 2-cyclobutyl-N-(4-(2-methyl-4-pyridinyl)- | 281 |
| | | | | | phenyl)acetamide | |
| 566 | 1 | H | CHCH₃ | 2-(4-morpholin-4-ylphenyl)- | N-(4-(2-methyl-4-pyridinyl)phenyl)-2-(2- | 479.1 |
| | | | | pyridin-4-yl | (4-(4-morpholinyl)phenyl)-4-pyridinyl)- | |
| | | | | | propanamide | |
| 567 | 0 | H | — | 4-chlorophenyl | 4-chloro-N-(4-(2-methyl-4-pyridinyl)- | 323 |
| | | | | | phenyl)benzamide | |
| 568 | 1 | H | CH₂ | 2,4-dimethylthiazol-5-yl | 2-(2,4-dimethyl-1,3-thiazol-5-yl)-N-(4-(2- | 338.1 |
| | | | | | methyl-4-pyridinyl)phenyl)acetamide | |
| 569 | 1 | H | CHCH₃ | 3-(6-dimethylaminopyridin-3- | 2-(3-(6-(dimethylamino)-3-pyridinyl)- | 437.2 |
| | | | | yl)-phenyl | phenyl)-N-(4-(2-methyl-4-pyridinyl)- | |
| | | | | | phenyl)propanamide | |
| 570 | 1 | H | CHCH₃ | 3-(4-methylimidazol-1-yl)- | 2-(3-(4-methyl-1H-imidazol-1-yl)phenyl)- | 397.1 |
| | | | | phenyl | N-(4-(2-methyl-4-pyridinyl)phenyl)- | |
| | | | | | propanamide | |
| 571 | 1 | H | CHCH₃ | 3-(pyrazol-1-yl)-phenyl | N-(4-(2-methyl-4-pyridinyl)phenyl)-2-(3- | 383.1 |
| | | | | | (1H-pyrazol-1-yl)phenyl)propanamide | |
| 572 | 1 | H | CHCH₃ | 4-N-acetyl-N-isopropylamino- | 2-(4-(acetyl(1-methylethyl)amino)phenyl)- | 416 |
| | | | | phenyl | N-(4-(2-methyl-4-pyridinyl)phenyl)- | |
| | | | | | propanamide | |
| 573 | 1 | H | CHCH₃ | 6-chloropyridin-3-yl | 2-(6-chloro-3-pyridinyl)-N-(4-(2-methyl-4- | 352 |
| | | | | | pyridinyl)phenyppropanamide | |
| 574 | 1 | H | CHCH₃ | 4-amino-3-methoxyphenyl | 2-(4-amino-3-methoxyphenyl)-N-(4-(2- | 362 |
| | | | | | methyl-4-pyridinyl)phenyl)propanamide | |
| 575 | 1 | H | CHCH₃ | 4-isopropylamino-3- | 2-(3-methoxy-4-((1-methylethyl)amino)- | 404 |
| | | | | methoxyphenyl | phenyl)-N-(4-(2-methyl-4-pyridinyl)- | |
| | | | | | phenyl)propanamide | |
| 576 | 1 | H | CHCH₃ | 3-bromo-5-methylphenyl | 2-(3-bromo-5-methylphenyl)-N-(4-(2- | 410.9 |
| | | | | | methyl-4-pyridinyl)phenyl)propanamide | |
| 577 | 1 | H | CHCH₃ | 4-fluoro-3-(4-morpholin-4- | 2-(4-fluoro-4′-(4-morpholinyl)-3- | 496.1 |
| | | | | ylphenyl)phenyl | biphenylyl)-N-(4-(2-methyl-4-pyridinyl)- | |
| | | | | | phenyl)propanamide | |
| 578 | 1 | H | CH[CH₂CH(CH₃)₂] | 6-bromopyridin-2-yl | 2-(6-bromo-2-pyridinyl)-4-methyl-N-(4-(2- | 438 |
| | | | | | methyl-4-pyridinyl)phenyl)pentanamide | |
| 579 | 1 | H | CHCH₃ | 4-piperidin-l-ylphenyl | N-(4-(2-methyl-4-pyridinyl)phenyl)-2-(4- | 400 |
| | | | | | (1-piperidinyl)phenyl)propanamide | |
| 580 | 1 | H | CHCH₃ | 6-piperidin-l-ylpyridin-3-yl | N-(4-(2-methyl-4-pyridinyl)phenyl)-2-(6- | 401 |
| | | | | | (1-piperidinyl)-3-pyridinyl)propanamide | |
| 581 | 1 | H | CHCH₃ | 5-methyl-3-(2-methoxy- | 2-(3-(2-methoxy-5-pyrimidinyl)-5-methyl- | 439 |
| | | | | pyrimidin-5-yl)phenyl | phenyl)-N-(4-(2-methyl-4- | |
| | | | | | pyridinyl)phenyl)propanamide | |
| 582 | 1 | H | CH₂ | benzoxazol-5-yl | 2-(1,3-benzoxazol-5-yl)-N-(4-(2-methyl-4- | 344.1 |
| | | | | | pyridinyl)phenyl)acetamide | |
| 583 | 1 | H | CH₂ | 2-pyridin-3-ylbenzoxazol-5-yl | N-(4-(2-methyl-4-pyridinyl)phenyl)-2-(2- | 421.1 |
| | | | | | (3-pyridinyl)-1,3-benzoxazol-5-yl)- | |
| | | | | | acetamide | |
| 584 | 1 | H | CH₂ | 2-pyridin-4-ylbenzoxazol-5-yl | N-(4-(2-methyl-4-pyridinyl)phenyl)-2-(2- | 421.1 |
| | | | | | (4-pyridinyl)-1,3-benzoxazol-5-yl)- | |
| | | | | | acetamide | |
| 585 | 1 | H | CH[CH₂CH(CH₃)₂] | 3,5-di(4-trifluoromethyl- | 2-(4,4″-bis(trifluoromethyl)-1,1′:3′,1″- | 647 |
| | | | | phenyl)phenyl | terphenyl-5′-yl)-4-methyl-N-(4-(2-methyl-4- | |
| | | | | | pyridinyl)phenyl)pentanamide | |
| 586 | 1 | H | CHCH₂cyclopropyl | 5-isopropylaminopyridin-2-yl | 3-cyclopropyl-2-(5((1-methylethypamino)- | 415 |
| | | | | | 2-pyridinyl)-N-(4-(2-methyl-4-pyridinyl)- | |
| | | | | | phenyl)propanamide | |
| 587 | 1 | H | CHCH₃ | 5-methyl-3-indol-5-ylphenyl | 2-(3-(1H-indol-5-yl)-5-methylphenyl)-N-(4- | 446 |
| | | | | | (2-methyl-4-pyridinyl)phenyl)propanamide | |
| 588 | 1 | H | CHCH₃ | 3-bromo-5-chlorophenyl | 2-(3-bromo-5-chlorophenyl)-N-(4-(2- | 430.9 |
| | | | | | methyl-4-pyridinyl)phenyl)propanamide | |
| 589 | 1 | H | CHCH₃ | 5-chloro-3-(2-methyl- | 2-(3-chloro-5-(2-methyl-5-pyrimidinyl)- | 443 |
| | | | | pyrimidin-5-yl)phenyl | phenyl)-N-(4-(2-methyl-4-pyridinyl)- | |
| | | | | | phenyl)propanamide | |
| 590 | 1 | H | CHCH₃ | 3-chloro-5-(1,5- | 2-(3-chloro-5-(1,5-dimethyl-1H-pyrazol-4- | 445 |
| | | | | dimethylpyrazol-4-yl)phenyl | yl)phenyl)-N-(4-(2-methyl-4-pyridinyl)- | |
| | | | | | phenyl)propanamide | |
| 591 | 1 | H | CHCH₃ | 3-chloro-5-(pyridin-3- | 2-(3-chloro-5-(3-pyridinyl)phenyl)-N-(4-(2- | 428 |
| | | | | yl)phenyl | methyl-4-pyridinyl)phenyl)propanamide | |
| 592 | 1 | H | CHCH₃ | 5-methyl-3-(4-cyanophenyl)- | 2-(4′-cyano-5-methyl-3-biphenylyl)-N-(4- | 432 |
| | | | | phenyl | (2-methyl-4-pyridinyl)phenyl)propanamide | |
| 593 | 1 | H | CHCH₃ | 5-methyl-3-(3-cyanophenyl)- | 2-(3′-cyano-5-methyl-3-biphenylyl)-N-(4- | 432 |
| | | | | phenyl | (2-methyl-4-pyridinyl)phenyl)propanamide | |
| 594 | 1 | H | CHCH₃ | 5-methyl-3-(2-cyanophenyl)- | 2-(2′-cyano-5-methyl-3-biphenylyl)-N-(4- | 432 |
| | | | | phenyl | (2-methyl-4-pyridinyl)phenyl)propanamide | |
| 595 | 1 | H | CH[CH₂CH(CH₃)₂] | phenyl | 4-methyl-N-(4-(2-methyl-4-pyridinyl)- | 359.1 |
| | | | | | phenyl)-2-phenylpentanamide | |
| 596 | 1 | H | CHCH₃ | 4-isobutylaminophenyl | 2-(4-((2-methylpropyl)amino)phenyl)-N-(4- | 388 |
| | | | | | (2-methyl-4-pyridinyl)phenyl)propanamide | |
| 597 | 1 | H | CHCH₃ | 4-N,N-di-isobutylaminophenyl | 2-(4-(bis(2-methylpropyl)amino)phenyl)-N- | 444 |
| | | | | | (4-(2-methyl-4-pyridinyl)phenyl)- | |
| | | | | | propanamide | |
| 598 | 1 | H | CHCH₃ | 3-cyano-5-methylphenyl | 2-(3-cyano-5-methylphenyl)-N-(4-(2- | 356 |
| | | | | | methyl-4-pyridinyl)phenyl)propanamide | |
| 599 | 1 | H | CHCH₃ | 4-(N-3,4,5-dimethoxybenzyl- | N-(4-(2-methyl-4-pyridinyl)phenyl)-2-(4- | 554 |
| | | | | N-n-propylamino)phenyl | (propyl(3,4,5-trimethoxybenzypamino)- | |
| | | | | | phenyl)propanamide | |
| 600 | 1 | H | CH[CH₂CH(CH₃)₂] | 6-chloropyridin-3-yl | 4-methyl-N-(4-(2-methyl-4-pyridinyl)- | |
| | | | | | phenyl)-2-(6-chloropyridin-3- | |
| | | | | | yl)pentanamide | |
| 601 | 1 | H | CH[CH₂CH(CH₃)₂] | 6-piperidin-1-ylpyridin-3-yl | 4-methyl-N-(4-(2-methyl-4-pyridinyl)- | 443 |
| | | | | | phenyl)-2-(6-(1-piperidinyl)-3- | |
| | | | | | pyridinyl)pentanamide | |
| 602 | 1 | H | CHCH₃ | 3-(6-methylaminopyridin-3- | 2-(3-(6-(methylamino)-3-pyridinyl)phenyl)- | 423.1 |
| | | | | yl)-phenyl | N-(4-(2-methyl-4-pyridinyl)phenyl)- | |
| | | | | | propanamide | |
| 603 | 1 | H | CHCH₃ | 3-[6-(2-methoxyethylamino)- | 2-(3-(6-((2-methoxyethyl)amino)-3- | 467.1 |
| | | | | pyridin-3-yl]-phenyl | pyridinyl)phenyl)-N-(4-(2-methyl-4- | |
| | | | | | pyridinyl)phenyl)propanamide | |
| 604 | 1 | H | CH₂ | 2-methylbenzimidazol-5-yl | 2-(2-methyl-1H-benzimidazol-5-yl)-N-(4- | 356.8 |
| | | | | | (2-methyl-4-pyridinyl)phenyl)acetamide | |
| 605 | 0 | H | — | indol-5-yl | N-(4-(2-methyl-4-pyridinyl)phenyl)-1H- | 328 |
| | | | | | indole-5-carboxamide | |
| 606 | 0 | H | — | benzothiophen-3-yl | N-(4-(2-methyl-4-pyridinyl)phenyl)-1- | 344.8 |
| | | | | | benzothiophene-3-carboxamide | |
| 607 | 0 | H | — | 6-methylsulfonylindol-2-yl | N-(4-(2-methyl-4-pyridinyl)phenyl)-6- | 406.8 |
| | | | | | (methylsulfonyl)-1H-indole-2-carboxamide | |
| 608 | 1 | H | CHCH₃ | 3-methyl-5-(2- | 2-(3-methyl-5-(2-methyl-5-pyrimidinyl- | 423 |
| | | | | methylpyrimidin-5-yl)-phenyl | )phenyl)-N-(4-(2-methyl-4- | |
| | | | | | pyridinyl)phenyl)propanamide | |
| 609 | 0 | H | — | 5-cyclopropylpyrazol-3-yl | 5-cyclopropyl-N-(4-(2-methyl-4-pyridinyl)- | 318.8 |
| | | | | | phenyl)-1H-pyrazole-3-carboxamide | |
| 610 | 0 | H | — | 7-methylimidazo[1,2- | 7-methyl-N-(4-(2-methyl-4-pyridinyl)- | 342.8 |
| | | | | a]pyridin-2-yl | phenyl)imidazo[1,2-a]pyridine-2- | |
| | | | | | carboxamide | |
| 611 | 0 | H | — | 8-methylimidazo[1,2- | 8-methyl-N-(4-(2-methyl-4-pyridinyl)- | 342.8 |
| | | | | a]pyridin-2-yl | phenyl)imidazo[1,2-a]pyridine-2- | |
| | | | | | carboxamide | |
| 612 | 0 | H | — | benzothiophen-2-yl | N-(4-(2-methyl-4-pyridinyl)phenyl)-1- | 344.8 |
| | | | | | benzothiophene-2-carboxamide | |
| 613 | 1 | H | C(CH₂CH₃)(CH₃) | phenyl | 2-methyl-N-(4-(2-methyl-4-pyridinyl)- | 345 |
| | | | | | phenyl)-2-phenylbutanamide | |
| 614 | 1 | H | CH[CH₂CH(CH₃)₂] | 3,4-methylenedioxyphenyl | 2-(1,3-benzodioxol-5-yl)-4-methyl-N-(4-(2- | 403.1 |
| | | | | | methyl-4-pyridinyl)phenyl)pentanamide | |
| 615 | 1 | H | CHCH₃ | 3-methyl-5-(4-pyridin-2- | N-(4-(2-methyl-4-pyridinyl)phenyl)-2-(3- | 492 |
| | | | | ylpiperazin-1-yl)-phenyl | methyl-5-(4-(2-pyridinyl)-1-piperazinyl)- | |
| | | | | | phenyl)propanamide | |
| 616 | 1 | H | CHCH₃ | 3-methyl-5-(2-methylpyridin- | 2-(3-methyl-5-(2-methyl-4-pyridinyl)- | 422 |
| | | | | 4-yl)-phenyl | phenyl)-N-(4-(2-methyl-4-pyridinyl)- | |
| | | | | | phenyl)propanamide | |
| 617 | 1 | H | CHCH₃ | 3-fluoro-5-(pyridin-3-yl)- | 2-(3-fluoro-5-(3-pyridinyl)phenyl)-N-(4-(2- | 412 |
| | | | | phenyl | methyl-4-pyridinyl)phenyl)propanamide | |
| 618 | 1 | H | CHCH₃ | 3-fluoro-5-(2-methylpyridin-4- | 2-(3-fluoro-5-(2-methyl-4-yridinyl)phenyl)- | 426 |
| | | | | yl)-phenyl | N-(4-(2-methyl-4-pyridinyl)phenyl)- | |
| | | | | | propanamide | |
| 619 | 1 | H | CHCH₃ | 3-difluoromethyl-5- | 2-(3-(difluoromethyl)-5-(trifluoromethyl)- | 435 |
| | | | | trifluoromethylphenyl | phenyl)-N-(4-(2-methyl-4-pyridinyl)- | |
| | | | | | phenyl)propanamide | |
| 620 | 1 | H | CHCH₃ | 3-fluoro-5-methylphenyl | 2-(3-fluoro-5-methylphenyl)-N-(4-(2- | 349 |
| | | | | | methyl-4-pyridinyl)phenyl)propanamide | |
| 621 | 0 | H | — | isoquinolin-5-yl | N-(4-(2-methyl-4-pyridinyl)phenyl)-5- | 340.1 |
| | | | | | isoquinolinecarboxamide | |
| 622 | 0 | H | — | 1-methylindol-5-yl | 1-methyl-N-(4-(2-methyl-4-pyridinyl)- | 342.1 |
| | | | | | phenyl)-1H-indole-5-carboxamide | |
| 623 | 0 | H | — | quinolin-5-yl | N-(4-(2-methyl-4-pyridinyl)phenyl)-5- | 340 |
| | | | | | quinolinecarboxamide | |
| 624 | 0 | H | — | benzo[d][1,2,3]thiadiazol-5-yl | N-(4-(2-methyl-4-pyridinyl)phenyl)-1,2,3- | 347 |
| | | | | | benzothiadiazole-5-carboxamide | |
| 625 | 0 | H | — | 4-morpholin-4-ylmethylphenyl | N-(4-(2-methyl-4-pyridinyl)phenyl)-4-(4- | 388.1 |
| | | | | | morpholinylmethyl)benzamide | |
| 626 | 0 | H | — | 1-methyl-3-ethylpyrazol-5-yl | 3-ethyl-l-methyl-N-(4-(2-methyl-4- | 321.1 |
| | | | | | pyridinyl)-phenyl)-1H-pyrazole-5- | |
| | | | | | carboxamide | |
| 627 | 0 | H | — | 3-ethoxyphenyl | 3-ethoxy-N-(4-(2-methyl-4-pyridinyl)- | 333.1 |
| | | | | | phenyl)benzamide | |
| 628 | 0 | H | — | 3-morpholin-4-ylmethylphenyl | N-(4-(2-methyl-4-pyridinyl)phenyl)-3-(4- | 388.1 |
| | | | | | morpholinylmethyl)benzamide | |
| 629 | 0 | H | — | quinolin-4-yl | N-(4-(2-methyl-4-pyridinyl)phenyl)-4- | 340.1 |
| | | | | | quinolinecarboxamide | |
| 630 | 0 | H | — | 3-difluoromethoxyphenyl | 3-(difluoromethoxy)-N-(4-(2-methyl-4- | 355 |
| | | | | | pyridinyl)phenyl)benzamide | |
| 631 | 0 | H | — | 4-methoxy-3-methylphenyl | 4-methoxy-3-methyl-N-(4-(2-methyl-4- | 333.1 |
| | | | | | pyridinyl)phenyl)benzamide | |
| 632 | 1 | H | CH₂ | 2,5-dimethylthiazol-4-yl | 2-(2,5-dimethyl-1,3-thiazol-4-yl)-N-(4-(2- | 338 |
| | | | | | methyl-4-pyridinyl)phenyl)acetamide | |
| 633 | 1 | H | CHCH₃ | 3-(6-cyanopyridin-3-yl)phenyl | 2-(3-(6-cyano-3-pyridinyl)phenyl)-N-(4-(2- | 419 |
| | | | | | methyl-4-pyridinyl)phenyl)propanamide | |
| 634 | 1 | H | CH[CH₂CH(CH₃)₂] | 3-bromophenyl | 2-(3-bromophenyl)-4-hydroxy-4-methyl-N- | 453 |
| | | | | | (4-(2-methyl-4-pyridinyl)phenyl)- | |
| | | | | | pentanamide | |
| 635 | 1 | H | CHCH₃ | 3-methyl-5-(4-methyl-3- | 2-(3-methyl-5-(4-methyl-3-oxo-1- | 443 |
| | | | | oxopiperazin-1-yl)phenyl | piperazinyl)phenyl)-N-(4-(2-methyl-4- | |
| | | | | | pyridinyl)phenyl)propanamide | |
| 636 | 1 | H | CHCH₃ | 5-methyl-3-(4- | 2-(3-(4-methoxy-1-piperidinyl)-5-methyl- | 444 |
| | | | | methoxypiperidin-1-yl)phenyl | phenyl)-N-(4-(2-methyl-4-pyridinyl)- | |
| | | | | | phenyl)propanamide | |
| 637 | 1 | H | CHCH₃ | 5-methyl-3-(4,4- | 2-(3-(4,4-difluoro-1-piperidinyl)-5-methyl- | 450 |
| | | | | difluoropiperidin-1-yl)phenyl | phenyl)-N-(4-(2-methyl-4-pyridinyl)- | |
| | | | | | phenyl)propanamide | |
| 638 | 1 | H | CHCH₃ | 3-(6-isopropylaminopyridin-3- | 2-(3-(6-((1-methylethyl)amino)-3- | 451.1 |
| | | | | yl)phenyl | pyridinyl)phenyl)-N-(4-(2-methyl-4- | |
| | | | | | pyridinyl)phenyl)propanamide | |
| 639 | 1 | H | CHCH₃ | 3-(4-carboxyphenyl)phenyl | 3′-((1R)-1-methyl-2-((4-(2-methyl-4- | 437.1 |
| | | | | | pyridinyl)phenyl)amino)-2-oxoethyl)-4- | |
| | | | | | biphenylcarboxylic acid | |
| 640 | 1 | H | CHCH₃ | 3-(6-chloropyrazin-2-yl)phenyl | 2-(3-(6-chloro-2-pyrazinyl)phenyl)-N-(4-(2- | 429.1 |
| | | | | | methyl-4-pyridinyl)phenyl)propanamide | |
| 641 | 1 | H | CHCH₃ | 3-(5-methoxypyrazin-2- | 2-(3-(5-methoxy-2-pyrazinyl)phenyl)-N-(4- | 425.1 |
| | | | | yl)phenyl | (2-methyl-4-pyridinyl)phenyl)propanamide | |
| 642 | 1 | H | CH[CH₂CH(CH₃)₂] | 5-bromo-2-methoxyphenyl | 2-(5-bromo-2-methoxyphenyl)-4-methyl-N- | 467 |
| | | | | | (4-(2-methyl-4-pyridinyl)phenyl)- | |
| | | | | | pentanamide | |
| 643 | 1 | H | CH[CH₂CH(CH₃)₂] | 5-bromo-2-fluorophenyl | 2-(5-bromo-2-fluorophenyl)-4-methyl-N-(4- | 454.9 |
| | | | | | (2-methyl-4-pyridinyl)phenyl)pentanamide | |
| 644 | 1 | H | CH[CH₂CH(CH₃)₂] | 3-bromo-4-fluorophenyl | 2-(3-bromo-4-fluorophenyl)-4-methyl-N-(4- | 455 |
| | | | | | (2-methyl-4-pyridinyl)phenyl)pentanamide | |
| 645 | 1 | H | CHCH₃ | 5-chloro-3-(3-cyanophenyl)- | 2-(5-chloro-3′-cyano-3-biphenylyl)-N-(4-(2- | 452 |
| | | | | phenyl | methyl-4-pyridinyl)phenyl)propanamide | |
| 646 | 1 | H | CHCH₃ | 3-chloro-5-(2-methoxy- | 2-(3-chloro-5-(2-methoxy-5-pyrimidinyl)- | 459 |
| | | | | pyrimidin-5-yl)-phenyl | phenyl)-N-(4-(2-methyl-4- | |
| | | | | | pyridinyl)phenyl)propanamide | |
| 647 | 1 | H | CHCH₃ | 3-chloro-5-(furan-3-yl)-phenyl | 2-(3-chloro-5-(3-furanyl)phenyl)-N-(4-(2- | 416.9 |
| | | | | | methyl-4-pyridinyl)phenyl)propanamide | |
| 648 | 1 | H | CHCH₃ | 5-chloro-3-(4-cyanophenyl)- | 2-(5-chloro-4′-cyano-3-biphenylyl)-N-(4-(2- | 453 |
| | | | | phenyl | methyl-4-pyridinyl)phenyl)propanamide | |
| 649 | 1 | H | CHCH₃ | 4,6-ditrifluoromethylpyridin-2- | 2-(4,6-bis(trifluoromethyl)-2-pyridinyl)-N- | 453.9 |
| | | | | yl | (4-(2-methyl-4-pyridinyl)phenyl)- | |
| | | | | | propanamide | |
| 650 | 0 | H | — | pyridin-4-yl | N-(4-(2-methyl-4-pyridinyl)phenyl)-4- | 334 |
| | | | | | pyridinecarboxamide | |
| 651 | 0 | H | — | indol-6-yl | N-(4-(2-methyl-4-pyridinyl)phenyl)-1H- | 328 |
| | | | | | indole-6-carboxamide | |
| 652 | 0 | H | — | 3-acetylaminophenyl | 3-(acetylamino)-N-(4-(2-methyl-4- | 346 |
| | | | | | pyridinyl)phenyl)benzamide | |
| 653 | 1 | H | CHCH₃ | 2,4-dimethylthiazol-5-yl | 2-(2,4-dimethyl-1,3-thiazol-5-yl)-N-(4-(2- | 352 |
| | | | | | methyl-4-pyridinyl)phenyl)propanamide | |
| 654 | 1 | H | CHCH₃ | 6-isopropylaminopyridin-3-yl | 2-(6-((1-methylethypamino)-3-pyridinyl)- | 375 |
| | | | | | N-(4-(2-methyl-4-pyridinyl)phenyl)- | |
| | | | | | propanamide | |
| 655 | 1 | H | CHCH₃ | 4-(N-4-methoxybenzyl-N- | 2-(4-((4-methoxybenzyl)(methyl)amino)- | 466 |
| | | | | methylamino)phenyl | phenyl)-N-(4-(2-methyl-4-pyridinyl)- | |
| | | | | | phenyl)propanamide | |
| 656 | 1 | H | CHCH₃ | 4-benzylaminophenyl | 2-(4-(benzylamino)phenyl)-N-(4-(2-methyl- | 422 |
| | | | | | 4-pyridinyl)phenyl)propanamide | |
| 657 | 1 | H | CH[CH₂CH(CH₃)₂] | 6-dimethylaminopyridin-3-yl | 2-(6-(dimethylamino)-3-pyridinyl)-4- | 403 |
| | | | | | methyl-N-(4-(2-methyl-4-pyridinyl)phenyl)- | |
| | | | | | pentanamide | |
| 658 | 1 | H | CHCH₃ | 3-(3-methoxypiperidin-1- | 2-(3-(4-methoxy-1-piperidinyl)phenyl)-N- | 430 |
| | | | | yl)phenyl | (4-(2-methyl-4-pyridinyl)phenyl)- | |
| | | | | | propanamide | |
| 659 | 1 | H | CHCH³ | 3-(4-tert-butoxyearbonyl- | tert-butyl 4-(3-(1-methyl-2-((4-(2-methyl-4- | 501.2 |
| | | | | piperazin-l-yl)phenyl | pyridinyl)phenypamino)-2-oxoethyl)- | |
| | | | | | phenyl)-1-piperazinecarboxylate | |
| 660 | 1 | H | CHCH³ | 3-(piperazin-l-yl)phenyl | N-(4-(2-methyl-4-pyridinyl)phenyl)-2-(3- | 401.1 |
| | | | | | (1-piperazinyl)phenyl)propanamide | |
| 661 | 1 | 3- | CH[CH₂CH(CH₃)₂] | 5-bromopyridin-3-yl | 2-(5-bromo-3-pyridinyl)-N-(3-methoxy-4- | 468 |
| | | OCH₃ | | | (2-methyl-4-pyridinyl)phenyl)-4-methyl- | |
| | | | | | pentanamide | |
| 662 | 0 | H | — | 1,3,4,5- | N-(4-(2-methyl-4-pyridinyl)phenyl)-1,3,4,5- | 400 |
| | | | | tetrahydrothiopyrano[4,3- | tetrahydrothopyrano[4,3-b]indole-8- | |
| | | | | 1)]indol-8-yl | carboxamide | |
| 663 | 0 | H | — | 1-methylindazol-3-yl | 1-methyl-N-(4-(2-methyl-4-pyridinyl)- | 343.1 |
| | | | | | phenyl)-1H-indazole-3-earboxamide | |
| 664 | 0 | H | — | 4-pyrrol-1-ylphenyl | N-(4-(2-methyl-4-pyridinyl)phenyl)-4-(1H- | 354.1 |
| | | | | | pyrrol-1-yl)benzamide | |
| 665 | 1 | H | CHCH₃ | 4-bromothiophen-2-yl | 2-(4-bromo-2-thiophenyl)-N-(4-(2-methyl- | 402.9 |
| | | | | | 4-pyridinyl)phenyl)propanamide | |
| 666 | 1 | H | CHCH₃ | 5-chlorothiophen-2-yl | 2-(5-chloro-2-thiophenyl)-N-(4-(2-methyl- | 357 |
| | | | | | 4-pyridinyl)phenyl)propanamide | |
| 667 | 1 | H | CHCH₃ | 4-N-benzyl-N-methylphenyl | 2-(4-(benzyl(methyl)amino)phenyl)-N-(4- | 436 |
| | | | | | (2-methyl-4-pyridinyl)phenyl)propanamide | |
| 668 | 1 | H | CH[CH₂CH(CH₃)₂] | 5-bromopyridin-3-yl | 2-(5-bromo-3-pyridinyl)-4-methyl-N-(4-(2- | 440 |
| | | | | | methyl-4-pyridinyl)phenyl)pentanamide | |
| 669 | 1 | H | CHCH₃ | 4-(2-methoxypyrimidin-5- | 2-(4-(2-methoxy-5-pyrimidinyl)-2- | 431 |
| | | | | yl)thiophen-2-yl | thiophenyl)-N-(4-(2-methyl-4-pyridinyl)- | |
| | | | | | phenyl)-propanamide | |
| 670 | 1 | H | CHCH₃ | 5-bromothiophen-2-yl | 2-(5-bromo-2-thiophenyl)-N-(4-(2-methyl- | 402.9 |
| | | | | | 4-pyridinyl)phenyl)propanamide | |
| 671 | 1 | H | CH[CH₂CF(CH₃)₂] | 3-bromophenyl | 2-(3-bromophenyl)-4-fluoro-4-methyl-N-(4- | 455.1 |
| | | | | | (2-methyl-4-pyridinyl)phenyl)pentanamide | |
| 672 | 1 | H | CH[CH₂CH(CH₃)₂] | 3-(2-methoxyethylamino- | 2-(3-(((2-methoxyethyl)amino)methyl)- | 446.1 |
| | | | | methyl)phenyl | phenyl)-4-methyl-N-(4-(2-methyl-4- | |
| | | | | | pyridinyl)phenyl)pentanamide | |
| 673 | 1 | H | CH[CH₂CH(CH₃)₂] | 3-(dimethylamino-methyl)- | 2-(3-((dimethylamino)methyl)phenyl)-4- | 416.1 |
| | | | | phenyl | methyl-N-(4-(2-methyl-4-pyridinyl)phenyl)- | |
| | | | | | pentanamide | |
| 674 | 1 | H | CH[CH₂CH(CH₃)₂] | 3-(piperidin-1-ylmethyl)phenyl | 4-methyl-N-(4-(2-methyl-4-pyridinyl)- | 456.1 |
| | | | | | phenyl)-2-(3-(1-piperidinylmethyl)phenyl)- | |
| | | | | | pentanamide | |
| 675 | 1 | H | CH[CH₂CH(CH₃)₂] | 3-hydroxymethylphenyl | 2-(3-(hydroxymethyl)phenyl)-4-methyl-N- | 456 |
| | | | | | (4-(2-methyl-4-pyridinyl)phenyl)- | |
| | | | | | pentanamide | |
| 676 | 1 | H | CH[CH₂CH(CH₃)₂] | 3-(morpholin-4-ylmethyl)- | 4-methyl-N-(4-(2-methyl-4-pyridinyl)- | 389.1 |
| | | | | phenyl | phenyl)-2-(3-(4-morpholinylmethyl)- | |
| | | | | | phenyl)-pentanamide | |
| 677 | 1 | H | CH[CH₂CH(CH₃)₂] | 5-methylpyridin-3-yl | 4-methyl-2-(5-methyl-3-pyridinyl)-N-(4-(2- | 458.1 |
| | | | | | methyl-4-pyridinyl)phenyl)pentanamide | |

### Embodiments

IA. In one embodiment, the compound of Formula (I′) is represented by the structure:

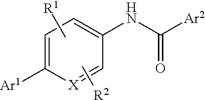

IB. In another embodiment, the compound of Formula (I) is represented by the structure:

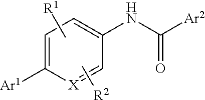

(a) Within embodiments IA and IB independently, one group of compounds is that wherein X is —N—.
(b) Within embodiments IA and IB independently, another group of compounds is that wherein X is —CH—.
(c) Within embodiments IA and IB independently, yet another group of compounds is that wherein R¹ and R² are hydrogen.

Within group (c), one group of compounds is that wherein X is —CH—.

Within group (c), another group of compounds is that wherein X is —N—.

Within group (c) and groups contained therein, in one group of compounds Ar¹ is a ring of formula (iv) where R⁵ is alkyl, more preferably methyl or ethyl, more preferably methyl, more preferably 2-alkylpyridin-4-yl, even more preferably Ar¹ is 2-methylpyridin-4-yl.

(d) Within embodiments IA and IB independently, yet another group of compounds is that wherein Ar¹ is a ring of formula (i) where R³ is alkyl, preferably methyl or ethyl, more preferably methyl.
(e) Within embodiments IA and IB independently, yet another group of compounds is that wherein Ar¹ is a ring of formula (ii).
(f) Within embodiments IA and IB independently, yet another group of compounds is that wherein Ar¹ is a ring of formula (iii) where R⁴ is alkyl, preferably methyl or ethyl, more preferably methyl.
(g) Within embodiments IA and IB independently, yet another group of compounds is that wherein Ar¹ is a ring of formula (iv) where R⁵ is alkyl, more preferably methyl or ethyl, more preferably methyl, more preferably 2-alkylpyridin-4-yl, even more preferably Ar¹ is 2-methylpyridin-4-yl.

Within groups (d) through (g) independently, in one group of compounds X is —CH—.

Within groups (d) through (g) independently, in one group of compounds X is —CH— and R¹ and R² are hydrogen.

Within group (g), in another group of compounds X is —N—.

Within group (g), in yet another group of compounds R¹ is hydrogen and R² is alkyl, alkoxy, or halo, preferably methyl, chloro, fluoro, or methoxy, more preferably where methyl, chloro, fluoro, or methoxy are located at carbon adjacent to the carbon substituted with Ar¹ group. Within this group, in one group of compounds X is —CH—

(i) Within groups (a) through (g) independently and groups contained therein, one group of compounds is that wherein Ar² is aryl optionally substituted as described in the Summary of the Invention.

Within this group, one group of compounds is that wherein Ar² is phenyl or naphthyl optionally substituted with R^{a} which is halo, alkyl, haloalkyl, or alkoxy or R^{b} which is selected from halo, alkyl, haloalkyl, alkoxy, cycloalkyl, aryl, aryloxy, amino, monosubstituted amino, disubstituted amino, cyano, acyl, or aralkyl.

Within this group, another group of compounds is that wherein Ar² is 3,5-diCF₃phenyl, 4-trifluoromethylphenyl, 3,4-dichlorophenyl, 2,4-dichlorophenyl, 2-trifluoromethylphenyl, 3-chlorophenyl, 3-chloro-6-methoxyphenyl, 3-trifluoromethylphenyl, 3-chlorophenyl, 2,6-dichlorophenyl, naphth-1-yl, 3,4-methylenedioxyphenyl, 4-chlorophenyl, 2-fluorophenyl, biphen-4-yl, phenyl, 3,5-difluorophenyl, 4-methoxyphenyl, 3-phenoxyphenyl, naphth-2-yl, 3-methoxyphenyl, 2-chloro-5-bromophenyl, 3-chloro-4-methoxyphenyl, 2-chlorophenyl, 3,4-ethylenedioxyphenyl, 3,5-dimethylphenyl, 2,3-dichlorophenyl, 3-dimethylaminophenyl, 3-cyanolphenyl, 3-chloro-4,5-dimethoxyphenyl, 3-methylphenyl, 6-methoxynaphth-2-yl, 3,5-dichlorophenyl, 2-methoxy-5-chlorophenyl, naphth-1-yl, 4-methylphenyl, 3-methylphenyl, 4-benzoylphenyl, 4-isobutylphenyl, or 3-fluoro-4-phenylphenyl.

(ii) Within groups (a) through (g) independently, and groups contained therein, another group of compounds is that wherein Ar² is heteroaryl optionally substituted as described in the Summary of the Invention. Within this group, one group of compounds is that wherein Ar² heteroaryl optionally substituted with R^{a} which is halo, alkyl, haloalkyl, or alkoxy or R^{b} which is selected from halo, alkyl, haloalkyl, alkoxy, cycloalkyl, aryl, aryloxy, amino, monosubstituted amino, disubstituted amino, cyano, acyl, or aralkyl. Within this group, another group of compounds is that wherein Ar² is 5-methylH-imidazo[1,2-a]pyridin-2-yl, 5-cyclopropylisoxazol-3-yl, 6-chloropyridin-2-yl, 5-chlorothiophen-2-yl, 5-bromopyridin-3-yl, 5-methylthiophen-2-yl, 2,6-dichloropyridin-4-yl, imidazol-1-yl, 3,5-dimethylimidazol-1-yl, 3,5-dimethylisoxazol-4-yl, benzothiophen-3-yl, 5-bromo-1H-indazol-3-yl, or 1H-indol-3-yl.
(iii) Within groups (a) through (g) independently, and groups contained therein, another group of compounds is that wherein Ar² is cycloalkyl or heterocyclyl optionally substituted as described in the Summary of the Invention. Within this group, one group of compounds is that wherein Ar² cycloalkyl or heterocyclyl optionally substituted with R^{a} which is halo, alkyl, haloalkyl, or alkoxy or R^{b} which is selected from halo, alkyl, haloalkyl, alkoxy, cycloalkyl, aryl, aryloxy, amino, monosubstituted amino, disubstituted amino, cyano, acyl, or aralkyl. Within this group, another group of compounds is that wherein Ar² is 1-(4-F-phenyl)cyclopent-1-yl, 3-methoxycyclohexyl, tetrahydrofuran-2-yl, 1-phenyl-2-oxopyrrolidin-4-yl, cyclopropyl, 1-acetylpiperidin-4-yl, 1-cyclohexyl-2-oxopyrrolidin-4-yl, 1-tert-butyl-2-oxopyrrolidin-4-yl, or 1-benzyl-2-oxopyrrolidin-4-yl.
(iv) Within groups (a) through (g) independently, and groups contained therein, another group of compounds is that wherein Ar² is fused cycloalkyl or fused heterocyclyl, preferably fused heterocyclyl optionally substituted as described in the Summary of the Invention. Within this group, one group of compounds is that wherein Ar² fused cycloalkyl or fused heterocyclyl, preferably fused heterocyclyl, optionally substituted with R^{a} which is halo, alkyl, haloalkyl, or alkoxy or R^{b} which is selected from halo, alkyl, haloalkyl, alkoxy, cycloalkyl, aryl, aryloxy, amino, monosubstituted amino, disubstituted amino, cyano, acyl, or aralkyl.
(v) Within group IB(g) and groups contained therein, yet another group of compounds is that wherein Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, hydroxyl, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, optionally substituted phenyl, optionally substituted heteroaryl, or optionally substituted heterocyclyl. Preferably, Ar² is aryl or heteroaryl where each of the aforementioned ring is substituted with R^{a} or R^{b} where R^{a} is alkyl, halo, haloalkyl, haloalkoxy, cyano, alkoxy and R^{b} is alkyl, halo, haloalkyl, haloalkoxy, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy where the aromatic or alicyclic ring in R^{a} and R^{b} is optionally substituted with R^{d} or R^{e} which are independently selected from alkyl, halo, haloalkyl, haloalkoxy, cyano, alkoxy, or hydroxy.

In another embodiment within this group, Ar² is aryl or heteroaryl substituted with R^{a} and R^{b} where R^{a} is alkyl, halo, haloalkyl, haloalkoxy, cyano, alkoxy, and R^{b} is aryl, heteroaryl, or heterocycly where the aromatic or alicyclic ring in R^{b} is substituted with R^{d} or R^{e} where Rd is alkyl, halo, haloalkyl, haloalkoxy, cyano, alkoxy, or hydroxyl and R^{e} is optionally substituted phenyl, optionally substituted heteroaryl, or optionally substituted heterocyclyl.

(vi) Within group IA(g) and IB(g) independently, and groups contained therein, yet another group of compounds is that wherein Ar² is aryl, heteroaryl, cycloalkyl, heterocyclyl attached to —NHCO— group via carbon atom, or fused heterocyclyl, preferably aryl or heteroaryl, where each of the aforementioned ring is optionally substituted with R^{a} where R^{a} is alkyl, halo, haloalkyl, or alkoxy; and substituted with R^{b} where R^{b} is alkyl, halo, haloalkyl, haloalkoxy, alkylthio, cyano, alkoxy, amino, monosubstituted amino, disubstituted amino, sulfonyl, acyl, alkoxycarbonyl, hydroxyalkyl, aryl, heteroaryl, cycloalkyl, heterocyclyl, aralkyl, or aryloxy, or when R^{b} and R^{c} are on adjacent atoms they can combine to form methylenedioxy or ethylenedioxy; where the aromatic or alicyclic ring in R^{a} and R^{b} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, or aminocarbonyl.
(vii) Within group IB(g) and groups contained therein, in another group of compounds Ar² is aryl, heteroaryl, cycloalkyl, heterocyclyl attached to —NHCO— group via carbon atom, or fused heterocyclyl, preferably aryl or heteroaryl, where each of the aforementioned ring is optionally substituted with R^{b} where R^{b} is alkyl, halo, haloalkyl, or alkoxy; and substituted with R^{c} where R^{c} is alkyl, halo, haloalkyl, haloalkoxy, alkylthio, cyano, alkoxy, amino, monosubstituted amino, disubstituted amino, sulfonyl, acyl, alkoxycarbonyl, hydroxyalkyl, aryl, heteroaryl, cycloalkyl, heterocyclyl, aralkyl, or aryloxy, or when R^{b} and R^{c} are on adjacent atoms they can combine to form methylenedioxy or ethylenedioxy; where the aromatic or alicyclic ring in R^{b} and R^{c} is optionally substituted with R^{d} or R^{e} where R^{d} and R^{e} are independently selected from alkyl, halo, haloalkyl, haloalkoxy, alkylthio, cyano, alkoxy, or hydroxyl and substituted with or R^{f} where R^{f} is optionally substituted phenyl, optionally substituted heteroaryl, or optionally substituted heterocyclyl.
(viii) Within group IB(g) and groups contained therein, yet another group of compounds is that wherein Ar² is 3,5-diCF₃phenyl; 1-(4-F-phenyl)cyclopent-1-yl; 3,4-dichlorophenyl; 2,4-dichlorophenyl; 2-trifluoromethylphenyl; 3-chlorophenyl; 3-chloro-6-methoxyphenyl; 3-trifluoromethylphenyl; 2,6-dichlorophenyl; naphth-1-yl; 3,4-methylenedioxyphenyl; 4-chlorophenyl; 2,3-dihydroinden-2-yl; biphen-4-yl; 5-methyl-1H-imidazo[1,2-a]pyridin2-yl; phenyl; 5-cyclopropylisoxazol-3-yl; 6-chloropyridin-2-yl; 5-chlorothiophen-2-yl; 3,5-difluorophenyl; 5-bromopyridin-3-yl; 4-methoxyphenyl; 3-phenoxyphenyl; naphth-2-yl; 3-methoxyphenyl; 3-methoxycyclohexyl; 2-chloro-5-bromophenyl; 3-chloro-4-methoxyphenyl; 5-methylthiophen-2-yl; 2-chlorophenyl; 3,4-ethylenedioxyphenyl;

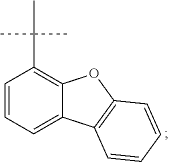

3,5-dimethylphenyl; 2,3-dichlorophenyl; 3-dimethylaminophenyl; 3-cyanophenyl; 3-chloro-4,5-dimethoxyphenyl; 2,6-dichloropyridin-4-yl; tetrahydrofuran-2-yl; 1-phenyl-2-oxopyrrolidin-4-yl; cyclopropyl; 1-acetylpiperidin-4-yl; imidazol-1-yl; 3,5-dimethylimidazol-1-yl; 3,5-dimethylisoxazol-4-yl; 1-cyclohexyl-2-oxopyrrolidin-4-yl; 1-tert-butyl-2-oxopyrrolidin-4-yl; 1-benzyl-2-oxopyrrolidin-4-yl; 3-methylphenyl;

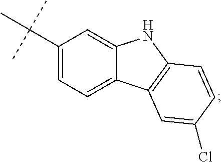

6-methoxynaphth-2-yl; 3,5-dichlorophenyl; 2-methoxy-5-chlorophenyl; 3-benzoylphenyl; 4-isobutylphenyl; 3-fluoro-4-phenylphenyl; benzothiophen-3-yl; 5-bromo-1H-indazol-3-yl; 1H-indol-3-yl; 6-methoxynaphth-2-yl; pyridin-2-yl; 2-biphenyl; 4-dimethylaminophenyl; 4,5,6,7-tetrahydrobenzthiazol-3-yl; cyclohexyl; 1-oxo-2-phenylisoindolin-7-yl; 2-cyclopentyl-1-oxoisoindolin-7-yl; 3-fluorophenyl; 2-chloro-6-fluorophenyl; 3,5-dimethoxyphenyl; 2,5-ditrifluoromethylphenyl; 2-fluoro-3-trifluoromethylphenyl; 4-trifluoromethoxyphenyl; 4-benzyloxyphenyl; 2-benzyloxyphenyl; 2-ethoxyphenyl; 9-methyl-9H-carbazol-2-yl; 2,3-dihydrobenzofuran-5-yl; 4-methoxy-3-methylphenyl; 3-chloro-2,6-difluorophenyl; 3-chloro-9H-carbazol-7-yl; 1-oxoisoindolin-2-yl; 3-bromophenyl; 2-phenylbenzoxazol-5-yl; quinolin-6-yl; 3-(4-morpholin-4-ylphenyl)-phenyl; 5-bromo-3-methylbenzimidazol-1-yl; 4-(4-morpholin-4-ylphenyl)-phenyl; 4-piperidin-1-ylphenyl; 3-(2-morpholin-4-ylpyridin-5-yl)-phenyl; 3-(1-tert-butoxycarbonyl-1,2,3,6-tetrahydropyridin-4-yl)-phenyl; 3-morpholin-4-ylphenyl; 3-(1-methylpiperazin-4-yl)phenyl; 3-(4-pyridin-2-ylpiperazin-1-yl)-phenyl; 3-[1-(2-hydroxyethyl)piperidin-4-yl]phenyl; 2-morpholin-4-ylpyridin-6-yl; 3-methylthiophenyl; 3-methylsulfonylphenyl; 3-(4-dimethylaminophenyl)phenyl; 2-(4-morpholin-4-ylphenyl)pyridin-6-yl; 4-phenylimidazol-1-yl; 3-(3-cyanophenyl)phenyl; 3-(3-methoxyphenyl)phenyl; 3-(2-methoxypyrimidin-5-yl)phenyl; 3-(4-dimethylaminocarbonyl-phenyl)phenyl; 3-(4-methylcarbonyl-phenyl)phenyl; 3-fluoro-5-trifluoromethyl-phenyl; 3-fluoro-5-(4-morpholin-4-ylphenyl)phenyl; 2-(4-trifluoromethylphenyl)-pyridin-4-yl; 3-phenylaminophenyl; 3-(2-methoxyethylamino)phenyl; 4-fluoro-3-morpholin-4-ylphenyl; S-1-tert-butoxycarbonyl-pyrrolidin-2-yl; 5-fluoro-3-(4-morpholin-4-ylphenyl)phenyl; 4-(cyclopropylmethylamino)-phenyl; 4-(N,N-dicyclopropylmethylamino)-phenyl; tetrahydropyran-4-yl; 3-thiophen-3-ylphenyl; 3-indol-5-ylphenyl; 4-N,N-dicyclobutyl-aminophenyl; 3-(2-morpholin-4-ylpyrimidin-5-yl)phenyl; 2-cyclopropylpyridin-4-yl; 4-(4-methoxybenzylamino)-phenyl; 2-(2-methoxyethylamino)-pyridin-6-yl; 3-(2-morpholin-4-ylthiazol-4-yl)phenyl; 2,2-dimethylcycloprop-1-yl; 2,2-difluorocycloprop-1-yl;

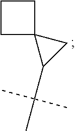

2-pyridin-2-ylbenzoxazol-5-yl; 4-tetrahydropyran-4-ylamino-phenyl; 6-chloroimidazo[1,2-a]pyridin-2-yl; imidazo[1,2-a]pyridin-2-yl; 1-methylindol-2-yl; 4,5,6,7-tetrahydro-1,2-benzisoxazol-3-yl; 6-bromoimidazo[1,2-a]pyridin-2-yl; 1,2,3,4-tetrahydronaphth-1-yl; biphen-3-yl; 4-bromophenyl; 6-chloro-9-methyl-9H-carbazol-2-yl; 3-(4-phenylpiperazin-1-yl)-phenyl; 3-iodophenyl; 4-isopropylaminophenyl; 3-benzyloxyphenyl; 3-(4-cyanophenyl)phenyl; 3-(2-cyanophenyl)phenyl; 3-(4-methoxyphenyl)phenyl; 3-(2-methoxyphenyl)phenyl; 3-(4-trifluoromethoxyphenyl)-phenyl; 3-[4-(1-tert-butoxypiperazin-1-yl)phenyl]-phenyl; 3-(4-piperazin-1-ylphenyl)-phenyl; 1-methylindol-3-yl; 4-morpholin-4-ylphenyl; 9-methyl-9H-carbazol-3yl; 1,3,4,9-tetrahydro-2H-beta-carbolin-2-yl); 8-fluoro-1,3,4,5-tetrahydro-2H-pyrido[4,3-b]indol-2-yl; 6-morpholin-4-ylpyridin-2-yl; 3-pyridin-3-ylphenyl; 3-pyridin-4-ylphenyl; 8-fluoro-1,3,4,5-tetrahydro-2H-pyrido[4,3-b]indol-2-yl; 4-di-n-propylaminophenyl; 4-diethylaminophenyl; 4-(4-morpholin-4-ylphenyl)-pyridin-2-yl; 2-(4-morpholin-4-yl-phenyl)-pyridin-4-yl; 3-methyl-5-(2-methylpyrimidin-5-yl)-phenyl; 5-cyclopropyl-1H-pyrazol-3-yl; 7-methylimidazo[1,2-a]pyridin-2-yl; 8-methylimidazo[1,2-a]pyridin-2-yl; pyridin-3-yl; 1-phenylcycloprop-1-yl; 1-(4-Clphenyl)cycloprop-1-yl; 2,4-dimethoxyphenyl; 2,5-dimethoxyphenyl; 2,5-dimethylphenyl; 3,4-dimethoxyphenyl; 3-trifluoromethoxyphenyl; 4-biphenyl; 3-methoxy-4-methylphenyl; 1-(2-Fphenyl)-cycloprop-1-yl; 1-(4-Fphenyl)cycloprop-1-yl; 1-(3-Fphenyl)cycloprop-1-yl; 1-(phenyl)-cyclobut-1-yl; 1-(phenyl)cyclohex-1-yl; 4-chloropyridin-2-yl; 1-(3-F-phenyl)cyclopent-1-yl; 1-(2-phenylethyl)-2-oxopyrrolidin-4-yl; 6-chloro-1H-imidazo[1,2-a]pyridin-2-yl; 3-methylpyridin-2-yl; 1H-imidazo[1,2-a]pyridin-2-yl; 2-methoxyphenyl; 1-(4-chlorophenyl)-2-oxopyrrolidin-4-yl; 1-benzylpyrrolidin-2-yl; 1-cyclohexylazetidin-2-yl; 1-benzylpyridin-4-yl; 1-(3-chlorophenyl)-2-oxopyrrolidin-4-yl; 6-fluorobenzthiophen-2-yl; 5-bromo-1H-imidazo[1,2-a]pyridin-2-yl; 2-phenylcyclopropyl (Jeff need stereochem); 2-fluorophenyl; 1,2,3,4-tetrahydronaphth-2-yl; 4H-chromen-4-one-3-yl; 4-methylphenyl; thien-2-yl; thien-3-yl; 2,3-dimethoxyphenyl; 1-(4-methoxyphenyl)cycloprop-1-yl; 1-(2,4-dichlorophenyl)-cycloprop-1-yl; 2-bromophenyl; 2-iodophenyl; 2-fluoro-6-trifluoro-methylphenyl; 2,4,6-trimethylphenyl; 4-fluoro-3-trifluoromethyl-phenyl; 2-methylphenyl; 2,4-ditrifluoromethylphenyl; 3-fluoro-4-methoxyphenyl; 2-chloro-5-fluorophenyl; 2-chloro-4-fluorophenyl; 2-bromo-5-chlorophenyl; 4-iodophenyl; 2-fluoro-5-trifluoromethyl-phenyl; 3-chloro-5-fluorophenyl; 4-n-butoxyphenyl; 4-tert-butylphenyl; 4-isopropylphenyl; 5-bromo-2-methoxyphenyl; 3-chloro-2-fluorophenyl; 2-fluoro-4-trifluoromethylphenyl; 3-fluoro-4-trifluoromethylphenyl; 2,6-difluoro-3-methylphenyl; 2-chloro-5-trifluoromethylphenyl; 2-trifluoromethoxyphenyl; 5-fluoro-2-trifluoromethylphenyl; indol-1-yl; 4-aminophenyl; 4-phenoxyphenyl; 3-(1,2,3,6-tetrahydropyridin-4-yl)phenyl; 3-(piperidin-4-yl)phenyl; 3-(morpholin-4-yl)phenyl; 3-fluoro-2-trifluoromethyl-phenyl; 6-fluoropyridin-2-yl; 3-(2-dimethylaminophenyl)-phenyl; 5-chloro-2-fluorophenyl; 3-morpholin-4-yl-5-trifluoromethylphenyl; 3-(4-methylsulfonylphenyl)-phenyl; 3-phenylimidazol-1-yl; 3-bromo-5-fluorophenyl; 3-methyl-4-phenylpyrazol-1-yl; 4-(4-bromophenyl)pyrazol-1-yl; 3-bromo-4-fluorophenyl; 3-piperidin-1-ylphenyl; 2-chloropyridin-4-yl; 4-fluoro-3-(2-methoxyethylamino)phenyl; 3-cyano-5-trifluoromethylphenyl; 3-bromo-5-trifluoromethylphenyl; 4-isopropylmethyl-aminophenyl; indol-5-yl; 3-bromo-4-fluorophenyl; 4-bromoimidazol-1-yl; 1,2,3,4-tetrahydroindol-5-yl; 4-trifluoromethylphenyl; 3-pyrimidin-5-ylphenyl; 3-(methylthiopyrimidin-5-yl)phenyl; 3-(2-aminopyrimidin-5-yl)phenyl; 3-(1-methylindol-5-yl)phenyl; 4-(cyclopentylamino)phenyl; 4-cyclobutylaminophenyl; 3R,5S-(5-phenyl)piperidin-3-yl; 4-N,N-diethylaminophenyl; 3-(2-hydroxypyridin-4-yl)phenyl; 4-(3,4,5-dimethoxybenzyl-amino)phenyl; 3-[4-(4-acetylpiperazin-1-yl)-phenyl)-phenyl; 4-cyanophenyl; 3-cyclopropylphenyl; 4-(ethylamino)phenyl; 4-(N-3,4,5-dimethoxybenzyl-N-ethylamino)phenyl; 4-(cyclopropylamino)phenyl; 4-(N,N-dicyclopropyl-amino)phenyl; 6-(4-morpholin-4-ylphenyl)-pyridin-2-yl; 3-(6-fluoropyridin-3-yl)phenyl; 6-bromopyridin-2-yl; 4-(N-3,4,5-dimethoxybenzyl-N-methylamino)phenyl; 3-(3-chloropyridin-4-yl)phenyl; 5-fluoro-3-(6-morpholin-4-ylpyridin-3-yl)phenyl; 4-sec-butylaminophenyl; 4-methylaminophenyl; 4-n-propylaminophenyl; 4-N,N-di-n-propylaminophenyl; 2-methylbenzimidazol-6-yl; 2-ethylbenzoxazol-5-yl; 2-methylbenzoxazol-5-yl; 3-methoxyquinolin-6-yl; cyclobutyl; 2-methylcyclopropyl; cyclopentyl; 3-pyrimidin-2-ylphenyl; 3-pyridin-2-ylphenyl; tetrahydrofuran-3-yl; 2,4-dimethylthiazol-5-yl; 3-(6-dimethylamino-pyridin-4-yl)-phenyl; 3-(4-methylimidazol-1-yl)-phenyl; 3-(pyrazol-1-yl)-phenyl; 4-N-acetyl-N-isopropylamino-phenyl; 6-chloropyridin-3-yl; 4-amino-3-methoxyphenyl; 4-isopropylamino-3-methoxyphenyl; 3-bromo-5-methylphenyl; 4-fluoro-3-(4-morpholin-4-ylphenyl)phenyl; 6-piperidin-1-ylpyridin-3-yl; 5-methyl-3-(2-methoxy-pyrimidin-5-yl)phenyl; benzoxazol-5-yl; 2-pyridin-3-ylbenzoxazol-5-yl; 2-pyridin-4-ylbenzoxazol-5-yl; 3,5-di(4-trifluoromethyl-phenyl)phenyl; 5-isopropylaminopyridin-2-yl; 4-methyl-3-indol-5-ylphenyl; 3-bromo-5-chlorophenyl; 5-chloro-3-(2-methyl-pyrimidin-5-yl)phenyl; 5-chloro-3-(1,5-dimethylpyrazol-4-yl)phenyl; 5-chloro-3-(pyridin-3-yl)phenyl; 5-methyl-3-(4-cyanophenyl)-phenyl; 5-methyl-3-(3-cyanophenyl)-phenyl; 5-methyl-3-(2-cyanophenyl)-phenyl; 4-isobutylaminophenyl; 4-N,N-di-isobutylaminophenyl; 3-cyano-5-methylphenyl; 4-(N-3,4,5-dimethoxybenzyl-N-n-propyl-amino)phenyl; 3-(2-methylaminopyridin-3-yl)-phenyl; 3-[2-(2-methoxyethyl-amino)pyridin-3-yl]-phenyl; 2-methylbenzimidazol-5-yl; 6-methylsulfonylindol-2-yl; 5-methyl-3-(2-methylpyrimidin-5-yl)-phenyl; benzothiophen-2-yl; 5-methyl-3-(4-pyridin-2-ylpiperazin-1-yl)-phenyl; 5-methyl-3-(2-methylpyridin-4-yl)-phenyl; 5-fluoro-3-(pyridin-3-yl)-phenyl; 5-fluoro-3-(2-methylpyridin-4-yl)-phenyl; 3-difluoromethyl-5-trifluoromethyl-phenyl; 3-fluoro-5-methylphenyl; isoquinolin-5-yl; 1-methylindol-5-yl; quinolin-5-yl; benzo[d][1,2,3]thiadiazol-5-yl; 4-morpholin-4-ylmethylphenyl; 1-methyl-3-ethylpyrazol-5-yl; 3-ethoxyphenyl; 3-morpholin-4-ylmethylphenyl; quinolin-4-yl; 3-difluoromethoxyphenyl; 3,5-dimethylthiazol-4-yl; 3-(6-cyanopyridin-3-yl)phenyl; 5-methyl-3-(1-methyl-2-oxopiperazin-4-yl)phenyl; 5-methyl-3-(4-methoxypiperidin-1yl)phenyl; 5-methyl-3-(4,4-difluoropiperidin-1-yl)phenyl; 3-(6-isopropylaminopyridin-3-yl)phenyl; 3-(4-carboxyphenyl)phenyl; 3-(6-chloropyrazin-2-yl)phenyl; 3-(5-methoxy-pyrazin-2-yl)phenyl; 5-bromo-2-fluorophenyl; 5-chloro-3-(3-cyanophenyl)-phenyl; 5-chloro-3-(2-methoxy-pyrimidin-5-yl)-phenyl; 5-chloro-3-(furan-3-yl)-phenyl; 5-chloro-3-(4-cyanophenyl)-phenyl; 4,6-ditrifluoromethylpyridin-2-yl; pyridin-4-yl; indol-6-yl; 3-acetylaminophenyl; 6-isopropyl-aminopyridin-3-yl; 4-N-4-methoxybenzyl-N-methylaminophenyl; 4-benzylamino-phenyl; 6-dimethylaminopyridin-3-yl; 3-(3-methoxypiperidin-1-yl)phenyl; 3-(4-tert-butoxycarbonyl-piperazin-1-yl)phenyl; 3-(piperazin-1-yl)phenyl; 1-methylindazol-3-yl; 4-pyrrol-1-ylphenyl; 4-bromothiophen-2-yl; 4-N-benzyl-N-methylphenyl; 4-(2-methoxy-pyrimidin-5-yl)thiophen-2-yl; 5-bromothiophen-2-yl; 5-bromophenyl; 3-(2-methoxyethyl-amino-methyl)phenyl; 3-(2-dimethylamino-methyl)phenyl; 3-(piperidin-1-ylmethyl)phenyl; 3-hydroxymethylphenyl; or 5-methylpyridin-3-yl.
(ix) Within groups IB(g) and groups contained therein, yet another group of compounds is that wherein Ar² is 3,5-diCF₃phenyl; 3,4-dichlorophenyl; 2,4-dichlorophenyl; 2-trifluoromethylphenyl; 3-chlorophenyl; 3-chloro-6-methoxyphenyl; 3-trifluoromethylphenyl; 2,6-dichlorophenyl; naphth-1-yl; 3,4-methylenedioxyphenyl; 4-chlorophenyl; biphen-4-yl; phenyl; 3,5-difluorophenyl; 4-methoxyphenyl; 3-phenoxyphenyl; naphth-2-yl; 3-methoxyphenyl; 2-chloro-5-bromophenyl; 3-chloro-4-methoxyphenyl; 2-chlorophenyl; 3,4-ethylenedioxyphenyl; 3,5-dimethylphenyl; 2,3-dichlorophenyl; 3-dimethylaminophenyl; 3-cyanophenyl; 3-chloro-4,5-dimethoxyphenyl; 3-methylphenyl; 6-methoxynaphth-2-yl; 3,5-dichlorophenyl; 2-methoxy-5-chlorophenyl; 3-benzoylphenyl; 4-isobutylphenyl; 3-fluoro-4-phenylphenyl; 6-methoxynaphth-2-yl; 2-biphenyl; 4-dimethylaminophenyl; 3-fluorophenyl; 2-chloro-6-fluorophenyl; 3,5-dimethoxyphenyl; 2,5-ditrifluoromethylphenyl; 2-fluoro-3-trifluoromethylphenyl; 4-trifluoromethoxyphenyl; 4-benzyloxyphenyl; 2-benzyloxyphenyl; 2-ethoxyphenyl; 4-methoxy-3-methylphenyl; 3-chloro-2,6-difluorophenyl; 3-bromophenyl; 3-(4-morpholin-4-ylphenyl)-phenyl; 4-(4-morpholin-4-ylphenyl)-phenyl; 4-piperidin-1-ylphenyl; 3-(2-morpholin-4-ylpyridin-5-yl)-phenyl; 3-(1-tert-butoxycarbonyl-1,2,3,6-tetrahydropyridin-4-yl)-phenyl; 3-morpholin-4-ylphenyl; 3-(1-methylpiperazin-4-yl)phenyl; 3-(4-pyridin-2-ylpiperazin-1-yl)-phenyl; 3-[1-(2-hydroxyethyl)piperidin-4-yl]phenyl; 3-methylthiophenyl; 3-methylsulfonylphenyl; 3-(4-dimethylaminophenyl)phenyl; 3-(3-cyanophenyl)phenyl; 3-(3-methoxyphenyl)phenyl; 3-(2-methoxypyrimidin-5-yl)phenyl; 3-(4-dimethylaminocarbonyl-phenyl)phenyl; 3-(4-methylcarbonyl-phenyl)phenyl; 3-fluoro-5-trifluoromethyl-phenyl; 3-fluoro-5-(4-morpholin-4-ylphenyl)phenyl; 3-phenylaminophenyl; 3-(2-methoxyethylamino)phenyl; 4-fluoro-3-morpholin-4-ylphenyl; 5-fluoro-3-(4-morpholin-4-ylphenyl)phenyl; 4-(cyclopropylmethylamino)-phenyl; 4-(N,N-dicyclopropylmethylamino)-phenyl; 3-thiophen-3-ylphenyl; 3-indol-5-ylphenyl; 4-N,N-dicyclobutyl-aminophenyl; 3-(2-morpholin-4-ylpyrimidin-5-yl)phenyl; 4-(4-methoxybenzylamino)-phenyl; 3-(2-morpholin-4-ylthiazol-4-yl)phenyl; 4-tetrahydropyran-4-ylamino-phenyl; biphen-3-yl; 4-bromophenyl; 3-(4-phenylpiperazin-1-yl)-phenyl; 3-iodophenyl; 4-isopropylaminophenyl; 3-benzyloxyphenyl; 3-(4-cyanophenyl)phenyl; 3-(2-cyanophenyl)phenyl; 3-(4-methoxyphenyl)phenyl; 3-(2-methoxyphenyl)phenyl; 3-(4-trifluoromethoxyphenyl)-phenyl; 3-[4-(1-tert-butoxypiperazin-1-yl)phenyl]-phenyl; 3-(4-piperazin-1-ylphenyl)-phenyl; 4-morpholin-4-ylphenyl; 3-pyridin-3-ylphenyl; 3-pyridin-4-ylphenyl; 4-di-n-propylaminophenyl; 4-diethylaminophenyl; 2-methyl-5-(2-methylpyrimidin-5-yl)-phenyl; 2,4-dimethoxyphenyl; 2,5-dimethoxyphenyl; 2,5-dimethylphenyl; 3,4-dimethoxyphenyl; 3-trifluoromethylphenyl; 4-biphenyl; 3-methoxy-4-methylphenyl; 2-methoxyphenyl; 2-fluorophenyl; 4-methylphenyl; 2,3-dimethoxyphenyl; 2-bromophenyl; 2-iodophenyl; 2-fluoro-6-trifluoro-methylphenyl; 2,4,6-trimethylphenyl; 4-fluoro-3-trifluoromethyl-phenyl; 2-methylphenyl; 2,4-ditrifluoromethylphenyl; 3-fluoro-4-methoxyphenyl; 2-chloro-5-fluorophenyl; 2-chloro-4-fluorophenyl; 2-bromo-5-chlorophenyl; 4-iodophenyl; 2-fluoro-5-trifluoromethyl-phenyl; 3-chloro-5-fluorophenyl; 4-n-butoxyphenyl; 4-tert-butylphenyl; 4-isopropylphenyl; 5-bromo-2-methoxyphenyl; 3-chloro-2-fluorophenyl; 2-fluoro-4-trifluoromethylphenyl; 3-fluoro-4-trifluoromethylphenyl; 2,6-difluoro-3-methylphenyl; 2-chloro-5-trifluoromethylphenyl; 2-trifluoromethoxyphenyl; 5-fluoro-2-trifluoromethylphenyl; 4-aminophenyl; 4-phenoxyphenyl; 3-(1,2,3,6-tetrahydropyridin-4-yl)phenyl; 3-(piperidin-4-yl)phenyl; 3-(morpholin-4-yl)phenyl; 3-fluoro-2-trifluoromethyl-phenyl; 3-(2-dimethylaminophenyl)-phenyl; 5-chloro-2-fluorophenyl; 3-morpholin-4-yl-5-trifluoromethylphenyl; 3-(4-methylsulfonylphenyl)-phenyl; 3-bromo-5-fluorophenyl; 3-bromo-4-fluorophenyl; 3-piperidin-1-ylphenyl; 4-fluoro-3-(2-methoxyethylamino)phenyl; 3-cyano-5-trifluoromethylphenyl; 3-bromo-5-trifluoromethylphenyl; 4-isopropylmethyl-aminophenyl; 3-bromo-5-fluorophenyl; 4-trifluoromethylphenyl; 3-pyrimidin-5-ylphenyl; 3-(methylthiopyrimidin-5-yl)phenyl; 3-(2-aminopyrimidin-5-yl)phenyl; 3-(1-methylindol-5-yl)phenyl; 4-(cyclopentylamino)phenyl; 4-cyclobutylaminophenyl; 4-N,N-diethylaminophenyl; 3-(2-hydroxypyridin-4-yl)phenyl; 4-(3,4,5-dimethoxybenzyl-amino)phenyl; 3-[4-(4-acetylpiperazin-1-yl)-phenyl)-phenyl; 4-cyanophenyl; 3-cyclopropylphenyl; 4-(ethylamino)phenyl; 4-(N-3,4,5-dimethoxybenzyl-N-ethylamino)phenyl; 4-(cyclopropylamino)phenyl; 4-(N,N-dicyclopropylamino)phenyl; 3-(6-fluoropyridin-3-yl)phenyl; 6-bromopyridin-2-yl; 4-(N-3,4,5-dimethoxybenzyl-N-methylamino)phenyl; 3-(3-chloropyridin-4-yl)phenyl; 5-fluoro-3-(6-morpholin-4-ylpyridin-3-yl)phenyl; 4-sec-butylaminophenyl; 4-methylaminophenyl; 4-n-propylaminophenyl; 4-N,N-di-n-propylamino-phenyl; 3-pyrimidin-2-ylphenyl; 3-pyridin-2-ylphenyl; 3-(6-dimethylamino-pyridin-4-yl)-phenyl; 3-(4-methylimidazol-1-yl)-phenyl; 3-(pyrazol-1-yl)-phenyl; 4-N-acetyl-N-isopropylamino-phenyl; 4-amino-3-methoxyphenyl; 4-isopropylamino-3-methoxyphenyl; 3-bromo-5-methylphenyl; 4-fluoro-3-(4-morpholin-4-ylphenyl)phenyl; 5-methyl-3-(2-methoxy-pyrimidin-5-yl)phenyl; 3,5-di(4-trifluoromethyl-phenyl)phenyl; 4-methyl-3-indol-5-ylphenyl; 3-bromo-5-chlorophenyl; 5-chloro-3-(2-methyl-pyrimidin-5-yl)phenyl; 5-chloro-3-(1,5-dimethylpyrazol-4-yl)phenyl; 5-chloro-3-(pyridin-3-yl)phenyl; 5-methyl-3-(4-cyanophenyl)-phenyl; 5-methyl-3-(3-cyanophenyl)-phenyl; 5-methyl-3-(2-cyanophenyl)-phenyl; 4-isobutylaminophenyl; 4-N,N-di-isobutylaminophenyl; 3-cyano-5-methylphenyl; 4-(N-3,4,5-dimethoxybenzyl-N-n-propyl-amino)phenyl; 3-(2-methylaminopyridin-3-yl)-phenyl; 3-[2-(2-methoxyethyl-amino)pyridin-3-yl]-phenyl; 5-methyl-3-(2-methylpyrimidin-5-yl)-phenyl; 5-methyl-3-(4-pyridin-2-ylpiperazin-1-yl)-phenyl; 5-methyl-3-(2-methylpyridin-4-yl)-phenyl; 5-fluoro-3-(pyridin-3-yl)-phenyl; 5-fluoro-3-(2-methylpyridin-4-yl)-phenyl; 3-difluoromethyl-5-trifluoromethyl-phenyl; 3-fluoro-5-methylphenyl; 4-morpholin-4-ylmethylphenyl; 3-ethoxyphenyl; 3-morpholin-4-ylmethylphenyl; 3-difluoromethoxyphenyl; 3-(6-cyanopyridin-3-yl)phenyl; 5-methyl-3-(1-methyl-2-oxopiperazin-4-yl)phenyl; 5-methyl-3-(4-methoxypiperidin-1-yl)phenyl; 5-methyl-3-(4,4-difluoropiperidin-1-yl)phenyl; 3-(6-isopropylaminopyridin-3-yl)phenyl; 3-(4-carboxyphenyl)phenyl; 3-(6-chloropyrazin-2-yl)phenyl; 3-(5-methoxy-pyrazin-2-yl)phenyl; 5-bromo-2-fluorophenyl; 5-chloro-3-(3-cyanophenyl)-phenyl; 5-chloro-3-(2-methoxy-pyrimidin-5-yl)-phenyl; 5-chloro-3-(furan-3-yl)-phenyl; 5-chloro-3-(4-cyanophenyl)-phenyl; 3-acetylaminophenyl; 4-N-4-methoxybenzyl-N-methylaminophenyl; 4-benzylaminophenyl; 3-(3-methoxypiperidin-1-yl)phenyl; 3-(4-tert-butoxycarbonyl-piperazin-1-yl)phenyl; 3-(piperazin-1-yl)phenyl; 4-pyrrol-1-ylphenyl; 4-N-benzyl-N-methylphenyl; 5-bromophenyl; 3-(2-methoxyethylamino-methyl)phenyl; 3-(2-dimethylamino-methyl)phenyl; 3-(piperidin-1-ylmethyl)phenyl; or 3-hydroxymethylphenyl.
(x) Within groups IB(g) and groups contained therein, yet another group of compounds is that wherein Ar² is 5-methyl-1H-imidazo[1,2-a]pyridin-2-yl; 5-cyclopropylisoxazol-3-yl; 6-chloropyridin-2-yl; 5-chlorothiophen-2-yl; 5-bromopyridin-3-yl; 5-methylthiophen-2-yl;

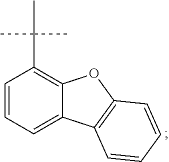

2,6-dichloropyridin-4-yl; imidazol-1-yl; 3,5-dimethylimidazol-1-yl; 3,5-dimethylisoxazol-4-yl;

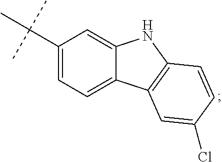

benzothiophen-3-yl; 5-bromo-1H-indazol-3-yl; 1H-indol-3-yl; pyridin-2-yl; 9-methyl-9H-carbazol-2-yl; 2,3-dihydrobenzofuran-5-yl; 3-chloro-9H-carbazol-7-yl; 2-phenylbenzoxazol-5-yl; quinolin-6-yl; 5-bromo-3-methylbenzimidazol-1-yl; 2-morpholin-4-ylpyridin-6-yl; 2-(4-morpholin-4-ylphenyl)pyridin-6-yl; 4-phenylimidazol-1-yl; 2-(4-trifluoromethylphenyl)-pyridin-4-yl; 2-cyclopropylpyridin-4-yl; 2-(2-methoxyethylamino)-pyridin-6-yl; 2-pyridin-2-ylbenzoxazol-5-yl; 6-chloroimidazo[1,2-a]pyridin-2-yl; imidazo[1,2-a]pyridin-2-yl; 1-methylindol-2-yl; 6-bromoimidazo[1,2-a]pyridin-2-yl; 6-chloro-9-methyl-9H-carbazol-2-yl; 1-methylindol-3-yl; 9-methyl-9H-carbazol-3yl; 6-morpholin-4-ylpyridin-2-yl; 4-(4-morpholin-4-ylphenyl)-pyridin-2-yl; 2-(4-morpholin-4-yl-phenyl)-pyridin-4-yl; 5-cyclopropyl-1H-pyrazol-3-yl; 7-methylimidazo[1,2-a]pyridin-2-yl; 8-methylimidazo[1,2-a]pyridin-2-yl; pyridin-3-yl; 4-chloropyridin-2-yl; 6-chloro-1H-imidazo[1,2-a]pyridin-2-yl; 3-methylpyridin-2-yl; 1H-imidazo[1,2-a]pyridin-2-yl; 1-benzylpyridin-4-yl; 6-fluorobenzthiophen-2-yl; 5-bromo-1H-imidazo[1,2-a]pyridin-2-yl; thien-2-yl; thien-3-yl; indol-1-yl; 6-fluoropyridin-2-yl; 3-phenylimidazol-1-yl; 3-methyl-4-phenylpyrazol-1-yl; 4-(4-bromophenyl)pyrazol-1-yl; 2-chloropyridin-4-yl; indol-5-yl; 4-bromoimidazol-1-yl; 6-(4-morpholin-4-ylphenyl)-pyridin-2-yl; 6-bromopyridin-2-yl; 2-methylbenzimidazol-6-yl; 2-ethylbenzoxazol-5-yl; 2-methylbenzoxazol-5-yl; 3-methoxyquinolin-6-yl; 2,4-dimethylthiazol-5-yl; 6-chloropyridin-3-yl; 6-piperidin-1-ylpyridin-3-yl; benzoxazol-5-yl; 2-pyridin-3-ylbenzoxazol-5-yl; 2-pyridin-4-ylbenzoxazol-5-yl; 5-isopropylaminopyridin-2-yl; 2-methylbenzimidazol-5-yl; 6-methylsulfonylindol-2-yl; benzothiophen-2-yl; isoquinolin-5-yl; 1-methylindol-5-yl; quinolin-5-yl; benzo[d][1,2,3]thiadiazol-5-yl; 1-methyl-3-ethylpyrazol-5-yl; quinolin-4-yl; 3,5-dimethylthiazol-4-yl; 4,6-ditrifluoromethylpyridin-2-yl; pyridin-4-yl; indol-6-yl; 6-isopropylaminopyridin-3-yl; 6-dimethylaminopyridin-3-yl; 1-methylindazol-3-yl; 4-bromothiophen-2-yl; 4-(2-methoxypyrimidin-5-yl)thiophen-2-yl; 5-bromothiophen-2-yl; or 5-methylpyridin-3-yl.
(xi) Within groups IB(g) and groups contained therein, yet another group of compounds is that wherein Ar² is 1-(4-F-phenyl)cyclopent-1-yl; 2,3-dihydroinden-2-yl; 3-methoxycyclohexyl; tetrahydrofuran-2-yl; 1-phenyl-2-oxopyrrolidin-4-yl; 1-acetylpiperidin-4-yl; 1-cyclohexyl-2-oxopyrrolidin-4-yl; 1-tert-butyl-2-oxopyrrolidin-4-yl; 1-benzyl-2-oxopyrrolidin-4-yl; 4,5,6,7-tetrahydrobenzthiazol-3-yl; cyclohexyl; 1-oxo-2-phenylisoindolin-7-yl; 2-cyclopentyl-1-oxoisoindolin-7-yl; 2,3-dihydrobenzofuran-5-yl; 1-oxoisoindolin-2-yl; S-1-tert-butoxycarbonyl-pyrrolidin-2-yl; tetrahydropyran-4-yl; 2-cyclopropylpyridin-4-yl; 2,2-dimethylcycloprop-1-yl; 2,2-difluorocycloprop-1-yl; 4,5,6,7-tetrahydro-1,2-benzisoxazol-3-yl; 1,2,3,4-tetrahydronaphth-1-yl; 1,3,4,9-tetrahydro-2H-beta-carbolin-2-yl); 8-fluoro-1,3,4,5-tetrahydro-2H-pyrido[4,3-b]indol-2-yl; 8-fluoro-1,3,4,5-tetrahydro-2H-pyrido[4,3-b]indol-2-yl; 1-phenylcycloprop-1-yl; 1-(4-Clphenyl)cycloprop-1-yl; 1-(2-Fphenyl)-cycloprop-1-yl; 1-(4-Fphenyl)cycloprop-1-yl; 1-(3-Fphenyl)cycloprop-1-yl; 1-(phenyl)-cyclobut-1-yl; 1-(phenyl)cyclohex-1-yl; 1-(3-F-phenyl)cyclopent-1-yl; 1-(2-phenylethyl)-2-oxopyrrolidin-4-yl; 1-(4-chlorophenyl)-2-oxopyrrolidin-4-yl; 1-benzylpyrrolidin-2-yl; 1-cyclohexylazetidin-2-yl; 1-benzylpyridin-4-yl; 1-(3-chlorophenyl)-2-oxopyrrolidin-4-yl; 2-phenylcyclopropyl; 1,2,3,4-tetrahydronaphth-2-yl; 4H-chromen-4-one-3-yl; 1-(4-methoxyphenyl)cycloprop-1-yl; 1-(2,4-dichlorophenyl)-cycloprop-1-yl; 1,2,3,4-tetrahydroindol-5-yl; 3R,5S-(5-phenyl)piperidin-3-yl; cyclobutyl; 2-methylcyclopropyl; cyclopentyl; or tetrahydrofuran-3-yl.

II. In another embodiment, the compound of Formula (I′) is represented by the structure:

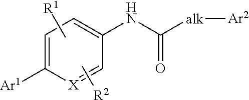

III. In yet another embodiment, the compound of Formula (I′) is represented by the structure:

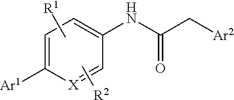

IV. In yet another embodiment, the compound of Formula (I′) is represented by the structure:

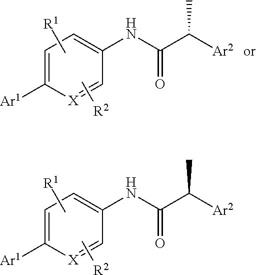

V. In yet another embodiment, the compound of Formula (I′) is represented by the structure:

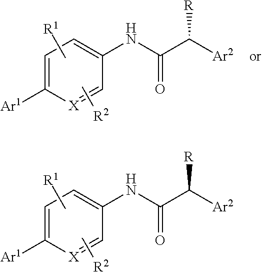

where R is n-propyl, n-butyl, or isobutyl.

VI. In yet another embodiment, the compound of Formula (I) where n is 1, preferably the compound is represented by the structure:

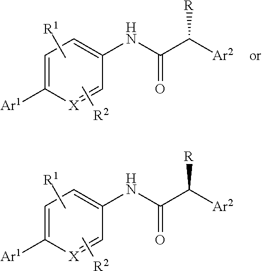

where R is hydrogen, methyl, n-propyl, n-butyl, isobutyl, cyclopropylmethyl, or —CH₂CF(CH₃)₂, preferably hydrogen, methyl or isobutyl.
(a) Within embodiments (II), (III), (IV), (V) and (VI) independently, and groups contained therein, one group of compounds is that wherein X is —N—.
(b) Within embodiments (II), (III), (IV), (V) and (VI) independently, and groups contained therein, another group of compounds is that wherein X is —CH—.
(c) Within embodiments (II), (III), (IV), (V) and (VI) independently, and groups contained therein, yet another group of compounds is that wherein R¹ and R² are hydrogen.

Within group (c), one group of compounds is that wherein X is —CH—.

Within group (c), another group of compounds is that wherein X is —N—.

Within group (c) and groups contained therein, in one group of compounds Ar¹ is a ring of formula (iv) where R⁵ is alkyl, preferably methyl or ethyl, more preferably methyl, more preferably 2-alkylpyridin-4-yl, even more preferably Ar¹ is 2-methylpyridin-4-yl.

(d) Within embodiments (II), (III), (IV), (V) and (VI) independently, yet another group of compounds is that wherein Ar¹ is a ring of formula (i) where R³ is alkyl, preferably methyl or ethyl, more preferably methyl.
(e) Within embodiments (II), (III), (IV), (V) and (VI) independently, yet another group of compounds is that wherein Ar¹ is a ring of formula (ii).
(f) Within embodiments (II), (III), (IV), (V) and (VI) independently, yet another group of compounds is that wherein Ar¹ is a ring of formula (iii) where R⁴ is alkyl, preferably methyl or ethyl, more preferably methyl.
(g) Within embodiments (II), (III), (IV), (V) and (VI) independently, and groups contained therein, yet another group of compounds is that wherein Ar¹ is a ring of formula (iv) where R⁵ is alkyl, preferably methyl or ethyl, more preferably methyl, more preferably 2-alkylpyridin-4-yl, even more preferably Ar¹ is 2-methylpyridin-4-yl.

Within groups (d) through (g) independently, and groups contained therein, in one group of compounds X is —CH—.

Within groups (d) through (g) independently, and groups contained therein, in another group of compounds X is —CH— and R¹ and R² are hydrogen.

Within groups (d) through (g) independently, and groups contained therein, in another group of compounds X is —N—.

Within groups (d) through (g) independently, and groups contained therein, in another group of compounds alk is —CH₂—.

Within groups (d) through (g) independently, and groups contained therein, in another group of compounds alk is —CH(CH₃)—.

Within groups (d) through (g) independently, and groups contained therein, in another group of compounds alk is —CH(isobutyl)-.

(i) Within groups (II), (III), (IV), (V), and (VI), (a) through (g) independently and groups contained therein, one group of compounds is that wherein Ar² is aryl optionally substituted as described in the Summary of the Invention.

Within this group, one group of compounds is that wherein Ar² is phenyl or naphthyl optionally substituted with R^{a} which is halo, alkyl, haloalkyl, or alkoxy or R^{b} which is selected from halo, alkyl, haloalkyl, alkoxy, cycloalkyl, aryl, aryloxy, amino, monosubstituted amino, disubstituted amino, cyano, acyl, or aralkyl. Within this group, another group of compounds is that wherein Ar² is 3,5-diCF₃phenyl, 4-trifluoromethylphenyl, 3,4-dichlorophenyl, 2,4-dichlorophenyl, 2-trifluoromethylphenyl, 3-chlorophenyl, 3-chloro-6-methoxyphenyl, 3-trifluoromethylphenyl, 3-chlorophenyl, 2,6-dichlorophenyl, naphth-1-yl, 3,4-methylene-dioxyphenyl, 4-chlorophenyl, 2-fluorophenyl, biphen-4-yl, phenyl, 3,5-difluorophenyl, 4-methoxyphenyl, 3-phenoxyphenyl, naphth-2-yl, 3-methoxyphenyl, 2-chloro-5-bromophenyl, 3-chloro-4-methoxyphenyl, 2-chlorophenyl, 3,4-ethylenedioxyphenyl, 3,5-dimethylphenyl, 2,3-dichlorophenyl, 3-dimethylaminophenyl, 3-cyanolphenyl, 3-chloro-4,5-dimethoxyphenyl, 3-methylphenyl, 6-methoxynaphth-2-yl, 3,5-dichlorophenyl, 2-methoxy-5-chlorophenyl, naphth-1-yl, 4-methylphenyl, 3-methylphenyl, 4-benzoylphenyl, 4-isobutylphenyl, or 3-fluoro-4-phenylphenyl.

(ii) Within groups (II), (III), (IV), (V) and (VI), (a) through (g) independently, and groups contained therein, another group of compounds is that wherein Ar² is heteroaryl optionally substituted as described in the Summary of the Invention. Within this group, one group of compounds is that wherein Ar² heteroaryl optionally substituted with R^{a} which is halo, alkyl, haloalkyl, or alkoxy or R^{b} which is selected from halo, alkyl, haloalkyl, alkoxy, cycloalkyl, aryl, aryloxy, amino, monosubstituted amino, disubstituted amino, cyano, acyl, or aralkyl. Within this group, another group of compounds is that wherein Ar² is 5-methylH-imidazo[1,2-a]pyridin2-yl, 5-cyclopropylisoxazol-3-yl, 6-chloropyridin-2-yl, 5-chlorothiophen-2-yl, 5-bromopyridin-3-yl, 5-methylthiophen-2-yl, 2,6-dichloropyridin-4-yl, imidazol-1-yl, 3,5-dimethylimidazol-1-yl, 3,5-dimethylisoxazol-4-yl, benzothiophen-3-yl, 5-bromo-1H-indazol-3-yl, or 1H-indol-3-yl.
(iii) Within groups (II), (III), (IV), (V), and (VI), (a) through (g) independently, and groups contained therein, yet another group of compounds is that wherein Ar² is cycloalkyl or heterocyclyl optionally substituted as described in the Summary of the Invention. Within this group, one group of compounds is that wherein Ar² cycloalkyl or heterocyclyl optionally substituted with R^{a} which is halo, alkyl, haloalkyl, or alkoxy or R^{b} is selected from halo, alkyl, haloalkyl, alkoxy, cycloalkyl, aryl, aryloxy, amino, monosubstituted amino, disubstituted amino, cyano, acyl, or aralkyl. Within this group, another group of compounds is that wherein Ar² is 1-(4-F-phenyl)-cyclopent-1-yl, 3-methoxycyclohexyl, tetrahydrofuran-2-yl, 1-phenyl-2-oxopyrrolidin-4-yl, cyclopropyl, 1-acetylpiperidin-4-yl, 1-cyclohexyl-2-oxopyrrolidin-4-yl, 1-tert-butyl-2-oxopyrrolidin-4-yl, or 1-benzyl-2-oxopyrrolidin-4-yl.
(iv) Within groups (II), (III), (IV), (V), and (VI), (a) through (g) independently, and groups contained therein, yet another group of compounds is that wherein Ar² is fused cycloalkyl or fused heterocyclyl, preferably fused heterocyclyl, more preferably carbazolyl optionally substituted as described in the Summary of the Invention. Within this group, one group of compounds is that wherein Ar² fused cycloalkyl or fused heterocyclyl, preferably fused heterocyclyl, optionally substituted with R^{a} which is halo, alkyl, haloalkyl, or alkoxy or R^{b} is selected from halo, alkyl, haloalkyl, alkoxy, cycloalkyl, aryl, aryloxy, amino, monosubstituted amino, disubstituted amino, cyano, acyl, or aralkyl. Within this group, another group of compounds is that wherein Ar² is:

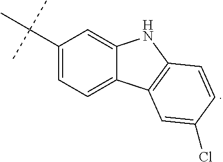

(v) Within groups (VI), (a)(VI), (b)(VI), (c)(VI), (d)(VI), (E)(VI), (f)(VI) and (g)(VI) independently, and groups contained therein, yet another group of compounds is that wherein Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, hydroxyl, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, optionally substituted phenyl, optionally substituted heteroaryl, or optionally substituted heterocyclyl. Preferably, Ar² is aryl or heteroaryl where each of the aforementioned ring is substituted with R^{a} or R^{b} where R^{a} is alkyl, halo, haloalkyl, haloalkoxy, cyano, alkoxy and R^{b} is alkyl, halo, haloalkyl, haloalkoxy, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy where the aromatic or alicyclic ring in R^{a} and R^{b} is optionally substituted with R^{d} or R^{e} which are independently selected from alkyl, halo, haloalkyl, haloalkoxy, cyano, alkoxy, or hydroxy.

In another embodiment within this group, Ar² is aryl or heteroaryl substituted with R^{a} and R^{b} where R^{a} is alkyl, halo, haloalkyl, haloalkoxy, cyano, alkoxy, and R^{b} is aryl, heteroaryl, or heterocycly where the aromatic or alicyclic ring in R^{b} is substituted with R^{d} or R^{e} where Rd is alkyl, halo, haloalkyl, haloalkoxy, cyano, alkoxy, or hydroxyl and R^{e} is optionally substituted phenyl, optionally substituted heteroaryl, or optionally substituted heterocyclyl.

(vi) Within groups Within groups (VI), (a)(VI), (b)(VI), (c)(VI), (d)(VI), (E)(VI), (f)(VI) and (g)(VI) independently, and groups contained therein, yet another group of compounds is that wherein Ar² is aryl, heteroaryl, cycloalkyl, heterocyclyl attached to —NHCO— group via carbon atom, or fused heterocyclyl, preferably aryl or heteroaryl, where each of the aforementioned ring is optionally substituted with R^{a} where R^{a} is alkyl, halo, haloalkyl, or alkoxy; and substituted with R^{b} where R^{b} is alkyl, halo, haloalkyl, haloalkoxy, alkylthio, cyano, alkoxy, amino, monosubstituted amino, disubstituted amino, sulfonyl, acyl, alkoxycarbonyl, hydroxyalkyl, aryl, heteroaryl, cycloalkyl, heterocyclyl, aralkyl, or aryloxy, or when R^{b} and R^{c} are on adjacent atoms they can combine to form methylenedioxy or ethylenedioxy; where the aromatic or alicyclic ring in R^{a} and R^{b} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, or aminocarbonyl.
(vii) Within group (VI)(g) and groups contained therein, in another group, Ar² is aryl, heteroaryl, cycloalkyl, heterocyclyl attached to —NHCO— group via carbon atom, or fused heterocyclyl, preferably aryl or heteroaryl, more preferably aryl, where each of the aforementioned ring is optionally substituted with R^{b} where R^{b} is alkyl, halo, haloalkyl, or alkoxy; and substituted with R^{c} where R^{c} is alkyl, halo, haloalkyl, haloalkoxy, alkylthio, cyano, alkoxy, amino, monosubstituted amino, disubstituted amino, sulfonyl, acyl, alkoxycarbonyl, hydroxyalkyl, aryl, heteroaryl, cycloalkyl, heterocyclyl, aralkyl, or aryloxy, or when R^{b} and R^{c} are on adjacent atoms they can combine to form methylenedioxy or ethylenedioxy; where the aromatic or alicyclic ring in R^{b} and R^{c} is optionally substituted with R^{d} or R^{e} where R^{d} and R^{e} are independently selected from alkyl, halo, haloalkyl, haloalkoxy, alkylthio, cyano, alkoxy, or hydroxyl and substituted with or R^{f} where R^{f} is optionally substituted phenyl, optionally substituted heteroaryl, or optionally substituted heterocyclyl.
(viii) Within groups (VI)(g) and groups contained therein, yet another group of compounds is that wherein Ar² is 3,5-diCF₃phenyl; 1-(4-F-phenyl)cyclopent-1-yl; 3,4-dichlorophenyl; 2,4-dichlorophenyl; 2-trifluoromethylphenyl; 3-chlorophenyl; 3-chloro-6-methoxyphenyl; 3-trifluoromethylphenyl; 2,6-dichlorophenyl; naphth-1-yl; 3,4-methylenedioxyphenyl; 4-chlorophenyl; 2,3-dihydroinden-2-yl; biphen-4-yl; 5-methyl-1H-imidazo[1,2-a]pyridin2-yl; phenyl; 5-cyclopropylisoxazol-3-yl; 6-chloropyridin-2-yl; 5-chlorothiophen-2-yl; 3,5-difluorophenyl; 5-bromopyridin-3-yl; 4-methoxyphenyl; 3-phenoxyphenyl; naphth-2-yl; 3-methoxyphenyl; 3-methoxycyclohexyl; 2-chloro-5-bromophenyl; 3-chloro-4-methoxyphenyl; 5-methylthiophen-2-yl; 2-chlorophenyl; 3,4-ethylenedioxyphenyl;

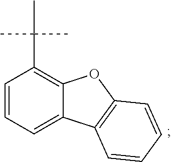

3,5-dimethylphenyl; 2,3-dichlorophenyl; 3-dimethylaminophenyl; 3-cyanophenyl; 3-chloro-4,5-dimethoxyphenyl; 2,6-dichloropyridin-4-yl; tetrahydrofuran-2-yl; 1-phenyl-2-oxopyrrolidin-4-yl; cyclopropyl; 1-acetylpiperidin-4-yl; imidazol-1-yl; 3,5-dimethylimidazol-1-yl; 3,5-dimethylisoxazol-4-yl; 1-cyclohexyl-2-oxopyrrolidin-4-yl; 1-tert-butyl-2-oxopyrrolidin-4-yl; 1-benzyl-2-oxopyrrolidin-4-yl; 3-methylphenyl;

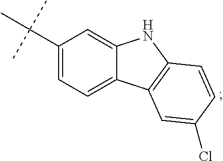

6-methoxynaphth-2-yl; 3,5-dichlorophenyl; 2-methoxy-5-chlorophenyl; 3-benzoylphenyl; 4-isobutylphenyl; 3-fluoro-4-phenylphenyl; benzothiophen-3-yl; 5-bromo-1H-indazol-3-yl; 1H-indol-3-yl; 6-methoxynaphth-2-yl; pyridin-2-yl; 2-biphenyl; 4-dimethylaminophenyl; 4,5,6,7-tetrahydrobenzthiazol-3-yl; cyclohexyl; 1-oxo-2-phenylisoindolin-7-yl; 2-cyclopentyl-1-oxoisoindolin-7-yl; 3-fluorophenyl; 2-chloro-6-fluorophenyl; 3,5-dimethoxyphenyl; 2,5-ditrifluoromethylphenyl; 2-fluoro-3-trifluoromethylphenyl; 4-trifluoromethoxyphenyl; 4-benzyloxyphenyl; 2-benzyloxyphenyl; 2-ethoxyphenyl; 9-methyl-9H-carbazol-2-yl; 2,3-dihydrobenzofuran-5-yl; 4-methoxy-3-methylphenyl; 3-chloro-2,6-difluorophenyl; 3-chloro-9H-carbazol-7-yl; 1-oxoisoindolin-2-yl; 3-bromophenyl; 2-phenylbenzoxazol-5-yl; quinolin-6-yl; 3-(4-morpholin-4-ylphenyl)-phenyl; 5-bromo-3-methylbenzimidazol-1-yl; 4-(4-morpholin-4-ylphenyl)-phenyl; 4-piperidin-1-ylphenyl; 3-(2-morpholin-4-ylpyridin-5-yl)-phenyl; 3-(1-tert-butoxycarbonyl-1,2,3,6-tetrahydropyridin-4-yl)-phenyl; 3-morpholin-4-ylphenyl; 3-(1-methylpiperazin-4-yl)phenyl; 3-(4-pyridin-2-ylpiperazin-1-yl)-phenyl; 3-[1-(2-hydroxyethyl)piperidin-4-yl]phenyl; 2-morpholin-4-ylpyridin-6-yl; 3-methylthiophenyl; 3-methylsulfonylphenyl; 3-(4-dimethylaminophenyl)phenyl; 2-(4-morpholin-4-ylphenyl)pyridin-6-yl; 4-phenylimidazol-1-yl; 3-(3-cyanophenyl)phenyl; 3-(3-methoxyphenyl)phenyl; 3-(2-methoxypyrimidin-5-yl)phenyl; 3-(4-dimethylaminocarbonyl-phenyl)phenyl; 3-(4-methylcarbonyl-phenyl)phenyl; 3-fluoro-5-trifluoromethyl-phenyl; 3-fluoro-5-(4-morpholin-4-ylphenyl)phenyl; 2-(4-trifluoromethylphenyl)-pyridin-4-yl; 3-phenylaminophenyl; 3-(2-methoxyethylamino)phenyl; 4-fluoro-3-morpholin-4-ylphenyl; S-1-tert-butoxycarbonyl-pyrrolidin-2-yl; 5-fluoro-3-(4-morpholin-4-ylphenyl)phenyl; 4-(cyclopropylmethylamino)-phenyl; 4-(N,N-dicyclopropylmethylamino)-phenyl; tetrahydropyran-4-yl; 3-thiophen-3-ylphenyl; 3-indol-5-ylphenyl; 4-N,N-dicyclobutyl-aminophenyl; 3-(2-morpholin-4-ylpyrimidin-5-yl)phenyl; 2-cyclopropylpyridin-4-yl; 4-(4-methoxybenzylamino)-phenyl; 2-(2-methoxyethylamino)-pyridin-6-yl; 3-(2-morpholin-4-ylthiazol-4-yl)phenyl; 2,2-dimethylcycloprop-1-yl; 2,2-difluorocycloprop-1-yl;

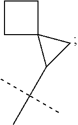

2-pyridin-2-ylbenzoxazol-5-yl; 4-tetrahydropyran-4-ylamino-phenyl; 6-chloroimidazo[1,2-a]pyridin-2-yl; imidazo[1,2-a]pyridin-2-yl; 1-methylindol-2-yl; 4,5,6,7-tetrahydro-1,2-benzisoxazol-3-yl; 6-bromoimidazo[1,2-a]pyridin-2-yl; 1,2,3,4-tetrahydronaphth-1-yl; biphen-3-yl; 4-bromophenyl; 6-chloro-9-methyl-9H-carbazol-2-yl; 3-(4-phenylpiperazin-1-yl)-phenyl; 3-iodophenyl; 4-isopropylaminophenyl; 3-benzyloxyphenyl; 3-(4-cyanophenyl)phenyl; 3-(2-cyanophenyl)phenyl; 3-(4-methoxyphenyl)phenyl; 3-(2-methoxyphenyl)phenyl; 3-(4-trifluoromethoxyphenyl)-phenyl; 3-[4-(1-tert-butoxypiperazin-1-yl)phenyl]-phenyl; 3-(4-piperazin-1-ylphenyl)-phenyl; 1-methylindol-3-yl; 4-morpholin-4-ylphenyl; 9-methyl-9H-carbazol-3yl; 1,3,4,9-tetrahydro-2H-beta-carbolin-2-yl); 8-fluoro-1,3,4,5-tetrahydro-2H-pyrido[4,3-b]indol-2-yl; 6-morpholin-4-ylpyridin-2-yl; 3-pyridin-3-ylphenyl; 3-pyridin-4-ylphenyl; 8-fluoro-1,3,4,5-tetrahydro-2H-pyrido[4,3-b]indol-2-yl; 4-di-n-propylaminophenyl; 4-diethylaminophenyl; 4-(4-morpholin-4-ylphenyl)-pyridin-2-yl; 2-(4-morpholin-4-yl-phenyl)-pyridin-4-yl; 3-methyl-5-(2-methylpyrimidin-5-yl)-phenyl; 5-cyclopropyl-1H-pyrazol-3-yl; 7-methylimidazo[1,2-a]pyridin-2-yl; 8-methylimidazo[1,2-a]pyridin-2-yl; pyridin-3-yl; 1-phenylcycloprop-1-yl; 1-(4-Clphenyl)cycloprop-1-yl; 2,4-dimethoxyphenyl; 2,5-dimethoxyphenyl; 2,5-dimethylphenyl; 3,4-dimethoxyphenyl; 3-trifluoromethylphenyl; 4-biphenyl; 3-methoxy-4-methylphenyl; 1-(2-Fphenyl)-cycloprop-1-yl; 1-(4-Fphenyl)cycloprop-1-yl; 1-(3-Fphenyl)cycloprop-1-yl; 1-(phenyl)-cyclobut-1-yl; 1-(phenyl)cyclohex-1-yl; 4-chloropyridin-2-yl; 1-(3-F-phenyl)cyclopent-1-yl; 1-(2-phenylethyl)-2-oxopyrrolidin-4-yl; 6-chloro-1H-imidazo[1,2-a]pyridin-2-yl; 3-methylpyridin-2-yl; 1H-imidazo[1,2-a]pyridin-2-yl; 2-methoxyphenyl; 1-(4-chlorophenyl)-2-oxopyrrolidin-4-yl; 1-benzylpyrrolidin-2-yl; 1-cyclohexylazetidin-2-yl; 1-benzylpyridin-4-yl; 1-(3-chlorophenyl)-2-oxopyrrolidin-4-yl; 6-fluorobenzthiophen-2-yl; 5-bromo-1H-imidazo[1,2-a]pyridin-2-yl; 2-phenylcyclopropyl (Jeff need stereochem); 2-fluorophenyl; 1,2,3,4-tetrahydronaphth-2-yl; 4H-chromen-4-one-3-yl; 4-methylphenyl; thien-2-yl; thien-3-yl; 2,3-dimethoxyphenyl; 1-(4-methoxyphenyl)cycloprop-1-yl; 1-(2,4-dichlorophenyl)-cycloprop-1-yl; 2-bromophenyl; 2-iodophenyl; 2-fluoro-6-trifluoro-methylphenyl; 2,4,6-trimethylphenyl; 4-fluoro-3-trifluoromethyl-phenyl; 2-methylphenyl; 2,4-ditrifluoromethylphenyl; 3-fluoro-4-methoxyphenyl; 2-chloro-5-fluorophenyl; 2-chloro-4-fluorophenyl; 2-bromo-5-chlorophenyl; 4-iodophenyl; 2-fluoro-5-trifluoromethyl-phenyl; 3-chloro-5-fluorophenyl; 4-n-butoxyphenyl; 4-tert-butylphenyl; 4-isopropylphenyl; 5-bromo-2-methoxyphenyl; 3-chloro-2-fluorophenyl; 2-fluoro-4-trifluoromethylphenyl; 3-fluoro-4-trifluoromethylphenyl; 2,6-difluoro-3-methylphenyl; 2-chloro-5-trifluoromethylphenyl; 2-trifluoromethoxyphenyl; 5-fluoro-2-trifluoromethylphenyl; indol-1-yl; 4-aminophenyl; 4-phenoxyphenyl; 3-(1,2,3,6-tetrahydropyridin-4-yl)phenyl; 3-(piperidin-4-yl)phenyl; 3-(morpholin-4-yl)phenyl; 3-fluoro-2-trifluoromethyl-phenyl; 6-fluoropyridin-2-yl; 3-(2-dimethylaminophenyl)-phenyl; 5-chloro-2-fluorophenyl; 3-morpholin-4-yl-5-trifluoromethylphenyl; 3-(4-methylsulfonylphenyl)-phenyl; 3-phenylimidazol-1-yl; 3-bromo-5-fluorophenyl; 3-methyl-4-phenylpyrazol-1-yl; 4-(4-bromophenyl)pyrazol-1-yl; 3-bromo-4-fluorophenyl; 3-piperidin-1-ylphenyl; 2-chloropyridin-4-yl; 4-fluoro-3-(2-methoxyethylamino)phenyl; 3-cyano-5-trifluoromethylphenyl; 3-bromo-5-trifluoromethylphenyl; 4-isopropylmethyl-aminophenyl; indol-5-yl; 3-bromo-5-fluorophenyl; 4-bromoimidazol-1-yl; 1,2,3,4-tetrahydroindol-5-yl; 4-trifluoromethylphenyl; 3-pyrimidin-5-ylphenyl; 3-(methylthiopyrimidin-5-yl)phenyl; 3-(2-aminopyrimidin-5-yl)phenyl; 3-(1-methylindol-5-yl)phenyl; 4-(cyclopentylamino)phenyl; 4-cyclobutylaminophenyl; 3R,5S-(5-phenyl)piperidin-3-yl; 4-N,N-diethylaminophenyl; 3-(2-hydroxypyridin-4-yl)phenyl; 4-(3,4,5-dimethoxybenzyl-amino)phenyl; 3-[4-(4-acetylpiperazin-1-yl)-phenyl)-phenyl; 4-cyanophenyl; 3-cyclopropylphenyl; 4-(ethylamino)phenyl; 4-(N-3,4,5-dimethoxybenzyl-N-ethylamino)phenyl; 4-(cyclopropylamino)phenyl; 4-(N,N-dicyclopropylamino)phenyl; 6-(4-morpholin-4-ylphenyl)-pyridin-2-yl; 3-(6-fluoropyridin-3-yl)phenyl; 6-bromopyridin-2-yl; 4-(N-3,4,5-dimethoxybenzyl-N-methylamino)phenyl; 3-(3-chloropyridin-4-yl)phenyl; 5-fluoro-3-(6-morpholin-4-ylpyridin-3-yl)phenyl; 4-sec-butylaminophenyl; 4-methylaminophenyl; 4-n-propylaminophenyl; 4-N,N-di-n-propylamino-phenyl; 2-methylbenzimidazol-6-yl; 2-ethylbenzoxazol-5-yl; 2-methylbenzoxazol-5-yl; 3-methoxyquinolin-6-yl; cyclobutyl; 2-methylcyclopropyl; cyclopentyl; 3-pyrimidin-2-ylphenyl; 3-pyridin-2-ylphenyl; tetrahydrofuran-3-yl; 2,4-dimethylthiazol-5-yl; 3-(6-dimethylamino-pyridin-4-yl)-phenyl; 3-(4-methylimidazol-1-yl)-phenyl; 3-(pyrazol-1-yl)-phenyl; 4-N-acetyl-N-isopropylamino-phenyl; 6-chloropyridin-3-yl; 4-amino-3-methoxyphenyl; 4-isopropylamino-3-methoxyphenyl; 3-bromo-5-methylphenyl; 4-fluoro-3-(4-morpholin-4-ylphenyl)phenyl; 6-piperidin-1-ylpyridin-3-yl; 5-methyl-3-(2-methoxy-pyrimidin-5-yl)phenyl; benzoxazol-5-yl; 2-pyridin-3-ylbenzoxazol-5-yl; 2-pyridin-4-ylbenzoxazol-5-yl; 3,5-di(4-trifluoromethyl-phenyl)phenyl; 5-isopropylaminopyridin-2-yl; 4-methyl-3-indol-5-ylphenyl; 3-bromo-5-chlorophenyl; 5-chloro-3-(2-methyl-pyrimidin-5-yl)phenyl; 5-chloro-3-(1,5-dimethylpyrazol-4-yl)phenyl; 5-chloro-3-(pyridin-3-yl)phenyl; 5-methyl-3-(4-cyanophenyl)-phenyl; 5-methyl-3-(3-cyanophenyl)-phenyl; 5-methyl-3-(2-cyanophenyl)-phenyl; 4-isobutylaminophenyl; 4-N,N-di-isobutylaminophenyl; 3-cyano-5-methylphenyl; 4-(N-3,4,5-dimethoxybenzyl-N-n-propyl-amino)phenyl; 3-(2-methylaminopyridin-3-yl)-phenyl; 3-[2-(2-methoxyethyl-amino)pyridin-3-yl]-phenyl; 2-methylbenzimidazol-5-yl; 6-methylsulfonylindol-2-yl; 5-methyl-3-(2-methylpyrimidin-5-yl)-phenyl; benzothiophen-2-yl; 5-methyl-3-(4-pyridin-2-ylpiperazin-1-yl)-phenyl; 5-methyl-3-(2-methylpyridin-4-yl)-phenyl; 5-fluoro-3-(pyridin-3-yl)-phenyl; 5-fluoro-3-(2-methylpyridin-4-yl)-phenyl; 3-difluoromethyl-5-trifluoromethyl-phenyl; 3-fluoro-5-methylphenyl; isoquinolin-5-yl; 1-methylindol-5-yl; quinolin-5-yl; benzo[d][1,2,3]thiadiazol-5-yl; 4-morpholin-4-ylmethylphenyl; 1-methyl-3-ethylpyrazol-5-yl; 3-ethoxyphenyl; 3-morpholin-4-ylmethylphenyl; quinolin-4-yl; 3-difluoromethoxyphenyl; 3,5-dimethylthiazol-4-yl; 3-(6-cyanopyridin-3-yl)phenyl; 5-methyl-3-(1-methyl-2-oxopiperazin-4-yl)phenyl; 5-methyl-3-(4-methoxypiperidin-1-yl)phenyl; 5-methyl-3-(4,4-difluoropiperidin-1-yl)phenyl; 3-(6-isopropylaminopyridin-3-yl)phenyl; 3-(4-carboxyphenyl)phenyl; 3-(6-chloropyrazin-2-yl)phenyl; 3-(5-methoxy-pyrazin-2-yl)phenyl; 5-bromo-2-fluorophenyl; 5-chloro-3-(3-cyanophenyl)-phenyl; 5-chloro-3-(2-methoxy-pyrimidin-5-yl)-phenyl; 5-chloro-3-(furan-3-yl)-phenyl; 5-chloro-3-(4-cyanophenyl)-phenyl; 4,6-ditrifluoromethylpyridin-2-yl; pyridin-4-yl; indol-6-yl; 3-acetylaminophenyl; 6-isopropylaminopyridin-3-yl; 4-N-4-methoxybenzyl-N-methylaminophenyl; 4-benzylaminophenyl; 6-dimethylaminopyridin-3-yl; 3-(3-methoxypiperidin-1-yl)phenyl; 3-(4-tert-butoxycarbonyl-piperazin-1-yl)phenyl; 3-(piperazin-1-yl)phenyl; 1-methylindazol-3-yl; 4-pyrrol-1-ylphenyl; 4-bromothiophen-2-yl; 4-N-benzyl-N-methylphenyl; 4-(2-methoxypyrimidin-5-yl)thiophen-2-yl; 5-bromothiophen-2-yl; 5-bromophenyl; 3-(2-methoxyethylamino-methyl)phenyl; 3-(2-dimethylamino-methyl)phenyl; 3-(piperidin-1-ylmethyl)phenyl; 3-hydroxymethylphenyl; or 5-methylpyridin-3-yl.
(ix) Within groups (VI)(g) and groups contained therein, in another group of compounds Ar² is 3,5-diCF₃phenyl; 3,4-dichlorophenyl; 2,4-dichlorophenyl; 2-trifluoromethylphenyl; 3-chlorophenyl; 3-chloro-6-methoxyphenyl; 3-trifluoromethylphenyl; 2,6-dichlorophenyl; naphth-1-yl; 3,4-methylenedioxyphenyl; 4-chlorophenyl; biphen-4-yl; phenyl; 3,5-difluorophenyl; 4-methoxyphenyl; 3-phenoxyphenyl; naphth-2-yl; 3-methoxyphenyl; 2-chloro-5-bromophenyl; 3-chloro-4-methoxyphenyl; 2-chlorophenyl; 3,4-ethylenedioxyphenyl; 3,5-dimethylphenyl; 2,3-dichlorophenyl; 3-dimethylaminophenyl; 3-cyanophenyl; 3-chloro-4,5-dimethoxyphenyl; 3-methylphenyl; 6-methoxynaphth-2-yl; 3,5-dichlorophenyl; 2-methoxy-5-chlorophenyl; 3-benzoylphenyl; 4-isobutylphenyl; 3-fluoro-4-phenylphenyl; 6-methoxynaphth-2-yl; 2-biphenyl; 4-dimethylaminophenyl; 3-fluorophenyl; 2-chloro-6-fluorophenyl; 3,5-dimethoxyphenyl; 2,5-ditrifluoromethylphenyl; 2-fluoro-3-trifluoromethylphenyl; 4-trifluoromethoxyphenyl; 4-benzyloxyphenyl; 2-benzyloxyphenyl; 2-ethoxyphenyl; 4-methoxy-3-methylphenyl; 3-chloro-2,6-difluorophenyl; 3-bromophenyl; 3-(4-morpholin-4-ylphenyl)-phenyl; 4-(4-morpholin-4-ylphenyl)-phenyl; 4-piperidin-1-ylphenyl; 3-(2-morpholin-4-ylpyridin-5-yl)-phenyl; 3-(1-tert-butoxycarbonyl-1,2,3,6-tetrahydropyridin-4-yl)-phenyl; 3-morpholin-4-ylphenyl; 3-(1-methylpiperazin-4-yl)phenyl; 3-(4-pyridin-2-ylpiperazin-1-yl)-phenyl; 3-[1-(2-hydroxyethyl)piperidin-4-yl]phenyl; 3-methylthiophenyl; 3-methylsulfonylphenyl; 3-(4-dimethylaminophenyl)phenyl; 3-(3-cyanophenyl)phenyl; 3-(3-methoxyphenyl)phenyl; 3-(2-methoxypyrimidin-5-yl)phenyl; 3-(4-dimethylaminocarbonyl-phenyl)phenyl; 3-(4-methylcarbonyl-phenyl)phenyl; 3-fluoro-5-trifluoromethyl-phenyl; 3-fluoro-5-(4-morpholin-4-ylphenyl)phenyl; 3-phenylaminophenyl; 3-(2-methoxyethylamino)phenyl; 4-fluoro-3-morpholin-4-ylphenyl; 5-fluoro-3-(4-morpholin-4-ylphenyl)phenyl; 4-(cyclopropylmethylamino)-phenyl; 4-(N,N-dicyclopropylmethylamino)-phenyl; 3-thiophen-3-ylphenyl; 3-indol-5-ylphenyl; 4-N,N-dicyclobutyl-aminophenyl; 3-(2-morpholin-4-ylpyrimidin-5-yl)phenyl; 4-(4-methoxybenzylamino)-phenyl; 3-(2-morpholin-4-ylthiazol-4-yl)phenyl; 4-tetrahydropyran-4-ylamino-phenyl; biphen-3-yl; 4-bromophenyl; 3-(4-phenylpiperazin-1-yl)-phenyl; 3-iodophenyl; 4-isopropylaminophenyl; 3-benzyloxyphenyl; 3-(4-cyanophenyl)phenyl; 3-(2-cyanophenyl)phenyl; 3-(4-methoxyphenyl)phenyl; 3-(2-methoxyphenyl)phenyl; 3-(4-trifluoromethoxyphenyl)-phenyl; 3-[4-(1-tert-butoxypiperazin-1-yl)phenyl]-phenyl; 3-(4-piperazin-1-ylphenyl)-phenyl; 4-morpholin-4-ylphenyl; 3-pyridin-3-ylphenyl; 3-pyridin-4-ylphenyl; 4-di-n-propylaminophenyl; 4-diethylaminophenyl; 2-methyl-5-(2-methylpyrimidin-5-yl)-phenyl; 2,4-dimethoxyphenyl; 2,5-dimethoxyphenyl; 2,5-dimethylphenyl; 3,4-dimethoxyphenyl; 3-trifluoromethylphenyl; 4-biphenyl; 3-methoxy-4-methylphenyl; 2-methoxyphenyl; 2-fluorophenyl; 4-methylphenyl; 2,3-dimethoxyphenyl; 2-bromophenyl; 2-iodophenyl; 2-fluoro-6-trifluoro-methylphenyl; 2,4,6-trimethylphenyl; 4-fluoro-3-trifluoromethyl-phenyl; 2-methylphenyl; 2,4-ditrifluoromethylphenyl; 3-fluoro-4-methoxyphenyl; 2-chloro-5-fluorophenyl; 2-chloro-4-fluorophenyl; 2-bromo-5-chlorophenyl; 4-iodophenyl; 2-fluoro-5-trifluoromethyl-phenyl; 3-chloro-5-fluorophenyl; 4-n-butoxyphenyl; 4-tert-butylphenyl; 4-isopropylphenyl; 5-bromo-2-methoxyphenyl; 3-chloro-2-fluorophenyl; 2-fluoro-4-trifluoromethylphenyl; 3-fluoro-4-trifluoromethylphenyl; 2,6-difluoro-3-methylphenyl; 2-chloro-5-trifluoromethylphenyl; 2-trifluoromethoxyphenyl; 5-fluoro-2-trifluoromethylphenyl; 4-aminophenyl; 4-phenoxyphenyl; 3-(1,2,3,6-tetrahydropyridin-4-yl)phenyl; 3-(piperidin-4-yl)phenyl; 3-(morpholin-4-yl)phenyl; 3-fluoro-2-trifluoromethyl-phenyl; 3-(2-dimethylaminophenyl)-phenyl; 5-chloro-2-fluorophenyl; 3-morpholin-4-yl-5-trifluoromethylphenyl; 3-(4-methylsulfonylphenyl)-phenyl; 3-bromo-5-fluorophenyl; 3-bromo-4-fluorophenyl; 3-piperidin-1-ylphenyl; 4-fluoro-3-(2-methoxyethylamino)phenyl; 3-cyano-5-trifluoromethylphenyl; 3-bromo-5-trifluoromethylphenyl; 4-isopropylmethyl-aminophenyl; 3-bromo-5-fluorophenyl; 4-trifluoromethylphenyl; 3-pyrimidin-5-ylphenyl; 3-(methylthiopyrimidin-5-yl)phenyl; 3-(2-aminopyrimidin-5-yl)phenyl; 3-(1-methylindol-5-yl)phenyl; 4-(cyclopentylamino)phenyl; 4-cyclobutylaminophenyl; 4-N,N-diethylaminophenyl; 3-(2-hydroxypyridin-4-yl)phenyl; 4-(3,4,5-dimethoxybenzyl-amino)phenyl; 3-[4-(4-acetylpiperazin-1-yl)-phenyl)-phenyl; 4-cyanophenyl; 3-cyclopropylphenyl; 4-(ethylamino)phenyl; 4-(N-3,4,5-dimethoxybenzyl-N-ethylamino)phenyl; 4-(cyclopropylamino)phenyl; 4-(N,N-dicyclopropylamino)phenyl; 3-(6-fluoropyridin-3-yl)phenyl; 6-bromopyridin-2-yl; 4-(N-3,4,5-dimethoxybenzyl-N-methylamino)phenyl; 3-(3-chloropyridin-4-yl)phenyl; 5-fluoro-3-(6-morpholin-4-ylpyridin-3-yl)phenyl; 4-sec-butylaminophenyl; 4-methylaminophenyl; 4-n-propylaminophenyl; 4-N,N-di-n-propylamino-phenyl; 3-pyrimidin-2-ylphenyl; 3-pyridin-2-ylphenyl; 3-(6-dimethylamino-pyridin-4-yl)-phenyl; 3-(4-methylimidazol-1-yl)-phenyl; 3-(pyrazol-1-yl)-phenyl; 4-N-acetyl-N-isopropylamino-phenyl; 4-amino-3-methoxyphenyl; 4-isopropylamino-3-methoxyphenyl; 3-bromo-5-methylphenyl; 4-fluoro-3-(4-morpholin-4-ylphenyl)phenyl; 5-methyl-3-(2-methoxy-pyrimidin-5-yl)phenyl; 3,5-di(4-trifluoromethyl-phenyl)phenyl; 4-methyl-3-indol-5-ylphenyl; 3-bromo-5-chlorophenyl; 5-chloro-3-(2-methyl-pyrimidin-5-yl)phenyl; 5-chloro-3-(1,5-dimethylpyrazol-4-yl)phenyl; 5-chloro-3-(pyridin-3-yl)phenyl; 5-methyl-3-(4-cyanophenyl)-phenyl; 5-methyl-3-(3-cyanophenyl)-phenyl; 5-methyl-3-(2-cyanophenyl)-phenyl; 4-isobutylaminophenyl; 4-N,N-di-isobutylaminophenyl; 3-cyano-5-methylphenyl; 4-(N-3,4,5-dimethoxybenzyl-N-n-propyl-amino)phenyl; 3-(2-methylaminopyridin-3-yl)-phenyl; 3-[2-(2-methoxyethyl-amino)pyridin-3-yl]-phenyl; 5-methyl-3-(2-methylpyrimidin-5-yl)-phenyl; 5-methyl-3-(4-pyridin-2-ylpiperazin-1-yl)-phenyl; 5-methyl-3-(2-methylpyridin-4-yl)-phenyl; 5-fluoro-3-(pyridin-3-yl)-phenyl; 5-fluoro-3-(2-methylpyridin-4-yl)-phenyl; 3-difluoromethyl-5-trifluoromethyl-phenyl; 3-fluoro-5-methylphenyl; 4-morpholin-4-ylmethylphenyl; 3-ethoxyphenyl; 3-morpholin-4-ylmethylphenyl; 3-difluoromethoxyphenyl; 3-(6-cyanopyridin-3-yl)phenyl; 5-methyl-3-(1-methyl-2-oxopiperazin-4-yl)phenyl; 5-methyl-3-(4-methoxypiperidin-1-yl)phenyl; 5-methyl-3-(4,4-difluoropiperidin-1-yl)phenyl; 3-(6-isopropylaminopyridin-3-yl)phenyl; 3-(4-carboxyphenyl)phenyl; 3-(6-chloropyrazin-2-yl)phenyl; 3-(5-methoxy-pyrazin-2-yl)phenyl; 5-bromo-2-fluorophenyl; 5-chloro-3-(3-cyanophenyl)-phenyl; 5-chloro-3-(2-methoxy-pyrimidin-5-yl)-phenyl; 5-chloro-3-(furan-3-yl)-phenyl; 5-chloro-3-(4-cyanophenyl)-phenyl; 3-acetylaminophenyl; 4-N-4-methoxybenzyl-N-methylaminophenyl; 4-benzylaminophenyl; 3-(3-methoxypiperidin-1-yl)phenyl; 3-(4-tert-butoxycarbonyl-piperazin-1-yl)phenyl; 3-(piperazin-1-yl)phenyl; 4-pyrrol-1-ylphenyl; 4-N-benzyl-N-methylphenyl; 5-bromophenyl; 3-(2-methoxyethylamino-methyl)phenyl; 3-(2-dimethylamino-methyl)phenyl; 3-(piperidin-1-ylmethyl)phenyl; or 3-hydroxymethylphenyl.
(x) Within groups (VI)(g) and groups contained therein, yet another group of compounds is that wherein Ar² is 5-methyl-1H-imidazo[1,2-a]pyridin-2-yl; 5-cyclopropylisoxazol-3-yl; 6-chloropyridin-2-yl; 5-chlorothiophen-2-yl; 5-bromopyridin-3-yl; 5-methylthiophen-2-yl;

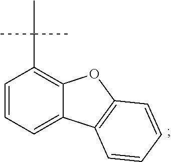

2,6-dichloropyridin-4-yl; imidazol-1-yl; 3,5-dimethylimidazol-1-yl; 3,5-dimethylisoxazol-4-yl;

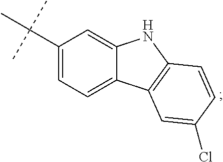

benzothiophen-3-yl; 5-bromo-1H-indazol-3-yl; 1H-indol-3-yl; pyridin-2-yl; 9-methyl-9H-carbazol-2-yl; 2,3-dihydrobenzofuran-5-yl; 3-chloro-9H-carbazol-7-yl; 2-phenylbenzoxazol-5-yl; quinolin-6-yl; 5-bromo-3-methylbenzimidazol-1-yl; 2-morpholin-4-ylpyridin-6-yl; 2-(4-morpholin-4-ylphenyl)pyridin-6-yl; 4-phenylimidazol-1-yl; 2-(4-trifluoromethylphenyl)-pyridin-4-yl; 2-cyclopropylpyridin-4-yl; 2-(2-methoxyethylamino)-pyridin-6-yl; 2-pyridin-2-ylbenzoxazol-5-yl; 6-chloroimidazo[1,2-a]pyridin-2-yl; imidazo[1,2-a]pyridin-2-yl; 1-methylindol-2-yl; 6-bromoimidazo[1,2-a]pyridin-2-yl; 6-chloro-9-methyl-9H-carbazol-2-yl; 1-methylindol-3-yl; 9-methyl-9H-carbazol-3yl; 6-morpholin-4-ylpyridin-2-yl; 4-(4-morpholin-4-ylphenyl)-pyridin-2-yl; 2-(4-morpholin-4-yl-phenyl)-pyridin-4-yl; 5-cyclopropyl-1H-pyrazol-3-yl; 7-methylimidazo[1,2-a]pyridin-2-yl; 8-methylimidazo[1,2-a]pyridin-2-yl; pyridin-3-yl; 4-chloropyridin-2-yl; 6-chloro-1H-imidazo[1,2-a]pyridin-2-yl; 3-methylpyridin-2-yl; 1H-imidazo[1,2-a]pyridin-2-yl; 1-benzylpyridin-4-yl; 6-fluorobenzthiophen-2-yl; 5-bromo-1H-imidazo[1,2-a]pyridin-2-yl; thien-2-yl; thien-3-yl; indol-1-yl; 6-fluoropyridin-2-yl; 3-phenylimidazol-1-yl; 3-methyl-4-phenylpyrazol-1-yl; 4-(4-bromophenyl)pyrazol-1-yl; 2-chloropyridin-4-yl; indol-5-yl; 4-bromoimidazol-1-yl; 6-(4-morpholin-4-ylphenyl)-pyridin-2-yl; 6-bromopyridin-2-yl; 2-methylbenzimidazol-6-yl; 2-ethylbenzoxazol-5-yl; 2-methylbenzoxazol-5-yl; 3-methoxyquinolin-6-yl; 2,4-dimethylthiazol-5-yl; 6-chloropyridin-3-yl; 6-piperidin-1-ylpyridin-3-yl; benzoxazol-5-yl; 2-pyridin-3-ylbenzoxazol-5-yl; 2-pyridin-4-ylbenzoxazol-5-yl; 5-isopropylaminopyridin-2-yl; 2-methylbenzimidazol-5-yl; 6-methylsulfonylindol-2-yl; benzothiophen-2-yl; isoquinolin-5-yl; 1-methylindol-5-yl; quinolin-5-yl; benzo[d][1,2,3]thiadiazol-5-yl; 1-methyl-3-ethylpyrazol-5-yl; quinolin-4-yl; 3,5-dimethylthiazol-4-yl; 4,6-ditrifluoromethylpyridin-2-yl; pyridin-4-yl; indol-6-yl; 6-isopropylaminopyridin-3-yl; 6-dimethylaminopyridin-3-yl; 1-methylindazol-3-yl; 4-bromothiophen-2-yl; 4-(2-methoxypyrimidin-5-yl)thiophen-2-yl; 5-bromothiophen-2-yl; or 5-methylpyridin-3-yl.
(xi) Within groups (VI)(g) and groups contained therein, yet another group of compounds is that wherein Ar² is 1-(4-F-phenyl)cyclopent-1-yl; 2,3-dihydroinden-2-yl; 3-methoxycyclohexyl; tetrahydrofuran-2-yl; 1-phenyl-2-oxopyrrolidin-4-yl; 1-acetylpiperidin-4-yl; 1-cyclohexyl-2-oxopyrrolidin-4-yl; 1-tert-butyl-2-oxopyrrolidin-4-yl; 1-benzyl-2-oxopyrrolidin-4-yl; 4,5,6,7-tetrahydrobenzthiazol-3-yl; cyclohexyl; 1-oxo-2-phenylisoindolin-7-yl; 2-cyclopentyl-1-oxoisoindolin-7-yl; 2,3-dihydrobenzofuran-5-yl; 1-oxoisoindolin-2-yl; S-1-tert-butoxycarbonyl-pyrrolidin-2-yl; tetrahydropyran-4-yl; 2-cyclopropylpyridin-4-yl; 2,2-dimethylcycloprop-1-yl; 2,2-difluorocycloprop-1-yl; 4,5,6,7-tetrahydro-1,2-benzisoxazol-3-yl; 1,2,3,4-tetrahydronaphth-1-yl; 1,3,4,9-tetrahydro-2H-beta-carbolin-2-yl); 8-fluoro-1,3,4,5-tetrahydro-2H-pyrido[4,3-b]indol-2-yl; 8-fluoro-1,3,4,5-tetrahydro-2H-pyrido[4,3-b]indol-2-yl; 1-phenylcycloprop-1-yl; 1-(4-Clphenyl)cycloprop-1-yl; 1-(2-Fphenyl)-cycloprop-1-yl; 1-(4-Fphenyl)cycloprop-1-yl; 1-(3-Fphenyl)cycloprop-1-yl; 1-(phenyl)-cyclobut-1-yl; 1-(phenyl)cyclohex-1-yl; 1-(3-F-phenyl)cyclopent-1-yl; 1-(2-phenylethyl)-2-oxopyrrolidin-4-yl; 1-(4-chlorophenyl)-2-oxopyrrolidin-4-yl; 1-benzylpyrrolidin-2-yl; 1-cyclohexylazetidin-2-yl; 1-benzylpyridin-4-yl; 1-(3-chlorophenyl)-2-oxopyrrolidin-4-yl; 2-phenylcyclopropyl; 1,2,3,4-tetrahydronaphth-2-yl; 4H-chromen-4-one-3-yl; 1-(4-methoxyphenyl)cycloprop-1-yl; dichlorophenyl)-cycloprop-1-yl; 1,2,3,4-tetrahydroindol-5-yl; 3R,5S-(5-phenyl)piperidin-3-yl; cyclobutyl; 2-methylcyclopropyl; cyclopentyl; or tetrahydrofuran-3-yl.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of formula (I) can be prepared as described in Scheme A below:

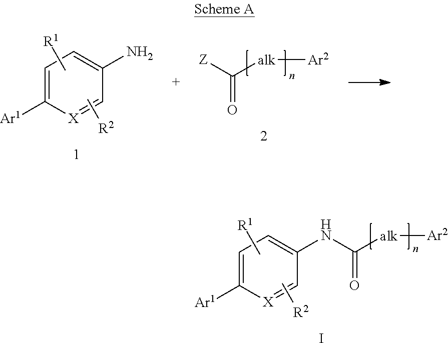

Compounds of Formula (I) can be synthesized by coupling an amino compound of formula 1 with an acid or acid derivative of formula 2 i.e., Z is hydroxyl or a suitable leaving group such as halo, imidazolyl, and the like, and other groups are as defined in the Summary of the Invention. The reaction is carried out in the presence of coupling reagents known to one skilled in the art of organic synthesis. When Z is hydroxyl, the coupling reaction can be carried out with coupling agents such as EDCI/HOBT, O-(7-azabenzotriazole-1-yl)-N, N,N′N′-tetramethyluronium hexafluorophosphate (HATU) and chlorodipyrrolidinocarbenium hexafluorophosphate (PyCIU) (see for example, Han, S-Y.; Kim, Y-A. Tetrahedron 20054, 60 (11), 2447-67). When Z is halo, the reaction is carried out in the presence of a non-nucleophilic amine such as triethylamine, pyridine, and the like. The acid chloride (Z is chloro) which is prepared in situ from the corresponding acid using either oxalyl chloride or thionyl chloride.

Compounds of formula 1 are either commercially available or they can be synthesized by methods well known in the art. A representative procedure is shown below from Suzuki coupling of the appropriate aryl boronic acids or esters V and VI with aryl halides IV and VII using conditions well known to one skilled in the art of organic synthesis, see

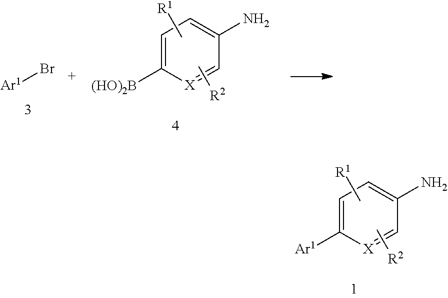

A heteroaryl bromide of formula 3 where Ar1 is as defined in the Summary of the Invention can be coupled with a boronic acid compound of formula 4 under Suzuki coupling reaction conditions to provide a compound of formula 1. The starting materials 3 and 4 are either commercially available or prepared according to literature procedures.

Compounds of formula 2 are also either commercially available or can synthesized be synthesized by methods well known in the art. For example, compound of formula 2 where n is 1 and alk is a substituted methylene can be prepared by alkylations of aryl acetic acid using strong base such as lithium diisopropylamide (LDA) (see for example, Advanced Organic chemistry, Jerry March, John Wiley & sons, 1985, 3^{rd} ed).

### Utility

The compounds of the invention are γ-secretase modulators and hence are useful in the treatment of Alzheimer's disease.

### Testing

The γ-secretase modulatory activity of the compounds of the present invention can be tested using the in vitro and in vivo assays described in Biological Examples 1 and 2 below.

### Administration and Pharmaceutical Composition

In general, the compounds of this invention will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day.

For oral administration, the compositions are preferably provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this invention, i.e., the active ingredient, will depend upon numerous factors such as the severity of the disease to be treated, the age and relative health of the subject, the potency of the compound utilized, the route and form of administration, and other factors.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

### Examples

The following preparations of compounds of Formula (I) and intermediates (References) are given to enable those skilled in the art to more clearly understand and to practice the present invention. They should not be considered as limiting the scope of the invention, but merely as being illustrative and representative thereof.

The following abbreviations are used:
AcOH, HOAc—acetic acid
AIBN—2,2′-azobisisobutyronitrile
CH₃CN, ACN, MeCN—acetonitrile
Aq.—aqueous
NH₃—ammonia
NH₄Cl—ammonium chloride
NH₄OH—ammonium hydroxide
BH₃—borane
BH₃SMe₂—borane-methyl sulfide complex
Br₂—bromine
CCl₄—carbon tetrachloride
CHCl₃—chloroform
DBU—1,8-diazabicyclo[5.4.0]undec-7-ene
DCM, CH₂Cl₂—dichloromethane
Et₂O—diethyl ether
DMAP—4-(dimethylamino)pyridine
Me₂NH—dimethylamine
DAST—diethylaminosulfur trifluoride
DPPA—diphenylphosphoryl azide
DMF—dimethylformamide
DMSO—dimethyl sulfoxide (also known as methyl sulfoxide)
DPPF—1,1′-Bis(diphenylphosphino)ferrocene
EtOAc—ethyl acetate
EDC—(3-dimethylamino-propyl)-ethyl-carbodiimide-HCl salt
EtOH—ethanol
HCO₂H—formic acid
g—gram
GC—gas chromatography
h—hour
HATU—O-(7-Azabenzotriazol-1-yl)-N,N,N′,N′-tetramethyluronium hexafluorophosphate
HCl—hydrochloric acid
H₂—hydrogen
HOAt—1-hydroxy-7-azabenzotriazole
HOBt—1-hydroxybenzotriazole
H₃PO₄—phosphoric acid
H₂SO₄—sulfuric acid
IPA, iPrOH—isopropanol
DIPEA, DIEA—Diisopropylethyl amine ISCO (column chromatography)
   column chromatography using Teledyne ISCO liquid chromatography system
K₂CO₃—potassium carbonate
KOtBu—potassium tert-butoxide
LAH—lithium aluminum hydride
LDA—lithium diisopropylamide
LiOH—lithium hydroxide
LHMDS—lithium bis(trimethylsilyl)amide, Li(NSiMe3)2
MTBE—Methyl tert-butyl ether
MeOH—methanol
mL—milliliter
min—minutes
MgSO₄—magnesium sulfate
Na2SO4—sodium sulfate
NBS—N-bromosuccinimide
N₂—nitrogen
NMM—N-methylmorpholine
NMP—1-methyl-2-pyrrolidone
Pd/C—palladium on carbon
Pd(OH)₂—palladium hydroxide
Pd(OAc)2—palladium acetate
KCN—potassium cyanide
KOH—potassium hydroxide
RP-HPLC—reverse phase high pressure liquid chromatography
RT—room temperature
SiO₂—silica
NaOAc—sodium acetate
NaN₃—sodium azide
NaHCO₃—sodium bicarbonate
NaBH₄—sodium borohydride
NaBH(OAc)₃—sodium triacetoxyborohydride
NaOH—sodium hydroxide
Na₂SO₄—sodium sulfate
PYBOP—Benzotriazol-1-yl-oxytripyrrolidinophosphonium hexafluorophosphate
SOCl₂—thionyl chloride
TBAF—tetrabutylammonium fluoride
TBTU—O-(Benzotriazol-1-yl)-N,N,N′,N′-tetramethyluronium tetrafluoroborate
TEA, Et₃N—triethylamine
TFA—trifluoroacetic acid
THF—tetrahydrofuran
TLC—Thin layer chromatography
TsCl—tosyl chloride
TsOH—toluene sulfonic acid
TEA, Et₃N—triethylamine
PPh₃—triphenylphosphine
H₂O—water
RT, rt—room temperature

### Examples

The following preparations of compounds of Formula (I) and intermediates (References) are given to enable those skilled in the art to more clearly understand and to practice the present invention. They should not be considered as limiting the scope of the invention, but merely as being illustrative and representative thereof.

### Reference A

### Synthesis of 4-(2-methylpyridin-4-yl)benzenamine

To a solution of 4-(4,4,5,5-tetramethyl-1,3,2-dioxaborolan-2-yl)benzenamine (11.7 g, 53.5 mmol), 4-bromo-2-methylpyridine (9.20 g, 53.5 mmol), Na₂CO₃ (2.83 g, 26.7 mmol) in 50 mL MeCN/50 mL H2O, was added Pd(PPh3)4 (1.85 g, 1.60 mmol), and the reaction mixture was refluxed at 130° C. for 15 h. The solution was cooled, and the solids were collected by filtration, and the residue was concentrated and extracted with ethyl acetate. Ethyl acetate solution was evaporated and the residue was combined with the solids obtained from filtration. The crude materials were subjected to silica gel chromatography with 25% EtOAc in CH₂Cl₂ gave 4-(2-methylpyridin-4-yl)benzenamine.

### Reference 2

### Synthesis of 3-methoxy-4-(2-methylpyridin-4-yl)benzenamine

### Step 1

4-Bromo-3-methoxyaniline (3.70 g, 18.3 mmol; Aldrich) was dissolved in N,N-dimethylformamide (40 mL, 500 mmol) and triethylamine (2.68 mL, 19.2 mmol) was added. Acetic anhydride (1.81 mL, 19.2 mmol) was added and the reaction mixture stirred for 5 hours. The reaction mixture was diluted with water and extracted with diethyl ether. The organic layer was dried over Na₂SO₄, filtered and diluted with hexames. The layer was concentrated to minimal volume and the resultant slurry was filtered and washed with hexanes to yield N-(4-bromo-3-methoxyphenyl)acetamide as a light peach solids.

### Step 2

N-(4-Bromo-3-methoxyphenyl)acetamide (700 mg, 3 mmol) and (2-methylpyridin-4-yl)boronic acid (400 mg, 3 mmol; Asymchem) were dissolved in 1,2-dimethoxyethane (22 mL, 220 mmol) and ethanol (6.3 mL, 110 mmol). To this mixture was added aq. sodium carbonate (920 mg, 8.7 mmol) dissolved in water (9.7 mL, 540 mmol). The reaction mixture was degassed and then tetrakis(triphenylphosphine)palladium(0) (200 mg, 0.1 mmol) was added. The reaction mixture was again degassed and then it was placed in an oil bath preheated at 95° C. After 2 hours, the reaction mixture was cooled and a second dose of 4-pyridineboronic acid and sodium carbonate, dissolved in water, was added and the reaction mixture degassed. A second dose of tetrakis(triphenylphosphine)palladium(0) was added and the reaction mixture again degassed and placed in a preheated oil bath at 95° C. overnight. The reaction mixture was cooled and the volatiles removed under vacuum. The mixture was diluted with water and DCM. After phase separation the water was extracted 5 additional times to completely removed the product from the water. The DCM layers were combined, dried over Na₂SO₄, filtered and concentrated and purified on a silica gel column eluting with 10-30% acetone in DCM to give N-(3-methoxy-4-pyridin-4-ylphenyl)acetamide.

### Step 3

N-(3-Methoxy-4-pyridin-4-ylphenyl)acetamide (570 mg, 0.0024 mol) was dissolved in ethanol (11 mL, 0.19 mol) and 2 M of sodium hydroxide in water (9.4 mL) and the reaction mixture heated under nitrogen overnight in an oil bath at 70° C. The volatiles were removed and the resultant mixture was diluted with water and DCM. The phases were separated and the water was extracted two additional times with DCM. The DCM layers were dried over Na₂SO₄, filtered and concentrated to crude dark residue which was purified on a silica gel column eluting with 0-8% MeOH in DCM to isolate 3-methoxy-4-(2-methylpyridin-4-yl)benzenamine which can be further purified by recrystallization.

### Reference 3

### Synthesis of 4-(2-chloropyridin-4-yl)-3-methoxyaniline

### Step 1

1-Bromo-2-methoxy-4-nitrobenzene (4.5 g, 0.019 mol; Alfa Asear), (2-chloropyridin-4-yl)boronic acid (3.10 g, 0.0197 mol; Combi-Blocks), and tetrakis(triphenylphosphine)-palladium(0) (1.9 g, 0.0016 mol) were added to a flask and the flask purged with nitrogen. 1,2-Dimethoxyethane (150 mL, 1.4 mol) was added and the solution degassed. Cesium carbonate (19 g, 0.060 mol) was dissolved in water (70.6 mL, 3.92 mol) and this solution was added to the reaction mixture and the reaction mixture again degassed. The dark red slurry was heated to reflux (oil bath at 95° C.) and the solids were all in solution. The reaction mixture was heated at reflux for 18 hours and then cooled overnight and held at room temperature for the weekend. The volatiles were removed under vacuum and the resultant slurry was diluted with DCM and additional water and placed in a separatory funnel. The phases were separated and the water extracted two additional times with DCM. The DCM layers were combined, dried over Na₂SO₄ and concentrated to a brown-red solid. To this was added MTBE and the mixture was triturated and then the volume was reduced to yield a slurry. The slurry was cooled in an ice bath, filtered and rinsed with 1:1 MTBE/hexanes and then hexanes to yield 2-chloro-4-(2-methoxy-4-nitrophenyl)pyridine. The filtrate from the above crystallization was concentrated to a dark red solid (3.5 g) and loaded onto 6 g silica gel (DCM) and purified by column chromatography eluting with a 15-55% ethyl acetate/hexanes gradient to give additional product.

### Step 2

2-Chloro-4-(2-methoxy-4-nitrophenyl)pyridine (4.11 g, 13.2 mmol) was dissolved in tetrahydrofuran (40 mL, 500 mmol) and water (20 mL, 1 mol), ammonium formate (20 g, 300 mmol), and iron (9 g, 200 mmol) were added. The reaction mixture was heated to reflux (oil bath at 82° C.) for 2 hours and then cooled to room temperature. The reaction mixture was assayed by TLC (50% ethyl acetate/hexanes) and the starting material was consumed.

The volatiles were removed and the mixture diluted with DCM and filtered through celite-545 rinsing with DCM. Water was added and the phases were separated. Subsequent DCM extractions were used to isolate the product although the mixture was a bit of an emulsion. The crude product (3.18 g) was loaded onto silica gel (6.2 g) and purified by column chromatography eluting with 15-50% ethyl acetate/hexanes gradient to give 4-(2-chloropyridin-4-yl)-3-methoxyaniline.

### Reference 4

### Synthesis of 3-methoxy-4-(4-methyl-imidazol-1-yl)phenylamine

### Step 1

To a stirred solution of 3,methoxy-4-bromo nitrobenzene (5 g, 0.026 mol) in DMF (50 ml) were added 4-methylimidazole (32 g, 0.038 mol), CuI (0.49 g, 0.0026 mol) and Cs₂CO₃ (21 g, 0.065 mol) at 25° C. Reaction mixture was stirred at this temperature for 30 min. To this 2,4-dimethyl hepta-3,5-dione (catalytic) was added and stirred at 90° C. overnight (modified procedures from Buchwald group J. Am. Chem. Soc., 2006, 128, 8742-8743). Reaction was cooled, partitioned between ethyl acetate (200 ml) and water. Aqueous part was extracted with ethyl acetate (200 ml). Organics combined, dried over sodium sulfate and concentrated under vacuum. Product was purified by column chromatography using silica gel (100-200 mesh) and 0-2% methanol, dichloromethane as eluent to obtain 2.1 g of 1-(2-methoxy-4-nitro-phenyl)-4-methyl-1H-imidazole

### Step 2

To a stirred solution of 1-(2-Methoxy-4-nitro-phenyl)-4-methyl-1H-imidazole (3.5 g, 0.0150 mol) in ethanol (45 ml) was added 10% Pd/C (1.6 g, 0.0015 mol) under nitrogen atmosphere. Then hydrogen atmosphere was applied using hydrogen balloon. Progress of the reaction was monitored by TLC. Reaction mixture was passed through celite, filtrate was evaporated off. Solid obtained was recrystallised from dichloromethane and pentane to obtain the title compound.

### Reference 5

### Synthesis of 2-(3-fluorophenyl)propanoic acid

To a solution of diisopropylamine (1 ml, 10 mmol) in THF (3 mL) at −50° C. was added butyllithium, 1.6M in hexanes (6 ml, 10 mmol) dropwise from a syringe. The solution was warmed to 0° C. with stirring for 30 minutes and a solution of 3-fluorophenylacetic acid (0.75 g, 5 mmol) in THF (5 mL) was added. After stirring at RT for 30 minutes, iodomethane (0.6 ml, 10 mmol) was added. The reaction mixture was stirred at rt for 18 h, diluted with 1N HCl solution, and extracted with DCM. The DCM layer was dried over Na₂SO₄, and concentrated in vacuo to give 2-(3-fluorophenyl)propanoic acid which was used without further purification.

### Reference 6

### Synthesis of 4-methyl-2-m-tolylpentanoic acid

To a solution of m-tolylacetic acid (500 mg, 3.329 mmol) (azeotroped with toluene) in 2 mL of dry toluene was added sodium bis(trimethylsilyl)amide, 1.0 M solution in tetrahydrofuran (8.3 mL of 1N solution in THF). After stirring at RT for 20 min, 1-iodo-2-methylpropane (674 μl, 3.662 mmol) was added dropwise. After 10 min, the reaction was quenched with 2 N HCl to pH=2, extracted with EtOAc, dried over Na₂SO₄, and filtered through a pad of silica gel to give crude 4-methyl-2-m-tolylpentanoic acid as a colorless oil. The crude product was used without further purification.

### Reference 7

### Synthesis of 2-(3-bromo-5-chlorophenyl)propanoic acid

### Step 1

A heavy-wall flask was charged with sodium hydride (9.5 g, 239 mmol) (60% oil dispersion) in NMP (75 ml) under a positive nitrogen flow. Ethyl 2-cyanoacetate (27 g, 239 mmol) was added in a dropwise manner. The reaction mixture was stirred until no more bubbling was observed. 1-Bromo-3-chloro-5-fluorobenzene (10.00 g, 48 mmol) was added to the reaction mixture. The flask was capped and the reaction mixture was heated to 115° C. After 15 hours, the reaction mixture was cooled down and diluted with water. The pH of the reaction mixture was adjusted to 3 with 2N HCl and extracted with ether. The organics were combined and washed with an aqueous saturated solution of sodium bicarbonate, then with water and then brine. The organic layer was then dried with sodium sulfate and purified by column chromatography on silica gel using a gradient of 5 to 40% EtOAc in hexanes to give ethyl 2-(3-bromo-5-chlorophenyl)-2-cyanoacetate (10.00 g, 69% yield) as a white semi solid.

### Step 2

A solution of TBAF 1M THF (73 ml, 73 mmol) was added to a solution of ethyl 2-(3-bromo-5-chlorophenyl)-2-cyanoacetate (10.00 g, 33 mmol) in DMSO (50 ml) at RT under nitrogen. After 10 mins of stirring, iodomethane (7.2 ml, 116 mmol) was added slowly to the reaction. The reaction mixture was stirred for 30 mins and then poured into saturated aq ammonium chloride solution. The reaction mixture was extracted with ethyl acetate. The organic layers were combined and washed with an aqueous saturated solution of sodium bicarbonate, then with water and then brine. The organic layer was then dried with sodium sulfate, evaporated and purified by column chromatography on silica gel using a gradient of 5 to 30% EtOAc in hexanes to give ethyl 2-(3-bromo-5-chlorophenyl)-2-cyanopropanoate (9.0 g, 86% yield) as an oil that turned into a white solid on standing.

### Step 3

Ethyl 2-(3-bromo-5-chlorophenyl)-2-cyanopropanoate (9 g) was suspended in 2N NaOH (100 ml) and the mixture was heated to 100° C. After 15 hours, the reaction was cooled down and adjusted to pH 2 with 5N HCl with cooling. The mixture was extracted with EtOAc. The organics were combined washed with an aqueous saturated solution of sodium bicarbonate, then with water and then brine. The organic layer was then dried with sodium sulfate, and evaporated to give 2-(3-bromo-5-chlorophenyl)propanoic acid as a white solid.

### Reference 7

### Synthesis of (±)-2-(3-bromo-5-methylphenyl)propanoic acid

### Step 1

Under an atmosphere of nitrogen, to a suspension of KOt-Bu (14.8 g, 133 mmol) in 1,4-dioxane (200 mL) was added ethyl cyanoacetate (6 g, 53 mmol) and 3,5-dibromotoluene (26.5 g, 106 mmol) sequentially, resulting in a yellow suspension. A pre-prepared solution of Pd(OAc)₂ (240 mg, 1.1 mmol, 2%) and DPPF (1.18 g, 2.1 mmol) in 1,4-dioxane (4-mL) was then added. The resulting mixture was heated at 70° C. for 1 h, when Gas chromatography (GC) analysis of the reaction mixture indicated the complete consumption of ethyl cyanoacetate. The reaction mixture was cooled to room temperature and methyl iodide (12 ml, 187 mmol) was added. The resulting mixture was stirred at ambient temperature for 16 h, and GC analysis indicated complete consumption of ethyl 2-(3-bromo-5-methylphenyl)-2-cyanoacetate. Hexanes (100 mL) were then added and the resulting suspension was passed filtered. The filtrate was concentrated and purified by column chromatograph (SiO₂, 100% hexanes to 10% EtOAc/Hexanes) to give ethyl 2-(3-bromo-5-methylphenyl)-2-cyanopropanoate as a colorless oil.

### Step 2

A mixture of ethyl 2-(3-bromo-5-methylphenyl)-2-cyanopropanoate (8.23 g, 28 mmol) in dioxane (27 ml) was combined with conc. HCl (25 ml, 306 mmol) and the reaction mixture was heated to 100° C. overnight. Reaction was monitored by Gas chromatography. Following complete reaction, the reaction mixture was cooled to room temperature, neutralized with sat. NaHCO₃, diluted with ethyl acetate and the layers were separated. The aqueous layer was extracted with ethyl acetate, the organic layers were combined and washed with saturated sodium chloride. The crude material was purified by flash silica gel chromatography using 0-10% hexanes/ethyl acetate gradient to give 2-(3-bromo-5-methylphenyl)propanenitrile as a racemic mixture.

### Step 3

2-(3-Bromo-5-methylphenyl)propanenitrile (2730 mg, 12182 μmol) was combined with 2N sodium hydroxide (22 mL, 43856 μmol) and heated to 100° C. overnight. The reaction mixture was allowed to cool to room temperature. 1N HCl was added to bring pH to 2-4. A white solid crashed out which was filtered, then taken up in DCM, washed with brine, dried over Na₂SO₄, filtered and concentrated and dried to give 2-(3-bromo-5-methylphenyl)-propanoic acid. The crude material was carried on without purification.

### Reference 8

### Synthesis of 2-(3-bromo-5-(trifluoromethyl)phenyl)propanoic acid

### Step 1

To a slurry of sodium hydride, 60% dispersion in mineral oil (572 mg, 14.9 mmol) in NMP (5 mL) in a microwave vial under a nitrogen atmosphere was slowly added ethyl cyanoacetate (1.59 ml, 14.9 mmol). The solution was stirred until H₂ evolution had ceased. 1-Bromo-3-fluoro-5-(trifluoromethyl)benzene (1.21 g, 4.98 mmol) was added to the vial which was then sealed. The reaction mixture was stirred at 110° C. for 16 h. The reaction mixture was cooled to RT and mixed with water and enough 2 N HCl to bring the pH to 2-3 and the product was extracted with ether. The organic layer was combined, dried (Na₂SO₄) and concentrated. The crude product was chromatographed through a Redi-Sep® pre-packed silica gel column (40 g), eluting with a gradient of 0% to 30% EtOAc in hexane, to provide ethyl 2-(3-bromo-5-(trifluoromethyl)phenyl)-2-cyanoacetate (908 mg, 54% yield) as a colorless oil.

### Step 2

A mixture of ethyl 2-(3-bromo-5-(trifluoromethyl)phenyl)-2-cyanoacetate (905 mg, 2.69 mmol) in aqueous sodium hydroxide (538 mg, 13.5 mmol in 10 mL of water) was heated at 95-100° C. for 6 h. The reaction mixture was cooled and enough 2N HCl was added to bring the pH to 2-4. The precipitates formed were collected by filtration, washed with water, and dried to provide 2-(3-bromo-5-(trifluoromethyl)phenyl acetic acid as off-white solid.

### Step 3

To a solution of 2-(3-bromo-5-(trifluoromethyl)phenyl)acetic acid (283 mg, 1.0 mmol) in THF (5 mL) at 0° C. was added 1.0 M lithium bis(trimethylsilyl)amide in THF (2.3 ml, 2.3 mmol) dropwise. After stirring for 30 min, the cooling bath was removed and the reaction mixture was gradually warmed to RT. Iodomethane (75 μl, 1.2 mmol) was then added dropwise. The reaction mixture was stirred at RT for 4 h. 5 N aqueous HCl was added to adjust the pH to 1. The product was extracted with EtOAc and the combined organic phase was washed with water, brine, dried over Na₂SO₄, and concentrated. The crude product was chromatographed through a Redi-Sep® pre-packed silica gel column (40 g), eluting with a gradient of 0% to 60% EtOAc in hexane, to afford 2-(3-bromo-5-(trifluoromethyl)phenyl)-propanoic acid (52 mg, 18% yield) as off-white solid.

### Reference 9

### Synthesis of 2-(3-(difluoromethyl)-5-(trifluoromethyl)phenyl)propanoic acid

### Step 1

To a solution of 3-fluoro-5-(trifluoromethyl)benzaldehyde (5 g, 26 mmol), DAST (10 g, 65 mmol), and DCM (2 mL) was added 1 drop of MeOH. The resulting mixture was stirred overnight at room temperature. Reaction was quenched by dropwise addition of the reaction mixture (via pippet) to a flask of water which was stirring vigorously. The resulting mixture was diluted with EtOAc and the biphasic layers were separated by separatory funnel. The organic layer was dried over sodium sulfate and evaporated to afford 1-(difluoromethyl)-3-fluoro-5-(trifluoromethyl)benzene.

### Step 2

A solution of 1-(difluoromethyl)-3-fluoro-5-(trifluoromethyl)benzene (6.6 g, 31 mmol), ethyl 2-cyanopropanoate (16 g, 124 mmol), and cesium carbonate (40 g, 124 mmol) in 31 mL of DMF was heated to 100° C. overnight. The reaction mixture was extracted with DCM and the organic layer was washed with water and brine to remove DMF. The organic layer was dried, concentrated and the residue was purification by Biotage (0-10% MeOH/DCM). The isolated material was then suspended in an excess of 1N NaOH solution and the resulting mixture was heated to 105° C. and stirred for 2 days. The reaction mixture was acidified with conc HCl and extracted with DCM and EtOAc to afford 2-(3-(difluoromethyl)-5-(trifluoromethyl)-phenyl)propanoic acid

### Reference 10

### Synthesis of 2-(benzo[d][1,3]dioxol-5-yl)-4-methylpentanoic acid

To a solution of 2-(benzo[d][1,3]dioxol-5-yl)acetic acid (1 g, 6 mmol) in THF under N₂ was added LHMDS (13 ml, 13 mmol). The resulting mixture was stirred at room temperature for 1 hr before addition of 1-iodo-2-methylpropane (0.6 ml, 6 mmol). The reaction mixture was stirred at room temperature until the reaction was complete. Reaction mixture was quenched with 1N HCl and extracted with DCM. Purification by Biotage (0-10% MeOH/DCM) produced the title compound.

### Reference: 11

### Synthesis of 5-Amino-2-(2-methylpyridin-4-yl)benzonitrile

### Step 1

To a mixture of potassium phosphate hydrate (2.3 g, 9.9 mmol), 2-methylpyridin-4-ylboronic acid (1.2 g, 8.8 mmol), 2-bromo-5-nitrobenzonitrile (1.0 g, 4.4 mmol) in dioxane (45 mL) and water (9 mL) purged with nitrogen was added bis(di-t-butylphenylphosphine)-dichloropalladium catalyst (0.096 g, 0.15 mmol). The reaction mixture was heated to 100° C. overnight. The reaction mixture was diluted saturated Na₂CO₃ and EtOAc. The organic phase was separated. The aqueous phase was extracted with a mixture of CHCl₃:i-PrOH (3:1) three times. The organic phase was dried over Na₂SO₄, and concentrated in vacuo. Purification by silica gel chromatography (25-100%, then 100% EtOAc-hexane) afforded 2-(2-methylpyridin-4-yl)-5-nitrobenzonitrile as a tan solid (0.67 g, 64%).

### Step 2

A mixture of 2-(2-methylpyridin-4-yl)-5-nitrobenzonitrile (0.67 g, 2.8 mmol), EtOH (22 mL), CH₃CN (8 mL) and palladium, 10 wt. % on activated carbon (0.30 g) was purged with hydrogen balloon and stirred at RT. The catalyst was filtered through celite and washed with DCM and the filtrate was concentrated in vacuo to give 5-amino-2-(2-methylpyridin-4-yl)benzonitrile as a light yellow solid (0.59 g, 100%).

### Reference 13

### Synthesis of 2-(9-Methyl-9H-carbazol-3-yl)propanoic acid

### Step 1

To a solution of 2-(9-methyl-9H-carbazol-3-yl)acetic acid (1.03 g, 4.30 mmol) in MeOH (2.0 mL), THF (2 mL) and toluene (10 mL) was added trimethylsilyl)diazomethane, 2.0 M in hexane (2.58 ml, 5.17 mmol) dropwise. The reaction mixture was stirred at RT for 30 min. The solution was concentrated in vacuo to give methyl 2-(9-methyl-9H-carbazol-3-yl)acetate as a white solid was used directly in the next step (1.09 g, 100%).

### Step 2

To a solution of methyl 2-(9-methyl-9H-carbazol-3-yl)acetate (1.40 g, 5.5 mmol) in THF (10 mL) was added sodium bis(trimethylsilyl)amide, 1.0M solution in THF (7.2 ml, 7.2 mmol) at 0° C. The reaction mixture was stirred for 30 min, and iodomethane (0.45 ml, 7.2 mmol) was added and stirring was continued for additional 2 hours. The reaction mixture was concentrated in vacuo, diluted with NaHCO₃ and extracted with EtOAc. The organic phase was dried over Na₂SO₄, filtered and concentrated in vacuo. The crude was purified by silica gel chromatography (0-20% in 10 min, then 20-25% in 15 min EtOAc-hexane) to give methyl 2-(9-methyl-9H-carbazol-3-yl)propanoate as a sticky oil (0.55 g, 37%).

### Step 3

To a mixture of methyl 2-(9-methyl-9H-carbazol-3-yl)propanoate (0.55 g, 2.1 mmol) in a mixed solvent of THF:MeOH:H₂O (10 mL, 3:1:1) was added lithium hydroxide hydrate (0.43 g, 10.0 mmol). The reaction mixture was stirred at rt overnight. The crude was concentrated in vacuo. 2N HCl was added until pH=2, followed by addition of diethyl ether. The precipitates were collected by filtration and washed with water and dried in vacuum oven overnight to give 2-(9-methyl-9H-carbazol-3-yl)propanoic acid as a tan solid (0.32 g, 61%).

### Reference 14

### Synthesis of 2-(3,4-dihydro-1H-pyrido[3,4-b]indol-2(9H)-yl)propanoic acid

### Step 1

To a mixture of 2,3,4,9-tetrahydro-1H-pyrido[3,4-b]indole (0.60 g, 3.48 mmol) in CH₃CN (10 mL) and DMF (5 mL) was added methyl 2-bromopropanoate (0.43 ml, 3.83 mmol) and N-ethyl-N-isopropylpropan-2-amine (1.21 ml, 6.97 mmol). The reaction mixture was stirred at RT overnight, diluted with EtOAc and washed with water and brine. The organic layers were dried over Na₂SO₄, filtered and concentrated in vacuo. Purification of the crude by silica gel chromatography (20-50% CH₃CN—CH₂Cl₂) afforded methyl 2-(3,4-dihydro-1H-pyrido[3,4-b]indol-2(9H)-yl)propanoate as a brown sticky solid (0.88 g, 98%).

### Step 2

To a mixture of methyl 2-(3,4-dihydro-1H-pyrido[3,4-b]indol-2(9H)-yl)propanoate (0.80 g, 3.1 mmol) in a mixture of solvent of THF:MeOH:H₂O (10 mL, 3:1:1) was added lithium hydroxide hydrate (0.52 g, 12 mmol). The reaction mixture was stirred at RT for 3 h and the concentrated in vacuo. 2N HCl was added until pH=2 and then diethyl ether was added. The precipitates were filtered, washed with water, and dried in vacuum oven overnight to give 2-(3,4-dihydro-1H-pyrido[3,4-b]indol-2(9H)-yl)propanoic acid as a tan solid (0.64 g, 85%).

### Reference 15

### Synthesis of 2-(3-bromophenyl)-4-methylpentanoic acid

To a solution of 3-bromophenylacetic acid (3.00 g, 14 mmol) (azeotroped with toluene) in 2 mL of dry toluene was added sodium bis(trimethylsilyl)amide, 1.0 m solution in tetrahydrofuran (32 mL of 1N solution in THF). After stirring at RT for 20 min, 1-iodo-2-methylpropane (2 ml, 15 mmol) was added dropwise. After 10 min, the reaction mixture was quenched with 2 N HCl to PH=2, extracted with EtOAc, dried over Na₂SO₄, filtered and evaporated to dryness. CC (SiO₂, DCM to DCM/EtOAc=10:1 to 4:1 to 2:1) gave 2-(3-bromophenyl)-4-methylpentanoic acid (3.43 g, 91% yield) as a colorless oil which upon standing overnight became an offwhite solid.

### Examples 1

### Synthesis of (S)-2-(6-methoxynaphthalen-2-yl)-N-(4-(2-methylpyridin-4-yl)-phenyl)propanamide

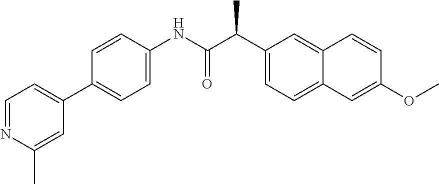

A solution of 4-(2-methylpyridin-4-yl)benzenamine (50 mg, 0.27 mmol) in NMP (0.5 mL) was added to a solution of (S)-2-(6-methoxynaphthalen-2-yl)propanoic acid (69 mg, 0.30 mmol), HATU (115 mg, 0.30 mmol), DIEA (0.81 mmol, 144 uL), and NMP (0.9 mL). The reaction mixture was shaken for 24 h and was purified directly by preparative HPLC (10-60% CH₃CN/H₂O modified with 0.1% TFA) to give (S)-2-(6-methoxynaphthalen-2-yl)-N-(4-(2-methylpyridin-4-yl)phenyl)propanamide as the TFA salt. [M+1]=397.

### Example 2

### Synthesis of 2-(3,5-bis(trifluoromethyl)phenyl)-N-(4-(2-methylpyridin-4-yl)-phenyl)propanamide

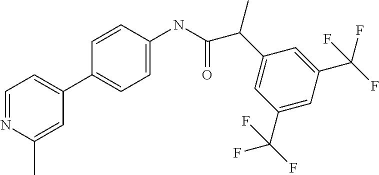

2-(3,5-bis(Trifluoromethyl)phenyl)propanoic acid (110 mg, 384 μmol), 4-(2-methylpyridin-4-yl)benzenamine (77 mg, 418 μmol) in DMF (10 ml) were added HOBt (40 mg) and EDC 0.14 g (2 eq). The reaction mixture was stirred for 18 h. Ethyl acetate was added, and the organic layer was washed with brine, dried over Na₂CO₃ and evaporated. Column chromatograph purification (40-70% EtOAc/hexanes) of the crude product gave the title compound.

### Example 3

### Synthesis of 4-methyl-N-(4-(2-methylpyridin-4-yl)phenyl)-2-m-tolylpentanamide

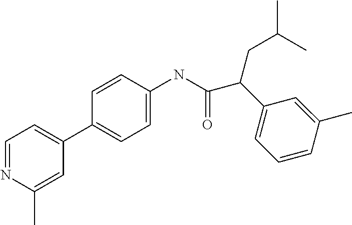

A solution of 4-(2-methylpyridin-4-yl)benzenamine (260 mg, 1.411 mmol), 4-methyl-2-m-tolylpentanoic acid (378 mg, 1.835 mmol), PYBOP (1175 mg, 2.258 mmol) and diisopropylethylamine (737 μl, 4.234 mmol) in 0.5 mL of DMF for 12 hours. The reaction mixture was quenched with sat. NaHCO₃, extracted with EtOAc/hexane=2:1, washed with brine, dried over Na₂SO₄, filtered and evaporated to dryness. Column chromatograph (SiO₂, DCM to DCM/EtOAc=4:1 to 3:1 to 2:1 to 1:1) afforded 4-methyl-N-(4-(2-methylpyridin-4-yl)phenyl)-2-m-tolylpentanamide (450 mg, 85.6% yield) as an offwhite gum. MS (ESI, pos. ion) m/z: 373 (M+1).

### Example 4

### Synthesis of 2-(3-bromo-5-chlorophenyl)-N-(4-(2-methylpyridin-4-yl)phenyl)propanamide

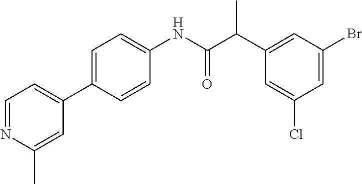

A mixture of 2-(3-bromo-5-chlorophenyl)propanoic acid (8.00 g, 30 mmol), 4-(2-methylpyridin-4-yl)benzenamine (5.6 g, 30 mmol), TBTU (11 g, 33 mmol) and TEA (6.3 ml, 46 mmol) in DMF (30 ml) was stirred at RT, under nitrogen overnight. The reaction mixture was diluted with water and extracted with DCM. The organic layer was washed with an aqueous saturated solution of sodium bicarbonate, then with water and then brine. The organic layer was then dried with sodium sulfate and purified by column chromatography on silica gel using a gradient of 0 to 3% MeOH in ethyl acetate to give 2-(3-bromo-5-chlorophenyl)-N-(4-(2-methylpyridin-4-yl)phenyl)propanamide as an off-white solid.

### Example 5

### Synthesis of 2-(3-chloro-5-(3,4-dihydro-2H-benzo[b][1,4]oxazin-6-yl)phenyl)-N-(4-(2-methylpyridin-4-yl)phenyl)propanamide

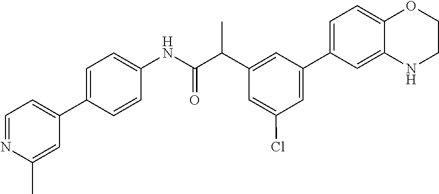

A vial was charged with 2-(3-bromo-5-chlorophenyl)-N-(4-(2-methylpyridin-4-yl)phenyl)propanamide (0.140 g, 0.33 mmol), tert-butyl 6-(4,4,5,5-tetramethyl-1,3,2-dioxaborolan-2-yl)-2,3-dihydrobenzo[b][1,4]oxazine-4-carboxylate (0.151 g, 0.419 mmol), sodium carbonate (0.064 g, 0.65 mmol), tetrakis(triphenylphosphine)palladium (0.0175 g, 0.016 mmol), water (0.4 mL) and dioxane (3.6 mL). The vial was capped and the reaction was heated to 70° C. After two hours, the reaction mixture was cooled down and diluted with ethyl acetate and water. The organic portion was washed with an aqueous saturated solution of sodium bicarbonate, then with water and then brine. The organic layer was then dried with sodium sulfate, reduced and purified by column chromatography on silica gel using a gradient of 40 to 100% EtOAc in hexanes to give the Boc protected product. This product was taken up in DCM (3 mL) and treated with TFA (1.5 mL). The reaction mixture was stirred overnight. The volatiles were removed under vacuum and the residue was taken in saturated sodium bicarbonate and DCM. The organic portion was concentrated and purified by RP-HPLC using a gradient of 5% ACN 0.1% TFA to 95% ACN 0.1% TFA in water 0.1% TFA. The pure fractions were neutralized with ammonium hydroxide and the volatiles were removed under reduced pressure. The solid that crashed out of the aqueous layer was filtered off, washed with water and dried in a vacuum oven to give 2-(3-chloro-5-(3,4-dihydro-2H-benzo[b][1,4]oxazin-6-yl)phenyl)-N-(4-(2-methylpyridin-4-yl)phenyl)propanamide as an off-white solid.

### Example 6

### Synthesis of 2-(3-chloro-5-(1-cyclopropyl-1H-pyrazol-4-yl)phenyl)-N-(4-(2-methylpyridin-4-yl)phenyl)propanamide

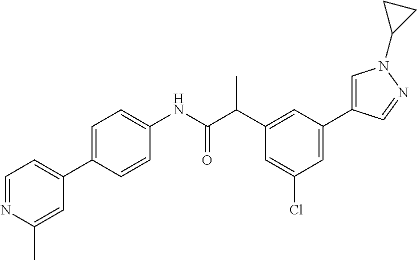

A clear microwave vial was charged with 2-(3-bromo-5-chlorophenyl)-N-(4-(2-methylpyridin-4-yl)phenyl)propanamide (0.150 g, 0.349 mmol), 1-cyclopropyl-4-(4,4,5,5-tetramethyl-1,3,2-dioxaborolan-2-yl)-1H-pyrazole (0.0981 g, 0.419 mmol), tetrakis(triphenylphosphine)palladium (0.0202 g, 0.0175 mmol), sodium carbonate (0.349 ml, 0.698 mmol) and dioxane (4 mL). The vial was flushed with nitrogen and capped. The vial was heated in a Personal Chemistry SmithSynthesizer to 110° C. for 10 minutes. The reaction mixture was diluted with ethyl acetate and water. The organic portion was washed with an aqueous saturated solution of sodium bicarbonate, then with water and then brine. The organic layer was then dried with sodium sulfate, reduced and purified by RP-HPLC using a gradient of 5% ACN 0.1% TFA to 95% ACN 0.1% TFA in water 0.1% TFA. The pure fractions were neutralized with ammonium hydroxide and the volatiles were removed under reduced pressure. The solid that crashed out of the aqueous layer was filtered off, washed with water and dried in a vacuum oven at 45 degrees to give 2-(3-chloro-5-(1-cyclopropyl-1H-pyrazol-4-yl)phenyl)-N-(4-(2-methylpyridin-4-yl)phenyl)propanamide as an off-white solid.

### Example 7

### Synthesis of (±)-2-(3-methyl-5-(4-(pyridin-2-yl)piperazin-1-yl)phenyl)-N-(4-(2-methylpyridin-4-yl)phenyl)propanamide

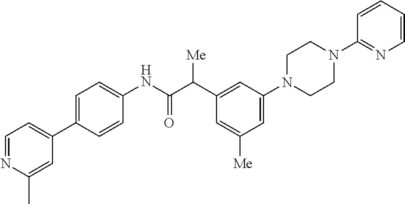

2-(3-Bromo-5-methylphenyl)-N-(4-(2-methylpyridin-4-yl)phenyl)propanamide (129 mg, 315 μmol), sodium t-butoxide (75.7 mg, 788 μmol), 2-dicyclohexylphosphino-2′,6′-dimethoxy-1,1′-biphenyl (12.9 mg, 31.5 μmol), and 1-(pyridin-2-yl)piperazine (55.2 μl, 378 μmol) in toluene was combined with tris(dibenzylideneacetone)dipalladium (o) (14.4 mg, 15.8 μmol). The vial was placed under Ar(g) and heated to 80° C. overnight. The reaction mixture was cooled to room temperature, filtered through a millipore filter, and purified by flash silica gel chromatography to give the title compound as a racemic mixture.

### Example 8

### Synthesis of (±)-2-(3-methyl-5-(2-methylpyridin-4-yl)phenyl)-N-(4-(2-methylpyridin-4-yl)-phenyl)propanamide

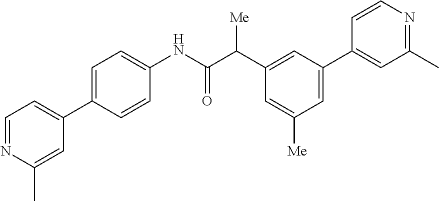

A mixture of potassium phosphate hydrate (215 mg, 0.934 mmol), 2-methylpyridin-4-ylboronic acid (171 mg, 1.246 mmol), 2-(3-bromo-5-methylphenyl)-N-(4-(2-methylpyridin-4-yl)phenyl)propanamide (170 mg, 0.415 mmol) in dioxane (0.75 mL) and water (0.25 mL) was purged with argon and then tris(dibenzylideneacetone)dipalladium(0) (44.1 mg, 62.3 μmol) was added. The reaction mixture was heated to 100° C. overnight and then cooled to room temperature, filtered through a millipore filter, and purified by flash silica gel chromatography using a 1.5-4.5% MeOH/DCM gradient to give the title compound as a racemic mixture.

### Example 9

### Synthesis of 2-(7-bromo-3,4-dihydroisoquinolin-2(1H)-yl)-N-(4-(2-methylpyridin-4-yl)-phenyl)propanamide

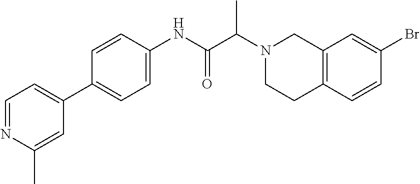

To the solution of 4-(2-methylpyridin-4-yl)benzenamine (540 mg, 2.93 mmol) and triethylamine (1.22 ml, 8.79 mmol) in DCM (5 mL) was added 2-bromopropionyl chloride (0.274 ml, 2.64 mmol). After stirring for 10 min, 7-bromo-1,2,3,4-tetrahydroisoquinoline (808 mg, 3.81 mmol) was added. The reaction mixture was stirred for overnight, diluted with DCM, washed with water, dried over MgSO₄. After filtration and concentration, the title compound was obtained through silica gel flash column chromatography (eluted with CM:EtOAc:MeOH, 4:1:0.1) as a yellow oil. MS m/z: 450 (M+1).

### Example 10

### Synthesis of 2-(7-(2-methylpyridin-4-yl)-3,4-dihydroisoquinolin-2(1H)-yl)-N-(4-(2-methylpyridin-4-yl)phenyl)propanamide

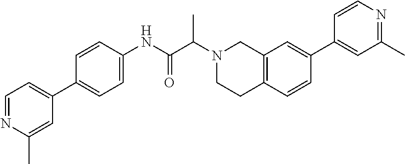

A mixture of 2-(7-bromo-3,4-dihydroisoquinolin-2(1H)-yl)-N-(4-(2-methylpyridin-4-yl)phenyl)propanamide (275 mg, 0.61 mmol), 2-methylpyridin-4-ylboronic acid (125 mg, 0.916 mmol) and potassium phosphate (907 mg, 4.27 mmol) in toluene (3 mL) in microwave reaction vessel was degassed with nitrogen for 30 seconds. Pd catalyst [(tBu)₂PPh]₂PdCl₂ (34 mg, 55 μmol) was added, the reaction vessel was sealed and the reaction mixture was stirred at 90° C. for 1 h. The solvents were removed under lower pressure, and the residue was diluted with water (20 mL), extracted with EtOAc, and dried over Na₂SO₄. After filtration and concentration, the desired product was obtained through silica gel flash column chromatography (eluted with DCM:EtOAc:MeOH, 4:1:0.1) as a semi-solid. MS m/z: 463 (M+1).

### Example 11

### Synthesis of 2-(4-(isopropylamino)phenyl)-N-(4-(2-methylpyridin-4-yl)phenyl)propanamide

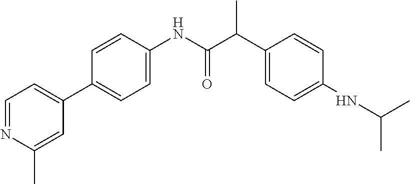

### Step 1

A mixture of 2-(4-nitrophenyl)propanoic acid (1.36 g, 6.97 mmol), CH₂Cl₂ (16 mL), oxalyl chloride (2.44 ml, 27.9 mmol) and 2 drops of DMF was stirred at RT overnight. The crude 2-(4-nitrophenyl)propanoyl chloride was concentrated in vacuo, dried on vacuum to give 2-(4-nitrophenyl)propanoyl chloride which was used in the next step directly.

### Step 2

A mixture of 4-(2-methylpyridin-4-yl)benzenamine (0.59 g, 3.2 mmol), CH₂Cl₂ (11 mL), 2-(4-nitrophenyl)propanoyl chloride (0.75 g, 3.5 mmol), N,N-diisopropylethylamine (1.1 ml, 6.4 mmol) was stirred at RT overnight. The reaction mixture was diluted with Na₂CO₃ (50 mL) and extracted with a mixture of CHCl₃:i-PrOH (3:1). The organic layers were dried over Na₂SO₄, filtered and concentrated in vacuo. Purification by silica gel chromatography (0-100%, then 100% EtOAc-hexane) afforded N-(4-(2-methylpyridin-4-yl)phenyl)-2-(4-nitrophenyl)propanamide as an off-white solid (0.93 g, 81% over two steps).

### Step 3

A mixture of N-(4-(2-methylpyridin-4-yl)phenyl)-2-(4-nitrophenyl)propanamide (0.44 g, 1.2 mmol), EtOH (12 mL) and palladium (10 wt. % on activated carbon, 65 mg, 0.061 mmol) was connected to a hydrogen balloon and stirred at RT for 3 h. The crude was filtered through celite and the filtrate was concentrated in vacuo. 2-(4-Aminophenyl)-N-(4-(2-methylpyridin-4-yl)phenyl)propanamide was obtained as an off-white solid (0.40 g, 99%).

### Step 3

To a mixture of 2-(4-aminophenyl)-N-(4-(2-methylpyridin-4-yl)phenyl)propanamide (100 mg, 0.30 mmol), acetone (0.044 mL, 0.60 mmol), and ClCH₂CH₂Cl (2 mL) was added sodium triacetoxyborohydride (96 mg, 0.45 mmol) at RT. The reaction mixture was stirred overnight, then diluted with saturated Na₂CO₃ and extracted with a mixture of CHCl₃:i-PrOH (3:1). The organic phase was dried over Na₂SO₄, and concentrated in vacuo. Purification by silica gel chromatography (0-10% MeOH—CH₂Cl₂) afforded 2-(4-(isopropylamino)phenyl)-N-(4-(2-methylpyridin-4-yl)phenyl)propanamide as a white solid. The product was further purified by reverse phase prep HPLC (Phenomenex Gemini C18 5 mm 100×30 mm, 5%-70% CH₃CN (0.1% TFA)-water (with 0.1% TFA) in 10 min, 30 mL/min) to give it as a white solid (78 mg, 69%). MS (M+1): 374.

### Example 12

### Synthesis of N-(4-(2-methylpyridin-4-yl)phenyl)-2-(6-(piperidin-1-yl)pyridin-3-yl)-propanamide

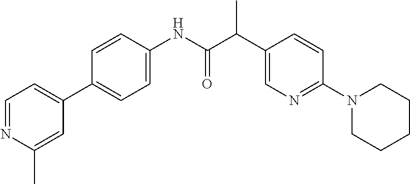

### Step 1

To a solution of 2-(6-chloropyridin-3-yl)acetic acid (1.00 g, 5.83 mmol) in THF (10 mL) was added sodium bis(trimethylsilyl)amide, 1.0 M solution in THF (13.4 ml, 13.4 mmol) dropwise at 0° C. The reaction mixture was stirred for 30 min. Iodomethane (0.44 ml, 6.99 mmol) was added dropwise and the cooling bath was removed. The reaction mixture was stirred for 2 h, and HOAc was added to the mixture until pH 4. The reaction mixture was diluted with CH₂Cl₂ and water. The organic phase was washed with water, brine, dried over Na₂SO₄, filtered and concentrated in vacuo to give 2-(6-Chloropyridin-3-yl)propanoic acid as a light yellow solid (0.89 g, 82%).

### Step 2

A mixture of 2-(6-chloropyridin-3-yl)propanoic acid (0.111 g, 0.597 mmol), DMF (1.5 mL), PyBOP (0.424 g, 0.814 mmol), 4-(2-methylpyridin-4-yl)benzenamine (0.1000 g, 0.543 mmol), and triethylamine (0.151 ml, 1.09 mmol) was stirred at RT overnight. The reaction mixture was heated to 60° C. for 3 h. The reaction mixture was diluted with saturated Na₂CO₃ and extracted with a mixture of CHCl₃:i-PrOH (3:1). The organic phase was washed with saturated brine, dried over Na₂SO₄, and concentrated in vacuo. Purification by silica gel chromatography (0-10% MeOH-EtOAc) afforded 2-(6-Chloropyridin-3-yl)-N-(4-(2-methylpyridin-4-yl)phenyl)propanamide with impurities as a yellow solid. The compound was further purified (0-50% CH₃CN-EtOAc) and the product was obtained as a white solid (36 mg, 19%).

### Step 3

A mixture of 2-(6-chloropyridin-3-yl)-N-(4-(2-methylpyridin-4-yl)phenyl)propanamide (23 mg, 0.066 mmol) and piperidine (0.5 mL) in a sealed tube was heated to 110° C. overnight. The crude was purified by reverse phase prep HPLC (Phenomenex Gemini C18 5 mm 100×30 mm, 15%-60% CH₃CN (0.1% TFA)-water (with 0.1% TFA) in 15 min, 30 mL/min) to give N-(4-(2-methylpyridin-4-yl)phenyl)-2-(6-(piperidin-1-yl)pyridin-3-yl)propanamide as a white solid (14 mg, 51%). MS: 401 (M+1).

### Example 13

### Synthesis of N-(4-(2-methylpyridin-4-yl)phenyl)-2-(4-(piperidin-1-yl)phenyl)propanamide

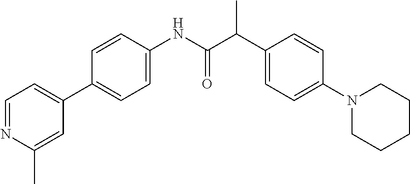

To a mixture of 2-(4-aminophenyl)-N-(4-(2-methylpyridin-4-yl)phenyl)propanamide (133 mg, 0.40 mmol), glutaraldehyde, 50% aq solution (105 mg, 1.00 mmol), ClCH₂CH₂Cl (12 mL) and DMF (0.5 mL) was added sodium triacetoxyborohydride (187 mg, 0.89 mmol) at rt. The reaction mixture was stirred overnight, diluted with saturated Na₂CO₃ and extracted with a mixture of CHCl₃:i-PrOH (3:1). The organic phase was dried over Na₂SO₄, and concentrated in vacuo. Purification by silica gel chromatography (5-50% EtOAc-hexane) afforded the product as a colorless oil. The product was further purified by reverse phase prep HPLC (Phenomenex Gemini C18 5 mm 100×30 mm, 15%-60% CH₃CN (0.1% TFA)-water (with 0.1% TFA) in 15 min, 30 mL/min) to give the title compound as a white solid (118 mg, 74%). MS: 400 (M+1).

### Example 14

### Synthesis of 3-cyclopropyl-2-(5-(isopropylamino)pyridin-2-yl)-N-(4-(2-methylpyridin-4-yl)-phenyl)propanamide

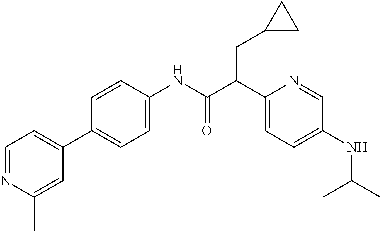

### Step 1

To a mixture of sodium hydride, 60% dispersion in mineral oil (1.8 g, 74 mmol) in THF (30 mL) and dioxane (20 mL) was added dimethyl malonate (8.4 ml, 74 mmol). The reaction mixture was stirred for 15 min, and a solution of 2-fluoro-5-nitropyridine (3.0 g, 21 mmol) in dioxane (10 mL) was added. The reaction mixture was added DMF (10 mL) and stirred overnight. 5N HCl was added to the mixture until pH 1 (40 mL). The reaction mixture was extracted with EtOAc and the organic phase was washed with water and brine, dried over Na₂SO₄, filtered and concentrated in vacuo. Purification by silica gel chromatography (0%-50% EtOAc-hexane) afforded dimethyl 2-(5-nitropyridin-2-yl)malonate as a yellow solid (5.1 g, 95%).

### Step 2

To a mixture of sodium hydride, 60% dispersion in mineral oil (0.404 g, 16.8 mmol) in THF (10 mL) was added a solution of dimethyl 2-(5-nitropyridin-2-yl)malonate (1.07 g, 4.21 mmol) in THF (10 mL) and DMF (10 mL) at 0° C. The reaction mixture was stirred for 5 min, then (bromomethyl)cyclopropane (1.63 ml, 16.8 mmol) was added. The cooling bath was removed and the mixture was heated to 70° C. and stirred overnight. The reaction mixture was concentrated in vacuo, neutralized with 5N HCl to pH 5 and saturated NaHCO₃ to basic, and extracted with EtOAc. The organic phase was washed with water and brine, dried over Na₂SO₄, filtered and concentrated in vacuo. The crude was purified by silica gel chromatography (10-30% EtOAc-hexane) to give dimethyl 2-(cyclopropylmethyl)-2-(5-nitropyridin-2-yl)malonate as yellow oil (0.697 g, 54%).

### Step 3

To a solution of dimethyl 2-(cyclopropylmethyl)-2-(5-nitropyridin-2-yl)malonate (0.690 g, 2.24 mmol) in DMSO (10 mL) was added lithium chloride (1.138 g, 26.86 mmol) and water (0.81 ml, 44.76 mmol). The reaction mixture was heated to 100° C. and stirred overnight. The reaction mixture was cooled and diluted with EtOAc and brine. The organic phase was washed with brine, dried over Na₂SO₄, filtered and concentrated in vacuo. The crude was purified by silica gel chromatography (10-15-50% EtOAc-hexane) to give methyl 3-cyclopropyl-2-(5-nitropyridin-2-yl)propanoate as yellow oil (0.209 g, 37%).

### Step 4

To a solution of methyl 3-cyclopropyl-2-(5-nitropyridin-2-yl)propanoate (0.100 g, 0.40 mmol) in MeOH (10 mL) was added palladium, 10% wt on carbon (0.043 g, 0.40 mmol). The reaction mixture was connected to a H₂ balloon and stirred for 2 h. The reaction mixture was filtered through celite. The filtrated was concentrated in vacuo to give methyl 2-(5-aminopyridin-2-yl)-3-cyclopropylpropanoate (0.080 g, 91%).

### Step 5

To a mixture of methyl 2-(5-aminopyridin-2-yl)-3-cyclopropylpropanoate (0.066 g, 0.30 mmol), acetone (0.066 ml, 0.90 mmol), ClCH₂CH₂Cl (2 mL), DMF (0.2 mL) was added sodium triacetoxyborohydride (0.19 g, 0.90 mmol) at rt. The reaction mixture was diluted with saturated Na₂CO₃ and extracted with a mixture of CHCl₃:i-PrOH (3:1). The organic phase was dried over Na₂SO₄, and concentrated in vacuo. Purification by ISCO (40 g, 5-50% EtOAc-hexane) afforded methyl 3-cyclopropyl-2-(5-(isopropylamino)pyridin-2-yl)propanoate as colorless oil (0.066 g, 84%). MS: 263 (M+1).

### Step 6

A mixture of methyl 3-cyclopropyl-2-(5-(isopropylamino)pyridin-2-yl)propanoate (0.066 g, 0.253 mmol), THF (1.5 mL), MeOH (0.5 mL), water (0.5 mL) and lithium hydroxide hydrate (0.080 g, 1.90 mmol) was stirred at RT overnight. The crude was concentrated in vacuo. Ether was added and evaporated to remove the water to give 3-cyclopropyl-2-(5-(isopropylamino)pyridin-2-yl)propanoic acid as off-white solid (0.063 g, 100%).

### Step 7

A mixture of 3-cyclopropyl-2-(5-(isopropylamino)pyridin-2-yl)propanoic acid (0.063 g, 0.25 mmol), DMF (1.5 mL), PYBOP (0.20 g, 0.38 mmol), 4-(2-methylpyridin-4-yl)-benzenamine (0.070 g, 0.38 mmol), and triethylamine (0.070 ml, 0.51 mmol) was stirred at RT overnight. The reaction mixture was diluted with saturated Na₂CO₃ and extracted with a mixture of CHCl₃:i-PrOH (3:1). The organic phase was washed with saturated brine, dried over Na₂SO₄, and concentrated in vacuo. Purification by silica gel chromatography (10-100% EtOAc-hexane to remove the impurities, and then 10% MeOH—CH₂Cl₂) afforded the crude product as yellow oil which was further purified by prep TLC (7% MeOH—CH₂Cl₂) to give 3-cyclopropyl-2-(5-(isopropylamino)pyridin-2-yl)-N-(4-(2-methylpyridin-4-yl)phenyl)-propanamide as a light yellow solid (0.020 g, 19%).

### Example 15

### Synthesis of 2-(2-bromophenylamino)-4-methyl-N-(4-(2-methylpyridin-4-yl)-phenyl)-pentanamide

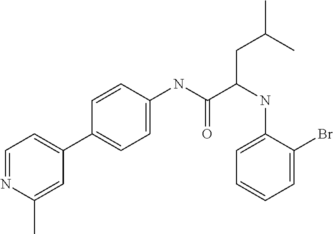

### Step 1

A mixture of 4-methyl-2-oxovaleric acid (1 g, 8 mmol) and 2-bromoaniline (3 g, 15 mmol) were combined in DCM and stirred at room temperature overnight. To the solution was then added sodium triacetoxyborohydride (2 g, 8 mmol) and the reaction mixture was stirred overnight. The reaction mixture was evaporated and purified by Biotage (0-10% MeOH/DCM) to give 2-(2-bromophenylamino)-4-methylpentanoic acid.

### Step 2

To a solution of 2-(2-bromophenylamino)-4-methylpentanoic acid (0.7 g, 2 mmol) in DMF (4 mL) was added HATU (0.9 g, 2 mmol), 4-(2-methylpyridin-4-yl)benzenamine (0.4 g, 2 mmol), and DIPEA (0.4 ml, 2 mmol) last. The resulting mixture was stirred at room temperature overnight. Ethyl acetate (50 mL) was added and washed with brine, dried, and evaporated. The residue was purified by Biotage (0-10% MeOH/DCM) and then by Biotage (0-100% EtOAc/Hexane) to give 2-(2-bromophenylamino)-4-methyl-N-(4-(2-methylpyridin-4-yl)phenyl)pentanamide.

### Example 16

### Synthesis of 2-(3-bromophenyl)-4-methyl-N-(4-(2-methylpyridin-4-yl)phenyl)pentanamide

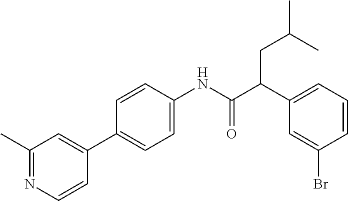

A solution of 4-(2-methylpyridin-4-yl)benzenamine (1000 mg, 5.43 mmol), 2-(3-bromophenyl)-4-methylpentanoic acid (1.62 g, 5.97 mmol), PYBOP (4.52 g, 8.68 mmol) and diisopropylethylamine (2.84 ml, 16.3 mmol) in 0.5 mL of DMF for 12 hours. The reaction mixture was quenched with Sat. NaHCO3, extracted with EtOAc/hexane=2:1, washed with brine, dried over Na2SO4, filtered and evaporated to dryness. Column chromatograph (SiO₂, DCM/EtOAc=4:1 to 3:1 to 2:1) afforded 2-(3-bromophenyl)-4-methyl-N-(4-(2-methylpyridin-4-yl)phenyl)pentanamide (2.31 g, 97.3% yield) as a offwhite gum.

### Example 17

### Synthesis of 2-(3-biphenyl)-4-methyl-N-(4-(2-methylpyridin-4-yl)phenyl)pentanamide

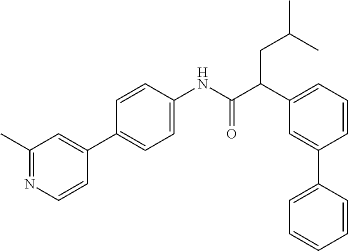

A mixture of 2-(3-bromophenyl)-4-methyl-N-(4-(2-methylpyridin-4-yl)-phenyl)pentanamide (230 mg, 526 μmol), phenylboronic acid (76.9 mg, 631 μmol), tetrakis(triphenylphosphine)palladium (60.8 mg, 52.6 μmol) and sodium carbonate monohydrate (58.0 μl, 1.05 mmol) in 3 mL of dioxane/water=2:1 was heated at 130° C. in microwave irradiation for 30 min. After cooling to RT, the reaction mixture was filtered through silica gel with excess of EtOAc and evaporated to dryness. The residual was submitted to flash chromatography (SiO2, hexane to DCM/EtOAc=3:1 to 2:1 to 1:1) to give 2-(3-phenylphenyl)-4-methyl-N-(4-(2-methylpyridin-4-yl)phenyl)pentanamide as a colorless oil.

### Example 18

### Synthesis of 4-methyl-N-(4-(2-methylpyridin-4-yl)phenyl)-2-(3-morpholinophenyl)-pentanamide

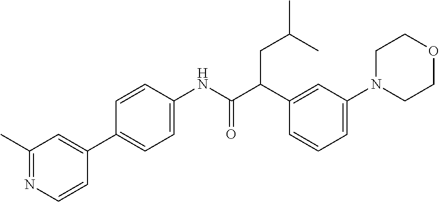

To a 50 mL dry flask was charged CuI (3.8 mg), Cs₂CO₃ (65 mg) and 2-(3-bromophenyl)-4-methyl-N-(4-(2-methylpyridin-4-yl)phenyl)pentanamide (44 mg, 101 μmol). To this mixture under N₂ was added morpholine (26 mg, 302 μmol) followed by 2-acetylcyclohexanone (5.2 μl, 40 μmol) (1 drop) and 0.1 mL of DMF. The reaction mixture was stirred at 100° C. overnight. After cooling down to it, the reaction mixture was directly loaded on to column (SiO2, DCM to DCM/EtOAc=2:1 to 1:1 to 1:2 to 1:3) to give 4-methyl-N-(4-(2-methylpyridin-4-yl)phenyl)-2-(3-morpholinophenyl)pentanamide as a yellow solid.

### Example 19

### Synthesis of 2-(6-fluoropyridin-2-yl)-N-(4-(2-methylpyridin-4-yl)phenyl)propanamide

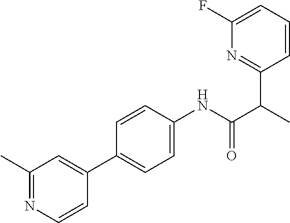

### Step 1

To a solution of 2,6-difluoropyridine (1.207 g, 10.486 mmol) and potassium bis(trimethylsilyl)amide (22 mL of 0.5 M solution in toluene) at RT was added dropwise ethyl propionate (1.151 ml, 10 mmol) in 15 min. After 20 min, the reaction mixture was quenched with sat. NH₄Cl, extracted with EtOAc, dried over Na₂SO₄, filtered and evaporated. Column chromatograph (SiO2, DCM to DCM/EtOAc=10:1 to 5:1 to 3:1) gave ethyl 2-(6-fluoropyridin-2-yl)propanoate (820 mg, 41.6% yield) as a colorless oil.

### Step 2

A solution of ethyl 2-(6-fluoropyridin-2-yl)propanoate (150 mg, 0.76 mmol) in 2 mL of 37% HCl solution was heated at 100° C. for 1 h. After cooling, the solvent was evaporated under high vacuum to give crude 2-(6-fluoropyridin-2-yl)propanoic acid hydrochloride (150 mg, 95.9% yield) as gum. This material was directly used in the next step.

### Step 3

A solution of 4-(2-methylpyridin-4-yl)benzenamine (70 mg, 0.380 mmol), 1-(3-dimethylaminopropyl)-3-ethylcarbodiimide hydrochloride (87 mg, 0.456 mmol), 1-hydroxybenzotriazole (15 mg, 0.114 mmol), triethylamine (79 μl, 0.570 mmol) and 2-(6-fluoropyridin-2-yl)propanoic acid hydrochloride (120 mg, 0.584 mmol) in 0.5 mL of DMF for 12 hours. The reaction mixture was quenched with sat. NaHCO₃, extracted with EtOAc/hexane=2:1, washed with brine, dried over Na₂SO₄, filtered and evaporated to dryness. Column chromatograph (SiO₂, DCM/) afforded 2-(6-fluoropyridin-2-yl)-N-(4-(2-methylpyridin-4-yl)phenyl)propanamide (100 mg, 78% yield) as an offwhite solid.

### Example 20

### Synthesis of N-(4-(2-methylpyridin-4-yl)phenyl)-2-(6-(4-morpholinophenyl)pyridin-2-yl)-propanamide

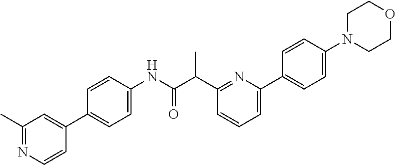

### Step 1

To a azeotroped mixture of propiononitrile (800 mg, 14.53 mmol) and 2,6-dichloropyridine (2579 mg, 17.430 mmol) was added dropwise potassium bis(trimethylsilyl)amide (34 mL of 0.5 M solution in toluene). After 1 h, the reaction mixture was quenched with 2 N HCl to pH=4, extracted with EtOAc, dried over Na₂SO₄, filtered and evaporated to dryness. Column chromatograph (SiO₂, DCM/hexane=1:1 to 2:1 to pure DCM) gave 2-(6-chloropyridin-2-yl)propanenitrile (840 mg, 35% yield) as a colorless oil and 2,2-bis(6-chloropyridin-2-yl)propanenitrile (80 mg, 2.0% yield) as a colorless oil.

### Step 2

A solution of 2-(6-chloropyridin-2-yl)propanenitrile (300 mg, 1.80 mmol) in 5 mL of 37% HCl solution was heated at 100° C. for 1 h. After cooling, the solvent was evaporated under high vacuum to give crude 2-(6-chloropyridin-2-yl)propanoic acid hydrochloride (360 mg, 90.0% yield) as gum. This material was directly used in the next step.

### Step 3

To a dry solution of 2-(6-chloropyridin-2-yl)propanoic acid hydrochloride (188 mg, 0.847 mmol) in 5 mL of DCM was added oxalyl chloride (116 μl, 1.30 mmol) followed by 2 drops of DMF. After 1 h, the solvent was evaporated and the residue was azeotroped with toluene and redissolved in 5 mL of dry DCM. To this was added 4-(2-methylpyridin-4-yl)benzenamine (120 mg, 0.65 mmol) followed by triethylamine (181 μl, 1.303 mmol). After stirring at RT for 15 min, the solvent was evaporated to dryness and the residue was submitted to Column chromatograph (SiO2, DCM/EtOAc=4:1 to 2:1 to 1:1) to afford crude 2-(6-chloropyridin-2-yl)-N-(4-(2-methylpyridin-4-yl)phenyl)propanamide (160 mg, 69.8% yield) as an offwhite solid.

### Step 4

A mixture of potassium phosphate (63 mg, 0.30 mmol), bis(phenyl-di-tert-butylphosphine)palladium chloride (II) (1.2 mg, 2.0 μmol), 4-morpholinophenylboronic acid (31 mg, 0.15 mmol) and 2-(6-chloropyridin-2-yl)-N-(4-(2-methylpyridin-4-yl)phenyl)propanamide (35 mg, 0.10 mmol) in 1.5 mL of dioxane/water=5:1 was heated at 120° C. under microwave irradiation for 30 min. After cooling to RT, the reaction mixture evaporated to dryness. The residual was submitted to flash chromatography (SiO₂, hexane to DCM/EtOAc=3:1 to 1:1 to pure EtOAc) to give N-(4-(2-methylpyridin-4-yl)phenyl)-2-(6-(4-morpholinophenyl)pyridin-2-yl)propanamide (20 mg, 42% yield) as a white solid.

### Example 21

### Synthesis of N-(4-(2-methylpyridin-4-yl)phenyl)-2-(2-(4-morpholinophenyl)pyridin-4-yl)-propanamide

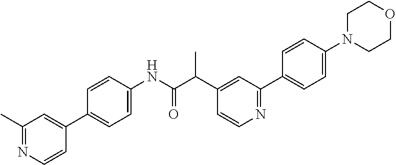

### Step 1

A mixture of 2-cyanopropionic acid ethyl ester (4 ml, 30 mmol) in 3 mL of NMP was cautiously added sodium hydride, 60% dispersion in mineral oil (0.8 ml, 31 mmol) (900 mg of 60% in mineral oil) and the reaction mixture was stirred at RT until hydrogen evolution was ceased. 2-Cyanopropionic acid ethyl ester (4 ml, 30 mmol) was added and the reaction mixture was sealed and heated at 75° C. over 3 h. After cooling, the reaction mixture was quenched with water, extracted with EA, dried over Na₂SO₄, filtered and evaporated to dryness. Column chromatograph (SiO₂, hexane/DCM=1:1 to 1:2 to 1:3) gave ethyl 2-(2-chloropyridin-4-yl)-2-cyanopropanoate (1.90 g, 27% yield) as a sticky oil.

### Step 2

A suspension of ethyl 2-(2-chloropyridin-4-yl)-2-cyanopropanoate (900 mg, 3.77 mmol) in 3 mL of 37% HCl was heated at 90° C. overnight. After cooling to rt, the reaction mixture was extracted with EtOAc, dried over Na₂SO₄, and evaporated to dryness. The residue was submitted to column chromatograph (SiO₂, DCM to DCM/MeOH=10:1 to 5:1) to give 2-(2-chloropyridin-4-yl)propanoic acid (640 mg, 91.4% yield) as a white solid.

### Step 3

A solution of 4-(2-methylpyridin-4-yl)benzenamine (255 mg, 1.38 mmol), 1-(3-dimethylaminopropyl)-3-ethylcarbodiimide hydrochloride (318 mg, 1.66 mmol), 1-hydroxybenzotriazole (56.1 mg, 0.415 mmol) and 2-(2-chloropyridin-4-yl)propanoic acid (257 mg, 1.38 mmol) in 0.5 mL of DMF was stirred at RT for 12 hours. The reaction mixture was quenched with sat. NaHCO₃, extracted with EtOAc/hexane=2:1, washed with brine, dried over Na₂SO₄, filtered and evaporated to dryness. Column chromatograph (SiO₂, DCM/EA=2:1 to 1:1 to 1:2) afforded 2-(2-chloropyridin-4-yl)-N-(4-(2-methylpyridin-4-yl)phenyl)-propanamide (460 mg, 94.5% yield) as an offwhite solid.

### Step 4

A mixture of potassium phosphate (217 mg, 1.02 mmol), bis(phenyl-di-tert-butylphosphine)palladium chloride (II) (6.4 mg, 10 μmol), 4-morpholinophenylboronic acid (141 mg, 0.68 mmol) and 2-(2-chloropyridin-4-yl)-N-(4-(2-methylpyridin-4-yl)-phenyl)propanamide (120 mg, 0.34 mmol) in 2 mL of dioxane/water=5:1 was heated at 120° C. under microwave irradiation for 30 min. After cooling to rt, the reaction mixture evaporated to dryness. The residual was submitted to flash chromatography (SiO₂, hexane to DCM/EtOAc=3:1 to 1:1 to pure EtOAc) to give N-(4-(2-methylpyridin-4-yl)phenyl)-2-(2-(4-morpholinophenyl)pyridin-4-yl)propanamide (32 mg, 20% yield) as a white solid.

### Example 22

### Synthesis of N-(4-(2-methylpyridin-4-yl)phenyl)-2-(4-(4-morpholinophenyl)pyridin-2-yl)-propanamide

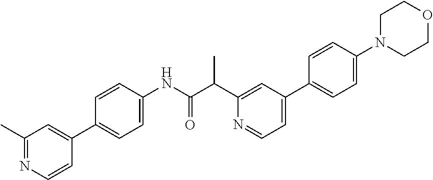

### Step 1

To a solution of ethyl 2-pyridylacetate (10.08 g, 61 mmol) in 100 mL of THF was added sodium hydride, 60% dispersion in mineral oil (1.6 ml, 64 mmol) in portions. After stirring at RT for 10 min, iodomethane (4.0 ml, 64 mmol) was added and the resulting mixture was stirred at RT overnight. The reaction mixture was quenched with brine, extracted with EtOAc, dried over Na₂SO₄, filtered and evaporated to dryness. Column chromatograph (SiO₂, H/E=10:1 to 10:2 to 1:1) afforded ethyl 2-(pyridin-2-yl)propanoate (4.2 g, 38% yield) together with recovered starting material.

### Step 2

A solution of ethyl 2-(pyridin-2-yl)propanoate (3.73 g, 20.8 mmol) and 3-chloroperoxybenzoic acid (4.67 g, 27.1 mmol) in 50 mL of DCM was stirred at RT for 3 h. After concentration, the reaction mixture was directly submitted to flash chromatography (SiO₂, DCM/EtOAc=2:1 to pure EtOAc to DCM/MeOH=10:1) to give 2-(ethoxycarbonyl-methyl)pyridine N-oxide (3.08 g, 75.8% yield) as a colorless oil.

### Step 3

A mixture of phosphorous oxychloride (3.6 ml, 38 mmol) and 2-(ethoxycarbonyl-methyl)pyridine N-oxide (1.25 g, 6.4 mmol) in toluene was heated at 100° C. for 5 h. After cooling, the reaction mixture was extracted with EtOAc, washed with sat. NaHCO₃, dried over Na₂SO₄, filtered and evaporated to dryness. The residue was submitted to column chromatograph (SiO₂, hexane/EtOAc=5:1 to 3:1 to 1:1) to give ethyl 2-(6-chloropyridin-2-yl)propanoate (0.320 g, 23% yield) and ethyl 2-(4-chloropyridin-2-yl)propanoate (0.16 g, 12% yield) as oil.

### Step 4

A solution of ethyl 2-(4-chloropyridin-2-yl)propanoate (100 mg, 0.47 mmol) in 4 mL of 37% HCl was stirred at 100° C. for 2 h. After HPLC showed complete conversion to acid, solvent was evaporated to dryness under vacuum. 4-(2-Methylpyridin-4-yl)benzenamine (86 mg, 0.47 mmol), 1-hydroxybenzotriazole (63 mg, 0.47 mmol) and N-(3-dimethylaminopropyl)-N′-ethylcarbodiimide hydrochloride (90 mg, 0.47 mmol), 0.5 mL of DMF and 0.5 mL of DIPEA were added and the resulting solution was stirred for 12 hours. The reaction mixture was quenched with sat. NaHCO₃, extracted with EtOAc/hexane=2:1, washed with brine, dried over Na₂SO₄, filtered and evaporated to dryness. Column chromatograph (SiO₂, hexane/EtOAc=2:1 to 1:1 to pure EtOAc) afforded 2-(4-chloropyridin-2-yl)-N-(4-(2-methylpyridin-4-yl)phenyl)propanamide (90 mg, 55% yield) as a white gum.

### Step 5

A mixture of potassium phosphate (53 mg, 0.25 mmol), bis(phenyl-di-tert-butylphosphine)palladium chloride (II) (1.6 mg, 2.5 μmol), 4-morpholinophenylboronic acid (34 mg, 0.17 mmol) and 2-(4-chloropyridin-2-yl)-N-(4-(2-methylpyridin-4-yl)phenyl)-propanamide (29.3 mg, 83 μmol) in 2 mL of dioxane/water=5:1 was heated at 120° C. unmicrowave irradiation for 30 min. After cooling to it, the reaction mixture evaporated to dryness. The residual was submitted to flash chromatography (SiO₂, hexane to DCM/EtOAc 3:1 to 1:1 to pure EtOAc) to give N-(4-(2-methylpyridin-4-yl)phenyl)-2-(4-(4-morpholinophenyl)pyridin-2-yl)propanamide (34 mg, 85% yield) as a white solid.

### Example 23

### Synthesis of 2-(6-bromopyridin-2-yl)-4-methyl-N-(4-(2-methylpyridin-4-yl)-phenyl)pentanamides

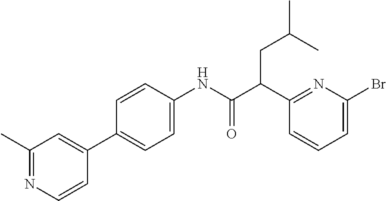

### Step 1

To a azeotroped mixture of isocapronitrile (2 ml, 15 mmol) and 2,6-dibromopyridine (4 g, 19 mmol) in toluene was added dropwise potassium bis(trimethylsilyl)amide (24 mL of 0.5 mL in toluene). After 1 h, the reaction mixture was quenched with 2 N HCl to pH=4, extracted with EtOAc, dried over Na₂SO₄, filtered and evaporated to dryness. Column chromatograph (SiO2, DCM/hexane=1:1 to 2:1 to pure DCM) gave 2-(6-bromopyridin-2-yl)-4-methylpentanenitrile (2.58 g, 66% yield) as a colorless oil.

### Step 2

A suspension of crude 2-(6-bromopyridin-2-yl)-4-methylpentanenitrile (2.58 g, 10.2 mmol) in 10 mL of 37% HCl was stirred at 90° C. for 3 h. The solvent was evaporated to dryness. To the residue was added 2 mL of sat. NaHCO₃ and DCM and the mixture was directly loaded onto column (SiO₂, hexane to DCM to DCM/EtOAc=3:1 to 1:1 to DCM/MeOH=100:5) to give 2-(6-bromopyridin-2-yl)-4-methylpentanoic acid (1.55 g, 55.9% yield) as a light yellow solid.

### Step 3

A solution of 4-(2-methylpyridin-4-yl)benzenamine (300 mg, 1.63 mmol), 1-(3-dimethylaminopropyl)-3-ethylcarbodiimide hydrochloride (375 mg, 1.95 mmol), 1-hydroxybenzotriazole (66.0 mg, 0.50 mmol) and 2-(6-bromopyridin-2-yl)-4-methylpentanoic acid (487 mg, 1.79 mmol) in 0.5 mL of DMF for 12 hours. The reaction mixture was quenched with sat. NaHCO₃, extracted with EtOAc/hexane=2:1, washed with brine, dried over Na₂SO₄, filtered and evaporated to dryness. Column chromatograph (SiO₂, DCM/EtOAc=#3:1 to 2:1 to 1:1 to 1:2) afforded 2-(6-bromopyridin-2-yl)-4-methyl-N-(4-(2-methylpyridin-4-yl)phenyl)-pentanamide (520 mg, 72.8% yield) as a off white solid.

### Example 24

### Synthesis of 4-methyl-N-(4-(2-methylpyridin-4-yl)phenyl)-2-(3-(morpholinomethyl)-phenyl)pentanamide

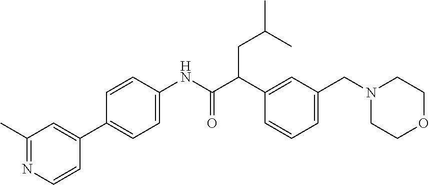

### Step 1

To a solution of 4-methyl-N-(4-(2-methylpyridin-4-yl)phenyl)-2-(3-vinylphenyl)-pentanamide (700 mg, 1.82 mmol), 4-methylmorpholine 4-oxide (235 mg, 2.0 mmol) and sodium periodate (0.40 ml, 7.23 mmol) in MeCN (20 mL) and water (10 mL) was added osmium tetroxide, 2.5 wt. %, solution in 2-methyl-2-propanol (1.9 μl, 36 μmol) (30 mg), and the reaction mixture was stirred at RT overnight. After evaporation of the solvent, the residue was diluted with water, extracted with EA, dried over Na₂SO₄ and evaporated to dryness. Column chromatograph (SiO2, DCM/EA=2:1 to 1:1 to 1:2) gave 2-(3-formylphenyl)-4-methyl-N-(4-(2-methylpyridin-4-yl)phenyl)pentanamide (420 mg, 60% yield) as a gum.

### Step 2

To a solution of 2-(3-formylphenyl)-4-methyl-N-(4-(2-methylpyridin-4-yl)-phenyl)pentanamide (19.3 mg, 50 μmol) and morpholine (13 mg, 0.15 mmol) in dichloroethane was added titanium(IV) propan-2-olate (28 mg, 0.10 mmol) and the resulting solution was stirred at RT overnight. To this was added 2 mL of MeOH followed by sodium tetrahydroborate (9.4 mg, 0.25 mmol). The reaction mixture was quenched with sat. NaHCO₃, extracted with EtOAc, dried over Na₂SO₄ and evaporated to dryness. Column chromatograph (SiO₂, EtOAc to EtOAc/MeOH=100:5 to 100:10 to 100:15 to 100:20) gave 4-methyl-N-(4-(2-methylpyridin-4-yl)phenyl)-2-(3-(morpholinomethyl)phenyl)pentanamide (15 mg, 66% yield).

### Example 25

### Synthesis of 6-bromo-2,2-dimethyl-N-(4-(2-methylpyridin-4-yl)phenyl)-3,4-dihydro-2H-chromene-4-carboxamide

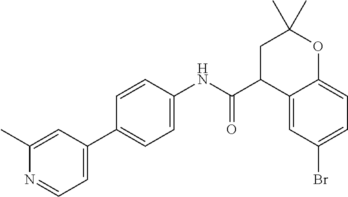

### Step 1

To a solution of 5″-bromo-2″-hydroxyacetophenone (10.0 g, 47 mmol) in toluene (30 mL) was added propan-2-one (7.0 ml, 70 mmol) and pyrrolidine (1.0 ml, 15 mmol). The reaction mixture was stirred at RT for 1 h, and then refluxed for 24 h. The solvent was removed under reduced pressure and the residue diluted with EtOAc and washed with 6N HCl, 2N NaOH and water, and dried over MgSO₄. After filtration and concentration, the residue was dried on vacuum overnight to give 6-bromo-2,2-dimethyl-2,3-dihydrochromen-4-one as a white solid. The product was used for next reaction without further purification.

### Step 2

To a mixture of 6-bromo-2,2-dimethyl-2,3-dihydrochromen-4-one (2.0 g, 7.8 mmol) and zinc iodide (106 μl, 1.6 mmol) in dichloromethane (30 mL) was added trimethylsilyl-cyanide (1.6 mL, 11.8 mmol) slowly. The mixture (a red solution) was stirred at RT. for 4 h. The resulting mixture was taken into dichloromethane and was washed with water, and dried over MgSO₄. After filtration and concentration, the solid residue was triturated in hexanes, and filtered to give 6-bromo-2,2-dimethyl-4-(trimethylsilyl)-3,4-dihydro-2H-chromene-4-carbonitrile as a brown solid.

### Step 3

To a mixture of 6-bromo-2,2-dimethyl-4-(trimethylsilyl)-3,4-dihydro-2H-chromene-4-carbonitrile (900 mg, 2.66 mmol) and tin (II) chloride (504 mg, 2.66 mmol) in glacial acetic acid (3.07 ml, 53.21 mmol) was added hydrochloric acid (3.3 ml, 106 mmol) slowly. The reaction mixture was stirred at reflux for 2 days, cooled to RT, and filtered to collect 6-bromo-2,2-dimethyl-3,4-dihydro-2H-chromene-4-carboxylic acid. It was used directly for next step without further purification.

### Step 4

To the mixture of 4-(2-methylpyridin-4-yl)benzenamine (165 mg, 894 μmol), 6-bromo-2,2-dimethyl-3,4-dihydro-2H-chromene-4-carboxylic acid (255 mg, 0.894 mmol, prepared according to the procedures described in Journal of Medicinal Chemistry, 49 (26), 7600-7602; 2006) and PYBOP (558 mg, 1.07 mmol) in DCM (1 mL) was added DIPEA (0.3 mL). The solution mixture was stirred overnight and quenched with water, extracted with DCM, washed with water and dried over MgSO₄. After filtration and concentration, 6-bromo-2,2-dimethyl-N-(4-(2-methylpyridin-4-yl)phenyl)-3,4-dihydro-2H-chromene-4-carboxamide was obtained through silica gel flash column chromatography (eluted with hexane:EtOAc, 4:1) as a solid. MS m/z: 450 (M+1).

### Example 26

### Synthesis of 2-(1H-indol-1-yl)-N-(4-(2-methylpyridin-4-yl)phenyl)propanamide

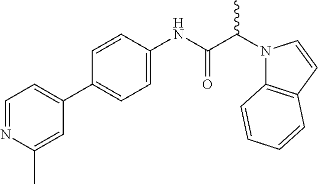

To a solution of 4-(2-methylpyridin-4-yl)benzenamine (0.5 g, 3 mmol) in DCM (5 mL) at RT was added trimethylaluminum, 2.0 m solution in toluene (2 ml, 4 mmol) dropwise from a syringe and after stirring at RT for 15 min, methyl 2-(1H-indol-1-yl)propanoate (0.577 g, 3 mmol) was added. The reaction mixture was stirred for 18 h and diluted with saturated Rochelle's salt solution, 50 ml, (caution, add Rochelle's salt dropwise to avoid reaction boiling over) and DCM (50 mL). The reaction mixture was stirred vigorously for 1 h and poured into a separatory funnel. The organic layer was separated, dried and concentrated. The residue was purified by ISCO column chromatograph using 0-4% MeOH in DCM to give 2-(1H-indol-1-yl)-N-(4-(2-methylpyridin-4-yl)phenyl)propanamide (0.36 g, 36% yield).

Following the procedure described above, the following compounds were prepared

| | | |
|---|---|---|
| 2-(1-methyl-1H-indo1-3- yl)-N-(4-(2-methyl- pyridin-4-yl)phenyl)- propanamide | 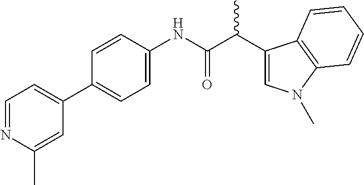 | MS (ESI, pos. ion) m/z: 370.1 (M + 1) |

The starting material for the above compound was prepared as follows:

### Step 1

To a solution of indole (1.7 ml, 17 mmol) in DMF (10 mL) at RT was added NaH (0.49 g, 20 mmol) followed by methyl 2-bromopropionate (4.3 g, 26 mmol). The suspension was stirred for 4 h, diluted with water and extracted with DCM. The DCM was dried and concentrated in vacuo. The residue was purified by ISCO column chromatograph using 0-20% EtOAc in DCM to give methyl 2-(1H-indol-1-yl)propanoate (2.82 g, 81% yield).

### Step 2

To a solution of methyl indole-3-acetate (1.85 g, 10 mmol) in THF (5 mL) was added lithium bis(trimethylsilyl)amide, 1.0 m solution in tetrahydrofuran (22 ml, 22 mmol) dropwise from a syringe at rt. After 15 min at rt, methyl iodide (1 ml, 21 mmol) was added. The reaction mixture was stirred at RT for another 2 h, diluted with 1N HCl solution, and extracted with DCM. DCM layer was dried (Na2SO4), concentrated and the residue purified by ISCO column chromatograph using 0-10% EtOAc in hexanes to give methyl 2-(1-methyl-1H-indol-3-yl)propanoate (0.2 g, 9% yield).

### Example 27

### Synthesis of 2-(3-(6-(dimethylamino)pyridin-3-yl)phenyl)-N-(4-(2-methylpyridin-4-yl)-phenyl)propanamide

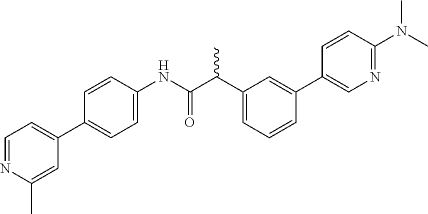

A solution of 2-(3-(6-fluoropyridin-3-yl)phenyl)-N-(4-(2-methylpyridin-4-yl)phenyl)-propanamide (0.150 g, 0.36 mmol) and dimethylamine, 2.0 m solution in methanol (20 ml, 377 mmol) was heated in a microwave at 140° C. for 1 h and concentrated. The residue was purified by ISCO column chromatograph using 0-50% EtOAc in DCM to give the title compound (0.040 g, 25% yield). MS (ESI, pos. ion) m/z: 437.2 (M+1)

Following the procedure described above, the following compounds were prepared

| | | | |
|---|---|---|---|
| 2321568 | 2-(3-(6-(methylamino)- pyridin-3-yl)phenyl)-N- (4-(2-methylpyridin-4- yl)phenyl)propanamide | 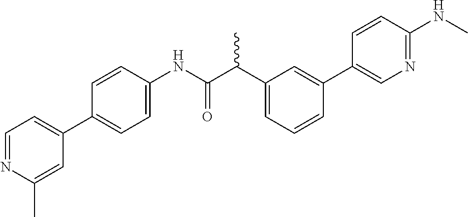 | MS (ESI, pos. ion) m/z: 423.1 (M + 1) |
| 2321569 | 2-(3-(6-(2-methoxyethyl- amino)pyridin-3-yl)- phenyl)-N-(4-(2-methyl- pyridin-4-yl)phenyl)- propanamide | 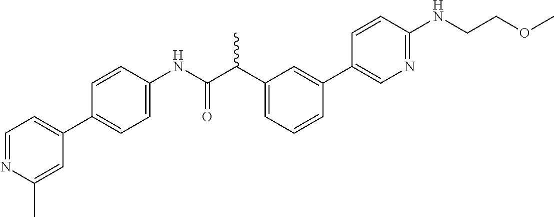 | MS (ESI, pos. ion) m/z: 467.1 (M + 1) |
| 2323180 | 2-(3-(6-(isopropyl- amino)-pyridin-3-yl)- phenyl)-N-(4-(2- methylpyridin-4-yl)- phenyl)propanamide | 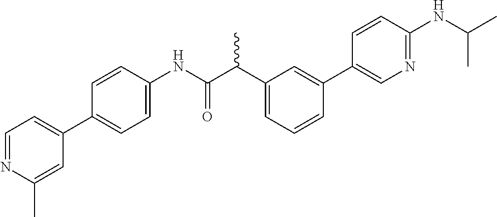 | MS (ESI, pos. ion) m/z: 451.1 (M + 1) |

### Example 28

### Synthesis of 2-(3-methoxyquinolin-6-yl)-N-(4-(2-methylpyridin-4-yl)phenyl)propanamide

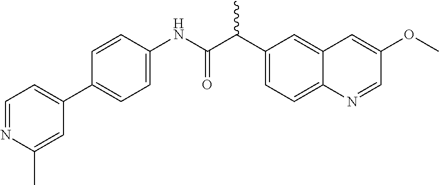

### Step 1

To a stirring solution of tert-butyl 2-(3-methoxyquinolin-6-yl)acetate (0.60 g, 2.2 mmol) in THF (10 mL) at −78° C. under nitrogen was added lithium bis(trimethylsilyl)amide, (1M in THF, 2.4 mL, 2.4 mmol). Iodomethane (0.145 mL, 2.3 mmol) was added after 15 minutes of stirring. The cooling bath was removed after 40 minutes and the reaction mixture was quenched with saturated ammonium chloride (5 mL), partitioned between ethyl acetate (75 mL), and 5% aqueous sodium bicarbonate (15 mL). The organic layer was dried over anhydrous sodium sulfate, concentrated to yield the crude product tert-butyl 2-(3-methoxyquinolin-6-yl)propanoate as a brown oil.

### Step 2

A 50 mL-round bottomed flask, containing a solution of tert-butyl-2-(3-methoxyquinolin-6-yl)propanoate (0.63 g, 2 mmol) in dichloromethane (8 mL) was treated with trifluoroacetic acid (4 mL, 54 mmol) and the reaction mixture was stirred at room temperature for 6 hours. During workup, the reaction mixture was concentrated to dryness to yield 2-(3-methoxyquinolin-6-yl)propanoic acid which was used for the next step.

### Step 3

A 50 mL, round-bottomed flask containing a solution of 2-(3-methoxyquinolin-6-yl)-propanoic acid (0.125 g, 0.54 mmol), 4-(2-methylpyridin-4-yl)benzenamine (0.100 g, 0.54 mmol), and benzotriazol-1-yloxytris(dimethylamino)phosphonium hexafluorophosphate (0.248 g, 0.56 mmol) in anhydrous DMF (7 mL) was treated with N,N-diisopropylethylamine (0.200 mL, 1.1 mmol) and the reaction mixture was stirred at room temperature for 12 hours. The reaction mixture was transferred to a scintillation vial, washed with methanol (5 mL) and evaporated to dryness to yield a reddish-brown gummy residue. The residue was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 2-(3-methoxyquinolin-6-yl)-N-(4-(2-methylpyridin-4-yl)phenyl)propanamide as an orange oil. MS (ESI pos. ion) m/z: 398 (M+1).

### Example 29

### Synthesis of 2-(3,5-bis(trifluoromethyl)phenyl)-N-(3-methoxy-4-(4-methyl-1H-imidazol-1-yl)phenyl)acetamide

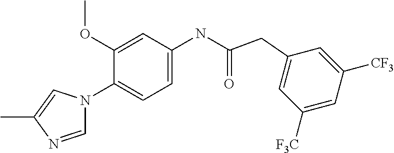

2-(3,5-Bis(trifluoromethyl)phenyl)acetic acid (76 mg, 280 μmol) and 3-methoxy-4-(4-methyl-1H-imidazol-1-yl)benzenamine (57 mg, 0.28 mmol) in 2 mL NMP was added 133 mg TBTU. The reaction mixture was stirred for 20 h and 50 mL of ethyl acetate was added and the mixture was washed with sodium biocarbonate, brine, dried and evaporated. Column chromatograph purification (0 to 2-5% MeOH/DCM) followed by RP-HPLC purification (20-100% ACN to TFA system) gave the title product.

### Example 30

### Synthesis of N-(3-methoxy-4-(4-methyl-1H-imidazol-1-yl)phenyl)-1-methyl-4-phenyl-1H-imidazole-2-carboxamide

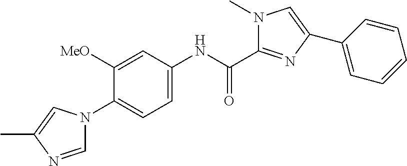

### Step 1

To a solution of trichloroacetyl chloride (13.6 ml, 122 mmol) in DCM (60 mL) was added 1-methylimidazole (9.69 ml, 122 mmol) in DCM (60 mL) dropwise for a period of 2 h. The reaction mixture was stirred for 4 h at RT, cooled 0° C., and triethylamine (16.9 ml, 122 mmol) was added dropwise over 1 h. After the addition, the solvent was evaporated in vacuo and the residue purified by ISCO using straight DCM to give 2,2,2-trichloro-1-(1-methyl-1H-imidazol-2-yl)ethanone (21.5 g, 77.6% yield).

### Step 2

To a stirred solution of 2,2,2-trichloro-1-(1-methyl-1H-imidazol-2-yl)ethanone (12.42 g, 54.6 mmol) in THF (40 mL) at −10° C. was added n-bromosuccinimide (6.95 ml, 81.9 mmol). The reaction mixture was kept at −10° C. for 2 h, warmed to RT with stirring for 12 h and concentrated. The residue was purified with ISCO using straight DCM. The solid obtained was dissolved in MeOH (40 mL), 60% NaH (80 mg) was added and the reaction mixture heated to 75° C. for 1 h. The reaction mixture was concentrated and the residue purified with ISCO using 0-10% EtOAc in DCM to give methyl 4-bromo-1-methyl-1H-imidazole-2-carboxylate (1.86 g, 15.6% yield).

### Step 3

4-Bromo-N-(3-methoxy-4-(4-methyl-1H-imidazol-1-yl)phenyl)-1-methyl-1H-imidazole-2-carboxamide was prepared from methyl 4-bromo-1-methyl-1H-imidazole-2-carboxylate and 3-methoxy-4-(4-methyl-1H-imidazol-1-yl)benzenamine according to Example 29.

### Step 4

N-(3-methoxy-4-(4-methyl-1H-imidazol-1-yl)phenyl)-1-methyl-4-phenyl-1H-imidazole-2-carboxamide was prepared from the Suzuki coupling of 4-bromo-N-(3-methoxy-4-(4-methyl-1H-imidazol-1-yl)phenyl)-1-methyl-1H-imidazole-2-carboxamide and phenyl boronic acid (e.g. according to Example 20 step 4).

Similarly, 1-methyl-N-(4-(2-methylpyridin-4-yl)phenyl)-4-phenyl-1H-imidazole-2-carboxamide and 4-bromo-1-methyl-N-(4-(2-methylpyridin-4-yl)phenyl)-1H-imidazole-2-carboxamide were prepared according to the above procedures.

### Example 37

### Chiral Separations

In cases where the final compounds have one or two chiral centers, they were synthesized as a mixture of stereoisomers, unless specified in the examples. These isomers could be separated using various chiral columns. Here are the conditions used to separate some of enantiomers mentioned in this application:

### Analytical and Preparative Supercritical Fluid

### Chromatography (SFC) Conditions:

### Analytical SFC Conditions:

Column: Chiralcel OD-H (250×4.6 mm, 5 μm)

Isocratic Conditions:
Mobile Phase: 80:20 (A:B)
   A: Liquid CO₂
   B: Isopropanol (0.1% diethylamine (DEA))

Flow Rate: 3.0 mL/min

Outlet Pressure: 100 bar

220 nm wavelength

Preparative SFC conditions:

Column: Chiralcel OD-H (200×20 mm, 5 μm)

Isocratic Conditions:
Mobile Phase: 85:15 (A:B)
   A: Liquid CO₂
   B: Isopropanol (0.1% DEA)

Flow Rate: 50.0 mL/min

Outlet Pressure: 100 bar

220 nm wavelength

The sample was dissolved in ethanol at a concentration of 30 mg/mL. Approximately 0.5 mL (15 mg of sample) of the solution was injected each time.

### BIOLOGICAL EXAMPLES

### Example 1

### Cell-Based Assays

GSM cell-based assays were designed to measure the modulation of Aβ 42 from HEK 293 cells over-expressing APP.

Three simultaneous cell-based assays, namely Aβ 42 inhibition or reduction, Aβ 40 inhibition or reduction and a measurement of cell viability of the cells from which the Aβ 42/40 readout was obtained, together gave an interpretation of gamma secretase modulation (GSM). The assays measure Aβ 42 and Ab 40 from conditioned medium of test compound treated HEK 293 cells.

HEK293 cells stably expressing full length Amyloid Precursor Protein (APP) were plated at a density of 100K cells/well in 96 well plates (Costar). The cells were cultivated for 6 hours at 37° C. and 5% CO₂ in DMEM supplemented with 10% FBS. The test compounds were then added to cells in 10-point dose response concentrations with the starting concentration being 10 μM. The compounds were diluted from stock solutions in DMSO and the final DMSO concentration of the test compounds on cells was 0.1%. After 24 hours of incubation with the test compounds the supernatant conditioned media was collected and the Aβ 42, Aβ 40 levels were determined using a sandwich ELISA. A cell viability test (CellTiter-Blue Cell Viability assay, Promega) on the cells from which the conditioned medium was harvested for Aβ 42 or Aβ 40 readouts gave an indication of cell survivability as a possible reason for false positive Aβ 42 or 40 reduction or inhibition readout. The IC₅₀ of the compound (for either Aβ 42 or Aβ 40) was calculated from the percent of control or percent inhibition of Aβ 42 or Aβ 40 as a function of the concentration of the test compound.

The sandwich ELISA to detect Aβ 42 or Aβ 40 was performed in 96 well microtiter plates, which were pre-treated with goat anti-rabbit IgG (Pierce). The capture and detecting antibody pair that were used to detect Aβ 42 and Aβ 40 from cell supernatants were rabbit monoclonal Antibody 42 (RabMAb 42) and affinity purified polyclonal Antibody 40 pAB40 (Biosource) as capture antibodies and biotinylated 6E10 (Signet Labs Inc.) as detection antibody. The optimal concentration for RabMAb 42 was 1 μg/ml in Superblock/TBS (Pierce) that was supplemented with 0.05% Tween 20 (Sigma). The optimal concentration for the pAb40 antibody was 3 μg/ml in Superblock/TBS (Pierce) that was supplemented with 0.05% Tween 20 (Sigma). Optimal concentration for the detection antibody 6E10-biotinylated was 0.5 μg/ml in Superblock/TBS (Pierce) that had been supplemented with 2% normal goat serum and 2% normal mouse serum.

Cellular supernatants were incubated with the capture antibody for 16-20 hours at 4° C., followed by 3 wash steps in TBS-tween (0.05%). The detecting antibody incubation was for 3 hours at 4° C., again followed by the wash steps as described previously. The final readout of the ELISA is Time-Resolved Fluorescence (counts per minute) using Delfia reagents Streptavidin-Europium and Enhancement solutions (Perkin Elmer) and the EnVision Multilabel plate reader (Perkin Elmer).

The cell viability assay used the CellTiter-Blue Cell Viability assay (Promega) using the manufacturers protocol.

In approximate IC₅₀ value of a representative number of compounds of Formula (I) in this assay is provided in the table below.

| Cpd # | IC₅₀ (uM) | Cpd # | IC₅₀ (uM) | Cpd # | IC₅₀ (uM) |
|---|---|---|---|---|---|
| 1 | 0.256 | 31 | 0.854 | 314 | >10 |
| 66 | 0.205 | 279 | >10 | 315 | 2.16 |
| 20 | 2.61 | 280 | >10 | 321 | >10 |
| 21 | 1.70 | 282 | >10 | 322 | 2.69 |
| 24 | 1.967 | 19 | 0.557 | 18 | 0.644 |
| 79 | 1.18 | 16 | 0.473 | 157 | 0.256 |
| 87 | 0.461 | 37 | 0.567 | 158 | 0.139 |
| 453 | 1.42 | 10 | 1.38 | 228 | 0.135 |
| 156 | 0.246 | 286 | 6.71 | 454 | 0.806 |
| 190 | 0.349 | 48 | >10 | 161 | >10 |
| 180 | 0.367 | 288 | 1.77 | 455 | 0.908 |
| 191 | 0.179 | 290 | >10 | 163 | 0.184 |
| 135 | 0.154 | 27 | 2.45 | 460 | 5.76 |
| 9 | 0.181 | 51 | >10 | 461 | 0.911 |
| 86 | >10 | 52 | >10 | 165 | 1.16 |
| 87 | 0.461 | 53 | >10 | 166 | 2.84 |
| 15 | 0.883 | 300 | >10 | 481 | 2.13 |
| 8 | 0.79 | 54 | >10 | 168 | >10 |
| 34 | 2.93 | 55 | >10 | 171 | 2.48 |
| 38 | 2.37 | 60 | >10 | 172 | 0.54 |
| 39 | 0.474 | 22 | 1.58 | 497 | >10 |
| 88 | >10 | 35 | 4.28 | 173 | >10 |
| 89 | >10 | 305 | 5.54 | 502 | >10 |
| 91 | >10 | 308 | >10 | 174 | 1.27 |
| 275 | >10 | 310 | 0.573 | 522 | 0.32 |
| 46 | >10 | 312 | >10 | 524 | 1.20 |
| 526 | 0.31 | 182 | >10 | 183 | 0.827 |
| 184 | 1.09 | 583 | 4.61 | 336 | >10 |
| 185 | 0.91 | 584 | >10 | 200 | 1.05 |
| 186 | >10 | 188 | 2.59 | 105 | 1.73 |
| 558 | >10 | 625 | >3.33 | 338 | 1.35 |
| 574 | >10 | 637 | >10 | 65 | 0.108 |
| 575 | 1.52 | 639 | >10 | 106 | >10 |
| 580 | 0.616 | 662 | 5.10 | 344 | >10 |
| 332 | 1.169 | 345 | 1.87 | 110 | 3.27 |
| 111 | 3.30 | 351 | 2.39 | 112 | 5.78 |
| 113 | >10 | 116 | 4.14 | 117 | 3.86 |
| 368 | 2.65 | 120 | >3.33 | 122 | 0.328 |
| 386 | 0.874 | 124 | 0.282 | 125 | 0.272 |
| 127 | 0.129 | 129 | >10 | 414 | 1.02 |
| 131 | 0.191 | 419 | >10 | 134 | 0.242 |
| 135 | 0.154 | 425 | >10 | 138 | >10 |
| 139 | 0.166 | 140 | >10 | 141 | 8.57 |
| 142 | 1.05 | 143 | 0.485 | 441 | 1.84 |
| 145 | >10 | 147 | 0.116 | 442 | 0.628 |
| 443 | 0.302 | 151 | 0.582 | 154 | 0.216 |

### Example 2

### Assay for In Vivo Efficacy. in Rats

Male Sprague-Dawley rats (175-200 g) were maintained on a 12-h light/dark cycle with unrestricted access to food and water until use. Rats were dosed orally with test compound at 30 mg/kg in 2% HPMC and 1% Tween 80 for a typical screening study. For a dose response study, compounds were dosed at 10, 30, 100 and 300 mg/kg. Tissue samples were collected 4 hours after dosing. Rats were euthanized with CO₂ inhalation for 2 minutes and cisterna magna was quickly exposed by removing the skin and muscle above it. CSF (50-100 ul) was collected with a 30-gauge needle through the dura membrane covering the cisterna magna. CSF samples with visible blood contamination were discarded. Blood was withdrawn by cardiac puncture and plasma was obtained by centrifugation for drug exposure. Brains were removed and, along with the CSF, immediately frozen on dry ice and stored at −80° C. until use. The frozen brains were subsequently homogenized in 10 volumes (w/v) of 0.5% Triton X-100 in TBS with protease inhibitors. The homogenates were centrifuged at 100,000 rpm for 30 min at 4° C. The supernatants were aliquoted and stored at −80° C. Aβ peptide levels were measured using a standard sandwich ELISA utilizing electrochemical luminescence detection (Meso Scale Discovery platform). Capture antibody was biotinylated-4G8 and detection antibodies for Aβ40 and Aβ42 were ruthenium labeled Fab40 and ruthenium labeled ConFab42 respectively. For pK analysis, blood and brain samples ere processed using a protein precipitation procedure with the remaining filtrate being analyzed via LC/MS/MS to determine drug exposure level, brain penetration and ED₅₀/EC₅₀ where appropriate.

### Mass Spec Analysis of Aβ Profile:

Cells were grown to 80-100% confluence in 10 cm dishes and re-fed with 10 ml fresh media (DMEM from Invitrogen (Gibco)) with 10% FBS and test compounds. Conditioned media was harvested after 16 h and centrifuged 5 minutes at 1,000×g. Supernatant was spiked with 0.5 ml 0.5 M Hepes, pH 7.3+20 mM EDTA and 1 nM Aβ1-28 followed by overnight incubation at 4° C. with anti-Aβ antibodies, 6E10 and 4G8 and 30 ul sheep-anti-mouse IgG magnetic beads (antibodies purchased from Covance Research Products and sheep anti-mouse e-beads from Invitrogen (Dynal)). Beads were collected using a magnetic separator and washed 4 times with: 1) 1×STEN, 2) 1×0.5 M NaCl—STEN, 3) 2×OTG buffer and 4) 5 mM Tris-HCl pH 7.5 0.1% OTG. Following the final wash, 4 ul MALDI matrix was added to the beads and mixed well. 2 ul was spotted on MALDI target plate (CM10) and air dried in the dark prior to analysis on a Ciphergen mass spectrometer. 100 femtomoles of bovine insulin was used as a calibrant.

### Formulation Examples

The following are representative pharmaceutical formulations containing a compound of Formula (I).

### Tablet Formulation

The following ingredients are mixed intimately and pressed into single scored tablets.

| | Quantity per tablet |
|---|---|
| Ingredient | mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this invention | 200 |
| lactose spray dried | 148 |
| magnesium stearate | 2 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1-19. (canceled)

20. A compound of Formula (I): 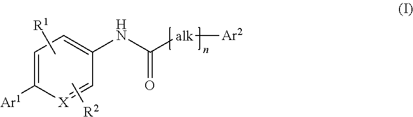
where:
n is 1;
alk is a straight or branched alkylene chain of 1 to 6 carbon atoms optionally substituted with cycloalkyl or one to three fluoro atoms;
X is —CH— or —N—;
R¹ and R² are independently hydrogen, alkyl, alkoxy, hydroxy, cyano, or halo;
Ar¹ is a ring selected from (i), (ii), (iii), or (iv); 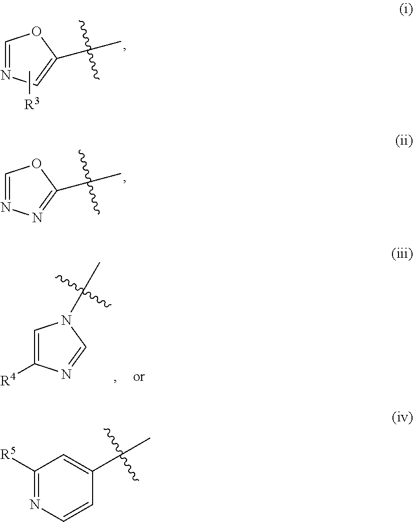
where:
R³ and R⁴ are hydrogen or alkyl;
R⁵ is alkyl; and
Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, hydroxyl, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, optionally substituted phenyl, optionally substituted heteroaryl, or optionally substituted heterocyclyl; or
a pharmaceutically acceptable salt thereof provided that (i) R^{b} or R^{c} is not substituted or unsubstituted hydantoin ring; (ii) R⁵ is not bonded to the nitrogen atom of the pyridinyl ring, (iii) when X is —CH—, R¹ and R² are hydrogen, n is 1, alk is —CHCH₃— or —CH(CH₂)CH₃—, then Ar² is not 6-chloropyridin-2-yl or 3-methylphenyl.

21. The compound of claim 20 the compound of Formula (I) is where:
alk is a straight or branched alkylene chain of 1 to 6 carbon atoms optionally substituted with one to three fluoro atoms;
X is —CH— or —N—;
R¹ and R² are independently hydrogen, alkyl, alkoxy, hydroxy, or halo;
Ar¹ is a ring selected from (i), (ii), (iii), or (iv); 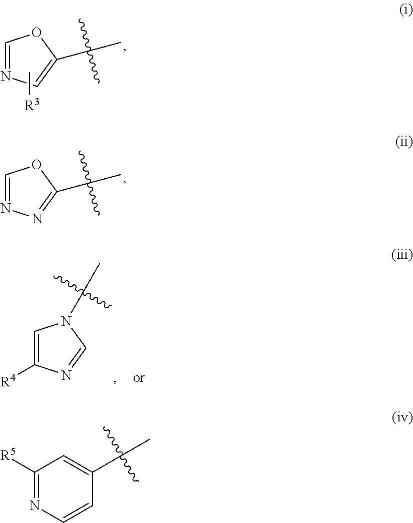
where:
R³ and R⁴ are hydrogen or alkyl;
R⁵ is alkyl; and
Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}. R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino.

22. The compound of claim 21 wherein alk is (R) or (S) —CH(CH₃)—.

23. The compound of claim 21 wherein alk is (R) or (S) —CH(iso-butyl)-.

24. The compound of claim 21 wherein Ar¹ is a ring of formula (iii) where R⁴ is alkyl.

25. The compound of claim 21 wherein Ar¹ is a ring of formula (iv).

26. The compound of claim 25 wherein Ar¹ is 2-methylpyridin-4-yl.

27. The compound of claim 24 wherein X is —CH—.

28. The compound of claim 26 wherein X is —CH—.

29. The compound of claim 24 wherein X is —N—.

30. The compound of claim 26 wherein X is —N—.

31. The compound of claim 26 wherein R¹ and R² are hydrogen.

32. The compound of claim 24 wherein Ar² is aryl optionally substituted with R^{a}, R^{b}, or R^{c}.

33. The compound of claim 26 wherein Ar² is aryl optionally substituted with R^{a}, R^{b}, or R^{c}.

34. The compound of 24 wherein Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, hydroxyl, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, optionally substituted phenyl, optionally substituted heteroaryl, or optionally substituted heterocyclyl; and
alk is (R) or (S) —CH(CH₃)— or —CH(iso-butyl).

35. The compound of 26 wherein Ar² is aryl, heteroaryl, cycloalkyl, spirocycloalkyl, fused cycloalkyl, heterocyclyl attached to —NHCO— group via carbon atom, or fused heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, heterocyclylalkyl, aryloxy, aralkyloxy, heteroaryloxy, heteroaralkyloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, hydroxyl, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, optionally substituted phenyl, optionally substituted heteroaryl, or optionally substituted heterocyclyl; and
alk is (R) or (S) —CH(CH₃)— or —CH(iso-butyl).

36. The compound of claim 26 wherein Ar² is 3,5-diCF₃phenyl; 1-(4-F-phenyl)cyclopent-1-yl; 3,4-dichlorophenyl; 2,4-dichlorophenyl; 2-trifluoromethylphenyl; 3-chlorophenyl; 3-chloro-6-methoxyphenyl; 3-trifluoromethylphenyl; 2,6-dichlorophenyl; naphth-1-yl; 3,4-methylenedioxyphenyl; 4-chlorophenyl; 2,3-dihydroinden-2-yl; biphen-4-yl; 5-methyl-1H-imidazo[1,2-a]pyridin2-yl; phenyl; 5-cyclopropylisoxazol-3-yl; 6-chloropyridin-2-yl; 5-chlorothiophen-2-yl; 3,5-difluorophenyl; 5-bromopyridin-3-yl; 4-methoxyphenyl; 3-phenoxyphenyl; naphth-2-yl; 3-methoxyphenyl; 3-methoxycyclohexyl; 2-chloro-5-bromophenyl; 3-chloro-4-methoxyphenyl; 5-methylthiophen-2-yl; 2-chlorophenyl; 3,4-ethylenedioxyphenyl; 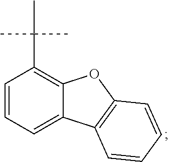
3,5-dimethylphenyl; 2,3-dichlorophenyl; 3-dimethylaminophenyl; 3-cyanophenyl; 3-chloro-4,5-dimethoxyphenyl; 2,6-dichloropyridin-4-yl; tetrahydrofuran-2-yl; 1-phenyl-2-oxopyrrolidin-4-yl; cyclopropyl; 1-acetylpiperidin-4-yl; imidazol-1-yl; 3,5-dimethylimidazol-1-yl; 3,5-dimethylisoxazol-4-yl; 1-cyclohexyl-2-oxopyrrolidin-4-yl; 1-tert-butyl-2-oxopyrrolidin-4-yl; 1-benzyl-2-oxopyrrolidin-4-yl; 3-methylphenyl; 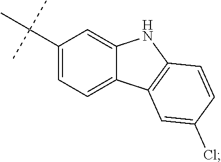
6-methoxynaphth-2-yl; 3,5-dichlorophenyl; 2-methoxy-5-chlorophenyl; 3-benzoylphenyl; 4-isobutylphenyl; 3-fluoro-4-phenylphenyl; benzothiophen-3-yl; 5-bromo-1H-indazol-3-yl; 1H-indol-3-yl; 6-methoxynaphth-2-yl; pyridin-2-yl; 2-biphenyl; 4-dimethylaminophenyl; 4,5,6,7-tetrahydrobenzthiazol-3-yl; cyclohexyl; 1-oxo-2-phenylisoindolin-7-yl; 2-cyclopentyl-1-oxoisoindolin-7-yl; 3-fluorophenyl; 2-chloro-6-fluorophenyl; 3,5-dimethoxyphenyl; 2,5-ditrifluoromethylphenyl; 2-fluoro-3-trifluoromethylphenyl; 4-trifluoromethoxyphenyl; 4-benzyloxyphenyl; 2-benzyloxyphenyl; 2-ethoxyphenyl; 9-methyl-9H-carbazol-2-yl; 2,3-dihydrobenzofuran-5-yl; 4-methoxy-3-methylphenyl; 3-chloro-2,6-difluorophenyl; 3-chloro-9H-carbazol-7-yl; 1-oxoisoindolin-2-yl; 3-bromophenyl; 2-phenylbenzoxazol-5-yl; quinolin-6-yl; 3-(4-morpholin-4-ylphenyl)-phenyl; 5-bromo-3-methylbenzimidazol-1-yl; 4-(4-morpholin-4-ylphenyl)-phenyl; 4-piperidin-1-ylphenyl; 3-(2-morpholin-4-ylpyridin-5-yl)-phenyl; 3-(1-tert-butoxycarbonyl-1,2,3,6-tetrahydropyridin-4-yl)-phenyl; 3-morpholin-4-ylphenyl; 3-(1-methylpiperazin-4-yl)phenyl; 3-(4-pyridin-2-ylpiperazin-1-yl)-phenyl; 3-[1-(2-hydroxyethyl)piperidin-4-yl]phenyl; 2-morpholin-4-ylpyridin-6-yl; 3-methylthiophenyl; 3-methylsulfonylphenyl; 3-(4-dimethylaminophenyl)phenyl; 2-(4-morpholin-4-ylphenyl)pyridin-6-yl; 4-phenylimidazol-1-yl; 3-(3-cyanophenyl)phenyl; 3-(3-methoxyphenyl)phenyl; 3-(2-methoxypyrimidin-5-yl)phenyl; 3-(4-dimethylaminocarbonyl-phenyl)phenyl; 3-(4-methylcarbonyl-phenyl)phenyl; 3-fluoro-5-trifluoromethyl-phenyl; 3-fluoro-5-(4-morpholin-4-ylphenyl)phenyl; 2-(4-trifluoromethylphenyl)-pyridin-4-yl; 3-phenylaminophenyl; 3-(2-methoxyethylamino)phenyl; 4-fluoro-3-morpholin-4-ylphenyl; S-1-tert-butoxycarbonyl-pyrrolidin-2-yl; 5-fluoro-3-(4-morpholin-4-ylphenyl)phenyl; 4-(cyclopropylmethylamino)-phenyl; 4-(N,N-dicyclopropylmethylamino)-phenyl; tetrahydropyran-4-yl; 3-thiophen-3-ylphenyl; 3-indol-5-ylphenyl; 4-N,N-dicyclobutyl-aminophenyl; 3-(2-morpholin-4-ylpyrimidin-5-yl)phenyl; 2-cyclopropylpyridin-4-yl; 4-(4-methoxybenzylamino)-phenyl; 2-(2-methoxyethylamino)-pyridin-6-yl; 3-(2-morpholin-4-ylthiazol-4-yl)phenyl; 2,2-dimethylcycloprop-1-yl; 2,2-difluorocycloprop-1-yl; 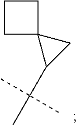
2-pyridin-2-ylbenzoxazol-5-yl; 4-tetrahydropyran-4-ylamino-phenyl; 6-chloroimidazo[1,2-a]pyridin-2-yl; imidazo[1,2-a]pyridin-2-yl; 1-methylindol-2-yl; 4,5,6,7-tetrahydro-1,2-benzisoxazol-3-yl; 6-bromoimidazo[1,2-a]pyridin-2-yl; 1,2,3,4-tetrahydronaphth-1-yl; biphen-3-yl; 4-bromophenyl; 6-chloro-9-methyl-9H-carbazol-2-yl; 3-(4-phenylpiperazin-1-yl)-phenyl; 3-iodophenyl; 4-isopropylaminophenyl; 3-benzyloxyphenyl; 3-(4-cyanophenyl)phenyl; 3-(2-cyanophenyl)phenyl; 3-(4-methoxyphenyl)phenyl; 3-(2-methoxyphenyl)phenyl; 3-(4-trifluoromethoxyphenyl)-phenyl; 3-[4-(1-tert-butoxypiperazin-1-yl)phenyl]-phenyl; 3-(4-piperazin-1-ylphenyl)-phenyl; 1-methylindol-3-yl; 4-morpholin-4-ylphenyl; 9-methyl-9H-carbazol-3yl; 1,3,4,9-tetrahydro-2H-beta-carbolin-2-yl); 8-fluoro-1,3,4,5-tetrahydro-2H-pyrido[4,3-b]indol-2-yl; 6-morpholin-4-ylpyridin-2-yl; 3-pyridin-3-ylphenyl; 3-pyridin-4-ylphenyl; 8-fluoro-1,3,4,5-tetrahydro-2H-pyrido[4,3-b]indol-2-yl; 4-di-n-propylaminophenyl; 4-diethylaminophenyl; 4-(4-morpholin-4-ylphenyl)-pyridin-2-yl; 2-(4-morpholin-4-yl-phenyl)-pyridin-4-yl; 3-methyl-5-(2-methylpyrimidin-5-yl)-phenyl; 5-cyclopropyl-1H-pyrazol-3-yl; 7-methylimidazo[1,2-a]pyridin-2-yl; 8-methylimidazo[1,2-a]pyridin-2-yl; pyridin-3-yl; 1-phenylcycloprop-1-yl; 1-(4-Clphenyl)cycloprop-1-yl; 2,4-dimethoxyphenyl; 2,5-dimethoxyphenyl; 2,5-dimethylphenyl; 3,4-dimethoxyphenyl; 3-trifluoromethylphenyl; 4-biphenyl; 3-methoxy-4-methylphenyl; 1-(2-Fphenyl)-cycloprop-1-yl; 1-(4-Fphenyl)cycloprop-1-yl; 1-(3-Fphenyl)cycloprop-1-yl; 1-(phenyl)-cyclobut-1-yl; 1-(phenyl)cyclohex-1-yl; 4-chloropyridin-2-yl; 1-(3-F-phenyl)cyclopent-1-yl; 1-(2-phenylethyl)-2-oxopyrrolidin-4-yl; 6-chloro-1H-imidazo[1,2-a]pyridin-2-yl; 3-methylpyridin-2-yl; 1H-imidazo[1,2-a]pyridin-2-yl; 2-methoxyphenyl; 1-(4-chlorophenyl)-2-oxopyrrolidin-4-yl; 1-benzylpyrrolidin-2-yl; 1-cyclohexylazetidin-2-yl; 1-benzylpyridin-4-yl; 1-(3-chlorophenyl)-2-oxopyrrolidin-4-yl; 6-fluorobenzthiophen-2-yl; 5-bromo-1H-imidazo[1,2-a]pyridin-2-yl; 2-phenylcyclopropyl; 2-fluorophenyl; 1,2,3,4-tetrahydronaphth-2-yl; 4H-chromen-4-one-3-yl; 4-methylphenyl; thien-2-yl; thien-3-yl; 2,3-dimethoxyphenyl; 1-(4-methoxyphenyl)cycloprop-1-yl; 1-(2,4-dichlorophenyl)-cycloprop-1-yl; 2-bromophenyl; 2-iodophenyl; 2-fluoro-6-trifluoro-methylphenyl; 2,4,6-trimethylphenyl; 4-fluoro-3-trifluoromethyl-phenyl; 2-methylphenyl; 2,4-ditrifluoromethylphenyl; 3-fluoro-4-methoxyphenyl; 2-chloro-5-fluorophenyl; 2-chloro-4-fluorophenyl; 2-bromo-5-chlorophenyl; 4-iodophenyl; 2-fluoro-5-trifluoromethyl-phenyl; 3-chloro-5-fluorophenyl; 4-n-butoxyphenyl; 4-tert-butylphenyl; 4-isopropylphenyl; 5-bromo-2-methoxyphenyl; 3-chloro-2-fluorophenyl; 2-fluoro-4-trifluoromethylphenyl; 3-fluoro-4-trifluoromethylphenyl; 2,6-difluoro-3-methylphenyl; 2-chloro-5-trifluoromethylphenyl; 2-trifluoromethoxyphenyl; 5-fluoro-2-trifluoromethylphenyl; indol-1-yl; 4-aminophenyl; 4-phenoxyphenyl; 3-(1,2,3,6-tetrahydropyridin-4-yl)phenyl; 3-(piperidin-4-yl)phenyl; 3-(morpholin-4-yl)phenyl; 3-fluoro-2-trifluoromethyl-phenyl; 6-fluoropyridin-2-yl; 3-(2-dimethylaminophenyl)-phenyl; 5-chloro-2-fluorophenyl; 3-morpholin-4-yl-5-trifluoromethylphenyl; 3-(4-methylsulfonylphenyl)-phenyl; 3-phenylimidazol-1-yl; 3-bromo-5-fluorophenyl; 3-methyl-4-phenylpyrazol-1-yl; 4-(4-bromophenyl)pyrazol-1-yl; 3-bromo-4-fluorophenyl; 3-piperidin-1-ylphenyl; 2-chloropyridin-4-yl; 4-fluoro-3-(2-methoxyethylamino)phenyl; 3-cyano-5-trifluoromethylphenyl; 3-bromo-5-trifluoromethylphenyl; 4-isopropylmethyl-aminophenyl; indol-5-yl; 3-bromo-5-fluorophenyl; 4-bromoimidazol-1-yl; 1,2,3,4-tetrahydroindol-5-yl; 4-trifluoromethylphenyl; 3-pyrimidin-5-ylphenyl; 3-(methylthiopyrimidin-5-yl)phenyl; 3-(2-aminopyrimidin-5-yl)phenyl; 3-(1-methylindol-5-yl)phenyl; 4-(cyclopentylamino)phenyl; 4-cyclobutylaminophenyl; 3R,5S-(5-phenyl)piperidin-3-yl; 4-N,N-diethylaminophenyl; 3-(2-hydroxypyridin-4-yl)phenyl; 4-(3,4,5-dimethoxybenzyl-amino)phenyl; 3-[4-(4-acetylpiperazin-1-yl)-phenyl)-phenyl; 4-cyanophenyl; 3-cyclopropylphenyl; 4-(ethylamino)phenyl; 4-(N-3,4,5-dimethoxybenzyl-N-ethylamino)phenyl; 4-(cyclopropylamino)phenyl; 4-(N,N-dicyclopropylamino)phenyl; 6-(4-morpholin-4-ylphenyl)-pyridin-2-yl; 3-(6-fluoropyridin-3-yl)phenyl; 6-bromopyridin-2-yl; 4-(N-3,4,5-dimethoxybenzyl-N-methylamino)phenyl; 3-(3-chloropyridin-4-yl)phenyl; 5-fluoro-3-(6-morpholin-4-ylpyridin-3-yl)phenyl; 4-sec-butylaminophenyl; 4-methylaminophenyl; 4-n-propylaminophenyl; 4-N,N-di-n-propylamino-phenyl; 2-methyl benzimidazol-6-yl; 2-ethylbenzoxazol-5-yl; 2-methylbenzoxazol-5-yl; 3-methoxyquinolin-6-yl; cyclobutyl; 2-methylcyclopropyl; cyclopentyl; 3-pyrimidin-2-ylphenyl; 3-pyridin-2-ylphenyl; tetrahydrofuran-3-yl; 2,4-dimethylthiazol-5-yl; 3-(6-dimethylamino-pyridin-4-yl)-phenyl; 3-(4-methylimidazol-1-yl)-phenyl; 3-(pyrazol-1-yl)-phenyl; 4-N-acetyl-N-isopropylamino-phenyl; 6-chloropyridin-3-yl; 4-amino-3-methoxyphenyl; 4-isopropylamino-3-methoxyphenyl; 3-bromo-5-methylphenyl; 4-fluoro-3-(4-morpholin-4-ylphenyl)phenyl; 6-piperidin-1-ylpyridin-3-yl; 5-methyl-3-(2-methoxy-pyrimidin-5-yl)phenyl; benzoxazol-5-yl; 2-pyridin-3-ylbenzoxazol-5-yl; 2-pyridin-4-ylbenzoxazol-5-yl; 3,5-di(4-trifluoromethyl-phenyl)phenyl; 5-isopropylaminopyridin-2-yl; 4-methyl-3-indol-5-ylphenyl; 3-bromo-5-chlorophenyl; 5-chloro-3-(2-methyl-pyrimidin-5-yl)phenyl; 5-chloro-3-(1,5-dimethylpyrazol-4-yl)phenyl; 5-chloro-3-(pyridin-3-yl)phenyl; 5-methyl-3-(4-cyanophenyl)-phenyl; 5-methyl-3-(3-cyanophenyl)-phenyl; 5-methyl-3-(2-cyanophenyl)-phenyl; 4-isobutylaminophenyl; 4-N,N-di-isobutylaminophenyl; 3-cyano-5-methylphenyl; 4-(N-3,4,5-dimethoxybenzyl-N-n-propyl-amino)phenyl; 3-(2-methylaminopyridin-3-yl)-phenyl; 3-[2-(2-methoxyethyl-amino)pyridin-3-yl]-phenyl; 2-methylbenzimidazol-5-yl; 6-methylsulfonylindol-2-yl; 5-methyl-3-(2-methylpyrimidin-5-yl)-phenyl; benzothiophen-2-yl; 5-methyl-3-(4-pyridin-2-ylpiperazin-1-yl)-phenyl; 5-methyl-3-(2-methylpyridin-4-yl)-phenyl; 5-fluoro-3-(pyridin-3-yl)-phenyl; 5-fluoro-3-(2-methylpyridin-4-yl)-phenyl; 3-difluoromethyl-5-trifluoromethyl-phenyl; 3-fluoro-5-methylphenyl; isoquinolin-5-yl; 1-methylindol-5-yl; quinolin-5-yl; benzo[d][1,2,3]thiadiazol-5-yl; 4-morpholin-4-ylmethylphenyl; 1-methyl-3-ethylpyrazol-5-yl; 3-ethoxyphenyl; 3-morpholin-4-ylmethylphenyl; quinolin-4-yl; 3-difluoromethoxyphenyl; 3,5-dimethylthiazol-4-yl; 3-(6-cyanopyridin-3-yl)phenyl; 5-methyl-3-(1-methyl-2-oxopiperazin-4-yl)phenyl; 5-methyl-3-(4-methoxypiperidin-1yl)phenyl; 5-methyl-3-(4,4-difluoropiperidin-1yl)phenyl; 3-(6-isopropylaminopyridin-3-yl)phenyl; 3-(4-carboxyphenyl)phenyl; 3-(6-chloropyrazin-2-yl)phenyl; 3-(5-methoxy-pyrazin-2-yl)phenyl; 5-bromo-2-fluorophenyl; 5-chloro-3-(3-cyanophenyl)-phenyl; 5-chloro-3-(2-methoxy-pyrimidin-5-yl)-phenyl; 5-chloro-3-(furan-3-yl)-phenyl; 5-chloro-3-(4-cyanophenyl)-phenyl; 4,6-ditrifluoromethylpyridin-2-yl; pyridin-4-yl; indol-6-yl; 3-acetylaminophenyl; 6-isopropylaminopyridin-3-yl; 4-N-4-methoxybenzyl-N-methylaminophenyl; 4-benzylaminophenyl; 6-dimethylaminopyridin-3-yl; 3-(3-methoxypiperidin-1-yl)phenyl; 3-(4-tert-butoxycarbonyl-piperazin-1-yl)phenyl; 3-(piperazin-1-yl)phenyl; 1-methylindazol-3-yl; 4-pyrrol-1-ylphenyl; 4-bromothiophen-2-yl; 4-N-benzyl-N-methylphenyl; 4-(2-methoxypyrimidin-5-yl)thiophen-2-yl; 5-bromothiophen-2-yl; 5-bromophenyl; 3-(2-methoxyethylamino-methyl)phenyl; 3-(2-dimethylamino-methyl)phenyl; 3-(piperidin-1-ylmethyl)phenyl; 3-hydroxymethylphenyl; or 5-methylpyridin-3-yl.

37. The compound of claim 26 wherein Ar² is heteroaryl where each of the aforementioned ring is optionally substituted with R^{a}, R^{b} or R^{c}.

38. The compound of claim 26 wherein Ar² is heterocyclyl attached to —NHCO— group via carbon atom, or fused heterocyclyl optionally substituted with R^{a}, R^{b} or R^{c}.

39. A compound selected from:
(2R)-2-(3,5-bis(trifluoromethyl)phenyl)-N-(4-(2-methyl-4-pyridinyl)phenyl)-propanamide
N-(4-(2-methyl-4-pyridinyl)phenyl)-2-(4-(trifluoromethyl)phenyl)acetamide
2-(3,4-dichlorophenyl)-N-(4-(2-methyl-4-pyridinyl)phenyl)acetamide
2-(3,5-bis(trifluoromethyl)phenyl)-N-(4-(2-methyl-4-pyridinyl)phenyl)propanamide
2-(2,4-dichlorophenyl)-N-(4-(2-methyl-4-pyridinyl)phenyl)acetamide
N-(4-(2-methyl-4-pyridinyl)phenyl)-2-(2-(trifluoromethyl)phenyl)acetamide
2-(3-chlorophenyl)-N-(4-(2-methyl-4-pyridinyl)phenyl)acetamide
N-(4-(2-methyl-4-pyridinyl)phenyl)-2-(3-(trifluoromethyl)phenyl)acetamide
2-(3-chlorophenyl)-N-(4-(2-methyl-4-pyridinyl)phenyl)propanamide
2-(3,5-ditrifluoromethylphenyl)-N-(4-(2-methyl-4-pyridinyl)phenyl)acetamide
2-(2,6-dichlorophenyl)-N-(4-(2-methyl-4-pyridinyl)phenyl)acetamide
N-(4-(2-methyl-4-pyridinyl)phenyl)-2-(1-naphthalenyl)acetamide
2-(1,3-benzodioxol-5-yl)-N-(4-(2-methyl-4-pyridinyl)phenyl)acetamide
2-(4-chlorophenyl)-N-(4-(2-methyl-4-pyridinyl)phenyl)propanamide
2-(4-biphenylyl)-N-(4-(2-methyl-4-pyridinyl)phenyl)acetamide
(2S)-N-(4-(2-methyl-4-pyridinyl)phenyl)-2-phenylpropanamide
2-(6-chloro-2-pyridinyl)-N-(4-(2-methyl-4-pyridinyl)phenyl)propanamide
2-(5-chloro-2-thiophenyl)-N-(4-(2-methyl-4-pyridinyl)phenyl)acetamide
2-(3,5-difluorophenyl)-N-(4-(2-methyl-4-pyridinyl)phenyl)propanamide
2-(5-bromo-3-pyridinyl)-N-(4-(2-methyl-4-pyridinyl)phenyl)acetamide
N-(4-(2-methyl-4-pyridinyl)phenyl)-2-phenylpropanamide
2-(4-methoxyphenyl)-N-(4-(2-methyl-4-pyridinyl)phenyl)acetamide
N-(4-(2-methyl-4-pyridinyl)phenyl)-2-phenylacetamide
N-(4-(2-methyl-4-pyridinyl)phenyl)-2-(2-naphthalenyl)acetamide
2-(3-methoxyphenyl)-N-(4-(2-methyl-4-pyridinyl)phenyl)acetamide
N-(4-(2-methyl-4-pyridinyl)phenyl)-2-(5-methyl-2-thiophenyl)acetamide
3-methyl-N-(4-(2-methyl-4-pyridinyl)-phenyl)-2-phenylbutanamide
(2R)-N-(4-(2-methyl-4-pyridinyl)phenyl)-2-phenylpropanamide
2-(2-chlorophenyl)-N-(4-(2-methyl-4-pyridinyl)phenyl)acetamide
2-(4-chlorophenyl)-2-methyl-N-(4-(2-methyl-4-pyridinyl)phenyl)propanamide
(2S)-2-(3,5-bis(trifluoromethyl)phenyl)-N-(4-(2-methyl-4-pyridinyl)phenyl)-propanamide
2-cyclopropyl-N-(4-(2-methyl-4-pyridinyl)-phenyl)acetamide
N-(4-(2-methyl-4-pyridinyl)phenyl)-3-(1H-pyrazol-1-yl)propanamide
3-(3,5-dimethyl-1H-pyrazol-1-yl)-N-(4-(2-methyl-4-pyridinyl)phenyl)propanamide
3-(3,5-dimethyl-4-isoxazolyl)-N-(4-(2-methyl-4-pyridinyl)phenyl)propanamide
4-methyl-2-(3-methylphenyl)-N-(4-(2-methyl-4-pyridinyl)phenyl)pentanamide
2-(6-chloro-9H-carbazol-2-yl)-N-(4-(2-methyl-4-pyridinyl)phenyl)propanamide
(2R)-2-(6-methoxy-2-naphthalenyl)-N-(4-(2-methyl-4-pyridinyl)phenyl)propanamide
2-(3-methylphenyl)-N-(4-(2-methyl-4-pyridinyl)phenyl)propanamide
2-(3,4-dichlorophenyl)-N-(4-(2-methyl-4-pyridinyl)phenyl)propanamide
N-(4-(2-methyl-4-pyridinyl)phenyl)-2-(3-(trifluoromethyl)phenyl)propanamide
(2S)-N-(4-(2-methyl-4-pyridinyl)phenyl)-2-(3-(phenylcarbonyl)phenyl)propanamide
(2S)-2-(4-(2-methylpropyl)phenyl)-N-(4-(2-methyl-4-pyridinyl)phenyl)propanamide
(2S)-2-(2-fluoro-4-biphenylyl)-N-(4-(2-methyl-4-pyridinyl)phenyl)propanamide
2-(1-benzothiophen-3-yl)-N-(4-(2-methyl-4-pyridinyl)phenyl)acetamide
2-(5-bromo-1H-indazol-3-yl)-N-(4-(2-methyl-4-pyridinyl)phenyl)acetamide
2-(1H-indol-3-yl)-N-(4-(2-methyl-4-pyridinyl)phenyl)acetamide
(2S)-2-(6-methoxy-2-naphthalenyl)-N-(4-(2-methyl-4-pyridinyl)phenyl)propanamide
(2R)-2-(2-fluoro-4-biphenylyl)-N-(4-(2-methyl-4-pyridinyl)phenyl)propanamide
2-(3-methylphenyl)-N-(4-(2-methyl-4-pyridinyl)phenyl)pentanamide
2-(3-methylphenyl)-N-(4-(2-methyl-4-pyridinyl)phenyl)hexanamide
2-(3,5-bis(trifluoromethyl)phenyl)-N-(4-(2-methyl-4-pyridinyl)phenyl)acetamide
N-(4-(2-methyl-4-pyridinyl)phenyl)-2-(2-pyridinyl)acetamide
2-(3,5-dimethyl-4-isoxazolyl)-N-(4-(2-methyl-4-pyridinyl)phenyl)acetamide
2-(3,5-bis(trifluoromethyl)phenyl)-N-(2-fluoro-4-(2-methyl-4-pyridinyl)phenyl)-acetamide
2-cyclohexyl-N-(4-(2-methyl-4-pyridinyl)-phenyl)acetamide
N-(4-(2-methyl-4-pyridinyl)phenyl)-3-phenylbutanamide
2-methyl-N-(4-(2-methyl-4-pyridinyl)-phenyl)-3-phenylpropanamide
(2S)-2-(3-fluorophenyl)-N-(4-(2-methyl-4-pyridinyl)phenyl)propanamide
(2R)-2-(3-fluorophenyl)-N-(4-(2-methyl-4-pyridinyl)phenyl)propanamide
(2S)-2-(6-chloro-2-pyridinyl)-N-(4-(2-methyl-4-pyridinyl)phenyl)propanamide
(2R)-2-(6-chloro-2-pyridinyl)-N-(4-(2-methyl-4-pyridinyl)phenyl)propanamide
(2S)-4-methyl-2-(3-methylphenyl)-N-(4-(2-methyl-4-pyridinyl)phenyl)pentanamide
2-(2-chloro-6-fluorophenyl)-N-(4-(2-methyl-4-pyridinyl)phenyl)acetamide
2-(3,5-dimethoxyphenyl)-N-(4-(2-methyl-4-pyridinyl)phenyl)acetamide
2-(2,5-bis(trifluoromethyl)phenyl)-N-(4-(2-methyl-4-pyridinyl)phenyl)acetamide
2-(2-fluoro-3-(trifluoromethyl)phenyl)-N-(4-(2-methyl-4-pyridinyl)phenyl)acetamide
N-(4-(2-methyl-4-pyridinyl)phenyl)-2-(4-(trifluoromethoxy)phenyl)acetamide
2-(4-(benzyloxy)phenyl)-N-(4-(2-methyl-4-pyridinyl)phenyl)acetamide
N-(4-(2-methyl-4-pyridinyl)phenyl)-2-(4-phenoxyphenyl)acetamide
2-(2-(benzyloxy)phenyl)-N-(4-(2-methyl-4-pyridinyl)phenyl)acetamide
2-(2-ethoxyphenyl)-N-(4-(2-methyl-4-pyridinyl)phenyl)acetamide
2-(9-methyl-9H-carbazol-2-yl)-N-(4-(2-methyl-4-pyridinyl)phenyl)acetamide
2-(2,3-dihydro-1-benzofuran-5-yl)-N-(4-(2-methyl-4-pyridinyl)phenyl)acetamide
2-(4-methoxy-3-methylphenyl)-N-(4-(2-methyl-4-pyridinyl)phenyl)acetamide
2-(3-chloro-2,6-difluorophenyl)-N-(4-(2-methyl-4-pyridinyl)phenyl)acetamide
2-(3,5-bis(trifluoromethyl)phenyl)-4-methyl-N-(4-(2-methyl-4-pyridinyl)phenyl)-
pentanamide
(2R)-2-(6-chloro-9H-carbazol-2-yl)-N-(4-(2-methyl-4-pyridinyl)phenyl)propanamide
N-(4-(2-methyl-4-pyridinyl)phenyl)-2-(4-(1-oxo-1,3-dihydro-2H-isoindol-2-yl)-
phenyl)propanamide
2-(3-bromophenyl)-3-cyclobutyl-N-(4-(2-methyl-4-pyridinyl)phenyl)propanamide
N-(4-(2-methyl-4-pyridinyl)phenyl)-2-(2-phenyl-1,3-benzoxazol-5-yl)acetamide
N-(4-(2-methyl-4-pyridinyl)phenyl)-2-(6-quinolinyl)acetamide
(2S)-4-methyl-N-(4-(2-methyl-4-pyridinyl)phenyl)-2-(4′-(4-morpholinyl)-3-
biphenylyl)pentanamide
(2R)-4-methyl-N-(4-(2-methyl-4-pyridinyl)phenyl)-2-(4′-(4-morpholinyl)-3-
biphenylyl)pentanamide
2-(3-bromophenyl)-4-fluoro-N-(4-(2-methyl-4-pyridinyl)phenyl)butanamide
2-(5-bromo-3-methyl-1H-indazol-1-yl)-N-(4-(2-methyl-4-pyridinyl)phenyl)-propanamide
(2R)-4-methyl-N-(4-(2-methyl-4-pyridinyl)-phenyl)-2-(4′-(4-morpholinyl)-4-
biphenylyl)pentanamide
(2S)-4-methyl-N-(4-(2-methyl-4-pyridinyl)phenyl)-2-(4′-(4-morpholinyl)-4-
biphenylyl)pentanamide
N-(4-(2-methyl-4-pyridinyl)phenyl)-2-(4′-(4-morpholinyl)-3-biphenylyl)propanamide
4-methyl-N-(4-(2-methyl-4-pyridinyl)-phenyl)-2-(4-(1-piperidinyl)phenyl)-pentanamide
N-(4-(2-methyl-4-pyridinyl)phenyl)-2-(3-(6-(4-morpholinyl)-3-pyridinyl)phenyl)-
propanamide
tert-butyl 4-(3-1-methyl-2-((4-(2-methyl-4-pyridinyl)phenyl)amino)-2-oxoethyl)-phenyl)-
3,6-dihydro-1(2H)-pyridinecarboxylate
4-methyl-N-(4-(2-methyl-4-pyridinyl)-phenyl)-2-(3-(4-morpholinyl)phenyl)-pentanamide
2-(3-(4-methyl-1-piperazinyl)phenyl)-N-(4-(2-methyl-4-pyridinyl)phenyl)propanamide
N-(4-(2-methyl-4-pyridinyl)phenyl)-2-(3-(4-(2-pyridinyl)-1-piperazinyl)phenyl)-
propanamide
2-(3-(1-(2-hydroxyethyl)-4-piperidinyl)-phenyl)-N-(4-(2-methyl-4-pyridinyl)-
phenyl)propanamide
N-(4-(2-methyl-4-pyridinyl)phenyl)-2-(6-(4-morpholinyl)-2-pyridinyl)propanamide
N-(4-(2-methyl-4-pyridinyl)phenyl)-2-(3-(methylsulfanyl)phenyl)propanamide
2-(3-bromophenyl)-N-(3-methoxy-4-(2-methyl-4-pyridinyl)phenyl)propanamide
2-(3-bromophenyl)-N-(3-methyl-4-(2-methyl-4-pyridinyl)phenyl)propanamide
4-methyl-N-(4-(2-methyl-4-pyridinyl)-phenyl)-2-(3-(methylsulfonyl)phenyl)-pentanamide
2-(4′-(dimethylamino)-3-biphenylyl)-N-(4-(2-methyl-4-pyridinyl)phenyl)propanamide
2-hydroxy-N-(4-(2-methyl-4-pyridinyl)-phenyl)-2-(4′-(4-morpholinyl)-3-
biphenylyl)ethanamide
N-(4-(2-methyl-4-pyridinyl)phenyl)-2-(6-(4-(4-morpholinyl)phenyl)-2-pyridinyl)-
propanamide
N-(4-(2-methyl-4-pyridinyl)phenyl)-2-(4′-(4-morpholinyl)-3-biphenylyl)acetamide
N-(4-(2-methyl-4-pyridinyl)phenyl)-2-(4-phenyl-1H-imidazol-1-yl)propanamide
(2R)-N-(4-(2-methyl-4-pyridinyl)phenyl)-2-(4′-(4-morpholinyl)-3-biphenylyl)-propanamide
(2S)-N-(4-(2-methyl-4-pyridinyl)phenyl)-2-(4′-(4-morpholinyl)-3-biphenylyl)-propanamide
2-(3′-cyano-3-biphenylyl)-N-(4-(2-methyl-4-pyridinyl)phenyl)propanamide
2-(3′-methoxy-3-biphenylyl)-N-(4-(2-methyl-4-pyridinyl)phenyl)propanamide
2-(3-(2-methoxy-5-pyrimidinyl)phenyl)-N-(4-(2-methyl-4-pyridinyl)phenyl)-propanamide
N,N-dimethyl-3′-(1-methyl-2-((4-(2-methyl-4-pyridinyl)phenyl)amino)-2-oxoethyl)-4-
biphenylcarboxamide
2-(4′-acetyl-3-biphenylyl)-N-(4-(2-methyl-4-pyridinyl)phenyl)propanamide
2-(3-fluoro-5-(trifluoromethyl)phenyl)-N-(3-methoxy-4-(2-methyl-4-pyridinyl)-
phenyl)propanamide
2-(5-fluoro-4′-(4-morpholinyl)-3-biphenylyl)-N-(3-methoxy-4-(2-methyl-4-pyridinyl)-
phenyl)propanamide
2-(3-bromophenyl)-N-(2-cyano-4-(2-methyl-4-pyridinyl)phenyl)propanamide
2-(3-bromophenyl)-N-(3-chloro-4-(2-methyl-4-pyridinyl)phenyl)propanamide
2-(3-bromophenyl)-3-cyclopropyl-N-(4-(2-methyl-4-pyridinyl)phenyl)propanamide
N-(4-(2-methyl-4-pyridinyl)phenyl)-2-(2-(4-(trifluoromethyl)phenyl)-4-pyridinyl)-
acetamide
N-(4-(2-methyl-4-pyridinyl)phenyl)-2-(3-(phenylamino)phenyl)propanamide
2-(3-((2-methoxyethyl)amino)phenyl)-N-(4-(2-methyl-4-pyridinyl)phenyl)-propanamide
2-(4-fluoro-3-(4-morpholinyl)phenyl)-N-(4-(2-methyl-4-pyridinyl)phenyl)propanamide
(2S)-2-(3-bromo-5-fluorophenyl)-N-(3-methoxy-4-(2-methyl-4-pyridinyl)-phenyl)-
propanamide
(2S)-2-(5-fluoro-4′-(4-morpholinyl)-3-biphenylyl)-N-(3-methoxy-4-(2-methyl-4-
pyridinyl)phenyl)propanamide
2-(4-((cyclopropylmethyl)amino)phenyl)-N-(4-(2-methyl-4-pyridinyl)phenyl)-propanamide
2-(4-(bis(cyclopropylmethyl)amino)-phenyl)-N-(4-(2-methyl-4-pyridinyl)-phenyl)-
propanamide
4-methyl-N-(4-(2-methyl-4-pyridinyl)-phenyl)-2-(tetrahydro-2H-pyran-4-yl)-pentanamide
N-(4-(2-methyl-4-pyridinyl)phenyl)-2-(3-(3-thiophenyl)phenyl)propanamide
2-(3-(1H-indol-5-yl)phenyl)-N-(4-(2-methyl-4-pyridinyl)phenyl)propanamide
2-(4-(dicyclobutylamino)phenyl)-N-(3-methoxy-4-(2-methyl-4-pyridinyl)-phenyl)-
propanamide
N-(4-(2-methyl-4-pyridinyl)phenyl)-2-(3-(2-(4-morpholinyl)-5-pyrimidinyl)-phenyl)-
propanamide
2-(2-cyclopropyl-4-pyridinyl)-N-(4-(2-methyl-4-pyridinyl)phenyl)propanamide
2-(4-((4-methoxybenzyl)amino)phenyl)-N-(4-(2-methyl-4-pyridinyl)phenyl)-propanamide
(2R)-2-(3-bromophenyl)-3-cyclopropyl-N-(4-(2-methyl-4-pyridinyl)phenyl)-propanamide
(2S)-2-(3-bromophenyl)-3-cyclopropyl-N-(4-(2-methyl-4-pyridinyl)phenyl)-propanamide
2-(6-((2-methoxyethyl)amino)-2-pyridinyl)-4-methyl-N-(4-(2-methyl-4-pyridinyl)-
phenyl)pentanamide
N-(4-(2-methyl-4-pyridinyl)phenyl)-2-(3-(2-(4-morpholinyl)-1,3-thiazol-4-yl)-phenyl)-
propanamide
N-(4-(2-methyl-4-pyridinyl)phenyl)-2-(2-(2-pyridinyl)-1,3-benzoxazol-5-yl)acetamide
N-(4-(2-methyl-4-pyridinyl)phenyl)-2-(4-(tetrahydro-2H-pyran-4-ylamino)phenyl)-
propanamide
(2R)-2-(3-bromophenyl)-4-methyl-N-(4-(2-methyl-4-pyridinyl)phenyl)pentanamide
(2R)-2-(3,5-difluorophenyl)-4-methyl-N-(4-(2-methyl-4-pyridinyl)phenyl)pentanamide
N-(3-methoxy-4-(2-methyl-4-pyridinyl)-phenyl)-2-(4′-(4-morpholinyl)-3-biphenylyl)-
propanamide
(2S)-2-(3-chlorophenyl)-N-(4-(2-methyl-4-pyridinyl)phenyl)propanamide
(2R)-2-(3-chlorophenyl)-N-(4-(2-methyl-4-pyridinyl)phenyl)propanamide
2-(3-methylphenyl)-N-(4-(2-methyl-4-pyridinyl)phenyl)butanamide
(2R)-2-(3,5-difluorophenyl)-N-(4-(2-methyl-4-pyridinyl)phenyl)propanamide
(2S)-2-(3,5-difluorophenyl)-N-(4-(2-methyl-4-pyridinyl)phenyl)propanamide
(2R)-2-(3-methylphenyl)-N-(4-(2-methyl-4-pyridinyl)phenyl)hexanamide
(2S)-2-(3-methylphenyl)-N-(4-(2-methyl-4-pyridinyl)phenyl)hexanamide
(2R)-4-methyl-2-(3-methylphenyl)-N-(4-(2-methyl-4-pyridinyl)phenyl)pentanamide
(2S)-4-methyl-2-(3-methylphenyl)-N-(4-(2-methyl-4-pyridinyl)phenyl)pentanamide
(2S)-2-(3,5-difluorophenyl)-4-methyl-N-(4-(2-methyl-4-pyridinyl)phenyl)pentanamide
(2R)-2-(3,5-difluorophenyl)-4-methyl-N-(4-(2-methyl-4-pyridinyl)phenyl)pentanamide
2-(3,5-dichlorophenyl)-N-(4-(2-methyl-4-pyridinyl)phenyl)propanamide
2-(3,5-dichlorophenyl)-4-methyl-N-(4-(2-methyl-4-pyridinyl)phenyl)pentanamide
(2R)-2-(3-bromophenyl)-4-methyl-N-(4-(2-methyl-4-pyridinyl)phenyl)pentanamide
(2S)-2-(3-bromophenyl)-4-methyl-N-(4-(2-methyl-4-pyridinyl)phenyl)pentanamide
(2R)-2-(4-bromophenyl)-4-methyl-N-(4-(2-methyl-4-pyridinyl)phenyl)pentanamide
(2S)-2-(4-bromophenyl)-4-methyl-N-(4-(2-methyl-4-pyridinyl)phenyl)pentanamide
(2R)-2-(3-cyanophenyl)-4-methyl-N-(4-(2-methyl-4-pyridinyl)phenyl)pentanamide
(2S)-2-(3-cyanophenyl)-4-methyl-N-(4-(2-methyl-4-pyridinyl)phenyl)pentanamide
2-(6-chloro-9-methyl-9H-carbazol-2-yl)-N-(4-(2-methyl-4-pyridinyl)phenyl)-propanamide
N-(4-(2-methyl-4-pyridinyl)phenyl)-2-(3-(4-phenyl-1-piperazinyl)phenyl)-propanamide
2-(3-iodophenyl)-N-(4-(2-methyl-4-pyridinyl)phenyl)propanamide
2-(4-((1-methylethyl)amino)phenyl)-N-(4-(2-methyl-4-pyridinyl)phenyl)propanamide
2-(3-(benzyloxy)phenyl)-N-(4-(2-methyl-4-pyridinyl)phenyl)propanamide
2-(4′-cyano-3-biphenylyl)-N-(4-(2-methyl-4-pyridinyl)phenyl)propanamide
2-(2′-cyano-3-biphenylyl)-N-(4-(2-methyl-4-pyridinyl)phenyl)propanamide
2-(4′-methoxy-3-biphenylyl)-N-(4-(2-methyl-4-pyridinyl)phenyl)propanamide
2-(2′-methoxy-3-biphenylyl)-N-(4-(2-methyl-4-pyridinyl)phenyl)propanamide
N-(4-(2-methyl-4-pyridinyl)phenyl)-2-(4′-(trifluoromethoxy)-3-biphenylyl)-propanamide
tert-butyl 4-(3′-(3-methyl-1-((4-(2-methyl-4-pyridinyl)phenyl)carbamoyl)butyl)-4-
biphenylyl)-1-piperazinecarboxylate
4-methyl-N-(4-(2-methyl-4-pyridinyl)-phenyl)-2-(4′-(1-piperazinyl)-3-biphenylyl)-
pentanamide
2-(5-fluoro-4′-(4-morpholinyl)-3-biphenylyl)-N-(4-(2-methyl-4-pyridinyl)phenyl)-
propanamide
(2R)-2-(3-fluoro-5-(trifluoromethyl)-phenyl)-N-(4-(2-methyl-4-pyridinyl)phenyl)-
propanamide
(2S)-2-(3-fluoro-5-(trifluoromethyl)phenyl)-N-(4-(2-methyl-4-pyridinyl)phenyl)-
propanamide
2-(1-methyl-1H-indol-3-yl)-N-(4-(2-methyl-4-pyridinyl)phenyl)propanamide
4-methyl-N-(4-(2-methyl-4-pyridinyl)-phenyl)-2-(4-(4-morpholinyl)phenyl)-pentanamide
2-(9-methyl-9H-carbazol-3-yl)-N-(4-(2-methyl-4-pyridinyl)phenyl)acetamide
(2R)-2-(3,5-dichlorophenyl)-N-(4-(2-methyl-4-pyridinyl)phenyl)propanamide
(2S)-2-(3,5-dichlorophenyl)-4-methyl-N-(4-(2-methyl-4-pyridinyl)phenyl)pentanamide
(2R)-2-(3,5-dichlorophenyl)-4-methyl-N-(4-(2-methyl-4-pyridinyl)phenyl)pentanamide
N-(3-methoxy-4-(2-methyl-4-pyridinyl)-phenyl)-2-(1,3,4,9-tetrahydro-2H-beta-carbolin-2-
yl)propanamide
2-(8-fluoro-1,3,4,5-tetrahydro-2H-pyrido[4,3-b]indol-2-yl)-N-(3-methoxy-4-(2-methyl-4-
pyridinyl)phenyl)propanamide
(2R)-2-(5-fluoro-4′-(4-morpholinyl)-3-biphenylyl)-N-(3-methoxy-4-(2-methyl-4-
pyridinyl)phenyl)propanamide
(2S)-2-(4-((1-methylethyl)amino)phenyl)-N-(4-(2-methyl-4-pyridinyl)phenyl)-propanamide
(2R)-2-(4-((1-methylethyl)amino)phenyl)-N-(4-(2-methyl-4-pyridinyl)phenyl)-propanamide
(2S)-2-(3,5-bis(trifluoromethyl)phenyl)-N-(3-methoxy-4-(2-methyl-4-pyridinyl)-
phenyl)propanamide
(2R)-2-(3,5-bis(trifluoromethyl)phenyl)-N-(3-methoxy-4-(2-methyl-4-pyridinyl)-
phenyl)propanamide
(2R)-2-(3-bromophenyl)-N-(4-(2-methyl-4-pyridinyl)phenyl)pentanamide
(2S)-2-(3-bromophenyl)-N-(4-(2-methyl-4-pyridinyl)phenyl)pentanamide
2-(6-chloro-2-pyridinyl)-4-methyl-N-(4-(2-methyl-4-pyridinyl)phenyl)pentanamide
4-methyl′-N-(4-(2-methyl-4-pyridinyl)-phenyl)-2-(6-(4-morpholinyl)-2-pyridinyl)-
pentanamide
N-(4-(2-methyl-4-pyridinyl)phenyl)-2-(3-(3-pyridinyl)phenyl)propanamide
N-(4-(2-methyl-4-pyridinyl)phenyl)-2-(3-(4-pyridinyl)phenyl)propanamide
(2R)-2-(8-fluoro-1,3,4,5-tetrahydro-2H-pyrido[4,3-b]indol-2-yl)-N-(3-methoxy-4-(2-
methyl-4-pyridinyl)phenyl)propanamide
(2S)-2-(8-fluoro-1,3,4,5-tetrahydro-2H-pyrido[4,3-b]indol-2-yl)-N-(3-methoxy-4-(2-
methyl-4-pyridinyl)phenyl)propanamide
2-(4-(dipropylamino)phenyl)-N-(4-(2-methyl-4-pyridinyl)phenyl)propanamide
2-(4-(diethylamino)phenyl)-N-(4-(2-methyl-4-pyridinyl)phenyl)propanamide
N-(4-(2-methyl-4-pyridinyl)phenyl)-2-(4-(4-(4-morpholinyl)phenyl)-2-pyridinyl)-
propanamide
N-(4-(2-methyl-4-pyridinyl)phenyl)-2-(2-(4-(4-morpholinyl)phenyl)-4-pyridinyl)-
propanamide
(2S)-3-cyclopropyl-2-(3,5-difluorophenyl)-N-(4-(2-methyl-4-pyridinyl)phenyl)-
propanamide
(2R)-3-cyclopropyl-2-(3,5-difluorophenyl)-N-(4-(2-methyl-4-pyridinyl)phenyl)-
propanamide
2-(3-methyl-5-(2-methyl-5-pyrimidinyl)-phenyl)-N-(4-(2-methyl-4-pyridinyl)-
phenyl)propanamide
N-(4-(2-methyl-4-pyridinyl)phenyl)-2-(3-pyridinyl)acetamide
2-(3,4-dichlorophenyl)-N-(4-(2-methyl-4-pyridinyl)phenyl)acetamide
2-methyl-N-(4-(2-methyl-4-pyridinyl)-phenyl)-2-phenylpropanamide
(2S)-2-(3,5-bis(trifluoromethyl)phenyl)-N-(4-(2-methyl-4-pyridinyl)phenyl)-propanamide
2-(3,5-difluorophenyl)-N-(4-(2-methyl-4-pyridinyl)phenyl)acetamide
2-(4-chloro-2-pyridinyl)-N-(4-(2-methyl-4-pyridinyl)phenyl)propanamide
(2R)-N-(4-(2-methyl-4-pyridinyl)phenyl)-2-phenylpropanamide
(2S)-N-(4-(2-methyl-4-pyridinyl)phenyl)-2-phenylpropanamide
2-(3-fluorophenyl)-N-(4-(2-methyl-4-pyridinyl)phenyl)propanamide
2-(2-fluorophenyl)-N-(4-(2-methyl-4-pyridinyl)phenyl)propanamide
2-(2-methoxyphenyl)-N-(4-(2-methyl-4-pyridinyl)phenyl)acetamide
(2S)-2-(3-chlorophenyl)-N-(4-(2-methyl-4-pyridinyl)phenyl)propanamide
(2R)-2-(3-chlorophenyl)-N-(4-(2-methyl-4-pyridinyl)phenyl)propanamide
(2S)-2-(2-fluorophenyl)-N-(4-(2-methyl-4-pyridinyl)phenyl)propanamide
(2R)-2-(2-fluorophenyl)-N-(4-(2-methyl-4-pyridinyl)phenyl)propanamide
2-(3-cyanophenyl)-N-(4-(2-methyl-4-pyridinyl)phenyl)acetamide
2-(3-methylphenyl)-N-(4-(2-methyl-4-pyridinyl)phenyl)propanamide
2-(4-methylphenyl)-N-(4-(2-methyl-4-pyridinyl)phenyl)propanamide
N-(4-(2-methyl-4-pyridinyl)phenyl)-2-(2-thiophenyl)acetamide
N-(4-(2-methyl-4-pyridinyl)phenyl)-2-(2-thiophenyl)acetamide
2-(2,5-dimethoxyphenyl)-N-(4-(2-methyl-4-pyridinyl)phenyl)acetamide
2-(2,3-dimethoxyphenyl)-N-(4-(2-methyl-4-pyridinyl)phenyl)acetamide
2-(2,3-dichlorophenyl)-N-(4-(2-methyl-4-pyridinyl)phenyl)propanamide
2-(2,4-dichlorophenyl)-N-(4-(2-methyl-4-pyridinyl)phenyl)propanamide
2-(2,6-dichlorophenyl)-N-(4-(2-methyl-4-pyridinyl)phenyl)propanamide
2-(3,5-bis(trifluoromethyl)phenyl)-2-methyl-N-(4-(2-methyl-4-pyridinyl)phenyl)-
propanamide
2-(3-methylphenyl)-N-(4-(2-methyl-4-pyridinyl)phenyl)butanamide
3-methyl-2-(3-methylphenyl)-N-(4-(2-methyl-4-pyridinyl)phenyl)butanamide
(2R)-2-(3,5-difluorophenyl)-N-(4-(2-methyl-4-pyridinyl)phenyl)propanamide
(2S)-2-(3,5-difluorophenyl)-N-(4-(2-methyl-4-pyridinyl)phenyl)propanamide
2-(4-bromophenyl)-N-(4-(2-methyl-4-pyridinyl)phenyl)acetamide
3-methyl-N-(4-(2-methyl-4-pyridinyl)-phenyl)-2-phenylpentanamide
2-(2-bromophenyl)-N-(4-(2-methyl-4-pyridinyl)phenyl)acetamide
(2R)-N-(4-(2-methyl-4-pyridinyl)phenyl)-2-phenylbutanamide
(2S)-N-(4-(2-methyl-4-pyridinyl)phenyl)-2-phenylbutanamide
2-(3-bromophenyl)-N-(4-(2-methyl-4-pyridinyl)phenyl)acetamide
2-(2-iodophenyl)-N-(4-(2-methyl-4-pyridinyl)phenyl)acetamide
2-(2-fluoro-6-(trifluoromethyl)phenyl)-N-(4-(2-methyl-4-pyridinyl)phenyl)acetamide
N-(4-(2-methyl-4-pyridinyl)phenyl)-2-(2,4,6-trimethylphenyl)acetamide
2-(3-iodomethyl-N-(4-(2-methyl-4-pyridinyl)phenyl)acetamide
2-(4-fluoro-3-(trifluoromethyl)phenyl)-N-(4-(2-methyl-4-pyridinyl)phenyl)acetamide
N-(4-(2-methyl-4-pyridinyl)phenyl)-2-phenylbutanamide
2-(2-methylphenyl)-N-(4-(2-methyl-4-pyridinyl)phenyl)acetamide
2-(3-methylphenyl)-N-(4-(2-methyl-4-pyridinyl)phenyl)acetamide
2-(4-methylphenyl)-N-(4-(2-methyl-4-pyridinyl)phenyl)acetamide
2-(2,4-bis(trifluoromethyl)phenyl)-N-(4-(2-methyl-4-pyridinyl)phenyl)acetamide
2-(3-fluoro-4-methoxyphenyl)-N-(4-(2-methyl-4-pyridinyl)phenyl)acetamide
2-(2-chloro-5-fluorophenyl)-N-(4-(2-methyl-4-pyridinyl)phenyl)acetamide
2-(3-chloro-4-fluorophenyl)-N-(4-(2-methyl-4-pyridinyl)phenyl)acetamide
2-(2-bromo-5-chlorophenyl)-N-(4-(2-methyl-4-pyridinyl)phenyl)acetamide
2-(4-iodophenyl)-N-(4-(2-methyl-4-pyridinyl)phenyl)acetamide
2-(2-fluoro-5-(trifluoromethyl)phenyl)-N-(4-(2-methyl-4-pyridinyl)phenyl)acetamide
2-(3-chloro-5-fluorophenyl)-N-(4-(2-methyl-4-pyridinyl)phenyl)acetamide
2-(3-(benzyloxy)phenyl)-N-(4-(2-methyl-4-pyridinyl)phenyl)acetamide
2-(4-butoxyphenyl)-N-(4-(2-methyl-4-pyridinyl)phenyl)acetamide
2-(2-chloro-4-fluorophenyl)-N-(4-(2-methyl-4-pyridinyl)phenyl)acetamide
2-(4-tert-butylphenyl)-N-(4-(2-methyl-4-pyridinyl)phenyl)acetamide
N-(4-(2-methyl-4-pyridinyl)phenyl)-2-phenylpentanamide
2-(2,5-dimethylphenyl)-N-(4-(2-methyl-4-pyridinyl)phenyl)acetamide
2-(4-(1-methylethyl)phenyl)-N-(4-(2-methyl-4-pyridinyl)phenyl)acetamide
2-(3,5-dimethylphenyl)-N-(4-(2-methyl-4-pyridinyl)phenyl)acetamide
2-(5-bromo-2-methoxyphenyl)-N-(4-(2-methyl-4-pyridinyl)phenyl)acetamide
2-(3-chloro-2-fluorophenyl)-N-(4-(2-methyl-4-pyridinyl)phenyl)acetamide
2-(2-fluoro-4-(trifluoromethyl)phenyl)-N-(4-(2-methyl-4-pyridinyl)phenyl)acetamide
2-(3-fluoro-4-(trifluoromethyl)phenyl)-N-(4-(2-methyl-4-pyridinyl)phenyl)acetamide
2-(2,6-difluoro-3-methylphenyl)-N-(4-(2-methyl-4-pyridinyl)phenyl)acetamide
N-(4-(2-methyl-4-pyridinyl)phenyl)-2-(3-(trifluoromethoxy)phenyl)acetamide
2-(2-chloro-5-(trifluoromethyl)phenyl)-N-(4-(2-methyl-4-pyridinyl)phenyl)acetamide
2-(3,5-difluorophenyl)-4-methyl-N-(4-(2-methyl-4-pyridinyl)phenyl)pentanamide
4-methyl-N-(4-(2-methyl-4-pyridinyl)-phenyl)-2-(3-(trifluoromethyl)phenyl)-pentanamide
2-(3-chlorophenyl)-4-methyl-N-(4-(2-methyl-4-pyridinyl)phenyl)pentanamide
(2R)-2-(3-methylphenyl)-N-(4-(2-methyl-4-pyridinyl)phenyl)hexanamide
(2S)-2-(3-methylphenyl)-N-(4-(2-methyl-4-pyridinyl)phenyl)hexanamide
(2R)-4-methyl-2-(3-methylphenyl)-N-(4-(2-methyl-4-pyridinyl)phenyl)pentanamide
(2S)-4-methyl-2-(3-methylphenyl)-N-(4-(2-methyl-4-pyridinyl)phenyl)pentanamide
N-(4-(2-methyl-4-pyridinyl)phenyl)-2-(2-(trifluoromethoxy)phenyl)acetamide
2-(5-fluoro-2-(trifluoromethyl)phenyl)-N-(4-(2-methyl-4-pyridinyl)phenyl)acetamide
2-(4-bromophenyl)-4-methyl-N-(4-(2-methyl-4-pyridinyl)phenyl)pentanamide
2-(3-bromophenyl)-4-methyl-N-(4-(2-methyl-4-pyridinyl)phenyl)pentanamide
2-(2-bromophenyl)-4-methyl-N-(4-(2-methyl-4-pyridinyl)phenyl)pentanamide
2-(4-biphenylyl)-4-methyl-N-(4-(2-methyl-4-pyridinyl)phenyl)pentanamide
2-(2-biphenylyl)-4-methyl-N-(4-(2-methyl-4-pyridinyl)phenyl)pentanamide
(2R)-N-(4-(2-methyl-4-pyridinyl)phenyl)-2-(3-(trifluoromethyl)phenyl)propanamide
(2S)-N-(4-(2-methyl-4-pyridinyl)phenyl)-2-(3-(trifluoromethyl)phenyl)propanamide
2-(3-biphenylyl)-4-methyl-N-(4-(2-methyl-4-pyridinyl)phenyl)pentanamide
2-(3-cyanophenyl)-4-methyl-N-(4-(2-methyl-4-pyridinyl)phenyl)pentanamide
N-(4-(2-methyl-4-pyridinyl)phenyl)-2-(2-naphthalenyl)propanamide
(2S)-2-(3,5-difluorophenyl)-4-methyl-N-(4-(2-methyl-4-pyridinyl)phenyl)pentanamide
(2R)-2-(3,5-difluorophenyl)-4-methyl-N-(4-(2-methyl-4-pyridinyl)phenyl)pentanamide
2-(3,5-dichlorophenyl)-N-(4-(2-methyl-4-pyridinyl)phenyl)propanamide
2-(3,5-dichlorophenyl)-4-methyl-N-(4-(2-methyl-4-pyridinyl)phenyl)pentanamide
2-dibenzo[b,d]thiophen-4-yl-N-(4-(2-methyl-4-pyridinyl)phenyl)propanamide
2-(9H-carbazol-2-yl)-N-(4-(2-methyl-4-pyridinyl)phenyl)propanamide
(2R)-2-(3-bromophenyl)-4-methyl-N-(4-(2-methyl-4-pyridinyl)phenyl)pentanamide
(2S)-2-(3-bromophenyl)-4-methyl-N-(4-(2-methyl-4-pyridinyl)phenyl)pentanamide
(2R)-2-(4-bromophenyl)-4-methyl-N-(4-(2-methyl-4-pyridinyl)phenyl)pentanamide
(2S)-2-(4-bromophenyl)-4-methyl-N-(4-(2-methyl-4-pyridinyl)phenyl)pentanamide
2-(3-bromophenyl)-N-(4-(2-methyl-4-pyridinyl)phenyl)propanamide
2-(3-bromophenyl)-N-(4-(2-methyl-4-pyridinyl)phenyl)butanamide
2-(3-bromophenyl)-N-(4-(2-methyl-4-pyridinyl)phenyl)pentanamide
N-(4-(2-methyl-4-pyridinyl)phenyl)-2-(1-naphthalenyl)propanamide
2-(1H-indol-1-yl)-N-(4-(2-methyl-4-pyridinyl)phenyl)propanamide
(2R)-2-(3-cyanophenyl)-4-methyl-N-(4-(2-methyl-4-pyridinyl)phenyl)pentanamide
(2S)-2-(3-cyanophenyl)-4-methyl-N-(4-(2-methyl-4-pyridinyl)phenyl)pentanamide
N-(4-(2-methyl-4-pyridinyl)phenyl)-2-(4′-(4-morpholinyl)-3-biphenylyl)propanamide
2-(6-chloro-9-methyl-9H-carbazol-2-yl)-N-(4-(2-methyl-4-pyridinyl)phenyl)-propanamide
2-(4-aminophenyl)-N-(4-(2-methyl-4-pyridinyl)phenyl)propanamide
4-methyl-N-(4-(2-methyl-4-pyridinyl)-phenyl)-2-(4-phenoxyphenyl)pentanamide
4-methyl-N-(4-(2-methyl-4-pyridinyl)-phenyl)-2-(3-phenoxyphenyl)pentanamide
2-(2,3-dihydro-1,4-benzodioxin-6-yl)-N-(4-(2-methyl-4-pyridinyl)phenyl)propanamide
2-(2,3-dihydro-1,4-benzodioxin-6-yl)-4-methyl-N-(4-(2-methyl-4-pyridinyl)phenyl)-
pentanamide
2-(9-methyl-9H-carbazol-3-yl)-N-(4-(2-methyl-4-pyridinyl)phenyl)acetamide
N-(4-(2-methyl-4-pyridinyl)phenyl)-2-(3-(1,2,3,6-tetrahydro-4-pyridinyl)phenyl)-
propanamide
2-(4-iodophenyl)-4-methyl-N-(4-(2-methyl-4-pyridinyl)phenyl)pentanamide
N-(4-(2-methyl-4-pyridinyl)phenyl)-2-(3-(4-phenyl-1-piperazinyl)phenyl)-propanamide
N-(4-(2-methyl-4-pyridinyl)phenyl)-2-(3-(4-piperidinyl)phenyl)propanamide
N-(4-(2-methyl-4-pyridinyl)phenyl)-2-(3-(4-morpholinyl)phenyl)propanamide
2-(3-fluoro-5-(trifluoromethyl)phenyl)-N-(4-(2-methyl-4-pyridinyl)phenyl)acetamide
2-(3-fluoro-5-(trifluoromethyl)phenyl)-N-(4-(2-methyl-4-pyridinyl)phenyl)-propanamide
2-(3-bromophenyl)-N-(3-fluoro-4-(2-methyl-4-pyridinyl)phenyl)propanamide
2-(3-bromophenyl)-N-(2-fluoro-4-(2-methyl-4-pyridinyl)phenyl)propanamide
2-(6-fluoro-2-pyridinyl)-N-(4-(2-methyl-4-pyridinyl)phenyl)propanamide
2-(2′-(dimethylamino)-3-biphenylyl)-N-(4-(2-methyl-4-pyridinyl)phenyl)propanamide
2-(3-iodophenyl)-N-(4-(2-methyl-4-pyridinyl)phenyl)propanamide
2-(4-fluoro-3-(trifluoromethyl)phenyl)-N-(4-(2-methyl-4-pyridinyl)phenyl)-propanamide
2-(4-(dimethylamino)phenyl)-N-(4-(2-methyl-4-pyridinyl)phenyl)propanamide
2-(3-chloro-2-fluorophenyl)-N-(4-(2-methyl-4-pyridinyl)phenyl)propanamide
2-(5-chloro-2-fluorophenyl)-N-(4-(2-methyl-4-pyridinyl)phenyl)propanamide
N-(4-(2-methyl-4-pyridinyl)phenyl)-2-(3-(4-morpholinyl)-5-(trifluoromethyl)phenyl)-
propanamide
N-(4-(2-methyl-4-pyridinyl)phenyl)-2-(4′-(methylsulfonyl)-3-biphenylyl)propanamide
2-(4-((1-methylethyl)amino)phenyl)-N-(4-(2-methyl-4-pyridinyl)phenyl)propanamide
2-(3,5-bis(trifluoromethyl)phenyl)-N-(3-methoxy-4-(2-methyl-4-pyridinyl)phenyl)-
propanamide
N-(4-(2-methyl-4-pyridinyl)phenyl)-2-(4-phenyl-1H-imidazol-1-yl)propanamide
2-(3-(benzyloxy)phenyl)-N-(4-(2-methyl-4-pyridinyl)phenyl)propanamide
2-(3-methoxyphenyl)-N-(4-(2-methyl-4-pyridinyl)phenyl)propanamide
2-(3-fluoro-4-methoxyphenyl)-N-(4-(2-methyl-4-pyridinyl)phenyl)propanamide
2-(3′-cyano-3-biphenylyl)-N-(4-(2-methyl-4-pyridinyl)phenyl)propanamide
2-(2′-cyano-3-biphenylyl)-N-(4-(2-methyl-4-pyridinyl)phenyl)propanamide
2-(4′-methoxy-3-biphenylyl)-N-(4-(2-methyl-4-pyridinyl)phenyl)propanamide
2-(2′-methoxy-3-biphenylyl)-N-(4-(2-methyl-4-pyridinyl)phenyl)propanamide
N-(4-(2-methyl-4-pyridinyl)phenyl)-2-(4′-(trifluoromethoxy)-3-biphenylyl)-propanamide
2-(3-fluoro-5-(trifluoromethyl)phenyl)-N-(3-methoxy-4-(2-methyl-4-pyridinyl)-
phenyl)propanamide
2-(3-methyl-4-phenyl-1H-pyrazol-1-yl)-N-(4-(2-methyl-4-pyridinyl)phenyl)-propanamide
2-(4-(4-bromophenyl)-1H-pyrazol-1-yl)-N-(4-(2-methyl-4-pyridinyl)phenyl)-propanamide
2-(3-chloro-5-fluorophenyl)-N-(4-(2-methyl-4-pyridinyl)phenyl)propanamide
2-(3-bromo-4-fluorophenyl)-N-(4-(2-methyl-4-pyridinyl)phenyl)propanamide
N-(3-methoxy-4-(2-methyl-4-pyridinyl)-phenyl)-2-(3-(1-piperidinyl)phenyl)-propanamide
2-(3-bromophenyl)-N-(3-cyano-4-(2-methyl-4-pyridinyl)phenyl)propanamide
(2R)-2-(3-bromophenyl)-N-(3-methoxy-4-(2-methyl-4-pyridinyl)phenyl)propanamide
(2S)-2-(3-bromophenyl)-N-(3-methoxy-4-(2-methyl-4-pyridinyl)phenyl)propanamide
2-(3-bromo-5-fluorophenyl)-N-(4-(2-methyl-4-pyridinyl)phenyl)propanamide
(2R)-2-(3-fluoro-5-trifluoromethyl)phenyl)-N-(4-(2-methyl-4-pyridinyl)phenyl)-
propanamide
(2S)-2-(3-fluoro-5-(trifluoromethyl)phenyl)-N-(4-(2-methyl-4-pyridinyl)phenyl)-
propanamide
2-(1-methyl-1H-indol-3-yl)-N-(4-(2-methyl-4-pyridinyl)phenyl)propanamide
2-(2-chloro-4-pyridinyl)-N-(4-(2-methyl-4-pyridinyl)phenyl)acetamide
2-(2-chloro-4-pyridinyl)-N-(4-(2-methyl-4-pyridinyl)phenyl)propanamide
2-(4-fluoro-3-((2-methoxyethyl)amino)-phenyl)-N-(4-(2-methyl-4-pyridinyl)-
phenyl)propanamide
2-(3-cyano-5-(trifluoromethyl)phenyl)-N-(4-(2-methyl-4-pyridinyl)phenyl)acetamide
2-(3-bromo-5-(trifluoromethyl)phenyl)-N-(4-(2-methyl-4-pyridinyl)phenyl)-propanamide
2-(3-bromo-5-(trifluoromethyl)phenyl)-N-(4-(2-methyl-4-pyridinyl)phenyl)acetamide
(2S)-2-(3,5-dichlorophenyl)-N-(4-(2-methyl-4-pyridinyl)phenyl)propanamide
(2R)-2-(3,5-dichlorophenyl)-N-(4-(2-methyl-4-pyridinyl)phenyl)propanamide
(2S)-2-(3,5-dichlorophenyl)-4-methyl-N-(4-(2-methyl-4-pyridinyl)phenyl)pentanamide
(2R)-2-(3,5-dichlorophenyl)-4-methyl-N-(4-(2-methyl-4-pyridinyl)phenyl)pentanamide
(2R)-2-(3-bromophenyl)-N-(4-(2-methyl-4-pyridinyl)phenyl)propanamide
(2S)-2-(3-bromophenyl)-N-(4-(2-methyl-4-pyridinyl)phenyl)propanamide
N-(3-methyl-4-(2-methyl-4-pyridinyl)-phenyl)-2-(4′-(4-morpholinyl)-3-biphenylyl)-
propanamide
2-(4-(methyl(1-methylethyl)amino)phenyl)-N-(4-(2-methyl-4-pyridinyl)phenyl)-
propanamide
2-(9-methyl-9H-carbazol-3-yl)-N-(4-(2-methyl-4-pyridinyl)phenyl)propanamide
N-(3-methoxy-4-(2-methyl-4-pyridinyl)-phenyl)-2-(9-methyl-9H-carbazol-3-yl)-
propanamide
N-(4-(2-methyl-4-pyridinyl)phenyl)-2-(2-(trifluoromethoxy)phenyl)propanamide
N-(4-(2-methyl-4-pyridinyl)phenyl)-2-(3-(trifluoromethoxy)phenyl)propanamide
N-(4-(2-methyl-4-pyridinyl)phenyl)-2-(4-(trifluoromethoxy)phenyl)propanamide
2-(1H-indol-5-yl)-N-(4-(2-methyl-4-pyridinyl)phenyl)propanamide
(2R)-2-(3-methyl-4-phenyl-1H-pyrazol-1-yl)-N-(4-(2-methyl-4-pyridinyl)phenyl)-
propanamide
(2S)-2-(3-methyl-4-phenyl-1H-pyrazol-1-yl)-N-(4-(2-methyl-4-pyridinyl)phenyl)-
propanamide
(2R)-2-(3-bromo-5-fluorophenyl)-N-(3-methoxy-4-(2-methyl-4-pyridinyl)phenyl)-
propanamide
(2S)-2-(4-((1-methylethyl)amino)phenyl)-N-(4-(2-methyl-4-pyridinyl)phenyl)-propanamide
(2R)-2-(4-((1-methylethyl)amino)phenyl)-N-(4-(2-methyl-4-pyridinyl)phenyl)-propanamide
2-(4-aminophenyl)-N-(3-methoxy-4-(2-methyl-4-pyridinyl)phenyl)propanamide
(2S)-2-(3,5-bis(trifluoromethyl)phenyl)-N-(3-methoxy-4-(2-methyl-4-pyridinyl)-
phenyl)propanamide
(2R)-2-(3,5-bis(trifluoromethyl)phenyl)-N-(3-methoxy-4-(2-methyl-4-pyridinyl)-
phenyl)propanamide
2-(4-bromo-1H-imidazol-1-yl)-N-(4-(2-methyl-4-pyridinyl)phenyl)hexanamide
2-(2,3-dihydro-1H-indol-5-yl)-N-(4-(2-methyl-4-pyridinyl)phenyl)propanamide
(2S)-2-(4-chlorophenyl)-N-(4-(2-methyl-4-pyridinyl)phenyl)propanamide
(2R)-2-(4-chlorophenyl)-N-(4-(2-methyl-4-pyridinyl)phenyl)propanamide
2-methyl-N-(4-(2-methyl-4-pyridinyl)-phenyl)-2-(4-(trifluoromethyl)phenyl)-propanamide
(2R)-N-(4-(2-methyl-4-pyridinyl)phenyl)-2-(3-(5-pyrimidinyl)phenyl)propanamide
(2R)-2-(3-(2-methoxy-5-pyrimidinyl)-phenyl)-N-(4-(2-methyl-4-pyridinyl)-
phenyl)propanamide
(2R)-N-(4-(2-methyl-4-pyridinyl)phenyl)-2-(3-(2-(methylsulfanyl)-5-pyrimidinyl)-
phenyl)propanamide
(2R)-2-(3-(2-amino-5-pyrimidinyl)phenyl)-N-(4-(2-methyl-4-pyridinyl)phenyl)-
propanamide
(2R)-2-(3-(1-methyl-1H-indol-5-yl)phenyl)-N-(4-(2-methyl-4-pyridinyl)phenyl)-
propanamide
N-(4-(2-methyl-4-pyridinyl)phenyl)-2-(4-(trifluoromethyl)phenyl)propanamide
2-(4-(cyclopentylamino)phenyl)-N-(3-methoxy-4-(2-methyl-4-pyridinyl)phenyl)-
propanamide
N-(3-methoxy-4-(2-methyl-4-pyridinyl)-phenyl)-2-(4-((1-methylethyl)amino)phenyl)-
propanamide
2-(4-(cyclobutylamino)phenyl)-N-(3-methoxy-4-(2-methyl-4-pyridinyl)-phenyl)-
propanamide
N-(4-(2-methyl-4-pyridinyl)phenyl)-2-((3R,5S)-5-phenyl-3-piperidinyl)acetamide
(2S)-2-(3-chloro-5-fluorophenyl)-N-(4-(2-methyl-4-pyridinyl)phenyl)propanamide
(2R)-2-(3-chloro-5-fluorophenyl)-N-(4-(2-methyl-4-pyridinyl)phenyl)propanamide
(2S)-N-(4-(2-methyl-4-pyridinyl)phenyl)-2-(4-phenyl-1H-imidazol-1-yl)propanamide
(2R)-N-(4-(2-methyl-4-pyridinyl)phenyl)-2-(4-phenyl-1H-imidazol-1-yl)propanamide
2-(4-(diethylamino)phenyl)-N-(3-methoxy-4-(2-methyl-4-pyridinyl)phenyl)-propanamide
(2S)-2-(3-(2-methoxy-5-pyrimidinyl)-phenyl)-N-(4-(2-methyl-4-pyridinyl)-phenyl)-
propanamide
2-(3-(2-methyl-4-pyridinyl)phenyl)-N-(4-(2-methyl-4-pyridinyl)phenyl)propanamide
2-(4-(cyclobutylamino)phenyl)-N-(4-(2-methyl-4-pyridinyl)phenyl)propanamide
N-(4-(2-methyl-4-pyridinyl)phenyl)-2-(4-((3,4,5-trimethoxybenzyl)amino)-phenyl)-
propanamide
(2R)-N-(4-(2-methyl-4-pyridinyl)phenyl)-2-(4′-(1-piperazinyl)-3-biphenylyl)-propanamide
(2R)-2-(4′-(4-acetyl-1-piperazinyl)-3-biphenylyl)-N-(4-(2-methyl-4-pyridinyl)-phenyl)-
propanamide
2-(4-cyanophenyl)-N-(4-(2-methyl-4-pyridinyl)phenyl)acetamide
2-(3-cyclopropylphenyl)-N-(4-(2-methyl-4-pyridinyl)phenyl)propanamide
2-(4-(ethylamino)phenyl)-N-(3-methoxy-4-(2-methyl-4-pyridinyl)phenyl)propanamide
2-(4-(ethyl(3,4,5-trimethoxybenzyl)amino)-phenyl)-N-(4-(2-methyl-4-pyridinyl)-
phenyl)propanamide
2-(4-(cyclopropylamino)phenyl)-N-(4-(2-methyl-4-pyridinyl)phenyl)propanamide
2-(4-(dicyclopropylamino)phenyl)-N-(4-(2-methyl-4-pyridinyl)phenyl)propanamide
2-(3-bromophenyl)-2-methyl-N-(4-(2-methyl-4-pyridinyl)phenyl)propanamide
(2R)-2-(3-bromophenyl)-N-(4-(2-methyl-4-pyridinyl)phenyl)pentanamide
(2S)-2-(3-bromophenyl)-N-(4-(2-methyl-4-pyridinyl)phenyl)pentanamide
2-(6-chloro-2-pyridinyl)-4-methyl-N-(4-(2-methyl-4-pyridinyl)phenyl)pentanamide
4-methyl-N-(4-(2-methyl-4-pyridinyl)-phenyl)-2-(6-(4-morpholinyl)-2-pyridinyl)-
pentanamide
4-methyl-N-(4-(2-methyl-4-pyridinyl)-phenyl)-2-(6-(4-(4-morpholinyl)phenyl)-2-
pyridinyl)pentanamide
2-(3-(6-fluoro-3-pyridinyl)phenyl)-N-(4-(2-methyl-4-pyridinyl)phenyl)propanamide
2-(3-biphenylyl)-N-(4-(2-methyl-4-pyridinyl)phenyl)propanamide
2-(6-bromo-2-pyridinyl)-N-(4-(2-methyl-4-pyridinyl)phenyl)propanamide
N-(4-(2-methyl-4-pyridinyl)phenyl)-2-(3-(3-pyridinyl)phenyl)propanamide
N-(4-(2-methyl-4-pyridinyl)phenyl)-2-(3-(4-pyridinyl)phenyl)propanamide
2-(3-(3-chloro-4-pyridinyl)phenyl)-N-(4-(2-methyl-4-pyridinyl)phenyl)propanamide
2-(1,3-benzodioxol-5-yl)-N-(4-(2-methyl-4-pyridinyl)phenyl)propanamide
N-(4-2-methyl-4-pyridinyl)phenyl)-2-(7-(3-pyridinyl)-3,4-dihydro-2(1H)-
isoquinolinyl)propanamide
2-(4-(ethylamino)phenyl)-N-(4-(2-methyl-4-pyridinyl)phenyl)propanamide
2-(4-((1-methylpropyl)amino)phenyl)-N-(4-(2-methyl-4-pyridinyl)phenyl)propanamide
2-(4-(methylamino)phenyl)-N-(4-(2-methyl-4-pyridinyl)phenyl)propanamide
N-(4-(2-methyl-4-pyridinyl)phenyl)-2-(4-(propylamino)phenyl)propanamide
(2-(4-(dipropylamino)phenyl)-N-(4-(2-methyl-4-pyridinyl)phenyl)propanamide
2-(4-(diethylamino)phenyl)-N-(4-(2-methyl-4-pyridinyl)phenyl)propanamide
2-(2-ethyl-1,3-benzoxazol-5-yl)-N-(4-(2-methyl-4-pyridinyl)phenyl)acetamide
2-(2-methyl-1,3-benzoxazol-5-yl)-N-(4-(2-methyl-4-pyridinyl)phenyl)acetamide
(2R)-2-fluoro-N-(4-(2-methyl-4-pyridinyl)-phenyl)-2-(6-quinolinyl)ethanamide
2-(3-methoxy-6-quinolinyl)-N-(4-(2-methyl-4-pyridinyl)phenyl)propanamide
2-cyclopentyl-N-(4-(2-methyl-4-pyridinyl)-phenyl)acetamide
3-cyclopropyl-N-(4-(2-methyl-4-pyridinyl)-phenyl)propanamide
N-(4-(2-methyl-4-pyridinyl)phenyl)-2-(3-(2-pyrimidinyl)phenyl)propanamide
N-(4-(2-methyl-4-pyridinyl)phenyl)-2-(3-(2-pyridinyl)phenyl)propanamide
3-cyclobutyl-N-(4-(2-methyl-4-pyridinyl)-phenyl)propanamide
2-cyclobutyl-N-(4-(2-methyl-4-pyridinyl)-phenyl)acetamide
N-(4-(2-methyl-4-pyridinyl)phenyl)-2-(2-(4-(4-morpholinyl)phenyl)-4-pyridinyl)-
propanamide
2-(2,4-dimethyl-1,3-thiazol-5-yl)-N-(4-(2-methyl-4-pyridinyl)phenyl)acetamide
2-(3-(6-(dimethylamino)-3-pyridinyl)-phenyl)-N-(4-(2-methyl-4-pyridinyl)-
phenyl)propanamide
2-(3-(4-methyl-1H-imidazol-1-yl)phenyl)-N-(4-(2-methyl-4-pyridinyl)phenyl)-propanamide
N-(4-2-methyl-4-pyridinyl)phenyl)-2-(3-(1H-pyrazol-1-yl)phenyl)propanamide
2-(4-(acetyl(1-methylethyl)amino)phenyl)-N-(4-(2-methyl-4-pyridinyl)phenyl)-
propanamide
2-(6-chloro-3-pyridinyl)-N-(4-(2-methyl-4-pyridinyl)phenyl)propanamide
2-(4-amino-3-methoxyphenyl)-N-(4-(2-methyl-4-pyridinyl)phenyl)propanamide
2-(3-methoxy-4-((1-methylethyl)amino)-phenyl)-N-(4-(2-methyl-4-pyridinyl)-
phenyl)propanamide
2-(3-bromo-5-methylphenyl)-N-(4-(2-methyl-4-pyridinyl)phenyl)propanamide
2-(4-fluoro-4′-(4-morpholinyl)-3-biphenylyl)-N-(4-(2-methyl-4-pyridinyl)-
phenyl)propanamide
2-(6-bromo-2-pyridinyl)-4-methyl-N-(4-(2-methyl-4-pyridinyl)phenyl)pentanamide
-N-(4-(2-methyl-4-pyridinyl)phenyl)-2-(4-(1-piperidinyl)phenyl)propanamide
N-(4-(2-methyl-4-pyridinyl)phenyl)-2-(6-(1-piperidinyl)-3-pyridinyl)propanamide
2-(3-(2-methoxy-5-pyrimidinyl)-5-methyl-phenyl)-N-(4-(2-methyl-4-pyridinyl)-
phenyl)propanamide
2-(1,3-benzoxazol-5-yl)-N-(4-(2-methyl-4-pyridinyl)phenyl)acetamide
N-(4-(2-methyl-4-pyridinyl)phenyl)-2-(2-(3-pyridinyl)-1,3-benzoxazol-5-yl)-acetamide
N-(4-(2-methyl-4-pyridinyl)phenyl)-2-(2-(4-pyridinyl)-1,3-benzoxazol-5-yl)-acetamide
2-(4,4″-bis(trifluoromethyl)-1,1′:3′,1″-terphenyl-5′-yl)-4-methyl-N-(4-(2-methyl-4-
pyridinyl)phenyl)pentanamide
3-cyclopropyl-2-(5-((1-methylethyl)amino)-2-pyridinyl)-N-(4-(2-methyl-4-pyridinyl)-
phenyl)propanamide
2-(3-(1H-indol-5-yl)-5-methylphenyl)-N-(4-(2-methyl-4-pyridinyl)phenyl)-propanamide
2-(3-bromo-5-chlorophenyl)-N-(4-(2-methyl-4-pyridinyl)phenyl)propanamide
2-(3-chloro-5-(2-methyl-5-pyrimidinyl)-phenyl)-N-(4-(2-methyl-4-pyridinyl)-
phenyl)propanamide
2-(3-chloro-5-(1,5-dimethyl-1H-pyrazol-4-yl)phenyl)-N-(4-(2-methyl-4-pyridinyl)-
phenyl)propanamide
2-(3-chloro-5-(3-pyridinyl)phenyl)-N-(4-(2-methyl-4-pyridinyl)phenyl)propanamide
2-(4′-cyano-5-methyl-3-biphenylyl)-N-(4-(2-methyl-4-pyridinyl)phenyl)propanamide
2-(3′-cyano-5-methyl-3-biphenylyl)-N-(4-(2-methyl-4-pyridinyl)phenyl)propanamide
2-(2′-cyano-5-methyl-3-biphenylyl)-N-(4-(2-methyl-4-pyridinyl)phenyl)propanamide
4-methyl-N-(4-(2-methyl-4-pyridinyl)-phenyl)-2-phenylpentanamide
2-(4-((2-methylpropyl)amino)phenyl)-N-(4-(2-methyl-4-pyridinyl)phenyl)propanamide
2-(4-(bis(2-methylpropyl)amino)phenyl)-N-(4-2-methyl-4-pyridinyl)phenyl)-propanamide
2-(3-cyano-5-methylphenyl)-N-(4-(2-methyl-4-pyridinyl)phenyl)propanamide
N-(4-(2-methyl-4-pyridinyl)phenyl)-2-(4-(propyl(3,4,5-trimethoxybenzyl)amino)-
phenyl)propanamide
4-methyl-N-(4-(2-methyl-4-pyridinyl)-phenyl)-2-(6-chloropyridin-3-yl)pentanamide
4-methyl-N-(4-(2-methyl-4-pyridinyl)-phenyl)-2-(6-(1-piperidinyl)-3-
pyridinyl)pentanamide
2-(3-(6-(methylamino)-3-pyridinyl)phenyl)-N-(4-(2-methyl-4-pyridinyl)phenyl)-
propanamide
2-(3-(6-((2-methoxyethyl)amino)-3-pyridinyl)phenyl)-N-(4-(2-methyl-4-pyridinyl)-
phenyl)propanamide
2-(2-methyl-1H-benzimidazol-5-yl)-N-(4-(2-methyl-4-pyridinyl)phenyl)acetamide
2-(3-methyl-5-(2-methyl-5-pyrimidinyl-)phenyl)-N-(4-(2-methyl-4-pyridinyl)phenyl)-
propanamide
2-methyl-N-(4-(2-methyl-4-pyridinyl)-phenyl)-2-phenylbutanamide
2-(1,3-benzodioxol-5-yl)-4-methyl-N-(4-(2-methyl-4-pyridinyl)phenyl)pentanamide
N-(4-(2-methyl-4-pyridinyl)phenyl)-2-(3-methyl-5-(4-(2-pyridinyl)-1-piperazinyl)-
phenyl)propanamide
2-(3-methyl-5-(2-methyl-4-pyridinyl)-phenyl)-N-(4-(2-methyl-4-pyridinyl)-
phenyl)propanamide
2-(3-fluoro-5-(3-pyridinyl)phenyl)-N-(4-(2-methyl-4-pyridinyl)phenyl)propanamide
2-(3-fluoro-5-(2-methyl-4-yridinyl)phenyl)-N-(4-(2-methyl-4-pyridinyl)phenyl)-
propanamide
2-(3-(difluoromethyl)-5-(trifluoromethyl)-phenyl)-N-(4-(2-methyl-4-pyridinyl)-
phenyl)propanamide
2-(3-fluoro-5-methylphenyl)-N-(4-(2-methyl-4-pyridinyl)phenyl)propanamide
2-(2,5-dimethyl-1,3-thiazol-4-yl)-N-(4-(2-methyl-4-pyridinyl)phenyl)acetamide
2-(3-(6-cyano-3-pyridinyl)phenyl)-N-(4-(2-methyl-4-pyridinyl)phenyl)propanamide
2-(3-bromophenyl)-4-hydroxy-4-methyl-N-(4-(2-methyl-4-pyridinyl)phenyl)-pentanamide
2-(3-methyl-5-(4-methyl-3-oxo-1-piperazinyl)phenyl)-N-(4-(2-methyl-4-pyridinyl)-
phenyl)propanamide
2-(3-(4-methoxy-1-piperidinyl)-5-methyl-phenyl)-N-(4-(2-methyl-4-pyridinyl)-
phenyl)propanamide
2-(3-(4,4-difluoro-1-piperidinyl)-5-methyl-phenyl)-N-(4-(2-methyl-4-pyridinyl)-
phenyl)propanamide
2-(3-(6-((1-methylethyl)amino)-3-pyridinyl)phenyl)-N-(4-(2-methyl-4-pyridinyl)-
phenyl)propanamide
3′-((1R)-1-methyl-2-((4-(2-methyl-4-pyridinyl)phenyl)amino)-2-oxoethyl)-4-biphenyl-
carboxylic acid
2-(3-(6-chloro-2-pyrazinyl)phenyl)-N-(4-(2-methyl-4-pyridinyl)phenyl)propanamide
2-(3-(5-methoxy-2-pyrazinyl)phenyl)-N-(4-(2-methyl-4-pyridinyl)phenyl)propanamide
2-(5-bromo-2-methoxyphenyl)-4-methyl-N-(4-(2-methyl-4-pyridinyl)phenyl)-pentanamide
2-(5-bromo-2-fluorophenyl)-4-methyl-N-(4-(2-methyl-4-pyridinyl)phenyl)pentanamide
2-(3-bromo-4-fluorophenyl)-4-methyl-N-(4-(2-methyl-4-pyridinyl)phenyl)pentanamide
2-(5-chloro-3′-cyano-3-biphenylyl)-N-(4-(2-methyl-4-pyridinyl)phenyl)propanamide
2-(3-chloro-5-(2-methoxy-5-pyrimidinyl)-phenyl)-N-(4-(2-methyl-4-pyridinyl)-
phenyl)propanamide
2-(3-chloro-5-(3-furanyl)phenyl)-N-(4-(2-methyl-4-pyridinyl)phenyl)propanamide
2-(5-chloro-4′-cyano-3-biphenylyl)-N-(4-(2-methyl-4-pyridinyl)phenyl)propanamide
2-(4,6-bis(trifluoromethyl)-2-pyridinyl)-N-(4-(2-methyl-4-pyridinyl)phenyl)-propanamide
2-(2,4-dimethyl-1,3-thiazol-5-yl)-N-(4-(2-methyl-4-pyridinyl)phenyl)propanamide
2-(6-((1-methylethyl)amino)-3-pyridinyl)-N-(4-(2-methyl-4-pyridinyl)phenyl)-propanamide
2-(4-((4-methoxybenzyl)(methyl)amino)-phenyl)-N-(4-(2-methyl-4-pyridinyl)-
phenyl)propanamide
2-(4-(benzylamino)phenyl)-N-(4-(2-methyl-4-pyridinyl)phenyl)propanamide
2-(6-(dimethylamino)-3-pyridinyl)-4-methyl-N-(4-(2-methyl-4-pyridinyl)phenyl)-
pentanamide
2-(3-(4-methoxy-1-piperidinyl)phenyl)-N-(4-(2-methyl-4-pyridinyl)phenyl)-propanamide
tert-butyl 4-(3-(1-methyl-2-((4-(2-methyl-4-pyridinyl)phenyl)amino)-2-oxoethyl)-phenyl)-
1-piperazinecarboxylate
N-(4-(2-methyl-4-pyridinyl)phenyl)-2-(3-(1-piperazinyl)phenyl)propanamide
2-(5-bromo-3-pyridinyl)-N-(3-methoxy-4-(2-methyl-4-pyridinyl)phenyl)-4-methyl-
pentanamide
2-(4-bromo-2-thiophenyl)-N-(4-(2-methyl-4-pyridinyl)phenyl)propanamide
2-(5-chloro-2-thiophenyl)-N-(4-(2-methyl-4-pyridinyl)phenyl)propanamide
2-(4-(benzyl(methyl)amino)phenyl)-N-(4-(2-methyl-4-pyridinyl)phenyl)propanamide
2-(5-bromo-3-pyridinyl)-4-methyl-N-(4-(2-methyl-4-pyridinyl)phenyl)pentanamide
2-(4-(2-methoxy-5-pyrimidinyl)-2-thiophenyl)-N-(4-(2-methyl-4-pyridinyl)-phenyl)-
propanamide
2-(5-bromo-2-thiophenyl)-N-(4-(2-methyl-4-pyridinyl)phenyl)propanamide
2-(3-bromophenyl)-4-fluoro-4-methyl-N-(4-(2-methyl-4-pyridinyl)phenyl)pentanamide
2-(3-(((2-methoxyethyl)amino)methyl)-phenyl)-4-methyl-N-(4-(2-methyl-4-pyridinyl)-
phenyl)pentanamide
2-(3-((dimethylamino)methyl)phenyl)-4-methyl-N-(4-(2-methyl-4-pyridinyl)phenyl)-
pentanamide
4-methyl-N-(4-(2-methyl-4-pyridinyl)-phenyl)-2-(3-(1-piperidinylmethyl)phenyl)-
pentanamide
2-(3-(hydroxymethyl)phenyl)-4-methyl-N-(4-(2-methyl-4-pyridinyl)phenyl)-pentanamide
4-methyl-N-(4-(2-methyl-4-pyridinyl)-phenyl)-2-(3-(4-morpholinylmethyl)-phenyl)-
pentanamide and
4-methyl-2-(5-methyl-3-pyridinyl)-N-(4-(2-methyl-4-pyridinyl)phenyl)pentanamide
or a pharmaceutically acceptable salt thereof.

40. A pharmaceutical composition comprising a compound of claim 20 and a pharmaceutically acceptable excipient.

41. A pharmaceutical composition comprising a compound of claim 39 and a pharmaceutically acceptable excipient.

42. A method of treating Alzheimer's disease by modulation of γ-secretase in a patient which method comprises administering to the patient a pharmaceutical composition comprising a compound of claim 20 and a pharmaceutically acceptable excipient.
